SEQUENCE LISTING (40 sequences)

SEQ ID NO: 1 - DNA, 4211 bp, Homo sapiens
aaatcagatgctctgtgattaatcgtggaggattcaggacacgaccacaaacgctgccagataagagtcccggctgcattatcagagcccggcagggcaccggcctccctgcaccagaaggaagactcggggcgcagcaggtcctcaaggcgatcttcccagagagcgggaccagcggctggtggccagtgtggatggaatttgcagagccctagctcgagtccgggagtcccgggccagatgggagcagacgcttgctggcggcaatagggaaagtgaggcagctgcaaggagggcggcgggactgcactcgagtgtccagacctgctcgatggtgaccaccatgtcggtgaggttgcggttcctgtcccctggggacacaggggccgtgggggtcgtgggccggagcgcctccttcgcaggcttcagcagtgcacagagccggaggatcgcaaagtccatcaacaggaactccgtgagatcgcgaatgcctgcaaaatcctccaagatgtacggcacgctgcggaaggggtcggtctgtgcagacccgaagccccagcaggtgaagaagatcttcgaagcattgaaaagaggcctcaaggagtatctgtgtgtgcagcaggctgagctggaccacctgtctggacgccacaaagacaccaggaggaattccaggctggctttctattatgacctggacaagcaaacgcgctgtgtggaaaggcacattcggaagatggagtttcacatcagcaaggtggatgagctgtacgaggactactgcatccagtgccgcctgcgcgacggcgcctccagcatgcagcgggccttcgcccggtgccccccgagccgcgcagcccgagagagcctgcaggagctgggccgcagcctgcacgagtgcgccgaggacatgtggctcatcgagggggccctggaggttcacctgggcgagttccacatcaggatgaaaggcttggtgggctacgcacgcctctgtcccggagaccactatgaggtgctcatgcgtctgggccgccagcgttggaagctcaagggtcggatcgagtcagatgacagccagacctgggacgaagaggagaaggccttcatccccacgctgcatgagaacctggacatcaaggtgacggagttgcggggcctgggctcgctggctgtgggtgcagtgacgtgtgacatcgccgacttcttcacgacgcggccgcaggtcatcgtggtggacatcacggagttgggtaccatcaagctgcagctggaggtgcagtggaacccgtttgatactgagagcttcctggtgtcacccagccccacgggcaagttttctatgggcagcaggaagggctccttgtacaactggacacccccgagcacccccagcttccgggagagatactacctgtctgtcctacagcagccaacacagcaggccttgctgctgggtggcccaagggccacctccatcctcagctacctgtctgacagcgacctccggggtcccagcctaagaagccagagtcaggagctgcctgagatggactccttcagctctgaggacccccgagacacggagaccagcacgtcggcgtccacctcagatgtgggcttcctgcccttgaccttcggtccccacgcctccattgaagaggaggctcgggaggaccccctgcccccaggtctcctgccagagatggcccacctctctggaggcccgtttgcagagcagcctggctggaggaacttaggaggggagagccccagcctgccacagggctccctgttccacagcggcacagcctcgagtagccagaacggccacgaggaaggggcaaccggggacagagaggacgggcctggcgtggccctcgaggggcctctgcaggaggtcctggagttgctgaggcccacggactccacccagccccagctccgggagctggagtaccaggtcctcggcttccgggaccggctgaagccctgcagagcacggcaggagcacacctcggccgagagcctgatggagtgcatcctggagagcttcgccttcctcaatgccgacttcgccctggatgagctgtccctgtttgggggctcccagggtctccgaaaggaccggcccctgcccccaccgtcatcactgaaagcgtcatccagggaactcacagccggtgccccagagctggacgtgctgctgatggtacacctccaagtctgcaaagctctgctgcagaaactggcctcccctaatttatcaaggctggtccaggaatgcctcctggaagaagtggcacagcaaaagcacgttctggagacactttctgtccttgactttgagaaggtcggcaaggcaacatccattgaagagatcatcccacaggcctcgcggacgaaggggtgcctgaagctgtggagagggtgcacagggcctggcagggtcctgtcctgccctgccacgacgctgctgaaccagctcaagaaaaccttccagcacagagtcagagggaagtacccaggacagctggaaatagcgtgccgcaggctcctggagcaggtggtcagctgtggtgggctgctccccggagctgggctcccagaagaacagatcattacctggttccagtttcacagctacctgcagaggcagagcgtctctgacctggagaagcacttcacccagctcaccaaggaagtgacactcatcgaggagcttcactgtgcgggacaggccaaggtggtccggaagctgcaggggaagcggctgggccagctccagcctctgccccagaccttaagagcctgggcgctgctccagctggacggcactccgagggtgtgcagggcggccagcgctcgcctggctggtgcagtcaggaacagaagcttccgggaaaaggctttgctgttctacaccaacgccctggcagagaacgacgcaaggctccagcaggccgcatgcctagcgctcaaacacctcaagggcattgaaagcatcgaccagactgccagcctgtgccagtctgacctggaggccgtgcgggcggcagcccgggaaaccacactgtcgttcggtgaaaaaggacggttagcttttgagaagatggacaagctctgctcagaacaaagagaagtcttttgccaggaggcagatgttgaaatcacaatattttaaaaaatcctggctgatgagcacaaatctcacatcgttttttttgctgctgcccagcctggacatagcctgcactctgggtaatggtgctgtgcactcctccaggagtgtgagctgcccagagctctacctgagactccggccattgacccagccccagggcatgggctggtcttttgtacagaggcagaaaaaagcaaggcaaaggtacagcattccaggggctgcacggcctcaacagagcgctcaacttctggctgagggtctgtgtgaccttccccgagatgcagagctgagccaaactaggtggccacctacaaaagggccaaggccaggcaagttgaggccctaaataaaaggctccaaggcaagtgtgtagaactccaggcctcgctgccggtcagctgctcggcacttctgcgtcaagaggcactggggatgcagcaggctggcaggtggctggccctgctaatgcaagactgctcaggccatttcagcagcagccaggtgtcaccttggtgagctggggaaggtgggaaggcacaaagccagggtttctacaaccacactctcagcccgactgacttgctgcgagtgctggtggagctcacagacggcggctggtggatggtggactgtgaacctcactttccctatgttcagcagcacaaagggaagaagccaccacatcagcccaggagccctgagcagcacaggcagtagggccactcactttggccatccgcacccaaatgcaatcaatcaacccagcttcggaagctaccctaggatctcgtcaataaactgctaagaagccatcaactggcctaaagaaagagttcactgaagaacgcaattgctttaaagaaagaaaaattagtttcctatttaagtcttaaaaaaaagcaaaccatgtcctgagatgtctgtgttaatagtgcagagagaacctagggtttgaggttgctgtagcaatggcattggagaactttaacttgaacattctcatcgatacttcctggacatattt

SEQ ID NO: 2 - protein, 946 aa, Homo sapiens
MSVRLRFLSPGDTGAVGVVGRSASFAGFSSAQSRRIAKSINRNSVRSRMPAKSSKMYGTLRKGSVCADPKPQQVKKIFEALKRGLKEYLCVQQAELDHLSGRHKDTRRNSRLAFYYDLDKQTRCVERHIRKMEFHISKVDELYEDYCIQCRLRDGASSMQRAFARCPPSRAARESLQELGRSLHECAEDMWLIEGALEVHLGEFHIRMKGLVGYARLCPGDHYEVLMRLGRQRWKLKGRIESDDSQTWDEEEKAFIPTLHENLDIKVTELRGLGSLAVGAVTCDIADFFTTRPQVIVVDITELGTIKLQLEVQWNPFDTESFLVSPSPTGKFSMGSRKGSLYNWTPPSTPSFRERYYLSVLQQPTQQALLLGGPRATSILSYLSDSDLRGPSLRSQSQELPEMDSFSSEDPRDTETSTSASTSDVGFLPLTFGPHASIEEEAREDPLPPGLLPEMAHLSGGPFAEQPGWRNLGGESPSLPQGSLFHSGTASSSQNGHEEGATGDREDGPGVALEGPLQEVLELLRPTDSTQPQLRELEYQVLGFRDRLKPCRARQEHTSAESLMECILESFAFLNADFALDELSLFGGSQGLRKDRPLPPPSSLKASSRELTAGAPELDVLLMVHLQVCKALLQKLASPNLSRLVQECLLEEVAQQKHVLETLSVLDFEKVGKATSIEEIIPQASRTKGCLKLWRGCTGPGRVLSCPATTLLNQLKKTFQHRVRGKYPGQLEIACRRLLEQVVSCGGLLPGAGLPEEQIITWFQFHSYLQRQSVSDLEKHFTQLTKEVTLIEELHCAGQAKVVRKLQGKRLGQLQPLPQTLRAWALLQLDGTPRVCRAASARLAGAVRNRSFREKALLFYTNALAENDARLQQAACLALKHLKGIESIDQTASLCQSDLEAVRAAARETTLSFGEKGRLAFEKMDKLCSEQREVFCQEADVEITIF

SEQ ID NO: 3 - DNA, 2278 bp, Homo sapiens
aaatcagatgctctgtgattaatcgtggaggattcaggacacgaccacaaacgctgccagataagagtcccggctgcattatcagagcccggcagggcaccggcctccctgcaccagaaggaagactcggggcgcagcaggtcctcaaggcgatcttcccagagagcgggaccagcggctggtggccagtgtggatggaatttgcagagccctagctcgagtccgggagtcccgggccagatgggagcagacgcttgctggcggcaatagggaaagtgaggcagctgcaaggagggcggcgggactgcactcgagtgtccagacctgctcgatggtgaccaccatgtcggtgaggttgcggttcctgtcccctggggacacaggggccgtgggggtcgtgggccggagcgcctccttcgcaggcttcagcagtgcacagagccggaggatcgcaaagtccatcaacaggaactccgtgagatcgcgaatgcctgcaaaatcctccaagatgtacggcacgctgcggaaggggtcggtctgtgcagacccgaagccccagcaggtgaagaagatcttcgaagcattgaaaagaggcctcaaggagtatctgtgtgtgcagcaggctgagctggaccacctgtctggacgccacaaagacaccaggaggaattccaggctggctttctattatgacctggacaagcaaacgcgctgtgtggaaaggcacattcggaagatggagtttcacatcagcaaggtggatgagctgtacgaggactactgcatccagtgccgcctgcgcgacggcgcctccagcatgcagcgggccttcgcccggtgccccccgagccgcgcagcccgagagagcctgcaggagctgggccgcagcctgcacgagtgcgccgaggacatgtggctcatcgagggggccctggaggttcacctgggcgagttccacatcaggatgaaaggcttggtgggctacgcacgcctctgtcccggagaccactatgaggtgctcatgcgtctgggccgccagcgttggaagctcaagggtcggatcgagtcagatgacagccagacctgggacgaagaggagaaggccttcatccccacgctgcatgagaacctggacatcaaggtgacggagttgcggggcctgggctcgctggctgtgggtgcagtgacgtgtgacatcgccgacttcttcacgacgcggccgcaggtcatcgtggtggacatcacggagttgggtaccatcaagctgcagctggaggtgcagtggaacccgtttgatactgagagcttcctggtgtcacccagccccacgggcaagttttctatgggcagcaggaagggctccttgtacaactggacacccccgagcacccccagcttccgggagagatactacctgtctgtcctacagcagccaacacagcaggccttgctgctgggtggcccaagggccacctccatcctcagctacctgtctgacagcgacctccggggtcccagcctaagaagccagagtcaggagctgcctgagatggactccttcagctctgaggacccccgagacacggagaccagcacgtcggcgtccacctcagatgtgggcttcctgcccttgaccttcggtccccacgcctccattgaagaggaggctcgggaggaccccctgcccccaggtctcctgccagagatggcccacctctctggaggcccgtttgcagagcagcctggctggaggaacttaggaggggagagccccagcctgccacagggctccctgttccacagcggcacagcctcgagtagccagaacggccacgaggaaggggcaaccggggacagagaggacgggcctggcgtggccctcgaggggcctctgcaggaggtcctggagttgctgaggcccacggactccacccagccccagctccgggagctggagtaccaggtcctcggcttccgggaccggctgaaggtatggccaccccgccccgggcggtggccctgctttgctgatggcatgatgactgggagtcgggggctctggggccacgcagcctgggccgacatcctggcctcacctctgcgtgacctgggtgggccgtgtctctctgggccttggtttcctcatctggcaagcggggataacaacagccctcatggggctcaggaagattttaagagttcacagtagataggctcatgcacatccagccagaaactggccccatctcgcaccttctgacctgggtgggcggggctg

SEQ ID NO: 4 - protein, 621 aa, Homo sapiens
MSVRLRFLSPGDTGAVGVVGRSASFAGFSSAQSRRIAKSINRNSVRSRMPAKSSKMYGTLRKGSVCADPKPQQVKKIFEALKRGLKEYLCVQQAELDHLSGRHKDTRRNSRLAFYYDLDKQTRCVERHIRKMEFHISKVDELYEDYCIQCRLRDGASSMQRAFARCPPSRAARESLQELGRSLHECAEDMWLIEGALEVHLGEFHIRMKGLVGYARLCPGDHYEVLMRLGRQRWKLKGRIESDDSQTWDEEEKAFIPTLHENLDIKVTELRGLGSLAVGAVTCDIADFFTTRPQVIVVDITELGTIKLQLEVQWNPFDTESFLVSPSPTGKFSMGSRKGSLYNWTPPSTPSFRERYYLSVLQQPTQQALLLGGPRATSILSYLSDSDLRGPSLRSQSQELPEMDSFSSEDPRDTETSTSASTSDVGFLPLTFGPHASIEEEAREDPLPPGLLPEMAHLSGGPFAEQPGWRNLGGESPSLPQGSLFHSGTASSSQNGHEEGATGDREDGPGVALEGPLQEVLELLRPTDSTQPQLRELEYQVLGFRDRLKVWPPRPGRWPCFADGMMTGSRGLWGHAAWADILASPLRDLGGPCLSGPWFPHLASGDNNSPHGAQEDFKSSQ

SEQ ID NO: 5 - DNA, 828 bp, Homo sapiens
ggagaggaagccagatgctcccagacactggggactgtcctgggcctccgtccccaaggtgtggctggaggaagcagagtctactcccgctaagtctgtccgctcactgctggccaaagctgccctgcgtctcctccccaccgccagccagagggaacctgcaatttcacctcatttagagcatccggagcccaggactgctcagtcaaccctctggaatgcccacaactccccacaggccagccggccttgggactcccgcacagccacgtgagccggtggagccgggtctgtttgctagtggaggctgttaacagcacgggaagtggtcaagggttcaacaagagatgagccatctggtcctccagaggttgtgacttcaatataccctctcatgagacctttcctggccccttatctgtggaggaggcacgtgacccacatggtctggccactgatgactgaacaagctatggacaccggaccccggagagaccattcactcactggccacgaacatgagttcagatacatgccccaaaaggatgagcctgggtactggattccctccctcagaaacgtgaatcaagagacacaggatgttcctgttggtccagatacttgagctaaaaggtgatggatacctggatgtggggtggtcattctggggagtacgtccatatagaaagaggagcaggtgctgtgggattctggatcccagtgatagagctaagtggctggatcaagcttcacctgaaacccactctacttgtcttagtccattttgtgttgctataaaagaatacctgcaactgggtaatgtataaa

SEQ ID NO: 6 - DNA, 597 bp, Homo sapiens
catccggagcccaggactgctcagtcaaccctctggaatgcccacaactccccacaggccagccggccttgggactcccgcacagccacgtgagccggtggagccgggtctgtttgctagtggaggctgttaacagcacgggaagtggtcaagggttcaacaagagatgagccatctggtcctccagaggtggaggaggcacgtgacccacatggtctggccactgatgactgaacaagctatggacaccggaccccggagagaccattcactcactggccacgaacatgagttcagatacatgccccaaaaggatgagcctgggtactggattccctccctcagaaacgtgaatcaagagacacaggatgttcctgttggtccagatacttgagctaaaaggtgatggatacctggatgtggggtggtcattctggggagtacgtccatatagaaagaggagcaggtgctgtgggattctggatcccagtgatagagctaagtggctggatcaagcttcacctgaaacccactctacttgtcttagtccattttgtgttgctataaaagaatacctgcaactgggtaatgtataaa

SEQ ID NO: 7 - protein, 32 aa, Homo sapiens
  VARIANT 2-8, 10-15, 17-22, 24-31: Examplary motif
LXXXXXXXLXXXXXXLXXXXXXLXXXXXXXXL

SEQ ID NO: 8 - DNA, 106707 bp, Homo sapiens
gcctgaggccaccctccaagtgtccccacagcgcaccacaagaccacaggagtgacctcctcactggcaggtatttggggaaacaactgctgtctactcttttgggtaaaaagtgaaacaccaatagtttaattgaaatttcagaaaattgaacatatgaacaaggcaaataaatactaagtaagttaaaaacacaaaatatgtccaggaagtatcgatgagaatgttcaagttaaagttctccaatgccattgctacagcaacctcaaaccctaggttctctctgcactattaacacagacatctcaggacatggtttgcttttttttaagacttaaataggaaactaatttttctttctttaaagcaattgcgttcttcagtgaactctttctttaggccagttgatggcttcttagcagtttattgacgagatcctagggtagcttccgaagctgggttgattgattgcatttgggtgcggatggccaaagtgagtggccctactgcctgtgctgctcagggctcctgggctgatgtggtggcttcttccctttgtgctgctgaacatagggaaagtgaggttcacagtccaccatccaccagccgccgtctgtgagctccaccagcactcgcagcaagtcagtcgggctgagagtgtggttgtagaaaccctggctttgtgccttcccaccttccccagctcaccaaggtgacacctggctgctgctgaaatggcctgagcagtcttgcattagcagggccagccacctgccagcctgctgcatccccagtgcctcttgacgcagaagtgccgagcagctgaccggcagcgaggcctggagttctacacacttgccttggagccttttatttagggcctcaacttgcctggccttggcccttttgtaggtggccacctagtttggctcagctctgcatctcggggaaggtcacacagaccctcagccagaagttgagcgctctgttgaggccgtgcagcccctggaatgctgtacctttgccttgcttttttctgcctctgtacaaaagaccagcccatgccctggggctgggtcaatggccggagtctcaggtagagctctgggcagctcacactcctggaggagtgcacagcaccattacccagagtgcaggctatgtccaggctgggcagcagcaaaaaaaacgatgtgagatttgtgctcatcagccaggattttttaaaatattgtgatttcaacatctgcctcctggcaaaagacttctctttgttctgagcagagcttgtccatcttctcaaaagctaaccgtcctttttcacctgaaatagcaaagggacctgtcagcgggttggatcctgccttggcacttccaactctcctgggccagggtggccctagtgcttagtgactgtgggtctcagtggtctctgcaaagcggcaggggagggagtatgtgcgggagcccccacctggtgactcacatggcctgggggccttgtctttacctctaggatgttccgctgaatgggaaccctgccttgcctctggcttctatcccaaaggtctaagaagacagcgaacactccctgccaccccagccatggaggaggcctgccttggcaggatgctacaaagggtggaggtcggctctgtgccagggctgctaacggtgcccatcccaggtgccccagagttgttctgcctgctgggagagctgggtgtggcctctcgcagattctaagggccccaggcaccccgctgcgctgcacagtttgtgccactttttaccgaacgacagtgtggtttcccgggctgccgcccgcacggcctccaggtcagactggcacaggctggcagtctggtcgatgctttcaatgccctgttcgagattaggagaaaaagaaccctttagggggccttctcaacagcaggtagagtccacttagtggccctgcagggccagtcctagcatggtctctggggcctcagccccttccttttctccaggcttccaggttttttaggtggcctcaggttcatgagaggcacctctggactctggaagcgtctcgcctcttcagcccttacacccgctagggagccaggctgttagcagaactcgtcatcctggatgcctgctgaaaggctagaattgaaaaggagacctgctgctttctggaccttcctgcctccctcacgctctccttgccctactctccaggacagcctgtgccagtacttcgcccaactcaggcacatgccccctggctgctcctgcaggccaaggaccggcatgcgctgcagcgccctctactgggcacctggccctcgctggttttctgatcctaaccagcttctcctcttagaatttcctgctgatccatcccagaatgaatgggagttcaatctgtactgaattatctttcatctagcaattgtgcaattccaaatgcaggtgaggttgagggaaagcgggcatcccctcacatccatgggatctatgtgtgggttgtatcaagagtctcaaaaatgctcatattctccggtcctagaattgggtctagcctaaggaaataattcagaactccatgtttttttaaagctttatgcacaaacatgatcataagacatgatttatgataaaaattggatgaagtaaactttcctatgaaagcagctgagtaggttaaattaaggtatacacttgatagccccttcataaagaattctcaagtgaaaaaaaaaaaaaaaaaaagaaaaaaaacaccacaaataaagaattctaggccgggcgcggtggctcatgcctgtaatcccagcactttgggaggccgaggcgggcagatcacgaggtcaggagatcgagaccatcctggctaacacaggtaaaaccccatctctactaaaaatacaaaaaaattagccgggcgtggtggtgggcgcctgtagtcccagttgctggggaggctgaggcaggagaatggtgtgaacccagaaggcagagcttgcagtgagccgagatcgcgccactgcactccagcctgggtgacagaacaagactccatctcaagaaaaaaaaaaaaaaaaaaaaaaaaaaaaagctaaagtgctgccagtctaacacaaaatgttcttattttgttgactgtttttaaactatgtttggatcaacaattttaaatgctcacacacacccacaaacttttaaaagaagcataccagaatgtcaaccattgtttctgctacattctaggatgatggataactttcttttctatctctgtattttgtaacttttttttttttttttttaagatggagtcttgctctgtcacccaggctgcagtgcagtggcacgatctcggctcactgcaacctccacctcccgggttcaagtcccgagtagctgggactacaggcatgtgccaccacgcctagctaattttgtgtgtgtgtattattagtagagatggagtttcgccatgttggccaggctggtttcaaactcctgacctcaggtgatctgcctgccttggcctcccaaagtgctgagattacaggcgtgagccatcacgcccagccccagtggaatcattttgaagttaactaaccttaaccctaaccacaagcaggcttttctatcagccactaatggggaaacgtcaggctcaccttgaggtgtttgagcgctaggcatgcggcctgctggagccttgcgtcgttctctgccagggcgttggtgtagaacagcaaagcctggggagagtaaggaggctgtgaatggaggggtaagcagaagtggagtccatggttccgggtccatcagccaccaggtgccgacagtaaggcacgctgtgcccatctttctctaaacaacgttcaggacacgatcggtccatctttgggccctgtgtacacagtcacaagatctatactgtggtgtttaatttatccctaaaaacagatgccagggctaatacaatgaagaaagctatttttgtctaataatattccggaagtgcattctgaaactgctgtctataaaatgctgaatcagagaaatcagatgcccagctcagaacaacagaagaactgatcatcccatgtgcgttgccctcttcagctgaaaacgggcaaggctgctgccttgggccagagggaaacctgcctattcccctcagccctcctgtccaatcccagcggtgaccctgccctcttcggtctcacgaccatggccacatcagttcatgtctttgggcctaacttgactcagctgaaaacaaagctggcacttgcttcatgtgttattgtagggtttgtcagaaagcacacagggttatgcccggcccgcagtggagtccagcgtctgcggctgcaaagggaaacccaggagtgggtttgccctcactcagtcgagtggctgcaccttcaactgcacgggtggggcgatggaggggcccaggtgtagagttggctccagggacctgggcccagagccaaaagagaatggccgcctttccatctgcaggtggctctccaaagtgtctgctgctttgggaagacagactgggagtaggatcgggtcctcccgctcctcctacacagggctctggtgaaggctgtgaggccacatcggtgtggaaatgtcacactgcccactgcctctaaccccagggtcccaggtcatggcagcctactccttcctcagcaccctcatctgaggccaagcaatctgtcactgggttggcccccacctcagtgtttcctactctctaaagtctgtcacatgaagatgaaggccctttttttttttttttttgagacagagtctcacttcgttgcccagcctggggtgcagtagcacaatctcggctccctgcagcctctgcttcccatggaggccttgtttatatacccccatacccaaaaaacaaaatacacctgacttcagtggatccttgaagccaactactagttttcaggaactacagaagacagagaaatacattaaactaccgttaaacttcctgcaagcagcaaagtctagatagtctaggtcaacctgggatcgatcaaattaattgcaagcagagaaagaaacggggaaaaaaacctttagattgattggaagccatcagccaatcacaatgtgtgtccttatgtagatactattttaacaaaaaataaaacaggatgcttgaggcttggaaatttgaacataagatatatgatataaaggaattgatagttaattttttagataagataatggtgttagagttgtgatttttgaaagaattcttaaccttttgacacatatagttaactatttaaggtcaaataggatgcctcaggttgcttcaaagtgatacaggggagtggaggggaaaggggcaggattggccatgggttgatggtggttgggcttgggttatgggtgcaggtggattcattatattgctctgtctacttttgcaagttcaaaagtctccaaataaagagttaaaaacaaccacaaagtaggcggatgggctccaagaagggctattggcaatggaactggagatttcctctctagtctggagctgagaccatcagtgtagactatgcccttgatgtcacccttcctgaacccctcagggtgtggcgccttaaacgtacagtagttacaggcaaagagtgaaaaagcagagaggtccactctcttggttttcaaatggactgaacacagtgacccattaccaggtagccatgaatattaattgaaagtaaataaggatgactatcaaaacactaagaaaggctgggcgcagtggctcacgcctgtaatcccagcactttgggaagccaaggcaggcggatcgcagggtcaggagtttgagaccagcctggccaacatggtgaaaccctgtatctactaaaattacaaaaattagctgggtatggtggtgggcgcctgtaatcccagctactcaggaggctgaggcaggagaattgcttgaacccaggaggtggaggttgcaagatcgtggcactggactccagcctgggcaacagagcagaactctgtctaaacagacagacaaaaaacgcactaagaaaaacattcagcgtgcaagtgacatctcagaggcctaacagatgtgttgctttggaagcagcaaggtgcattcatgtgtgttagatcgtagcccaggtccctccatcaaaatagctcacagctactgcagcccctggcactcacttctttgtacttttccatgagaacaccagcttacctatgcccacacatctgtggccagggtctgccacctgccctggacaacgtaccttttcccggaagcttctgttcctgactgcaccagccaggcgagcgctggccgccctgcacaccctcggagtgccgtccagctggagcagcgcccaggctcttaaggtctggggcagaggctggagctggcccagccgcttcccctgcagcttccggaccaccttggcctgtcccgcacagtgaagctcctcgatgagtgtcactagaagacaggaaagaggtgggcccaggtcccctgaatgggagtttggcaggacagctgcaagtttgcttggctgctgccagtagccacagaggaacaaatcccagacccaccgggatgatcaccaaggcccagcctggactaatttcacagggagctcctggaatctccaggaaggccttttaacaaggggtcaaatatgctccataaattaataaaaccacagcccacacttccagggactctggccagccaagatcaccctcctacccagctctgacctctgttccgtgctttttaaagctgacttcccttggagctaatatcagcccccatcggctgaacgcagaatctcattaaatcggggttcccaaaagaacagttggcggggatggatgtagtggttctgaattataacctgagaaactgcatgtgacagggctccgtggatattcctctgctatgacagccacccaccccagtcttaccttccttggtgagctgggtgaagtgcttctccaggtcagagacgctctgcctctgcaggtagctgtgaaactggaaccaggtaatgatctgttcttctgggagcccagctccggggagcagcccaccacagctgaccacctgctccaggagcctgcggcacgctggccaaaggggagagtacatcaggagaaactgagacctcgaccctccacgcttctcagctgggagtagcctggtcagctaaaaggctttctgggccgggcgcagtggctcgcacctgtaaccccagcactttgggaggccaaggtgggcagatcacctgaggtcaggagttcgagaccagcctgaccaacatggtgaaaccccatctctactaaaaatacaaaattagctgggtgtggtggtgcctgcctgtttcccagcaactctggaggctgaggcaggagaatcgcttgaacccgggaggtggaggttgtagtgagccaagattgcgccactgcattccagtctgggcaaccatgagtgaaaccccatctcaaaaaaaaaaagggtttctgatggcacgagggcaggtgtccctcactgcattccctgtgctgtaggggaggagtgtgcccagctagagtcaggactgtgactccaactcaccctgagtcagaccgtgttgggttcatccccatgccctgggccccacaccacacctggatcaaaatcccggaggcagggcctgggaatatgcatgtcaacaagcagtccaggtggtgcttggaacatgactgtcacctttcacttgctccacagagaaaggcaaattctggggaagaacgcagtccagccagcatttctagataccctcgtgggcccctgcctgcctcccttcccataaggttcattctttacccgtcaggctctggtgggaggcatggaatctgtccccagaaaaatgctctggcggccgggcgcagtggctcatgcctgtaatcccagcactttgggaggccgaggcgggcgaatcacaaggtcaggagttcgagaccagcctggccaatatggcgaaaccccgtctctactaaaaatacaaaaaaagaaaattagccaagcgtggtggtgcacacctgtaatcccagctactcgggaggctgaggcagaagaatcgcttgaacccgggaggtagaggttgcagtgagccatgattgtgccacagcactatagcctgggcgacagagtgagactccatctcaaaaaaaaaaaaaaaaaaaaatgcaccggcccaccaagtttgcacgcagcttcagggctccacagactgctcagaaggccccacgtggaggccccttctcgcccggtgaaagggcggtgactcgcactgaagctgagaaagctcctccgtccgatggcatgaagacacagaggtgaagacagggctgaaatgaggccagctgtggccaccctgaaggccctggagactcaatacagcctttctgtgggaggggacgacaggacagaagggaacccacctatttccagctgtcctgggtacttccctctgactctgtgctggaaggttttcttgagctggttcagcagcgtcgtggcagggcaggacaggaccctgccaggccctgtgcaccctctccacagcttcaggcaccccttcgtccgcgaggcctgtgggatgactgaaagccccagttcagaaacctgaatggtgactcggggagagcacatgacaaggacccgaaaggtttccctccactaggacctgagggggtagggaggaggggacggtgtggcttgatgcgaggtcccttccctgcattcccatgtgacacgtgagcaactttggctctaagcatcttaccagggccaccacctgcagtccccacaacaacctgggaggggctgctgtcaccagcctctccttacagacaaggaacctggccttctgaggggaggtcccacggggcagaggcacagctgggatcacagctactgtttgacggcacattctgcaccttgaatgtggcctggggttacctcactgaaccccgtgcagtgccctcctcctatgcagatagggaagcagaggctcagagatgtgaatcatttgcctagagtcacacagctgactgaagagtgtgctgcaactccaggacttgtctcccttacctccccacaaagagtgtgtatctctgagcccagcccagccacagcctccactctgggccccgattaactctggctattaggaaggcagaagaggctccccgagctttgatcccgtccccgtgccgctcacataggcagcccctgggtggcaggcagctacttactctcttcaatggatgttgccttgccgaccttctcaaagtcaaggacagaaagtgtctccagaacgtgcttttgctgtgccacttcttccaggaggcattcctggaccagccttgataaattaggggaggccagtttctgggaagcagcccagatgctccagattagcatggaacatgcccaccgaggtcagctgttacggctgtggctcccacctgcttgtgcccatctcttctgtcccgggggcagctccctgactgtcatttggggattcccctttcaccaaggtggctgagctaatgggatgcaggctgggctgccagggaccacctttgcccccagaaggaggaccaacctgcctgaaagtgaagccaacccacattgcaaagcagaactgagagagacctagtcctgatagcatttgaacccgtgcatccagccgagcctgaagcctacctctgaggctttcagtaccatgagccgataaattttcctgttggctgagccagtttgcattagggttctggcacctataaatgagagtcaccaccaaagcctttggtttagggcttggcctgtgtaagagccacacgagtattcacccagaccctggtctgcctctcttccactggggaatagcttcagtctcacgggcttccaggatgcaggtctgtcaccccttcatgcttcccaccaccttcaagatgagcctttgcaaagaggactccaaacccctgtctgcccctccctgacaagtcccctagcccagccacctgcagcagagctttgcagacttggaggtgtaccatcagcagcacgtccagctctggggcaccggctgtgagttccctggatgacgctttcagtgatgacggtgggggcaggggccggtcctttctgagttgaattgagaactgggtgaccattccagttagcagttcagctccctctgagggtgggggaaccctcccttcccagcgagccccaggtcaggaggcccactccaggtcccaggaagttcaactgaagaggaaggggaaaggaacaaagggtggcagcaactcgaaacagagctgggaggtgtggccaagggcctggggaaggcagggcgggcagtcgtcttccaggatgtctcgtggaggtagaagagaattccaactcatcccagctctgtgacgctggacaagtctgcctccccaaggctcaggtgactcatctatcaagggaggcaggagccccacactcaaggccgtgaggggtgcatgaaatacggtgggcaagagcgcctcacaccaagtccactctgtggtagaggctagacctgctgcccgacccactcagccactcaggaagggtggaagcaagtgtggcacgaaacaccgcactcgtccgtgggattgtgagaggcgaggagaaggaattgagcctctgctcttccatgtggtgagatgcggatgcagaagccctcgggggctggggcaggtgaggctgcgtagcctcgagggaagctgtcatgagaggtgtgggccctgtggtgggacagagggaacagcactggatactgctgcaccttggttaggggctaagagctttttttttctttttctttttctttttttgagacagagtcttactttcgcccaggctgaaatgtagtggtgccatcttggctcactgcaacctccgcctcccgggtccgagtgattctcctgcctcagcctcctgagtagctgagattacaggctcttgccaccatgcccagctaatttttgtatttttagtagagacggggtttcatcatgttggccaggctggtcttgaactcctgacctcaggtaatccgcctgcctcggcctcccaaagtgttgggattacaggcgtgagccactgtgcctggcctcaaagaacttctactatatacttggtgattatttttaaggtttaaaaaataatactaaaagctggttccttggagcagaaggctcaggggcaggctggggtgatctcacccacttcgtggtctctgaggaccggctctgaggcaagtgggggatgtgcggggatggcatggggaagggtgcacgatagagtgacaagagctgagccaaggacagtgggagaaacagacggggaggctggcaggaaacgtggagctcgggtcacccggtgggagtggtggccactgggtcactgctggaaggaggtgcactcaccggagaccctgggagcccccaaacagggacagctcatccagggcgaagtcggcattgaggaaggcgaagctctccaggatgcactccatcaggctctcggccgaggtgtgctcctgccgtgctctgcagggctgtggacgaagtggccagacctgagggcaacaccgggccccacccacccgactgggacactggccaggggcctcacggcagacttgggcaatgtcccggtcccaagccccaatcccacacaccgtcccccagcagaagcccagccgctggcccaaggcgtggcactgggcgtgtcaccccccaaagtccctcctgaatggcctgtgacaaggtaggagcagaatcttgagggggacagatctgaaaaccctcccctgtatctcagacacccacggggacaaggccaatggcaggagtgagtgagctgccccagcggggaccagggaggcaggagcgcaggtgcctcccctaagagggggccacgctcatagatcgcccagttctaagaaaaccctaaaacatggatatttatgggaaactgcccagctttgaaataccaacaacaaatttcaaagtattttaaatacatggtgtaggtcaggcaaacgcttcttcacgaggctcagaggcctgtgggctccaggttcctcctccctgttgggtttctctccccaacagattaagggagagtgttggggccaccaacccgccactggtcaagtcaccagagcctccagtgccagctccctccctggagatttctgtcttgtccccaactcgtggctggctctggggctggttcctgcccccgttcccagcacaccccacccaggccttgtgtccaacacaaactgcagccaaggcacagcccaagcagaagccataaaaccaaaccaggatcctaactgcggagccaaagagaaaatcttgaggacgaagggaataacaaggctagttgcagagatcacattttccaatacaaaaatataggcatacatgtgtatgtaagtgtgtatcaatacacacatgcatgtagactgctgattctcaatagtcaaggtagttatgttctagaggccaaggcgggcagatcacttgaggtcaggagttcaagaccagcctggccaacatgatgaaactgcatctctactaaaaatacaaaaattagccgggtgtggtggcacagacctgtagcctcagctactcaggaggctgaggcaggagaattgcttgacccggggaggcggaggttgcagtgagccaagattgcaccactgcgctccagcctgagcgacagagcaagactccatctcagaaaaaaaaaaaaaaaggagggggttatgttctataaaatcacagcaaaaaaactgaatcagcaaaagctgaaccattgctcctaagggagttactgggttaggttcctgtgaacctctggtcacagcagttttatcaactcagcaatgcagaactttgtatgtgtcttttggtttaaagacaccttatttaatagctattgttggctgggtgtggtggctcacacctgtaatcccagcactctgggaggccgaggcaggtggatcacctgatgtccggagttcaagaccagcctagccaacatagtgaaaccctgtctctactaaaaatacaaaaattagccgggcatgatggtacgtgcctgtaaccccaggtactagggaggctgaggcaagagaatcacttgaacccaggaggtggaggttgcagtaagctgagatcgtgccactgcactccagcctggacaacagagtgagactccgtctccaaaaaaaaaggaaaaaaaaaagctatttttgattcattaacattgaactcaacagccagcatcgctacaactcatgcctgaaggaagctcatctaacacacattttctctgtaaggtatttcacaggcttcctggactgaggaacaccagccggcactgaagctctgggcttgggaggcatttaaacagtgaaactgtcaacaaaaagcacaaaaacttgaaaaacatggcattaaatagaccatgaggacacttgtttaccatttgggcattgaaacaggaaggcaaagcattgcctcgcttgacctcagctgggaatgtgtgctttgagcagctcagattttctatcactctgcccggccccaaaacccactttggaatcgcctcgagtattgatttgggggttagaaataaattttagcaagtaagtaaattccaaaatgcagaatccacaaataatgaggataaatcatgtatatgtatgtgtgtatgtatgcgctcacacacacacttttttttcttttgagagagagagtctcgctctgttgcccaggctggagtgcattggtgcgatcttggctcactgtaacctccgcctcccgggttcaagtgattctcctgcctcagcctcccaagtagctgggactacaggtgcacgctaccatgctcagctaattcttttgtctttttagtagaaacggggttttgccatgttggccaggctgctgttgaactcgtagcctcaagcaatccacctgccttggcctcccaaagtgctgggattacagatgtgagccactgcgcccaccatcatatacacatagatttgcataaatgtgtgtgtgtgtaatttctctccagaaagtcaaaccaaaaaccacggtctgtaatcaatttgcattgttctgacttcgtttgccaaaaaaagaaaagttccttaaagacgtttaaaatgattacattattgcggcattaacatttttatgtaaattgggtgtaatttttcaaaatacaagtatgtgacaaatgtgcatgccgactcaaattagtctacaaaaaaaggctgttaaaaagtacaaaaaaggtacaggcacgatggctcacgcctgtaatcccaacgcttcgggaggctgaggcacgaggattgcatgagtccaggagtttgagaccagcctgggcaacaaagtgagaactcgtctctataaaaaataaacaaaattagccaggcatggtgatgtgtgcctgtagcctcaactagtcaggaggctgaggggggaggatcgtgtgatcccaggaggcagaggttgcagtgagccaagattgcaccactgaactccagcctgggcaacagagtgagaccctgtctccagaaaaatgaaaaacatgaagtaccaaaaagttgacattatttcttaaatttttttctggaaattttcaggcaggtcctttttaacaaccatagtagtggtaaacggaacatattttaaagttatggatgtaaaatgagaaattaaactttttttcccctatgttgatttgtcctgttggaggtgtggttggcgataaaatgtgtttgaagagagctgttaactatttcagtgccaaagtgtatcttacacaaaagaggggaaaacagaaaacagcaaaatcagagaagtgcacagccagtctgagggcataaggaccgagggaacacagagcaggaagggatgggcctgccggggccaggtgggagttgccatttaaaagagggtgtccaggccaggtgcagtggctcacgcctgtaatcccggcactttgggaggctgaggcaggcggatcacttgaggtcaggagtttgagaccagcctggccaacatggcgaaaccccatctctactaaaaatacaaaaattagccaggcgtggtggtgtgcacctgtaatcccagctactcgggacgctgaggcaggagaaatgcttgaacctgggaggaggaggttgcagtgagccgagatggcgccgctacaactctgttgccagcctgggcaacagagcgagacttcatctcaaaaaataaatacataaaaaataaaagagcgtgttcagggaaggacacccttaagttaattgatgtattagtaggtactagtggtatacatttttttgatgtacattcaaatttgtgtaaacacgagaggagaatgtaataatttgcctaggtcagcacctctgaaagccacctgccacctaagccctcctgtgaactgagaacagccccggggtatgagccctgtggagaaggttcagtgcggggtgcgtggatgaggcaaacagggaactgtgtggggccttccatggagccagctgcgaccctgacaacactccaagtttgggtttgtagccacaagccacagccccgcccacccaggtcagaaggtgcgagatggggccagtttctggctggatgtgcatgagcctatctactgtgaactcttaaaatcttcctgagccccatgagggctgttgttatccccgcttgccagatgaggaaaccaaggcccagagagacacggcccacccaggtcacgcagaggtgaggccaggatgtcggcccaggctgcgtggccccagagcccccgactcccagtcatcatgccatcagcaaagcagggccaccgcccggggcggggtggccataccttcagccggtcccggaagccgaggacctggtactccagctcccggagctggggctgggtggagtccgtgggcctcagcaactccaggacctcctgcagaggcccctcgagggccacgccaggcccgtcctctctgtccccggttgccccttcctcgtggccgttctggctactcgaggctgtgccgctgtggaacagggagccctgtggcaggctggggctctcccctcctaagttcctccagccaggctgctctgcaaacgggcctccagagaggtgggccatctctggcaggagacctgggggcagggggtcctcccgagcctcctcttcaatggaggcgtggggaccgaaggtcaagggcaggaagcccacatctgaggtggacgccgacgtgctggtctccgtgtctcgggggtcctcagagctgaaggagtccatctcaggcagctcctgactctggcttcttaggctgggaccccggaggtcgctgtcagacaggtagctgaggatggaggtggcccttgggccacccagcagcaaggcctgctgtgttggctgctgtaggacagactggagaggggacacgggagcggcctcaccagccaccccagggaccacagcaagtcccccagaggggcttcccctgacagacacccgctgtgcatgcccatgttctcaggatgactgaggcctgccgaggtgaccagcatcccagaggtgcagaaaaaaccctgtcccctctctgcacttatcccccatcccgcctccaactcactccccccaacctcaacagcctcctggctattgctcatgccctgtgggctgggccccacgttcctgcctcagggcctttgcactgactgtgctctcgtcagaacactcttccccatgtgccttcatggcttgtgccctcgcctcctcaggaccttacataggtgtcacctctcctgagaaccctgtcctggacagccttttttaagtggcaatttctacctggccccagtgggccccttcccttcctgctgtctcccacagtccttatgcccctggggctggctgtgcacttctcggattttgatatcatctgtttcccctgtagaatttcagctccctgagggcacagtctttgcctggcctggagcagaggctggcatatagtttcggtttttgttttgttttgttttgaaatagtctcgctctgttgcccaggctggagtgcagtggcactatcttggctcactacaaccttcatctcctgggctcaagcaattctcctgcctcagcctcctgagtagctgggattacagtggcactccatgacgcctggctaatttttgtatttttagtagagacggggtttcaccatgttggccaggctggtctcaaactcctgacctcaggtgatccacccgcctcggcctctcaaagtactgggattacaggcatgagccactgcacccagcctagttttggatgaatgaagtgaatgaatgaatgaagggcctgacaaacacaccatagaaacagcagcagctgtcattaagtattctgaggttgccggccctattccagggccttcccattaccctcaccacaccccatgggggtggcactgccctaacctcggagaggtccttaggccacacgggtggtgagtacagtgccacagtgtgaatctgggtacacggacacgaacccttacctctcatcagctgaaccaatgataatggttgatgttcattaactcagttaactctcacagtaatcccataagggcagtgctgttattctcacttctctgatacagaaattgaagtccagagagggctgggcacggtggctcatgcctgtaatcccagcactttgggaggccgaggcgggtggatcacctgacgtcaggagttcaagaccagcctggctaacatggtgaaaccccgtctctactaaaaatacgaaaattagccagatgtggtggcgtgtgcctgtaatcccagctactcgggaggctgaggcaggagaatcgcttgaacctgggaggaggtggaggttgcagtgagctgacattgcgtgactgcactccagcctgggagacaagagcgaaactccgtctcaaaaaaaaaaaacaaagaaaaaaaaaggcgtatacacttgctggtagatggattgctgtctctggaagaatatggaagaaactgatgacaggagttggtattaaggaggggaccctgatccatggagatcaacaggaaagatgttttactgggcactctgtcaagtactactactagaaacgtatatattaaggccaaggccatgactggggtgctagaaccatccagagagcccactgcagatgtcctgaagaaccagccaagcccagaacccaggctaggttggaggctggcagcagaggaagaaagtgcacaggaaaacacccaggatcgcctcaaacggaagctgagcccgaggcttgccatgtctgggagggccagactgctcagcccagctcctgcgtgctgcccccatcccagggtgaccacagcggccctgccccgggaaggctcactcaccaggtagtatctctcccggaagctgggggtgctcgggggtgtccagttgtacaaggagcccttcctgctgcccatagaaaacttgcccgtggggctgggtgacaccaggaagctctcagtatcaaacgggctggaggagagaacagaaggtcaggatgccatcggcacccagaggccatttcaggcccagacggcccacagggctcttaggttatgcaggtagatggtcttcatcttacaatacaatagcatggatggtgtgtgaggagctctgctctaaactgttgcttgtttttgagacagggtcttgctgtgatgcccaggttggaatgcagtggtgccaccacagctcactgcatccttgaactcctgggttcaagcgatcctcctgcctcagcctcctgtgtagctatgaccacaggcatgtaccaccatgctggactaatttttaaatttatttttattttttgtaaagacagcgtcttgccatgttgcccaggctggtctcgaacttctgggctcaagcaatcctcctgcctcagcctcccaaagtgctgggattacaggccatgagccactgagcccagcctctactaaactctttacacaaatccttatttccacccccaagacaaccctctgtggcctggagaattatttacaggggaggaaaggaaggctctgagagatgagtgacttgcttagggcagtgtcacaaccaaccacgcgagacggaactgaattcacacacagattttctctgaccccaaagccttaaagatcactaagagtgatgcttactcttcagacatcactgtacttaatggatttaagaggaaatgggctgggtgctgtggctcacacctgtaatcccaacacttcgggaagccaaggcaggcagatgacttgagctcaggagttcgagaccagcctccataacctggcaaaacccccgtctctacaaaaaatgaatacattagctgggctaatgtggtgaccaacagacacttgtagtcccagctactcaggaggcagaggtggaaggagcacctgagcctgggaggtggaggctgcagtgagccgagatcatgccactgctctccagtctggggcaacagagcaagactctatctcaaaaaaaaagaaaaaaaaaaaaagcaaaataaaacaggaaatggaggcttgggcctccaagtccagggccttgcccatggtcacaggtgcagcctaggaactccaggttacatgacctctacccctttagaaacctttctcaaggctgggcgtggtggctcatgcctgtaatcccagcactttgggaggccaaggtgggtggatcgcctgaggtcaggagttcgagaccagcctggccaacatggcgaaaccccatctctcctaaaaacataaaaaaattagctgggcatggtggcaggcacctgtgatcccagctacttgagaggctgaggctggggaatcgcttaaatctgggaggcagatgttgcagtgagccgagattgcgccattgcactccagcctgggcgacagagcgagactctgtctcaaaaagaaaaaaagaaacctttctcagactctgaccgccctgagggcccttagccagatggtgagggacagtgactgtgagcaggagagcaggatctggaggcaggaaacctcaggtcaattcatgctaaatcaaggaaagacaccaaggtctgaagggacagggaatctaaggccaattaacgcaatcttcctaaagctaacccaaaaggaaaaaccccgtctccccacactgagtagtaaaggatcaaaggcaacgctccctacagccctcctgcctccaaccatggctcagatggaaagggagggtgtatggatgggccgctggcgaaacagggaccatccctctatctgcatagggcgccatccacctcagcctctaaccacagaccaaatcctttatccagaaaaggggcagcccataggaacctcaaacagggtacttaaagcccagaaactttgaaaccatgcccttgagccacatgctcgggcccactcccaccctgtggagtgctttcttgccttttttttttttctttttctctgagactgtcttgctctgtcacccaggctggagtgcggtggcgccatcttggctcactgcagcctccgcctccccggttcaagcgattctcctgcctcagcctcccaagtagctgaaactataggcccgtgccaccacgcctggctaatttttgtgtttttagtagagatggggtttcgccttgttggccaggctggtctcgaactcctgacctcgggtgatctgcccacctacgcctctcaaagtgctgggattataggtgtgagccaccgcgcccaacctgctttcttgctttaataaagtcctgctgcttcattcctgcgtttcattcccctgctccttttctgcattttgttcaagtctttgttcaaaatgccagggacttggacaactcattgtcaagaccctccaccagtaacaactggacacccccagttagaggcccctttgagaagctcagccgatgagcaggggacactcggttcagaccccttgtctgtaaaaggggtgcgcttacagaagaacccccatgtggaacatgcacagggaagggtgggaatccaggcgagcgcatgggagcaccgaggcaagtgatgctcagcctggcccgtgatgctcagcctggcctgtgtccagccttgcactcggagtaggtaggtccccatagatccatttgcctcgaagaaacttgcttataattatttgcacaaaggcctggctcgtggcacctgggcctggcctgaccagaaggcctagagctcagaggctctgcaagtgcccacttctctgcccagggtgtctgcagacccgcaggcaccctgggtcctaggcttggatggcactgagctgcccgccaccctgtacacttcaggggcccctgggcgaggggtattctcagccctaccagacaggtgaggaccccgagggtgcaggtggtcgctcaacagaattcacccagctcacgagggagagggcagcagctttgtgacccccagctcggacacccaccctagggagagaggagagagaccagccccgactggccagagcaagcacagagcagaggagtgtcctcagccctcccttctgctgcctctgcgctgaggaggaggaaaggaacaccctggaaatgcaaggcttccttagaacagagagctctggagccctccacgctcatgggcatgcagtccagggagtgatctcatggacatgggcttcatgggtctgcagccgagagggggacgcaggtgaccgctcagccccaggccaagggggcagccagggtcggccaggaatgagggggtgggagcagcaaggagggccttcctggagaggtgagcctcctccaaggagccaagcccgggaggccccggaaagtgtccccccaaccttccctgtggctttaatagtcaggggttcagaagcaagaagggaagcaaaaccagaaaggcaggcaggggctacagagaagttctaggcctcgacctgactgaggggtgggagtgagggacagatgaggacccgtgagggcagggacacccttggcagaggctggtgcaggaacccagggccagagtgtggccaggccaccaggggcagccagccaggcctccgctctccccaggctggacgggactcacttccactgcacctccagctgcagcttgatggtacccaactccgtgatgtccaccacgatgacctgcggccgcgtcgtgaagaagtcggcgatgtcacacgtcactgcacccacagccagcgagcccaggccccgcaactccgtcacctgggggtgggggctggagggtggtgtctgagccgaacacccaggcaccccagccctgcccccgggccccatccccaccttgatgtccaggttctcatgcagcgtggggatgaaggccttctcctcttcgtcccaggtctggctgtcatctgactcgatccgacccttgagcttccaacgctggcggcccagacgcatgagcacctgtgaaccagcccgagaggggccgcgtcagcccaggtgggtgtccccttgctgtccgcccagggccctccctgccaggcagagccccagctgcaaccctggttcccagcagctcccgtcccccaaagacctggcggggagccctgaggattgaccccagagagtggccgtacctcatagtggtctccgggacagaggcgtgcgtagcccaccaagcctggaacacagacatggccggtctcccctccgccttccactctccctgacctgggaccacaggtcctctctggggttcccccgagtatagattttcagtttcagtggggtgaggatgggggggaggtacaccatcatgatggaaaatggacagagggtgctgggccctcacaccaggctcagagaggggtgggacttgccagaagtcacatgtcacatggatgcaaaagccagggctgggctcagacccctgggattctggccaattcccgtgcccctcagcagaagtctcagggcctccagaaaggcctccgcccaccccctctcagccctgttacctttcatcctgatgtggaactcgcccaggtgaacctccagggccccctcgatgagccacatgtcctgcaaagccccggaggtggctcagctggctgcctggggctaggccacgagggcctctaaccatccctgcagccagacagaggccacaggcagagagacgcctccttggggcccagaacacctcctccagcccccactggcccagctctcgatgtccccactgcccggcccagctcttgctgcccctgctgcccagcccagcttggcccggcccacctcggcgcactcgtgcaggctgcggcccagctcctgcaggctctctcgggctgcgcggctcggggggcaccgggcgaaggcccgctgcatgctggaggcgccgtcgcgcaggcggcactggatgcagtagtcctcgtacagctcatccacctgtggtgggcacacgggctggtggcgctgcccacgcggaggggcggccccacacctgcctgtcgacttctcctctctgggagaggccctccctgagctaagcaccccgctagcccagcccatggtgacagtcactacctgtccagtcccattcaaagcagtcacccctggccccagtagaacatgaacccccataggcagggaccacatctgcctcacctgcctcacctgccaccactgcctcacctgccacccctaccacccctgcctcacctgccacccctgcctcacctgccacccctgcctcacctgccacccctgcctcccctgcctcccctgccacccctgcctcacctgccacccctgcctcacctgccacccctgcctcacctgccaccccggcctcacctgccaccccggcctcacctgccacccctgccaaccctgtctcacctgcctctcctgcctctcctgccacccctgccaaccctgtctcacctgcctctcctgcctctcctgccacccctgcctcacctgctgcatacccagctcctggcagcgactggcacacagtgtgcacagcaaaaatgagtgaaagggacaagggaatctgtcccttcacctcctctgcctggtttttcaacaatgaaatgggagatgactttgtgataacctgccacccactgggcagtgtggggagtaaagcaagatcatgaaaccgtttgcagactctaaagcttacagatctgctatgcgaccttgggccaacccatgttcatctctggacctctgcttttccaactgtacaatgggctgggagggctcaaccttcccagccatctaagactgagcatgaggtctttctgcataaactgaagaaagagcccacacagtcctcagggagtcccacctcagggctgagccccctgactcccacctcagggctgagcccctgctgtccttcaactggcccgaggcccctgctcatccttagcctcctgcagctgccccatacccagaggccctgatccctgtttcgagggcacctccccagctcctgctaacctagctgaggcccagcaagctggcactgccccaccccaccctgcaacatccacgagccagctgaccttgctgatgtgaaactccatcttccgaatgtgcctttccacacagcgcgtttgcttctcccggaaaaagggaagatgtttgcaaagttgcctgggccacccacctgccccgcttgcccctgccaccctcctacaggtcctaactcagagaatggggcccctcaccatccctgaggaaggctcatcgcagagactcagccttcccattcctaaaatggggaggagacccaggttttctgcccatcaggcagccaggaagatgcaatgaggcacagtcattctcatccagccaggcccagcccacctcactcaccgtatgcagactcaccttgtccaggtcataatagaaagcctgtgagggaggaaaggagggcggaagaagctgtcagagtcccacatgttcctccaaggcctatgaggctctatgctggcggcgcctgagctcagagtcagaggacagaaagccatgtctacagccacccccacccactccttcctctgccaacggcagactgctgtccacgccaaggacagcactgattaaacacatgcacgtgggatgaggcagttctaggtctggctcttctatttcccagctgcgtgtcccaggcaagtcattcagcctttctgggcctccgtttcctcctatgtaaagcagggtaggagaagcgcctacctcacagggagaaaaaaggacacagtaggcccttgacaaaatgggaaccattgagattgaaggaatggcctcggccatgacaaaagaacatgagggggatggaagcgggaggggcacatggcacagcagttcaggccttaatggtgaattcctaagtctatccattccaggggccagggtgcgaccaacacaaataatatatcatcagggcaggcaaacctttctataggtttgtaaagggccagagagtaaatatttcaggcttggcaggccacggcgccccatgccaagatctataaagtaggaccatcctggccaggcgcagtggctcacgcctgtaatcccagcactttgggaggctgaggtggtcagatcacttgaggtcaggagtttaagtccagcctggccaacgtggcgaaaccccatctctactaaaaatataaaaagtagctgggtgtggtggtgcatgcctgtaatcccagctacttgccagacttgaggcaggagaatcacttgaacctcagaggtggaggttgcagtgatctaagaccgtgccactgtactccagcctgggtgacagagcgagactccatctcaaaaaaaaaaaaaaaagaaaaaaagtaggaccatctaaaacatacacacctttgcctaccttagagttacttctagaaccaagaaagagtgtgaagtggacctaagacatgggaagtgctgtacctgcctgtggggttaaattcatcagggtcaaagttttgtcctaactaagcaactctgccatcatagtgggaaagcagccacagacaatatgtacatgaacaagtgtggtcaagaaacatttttttaaacaagtgtttaaaaaaactcttgttttttaagtgactctaattttaaaatatagtgactctaattttcactggctgttaatttccagtggagattagagtcactatgtctgagtctggaggggttaccatggggttgtagatccaccagggaggaaaagtaagctgtaggagagtatgtaaattgacatttcatttttgtaagacaattttaactcacaattgctaaacacatttacatgattatgtgaccagagagaaaaatatggaaagatcaataaaagattgttctcaagggttaaacgggatgggacacggaagattgaatagaggagggcgtcaatcgagaaagaaagaaggattcaaccccaaaaagtcccatgtatgtatttatgtggaattgtacatatgcgataatgaaataaaaattccatttaagccaggtgaagtggctcacacctgtaatcctagcactttgggaggccaaggcaggaggatcacctgagcccaggcattcaagaccagtctgggcaacacagcaagaccccatctctacaaaaaatttaaaaattaactgggcatggtggcacacacctgtagtcccagctacttgagaggctgaggtaagaggattgcttgagcccaggagtttgaggttacggagagctatgatcgtaccactgcacttcaccctgggtgacagagtaagaccttgtcactttaagaataaataaataaaatctttttaaaattaagaaacaattaaaactccatttaagaaataaagaaacaggatgggtgtggtggctcatgcctataatcctagccttttgggaggccgaggtgagtggatcacctgaggtcaggagttggagaccagcctggccaacatggtgaaaccccctctctactaaataaaaaatacaaaaattatcctggcatgattgtgcgcgcctgtaatcctagctactcaggaggctgaggcaggagaatcacttgaacctgggaggcagagtttgcagtgaatggagatcgtgacactggactccagcctgggtgacagagcgagaatccatcccgaaaaaaaagaaaagaaaagaaaagaaataaataaagaaagaggaaagatcccaagcctttgaaaagaagagtgactacccagcaacgtctcaaaaaccaacatcttgtttcaatgagaaccagcagtgtcatgggagcaggactcgacatgggcatcttgctgtgtggctccagtgcggtcactgtgccctggcctcagtgtgcccatctatgacatgggagcagctaaaacatacaagtatttatactcttgcctgctttagagttacttctagaatggaaagaatgggaaacgaagtccacggggtgagactcactgtacccatgtgagaagaggggcaggtgctggagctgggtgatatcttggcagcaccttctcccaccatctgctgctggtcacccgcccaactgtccactaagggtgaggcatgctgagcttggcaggcctttccctccccaagtccctggcacacacatgaggctgggtccttgcctctcctaccctgcccaccccgaggccctctctgctggagccaggatacagagacggccgcttgtttgcttgcttatttcattcattcattcattcattcattcattgaaacagagtctggctctatcgtccaggctggtgtgcagtggtacgatctcacctcactgcatcctccgcctcctgggttcaagctattctcctgcctcagcctcccgagtagctgggactacaggcacccgccaccacgcctggctaatttttgtatttttagtacagatggggtttcaccatgttggccaggctggtctcaaactcctgatctcgagtgatccacccgccttggcctcccaaagtgctgagactacaagcgtgagccactgtgctgagccactggatgctattttggtagaaaaactcatctcccctcctcgctttcccagtccctcagtcaagctggtggcctgttctgcacattggtgctttggaggagggattctgggaaacactggctgggaggagaatcccacagtagcaaggcctccttgcttttgtctccaaatctaattcattaaggacatgtggggttttctcaaggcagcagcggagagacttcccagatgaaaggaagatcctcctgctgaaaggatgaagtccctgagcaatgggatcctagcagtgtcaccagataaaatacaggacacctaattaaaattaaaatttccaatcaacaaggaacaattattttagcataagtatgtcccaaagattacatgggatatacttatacctaaacaaatttgtgatttgtctgaaaaaccaatttaatgggaggccgggcacagtggctcatgcctgtaatcccagaactttgggaggcagaggcaagcgaatcacttgaggtcaggagtttgagaccagcttggccaacgtgaaaccttgtctctactaaaaatgtgaaaaaacagctgggcgtggtggcgggtgcctgttatcccagctactcaggaggctgaggcaggacaatcgcttgaatctgggaggcagaggttgcagggagccgatatcacaccattgcactccagcctgggcaacagagtgagactccatctcaaaaacaaaacaaaacaaaacaaacaaatttaatggggcatgctatttgtgagtgtgtgtgtgtgtgtgtgtgtgtgtgtgtgtgtgtgttgctaagtcggtctctgctaagggagaggcaaaaaaccagagcaggtgagccttgggcaacacagggcccagggccagcctgaaagacactgccttggctggcacagtgcctcacacctgtaatcccagcactttgggaggccgacacaggaggattgctggagcccaggagttcaagaccagcctaggcaacatgatgagaccctgtatctacaaaaaatttaaaaaattagctgggcatggtggtgcatgcctatagtctcagttacttgggaggctgaggtaaaaggatcgtttgagcccaggaggtggaggttgcagtgagccgggattgtgccactgcactccagcctggacaacaaagggaaatcctgtctcaaaaaaaaaaaaaaaaaaaaaagacactgccctgatgatggcaggaaacgctgcctgtcagctgggcattgaaggaacacgcaggcttcatcaaggacagcggcagaggccactgtgacatacccaagatgtgacacctgacccactttcctggcattacagaagccatcccaagtccaggtcacctgatggccaaggtctataaaataggaccacctaaaagaaatgcacctccatacactgcccaccttagcattacttctagaaccgagagacagtgtgacatgggcctaaaacgtgtgaactgctgtacgtgccaaagtgaagttaactcagtgcaacgtgaagaggctattccataaacctctagttctgagaaagagtcacaccgtgacataggctagaaggaacgcagggttcatcttttactcctggccaaggctatctgggtgggaagcaggcagggaggggtctcaccagcctggaattcctcctggtgtctttgtggcgtccagacaggtggtccagctcagcctgctgcacacacagatactccctgcaaggaaagcaaggaagagtttcaaatggaagaggaagaagaaagggaaaggaagtaggccaaatggacaatgtccccacgtggagagcagacacggggctcagcggggctcagaggcaggtaggaagccaggctccctcacactgggggctgtgtgcccagcaccacacaaaattggttacagggaagttcctagttaattcccccaccaggcccacaagtagcttctactgttattatatgcattttctttctttctttacttatttattgtttgagacaaggtcccccctctctcgcccaggctggagtgcagtggtgtgatcacagctcactgcaacctctacttcctgggttcaaatgattcttatatctcagcctcttgagcagctgggactacaggtgtgtgccaccacacctggctgatttttgtatttttagtagagatggggtttcactatgttggccaggctggtctcgaactccttgacctcaggtgatctgtctgccttggcctccgaaagtgctgggattacaggggtgagctaccgcatccaacctagtatatgcattttaaagatagacaaaccaacccttgaaagcagcaagtaccccagccccggtcacacagcagagaagaggctgaacccacgtctgaaaccacacagtggccttctgagcccaaaatcttcatccccatgttataccccctcccaagctggggcccagctcatggggaggtcatcatggcacacctccctcaaggtcaagggtctaggccgggcgccgtggctgacgcctgtaatcctaacactttgggaggccgaggtgggcggatcacaaggtcaggtaatcgagaccatcctggctaacacagtgaaaccccatctctcctaaaaatacaaaaacttagccgggcatggtggcacacacctgtagtcccagctactcgggaggctgaagcaggagaattgcttgaacctgggaggcagaggttgcagtgagccgagattgcaccactgcaccccacctgggcaacagagcgagacttcattccaaaacaaagaaggtggggggcgtctagatttgggggccaaggcagggagtcccatcttgctggatttgctgagtcatcccaggaaggtcactcacactctctgggctctactgacaccacctcaaatgtggaaaagatagcttctgccagactgctactcactgaggtattgagctctctgggaagaaggggctgccttggccccagacaagtctcaaacatgtggtccttaaaggacacagaatctgtccttcagaaacatgctctatcgtccatgcacatcccagctcccactctgcacaatcccagcttgctgcagcctccacctctacaggattttgccatattcccatgctggtcttgaactcctaggctcaaggaatctgcctgcctcggccccccaaagtgctgggattacagggtagccaccgctgtgcccagccaaggatgtctattttgaaaaccatttcattctaaggtggtttccctttttttttttttttttttttgagtcttgctctgtctcctaggctggagtgcaataacgcgatcttggctcaccacaacccccacctcccgggttcaagtgattctcctgcctcagcctcccgagtagctgggattgcaggcatgccccaccacgccggctaattttgtatttttagtagagacggggtttcaccatgttggccacgctggtctcaaactcctgaattcgtgatccgcctgcctccgcctcccaaagtgctaagattacaggcccactgcgtctggcctggtttcaacttttacttgactctggttcctctttggggtgccccatctataaataagagatgtaggggctgggcgtggtggcttatgcctgtaatcccagcactttgggaagccaaagcaggtggatcacaaggtcaggagttcaagaccatcctgaccaatatggtgaaaccacgttctattaaaaatacaaaaacaaattagccgggcgtggtggcagatgcctgtagtcccagctcctcagaagtctgaggcaggagaatagcttgaacccaggaggcagaggttgcggtgagctgagtgagatcacaccaccgcactccagcctgggtgacagagtgagactccgtctcaaaaaaaaaaaaaaaaaaaaaaaaaaaaaaaggcgtaagtaggagagaatttagggtctaagtaccactatatctggggaaaatcactccagaaaccactctttggcccacagggccccctgtcatctggtccccattgccccataacctcatcctctttcctttggctctagccacgctggcctccttgctgcgccccagggcctttgtgtatgtggctaccctggcctggaatgttcttttccccttcaggagtgtgctccaatgtcaccttcttagcaaggccttccttccaactgcccaacttaaaatgaaccctccctgcctctgcctgcttaccctggcctgttttcctctgtggcccttactaccatctgatattctctgtacttttcttttctgtattgtgtattgtctgtctctcccccactaggagtcagctccacaggaggtagggattttgtgtgtttagtttacttctgtcccctggcctggcactctgtaagcccaataaatatttgtgggctgggcacagtggcttatgtctgtaatcccagcactttgggaggccaaggcaggcagatcacttgagtcaggagttcaagaccagcctggccaacatggcgaaacccagtctctactaaaaatacaaaatattggctgggcgcggtagctcacacctgtaatctcagcactctgggaggccgagatggacagatcacctgaggtcaggagttcgagaccagcctggccaacgtggtgaaaccctatctctactaaaaatacaaaaaattagccaggcgtggtggcaggtgcctgtaatcccagctactcggtaggctgaggcaagagctactctcagcctctgctttctcatctgtaaaataaggaggctatgccaggtgcagtggctcacgcttgaacccaggaggcggaggttgcagtgagctgagatcacaccattgcactccagcctgggcaacaagagcaaaactccgtctcaaaaaaagaaaaaaaaaaattagccaggtgtgatggtgcgtacctgcagtcccagctagttgggaggctgaggcaggagaattgcttgaacccaagaggcagaggttgcagtgagctgagattgtgccactgcacttgagcctgggcaacagagcgagactccatctcaaaaaaaaaaaaaaaaaaaatttgtggagtgactcatccttcttgtgcaggcctcggcccagctcatcagttggtctctgagcaagtctgtccttcactcaaacacccaccgccctgacctcttgcgtgtgtgggctcacagtgcaggctcctactgtggggcctttgcccacactgttgcctgtctgccgaggccctcgacgcactgtctctctgttacctttcttcattgcactcagcacaggtggaagttctatgattgattgcattgcttcttgattgattgcattgaatctgcccctctgcagtgcgtgctccacaagatcagagtcctcctgccttagtcactgccaggtttccagtgcccaaggaccgggctgagcacgcggctgcaccctgacatacttgcttactaaacgaatgaccaggaacttaacctgtcacctcttgtagacaagacccatccacgcttccccaggaagagacagagaggaggcgaggtagaggaatgcacttcttaaaggcagcacacagcccagccttacttgaggcctcttttcaatgcttcgaagatcttcttcacctgctggggcttcgggtctgcacagaccgaccccttccgcagcgtgccgtacatcttggaggattttgcaggcattcgcgatctcacggagttcctgttgatggactttctgtgagaagggttggagggcaagagaagtcagagaagggccctgacaaagccctccccaggggcaggcactttggaaatagtgaccagagccacagggagtcaggagacccggctcagtcccacccccatcaccaccaagcagtgtggtttccagaaagttatggagcctctctgggtctctgctttctcatctgtaaaattaggatcctgggccaggtgcggtggctcacacttgtaatcccagcacttcgggaagctgaggtgggtggatcacctgagatcaggggttcaagaccagtctggccaacatggcgaaaccctgtctctactaaaaatacaaaaattagccggatgtggtggtacgtgcctgtaatcccacttactccggaggctgaggcacaagaatcgcttgaacccgggaggtggaggtttcagtgagccgagattgcatcactgcactccaacctgggtgacagagtgagactcagtcttaaaaataaataaataaataaataaataaataaataaataaataaataaataaaaataaggagcctagatttgaggattaaaagaagagtaataaagcttttccaccatggctgccactggagagcagcagccatggctctgcgctaccctatggccatgggcctcaacaagggccacaaggtgaccaagaacgtgagcaagcccaggcacagccgctgcagtgggtgtctgaccaaatacactgagtttgtgcgggacatgatccgggaggtgtgtggctttgccctgtacaagcagcatgctatggagttactgaaggtctccaaggacaaacaggccctcaagttcatcaaggaaagggtggggacacacatccacgccaagaggaagcaggaggagccgagccaatgtcctggccgccatgaggaaagccactgccaggaaagactgagccccctcccctgccctctcccggaaatatagaacagcttgacaaaaaaaaaaaaaagaacagtaataaaaatctggtatcagaaatgaacttacaggaagaaatacagtcaagtagcccaaatgccaatgctctctgatcaccatgctctgcctgtgcaggcaatgccgtgtgggaggccaagtcatagtcctgtgctttacccttggggcagcatctgttggctttacctgcccagcatccatcccctccctctagtagtagcacctcaattttcctctggggcacctccccagctctgctttttatacttgtggtttgggggaaaggtagcctgactaatcaacatgcacacacacatttgcacatgcacacatgcacacgggattgtttggcaaatccacattccaggcctgcgttagtcaacatattctgctcccctgggccaagaagtatggggatcaagcctggccagtagccagccaggagttcagaattcacagaagggagaagtgttttttcccctggcattgctaacctggggaacatatacctgggacttccagcctcctccttttgccaccatgtagggaaactggggccaacacagagaggaacaaacagagtcagaccaaatctccatgacagtgagttcctggatctagctatgtctaaagctgaacctgcccgtggactttgcagttacatgagccaactggctctcttttttagcttaagccagctggagttgggagtgtggactggatgatcctaaaaactgcctttcagtggtgatggctgggtccctcaacatttagagatgtagcagcatctcaagactgattataggagtacgaggccagggcaccctcatcacagcacagagctggtttccctggcatctaagcctcttctcaggatcccataacttatccatgaggctggctgatgcagcctttgctcaccaacagatgtgttgaattctgctcttagccctctaaagccatcagccaggcgccctggcaccaggcatcacttaatgacaacattctcacaaaagagacatggtggaaatgactcttagatctaactttggcatcagttctctttttttttttttttttgagatggagtctcactgtcacccagcctggagtgcaatggtgcaatctcggctcactgcaacctccacctcctgggttcaagcgattctcctgcctcagcctcccaagtagctgagattacaggcatgcaccaccatacctggataatttttgtatttttagtagagacagggtttcaccatgttgtccaggctggtctcgaactcctgacctcaaatgatccacctgcctcagcctcccaaagtactaggattacaggtgtgagccaccgtgcctggctcagcatcagttattataggggactactggcccttctcttccaactcctcctcttcccaggggcgggaataacggtatgctggaagcagcttcaacccacccctaaaagctggctgaacaacaagagagaaggaacaggccctgaatagcctcgtgaagcagacctcccaacacacacacatgccctcatccaccccttggctctggactgcagagagagagagagaggaataaccaatctcaagtaagcacctgcattttgggggtccttttatcaaagcaacttcacctctacattaacaacacactacccaaggctctgtcttttcactggtaaaatgggaccacgtaatacatcagaggatggtgctgagaattctattagatgccaggcccaaaatgtggcacaaaggagaccttttacatgcaagctgttgttagaatcatcacatcctatctgtatctttcccctggctcacagcttagaaaacattaggtgcaggccaggcactgtggctcacacctataatcccagcacttcgggaggccaaggtgggtggatcaactgaggtcaggagttcaagaccagcctgaccaacctggtgaaaccccgtctctactaaacaaaaaattagccaggcgtggtggtggacacctgtaatcccagctactcgggaggctgaggcaggagaatcacttgaaccgggaggcagaggttgcagtgagccgagatcatgccacttcactccagcctgggcagcaagagcaaaactctgccttaaaaaaaaaaaaaaaaaaagaaaaaaaagaaaatattaggagtttggtaaatattaagctcagctgaacgggggaaaatacagtatcccaaggggattagagaacagagaacctggcccctgcaggcagagcgggatagcgagtgcacccctgggtgttcccagctggagacagagggagcaaaggtggagaatgggacctggtatgctctgggagtggatgcaaggagcaaggtttgacccaggcagaagtgggagtctgcagaggggcctggacctggggcctctctagttttggggacccaggatgtgtgagaggaggagaagggcagctcccagagaaagtccagcttccaacacctttacaatgacaagtaatgtctctgtagagcagagaaactaagctcaggccagggctagtgggctgctgaacagctgacaccacaagcccaagggcctcaggagcctggtgagaacacgtgagcctaagtaactgggggcacctgctgccgccaggagctcgctaagggctttatagaaatatctcgactttcacaaccaccctaggagacaggtattattcttattattattttgagatgaaatttcgctctgtcgacaggctggagtgcagtggcacgatctctgctcactgcaacctccgcctcccaggttcaagcgattctcctgcctcagcctacccagtagctgggattacaggcatctgctaccacgtccagtaatttttgttttttttagtagagacagggcttcaccttgttggccaggctggtcttgaactcctgacctcaggtgatctgcccgccttggcctcccaaagtactgggattgcaggcatgagccactgcacccagccatttgtatatatttaatgttaagtgatgctttccaaagcccacaggggctgtgctccctcttccccttgccctccctgaggccccatcacccacctcttgaaccgggccctccgcaagtttgccatcttgaggctggcagagacggtcagggctgcagactcgggaaaaggcaggtttctgagaggttagggaccccggcaggtgggcagcaggcagtggggcaggagctcgctcactcccagctcctgcctccagcccccaacaggtgtgcaccgttggcccagccccgcttccatccacctggggaccttataccctcgctgctgcagccacacctggatgcacctgctcccgggaaagctctgagcctagtgctccttgtgtgaggtttaacaggacaggctcagtggccactctgagagcccgcccacccggggaaggtgatgcacatgcagcctccagatggccaaatcaggcagcatgtctggcccaggtgtcacagaagccggggcaggaagagcctctggggccggatgtttcaccaggtctgggaggactcagtaaatattaaacagcccctggcatgccagacaagcttccagacgggcacgtgcaacctgccggccccagccctcacgtgaggttgctacagcagtccttctgcctggtgtggttggcagaggccttggtactcggctgttgcaagtgcaggctctggaggcagacagggccgggctcaagtcctgcacctgcccccagcctccaggcgggacaattataggactgactgcacaggcttcaggtgaagactccatgcaacaaggacgtgagccaaccatttacccaggtgcctggcggtgctggccactgggtgatgatgacaggcatgcactcggcacttgccaagctcaaattctgctctcagtgcttttgttttttttttttttaattgagacaaggtcttgctctgtctcccaggctggaatgcagtagcgtgatctcagctcactgtagcctccgcctcctgggctcaagtgatcctcccacctcagcctcctgagtagctgggaccacaggagtgcacaattacactcggctaattttttgtagttttggtggagacagggtttcaccatgttgcccaggctggtcttgaattcctgagctcaagtgatccgcctgccttggcctcccaaagtgctgggattgcaggcgtgagccaccacgcccgcctggcctgctcttggtgctttacatgtatggactcatttctcctgacacagtctttgaggtcaggccccctggtaccgtccaggaaactgaacttgcatcactcgcctctggctccagagtctgtgtgcttgacagcttcacaggcagggctggatatgagccgcctctttctccagcctcctctccttgaaacttaatggctgctatttcgttttcacacccacacttcttagccatcccaacagaggaattcccaagctgaggaggatgtcccagtggctcctggctcggggagtacctgcctgactgcctgggggagggagacctggctgaggaggggcaggaaagggggaagggcaaccatgcctgtcaactggggcagagtaggacagtatcggggcctggccctctcctccttcatcctcactgtttatcctctcatctctctatcctcccaccccccaggtccagctcttgggaaatggccttattatgtcaatcattcacaccttagtataaaatttccccaccaggttacttccccaagtgagccatctgactgtggagtaaaaatccctgtctatagtgaaaggggtttcaaggtgccagagtaggggtcaaggtggtgatgggaagagaaggggcatcagcccaccctctccatgcagagccctgccctcctgccaggttgtttgccttgacagtgcggctgcgcttcggcctgctcgggttaccagggaacaaggccagaaggtggggcctgaaacccaatcaggctccagccctggctccaagggttcagaccccagggagctcgacggagacaggaagttaaaaatagatgcaccgcttccccgtcggtgtgggcagcttcttccttgccctcacacaagggggcggcagaggccaaggccaggtgggggctgcccagggacctccgaaccagcccaggcccagcatcctctggcatccttgacaatcagatgggggacaggaggggtctgagatattagagccatcagggctgtctttcctagactaggacttggacactgtgggacccttgctgacggccagcgagccctggtgtcaaggggatggatcacctctgtcctccttcctcccaaagagctgatcccaggaatccaggaaggggccaacgagaggcagaggcctaccgtgggggcactctctgggcagagctcccctctagggcctgaggggacatgtcccatggatgcaggacattcagaggcccccacaggcctggcaggaggagagctgcaggcagggccagttgtgaaatttgtggggtctagtgcgaaataaaaatgcaaggctctttattcaaaatttatcaagaattttgggccaggcgcggtggctcacgtctgtaatcccagcagtttgggaggccgaggtgggtggatcacctgaagtcaggagttccagaccagcctggccaacatggtgaaaccctgtctctactaaaaattagctgggcatggtggcacatgcctgtgatcccagctactcgggaggctgaggcaggagaatcacttgaacccggcaagtggaggttatggtgagctgagattgtgccattgcactccagcctgggcaacaagagcaaaagtccgtctcaagaaaaaaaaaaaaaaaaatagccagtgtggtggcccatgcctgtagtcccagctactcaggaggctgaggcagaagaattgcttgaacctggaaggtggagcttgcagtgagcctaaattgcaccactgcacacctgggtgacagagcaagactctgtctcaaaacacacacaaaaaaattgtattaagaatttctatttggtgccaggcacagtggctcacacctgtaatcccagcattctgggaggccgaggcaggtggatcacctgaggtcaggagttcgagaccagcctgaccaatatggtgaaaccctgtctctactaaaaatacaaaaaaaaaaaaaaattaactggcatggtgaggcacacctgtaatcccagctactcaggaggctgagacaggagaattgcttgaactcaggaggcggaggttgcagtgagccgagaatatgccactgcactccagcctgggcaacagagcgagactctgtctcaaaagaaaaaaaaaaatacaaaaattagccgggcgtggtggcacgctcctgtaatcccagctactcaggaggctgaggcaggagaatcacttgaacctgggaggcggaggttgcagtgagccaagatcgcgccattgcactccagcccgggcgagagagagaaactgtctcaaaaaaaaaaaaattttgggaggctggggtgggaggatcatttgaaccaaggaggtcaatgctgcagtgaatcgagatcgcaccactgcactacaacctgggcgacagaatgacacccacaaaaatttctagagcacaacagcagagcgttcaatcaaagtacagagcacaggctacacgctcatgaagccgcccttgggtacagggtctgcagaccctacccctccttccagaccacacaagggtccctacagtgcttcagtggaccagccccactccagggcacacagctgggagagggtcacctgggccggatggccccctggccaggtaacccgcacagctgaccttcccagccttgattacagacccctgcaagaaagtgggggactctgataaagcccaggaagaggcagctcagaaatggttaagttgagaaacagcatcatttcctggccggtttatacttaaccccctctttgcagcacttatggagtgcctgctgtgtgcctttctcaggcagcacccactctttctgttctctgaggcttggagttggggcactgagcactaactgctctggagcctgggtcaaattcttctcgtcttggggcatcaacttaaacccttcccaggctcccctccactgagaatgtgtctcaaggcctcactgcagcccatgaggctccgcagggtcctcctccctccctgactgctgtcacgcatgccagccgcacacctgctttctgtcccttaaagctcattcccacccaggacatctgcactcgcagctgcctcccgccgccgaaggcttcccggcccacccccatctgcacacgcgcagatccacttcttctgtcccttcctgcctccactccccatgcccctgtctcgtcaggctctcccaggagaccatgggtgccctcccccacccccagttcagttccctcacagcactgccaccagctggatctgtctcaattatcactggcttattgtttgctgccatcagctcccaggacagcagggcctgggtctgtccccagagcccaggacagggccagagtaggtgctccgtgaatatctgctgcgtgaacagggattcctaaggtgcttccagctgggacactccaggatcttaaccctggggtcccggcaccaccacccatgggaagggagcccccagggaaaggttagtgagctgggagggctgacctcaggggggtggaggtggggtcctatccccgcagcaaatgcccctgggaaggagctcagggagcacacacaggagggacctggcctggtctggagctcaggggtcctcccccaggacctagcaggaagccaatgcctgcaggtgattcaacgggaactggaggtggaggagtggggtaggagctccagcgggaggacacacatgtgctgaggccctgaggccgggaggggagggaggtaggctgctggggccaggtcccaggggcctctagccaggagggcgctgggtttattctaagtaggttgggaatctttgcagggtttcaagtcggggaagagcatggtgggattttattgttgactgactcactgacctactgagttctgcactctgctctggctggggaccccgacctgtccaccccgttggctcagacagacagaggtttggccccttcaggatgtcgataaactgccccagtccccaggcctgcctgctcgtaaatggttccctgggggcttgcacagtgcctggcatgaggccctggagcgagggggcagcaggcctgagctcatcgggctgtggcctccagaagcagaaacaaccttcctcccagggacttagtacctaaagccggaggagacacaggacggggcagcaggcagggcctggcccggccagcacccccagggaactcagccacagggtcaatgctgccccccaggcccatctgtgccaagcctgctccctcatccggtaaccccccacagcaccagctgcaccggctgctggccctgcctccctcgtggttccttcccgacagccccgtgagcagggtgcacccacaattcccattgttcagatgaagacaaggctgaagaggtgaggtcacttgccccaggtcacagagccagaagaggtggccccattgcctagtcttccagacaggaagaacattccttcccaaccccgcccgtggaagacccaaccctgggccagttttgctgtgtgaccttgggccagtggctccacctatctgagcctctggttttttggtttttcttttttggtgtttttgtgggtttttttgagatggcatcttgctctgtcacccaggctggagtgcagtggtgtgatctcggtacactgcaacctccacctcctgggttcaaataatcctcccacctcagctttccaagtagctgggattacaggcatgtgccaccacatccaactaatttttgcatttttagtagagacagggcttcaccgtgttggccagggtggtctccaacttctgacctcaagtgatccgcccgccttggactcccaaagtgctaggattacaggcatgagccaccgtgcccagcccaattttctggtttttcaaaaaaatttttgtagagattgggttgtgctatgttgcccaggctggtcttgagttcctgggctcaagtgatcctcctgcctcggcctcccaaagtgctgggattataggcgtgagtcactgtgcccagccagagcctcagattttttatctgccaagtggacctgctaagctcaggcagatcaacttctggagactttgaaatgataactgttcaggtttcaaggaaatgataggtttatcccaacactaagaaactctgtctctctgtctctctctcaatctcaatctctctctctctctctctctctctctctgtctctctcgggctgcaagccggcctgagagtgggccttcgggtctctggcagcaggaggaggatgaccctgcccttccccagcccattaacaagccccacccctgcaccccgaaacaggacacggggggaacttacgcgatcctccggctctgtgcactgctgaagcctgcgaaggaggcgctccggcccacgacccccacggcccctgtgtccccaggggacaggaaccgcaacctcaccgacatggtggtcacctgcaaggagaggacaggagagtcagcctggcatcaccatccagcgagtgccgtccgcaggccagccaccttccaccaacacctacagacctcacaatggccccagagtgttgtgggggctggtactgtgcccagctcacaggtcagcaagctgaggtcccatgaggttaggggacctgatgggggacagtgatgggaatggaattcgaacccaggtgtctgtactctgcactctgtgctcaggtccttagtgccgggcaatgctgcctcccagagcaaggggacagcacctcctgaacacagccctctcaccccccgagcttctcctctcccctcctccagccctccctgagcacctaacacaggtgaactctgcaaggcaggccaagtccccccatcctcaagggctccagcccagcagggaggcccacctactgccgaacagttacgatacagagtccatgctggagttatgggggtcccagggggaatctgacccagccatgaagaggcgcccaagagctgagtaggaaggctgacgtggggtgaaggggaagggtgtctaaggccgagggaacagcctgggcaaaggccaggatgcatgaggggacttggtgtcatccagaaacagcaagaagctcagtgggctaaagacgaggtcagagaggccggcagggctcaggtcccaagaggcattggtagccccaagcaggggcttggccttacaatggggcaagggtgactgggagccatgggagggctttcagcagggggtggaaaagtccaattcacatgaagccagactgctggtgtcaagggctgcttccaccccgatccattgcccagtctccgacccgccctgcaatggggccaaccccgagccctggctgtcttctcttttttccaaaccatctctagattgagaaaatgccaccctatcccttgggaatataggtcagcatcttccagcaagggcagctggaaagttctgcttcatatctaacctaaatctctgctgctacagtttcatactttgctcctttgccagggtggtgtcaatccaaggattccagggaccccattgcccagacaaaacctacgaaaaagctccagtccacctcatcttcctgcacgatgagacccccatgttccaacatctctttcccccaggggccaagtccaccccactacacctctgctcacttgggggtctctgcctggaaaaacttcctgtatcgagccacatctgcccagcatctactactgcacctcctggcagatctgtgcagcctccacaagcctcctcctcccatatatcccccaccccacagccccacatgtgccgtacataggaaagcttggcaacaaagaaggtggacctgggctcaaatcccaactgtgccactcaggctctgggcctcagtgatcacaagagtaaaaggggaattcagaaagaacctcccacggagactgctggaaggaccaaggaaagcatgtcccgagagcccacagtgtgcaagagacatgtcggaagagctggctgtctctgtgcaattggctaagaccccagctctgcaggtgagccaggtgacccagataagccaggtgacccagggagagccaagtgagaagtgagcggcttctagcccggatggaggaaggtggcaagcatttgctgaccactcgctgtgtggcaagcctggtgccccatgagtggctacgaatccatgaacagctatgaacccatgcccagcagtgctgtgtacagatggggaagttgaggcatcattcatcaagtgctcattactaagctggccctgcactaaatgctttatgtaaattatgaaatccatacaaccctcttaagagcaggagcttttgatgatctaattccatagatgaggaaactgaggctcagagaggtgatgagactagcccaggagccccagcagggcacctgagcactgggtttagaacccgaggatgcccgatgtcagagcttgtcttacctgcggtcaggagagccttgcactggggccttgcccctgcccccataaagaggtcccctacagtcccctcctgtgctgtgctgagccagcttgtaccagctcccaacagtcaactgggccaggcacagtggctcacacctgtaatcctagcactttgggaggccgaagcaggcagattgcctgagctcaggagttcaagaccagcctgggctacatggtgaagccccgtctactaaaatacaaaaaattagccaggcttggtggcaggcgcctgtaatcccagctactcgggaggctgaggcaggagaatcacttgaacctgggaggtggaggttgcagtgagctgagatcatgccactgcactccagcctgggcaacagagcaagactctgactaaaaaaaacaaaaaacaaaaaacaaaaaatagtcaattgttaaatgttcaggaattttgtgagcctgttgacatcacgttggtggtttgaaatcagccacagtaaacgtatttacaccacagaaagcggcaagttctacaagttagggtttctgtctgctggttgttaaacacgtatgagctcctcactgctgttacccctatcagcacctatgcagggcctgagaagctgctcaaactgcttgatccccccagccaagccaggcaagagaataaggacggagtagggagggattcccaaaggtgagtagttgagacgtactccggagccagcctgggcactggagccggaaggggcttccccggcccctccctctgcaccttcccatcagaagccttctgggccgttcctggagcttcaccccagtcactccacttcaaggtcagagagaaggacaattgctaagcagttcctcccgatgcaaagctcaaaacaagccccaggtcctcctgctcagtgtgagagagaggacgacgaaggagggaaactaaggctcggagcagacctgcagaacctgacagcggattcatcactcatagcactgtgaggttcaatggccccatttttcatatgaggaaagaaaggctctgagaggtgagaggcgactcagggacacacatatttctttctttatttccttttccttctttctttcttttcttttttttttttttttttttagacagggtctgactctattgcccaggctggcgtgcagtgacacgatctcagctcactgcaacctccacctgctgggctcaagccatcctcccacatcagcctttcacgtggctgggactacaggcacacaccaccattcccagctaatttttgtattcattgtagagatagggtttcgccatgttgtccaggctgttctcgaactcctgagctcaagcgatccacccacctcagcttcccaaagtgctaggattatagccgtgagccaccgcgcctgaccaggacactcatttttcaaactgaagtctggctcttccttcttccacgtgccatggccacatggattggcttcctggctaaaccctcctcatctttcagcccagatgtcccctcccccaggaagccttcctggacacctccccactcctagctgtgtctggtgtttcctctggatgcccctgacctcctctgttatagtggactgtgaatggtgacttgtctatctctccatcctagacagggagctccctgcaggcagggaccaggtgcatggaagattcataacacatctttattgaaccagaaaacaatgaaggaaaggttgacgctgcttcccctacttcaccaggctgtgcagcccaagaggaaggtgctggaataataacagtaatactaatactaataaccactatgtgctagactctaaggcaaagatggacaaatgttttctgtaaaaagccagatagaaaggctgagcgcagtggctcacacctgtaatcccagtactttgggaggccaaggtgggtggatcacctgaggtcaagaattcaagatcagcctggacaacatggtgaaaccctgtctctactaaaaacacaaaaattagccgggcgtggtggcgcatgcctgtagtcccagctactctggaggctgaggcaggagaatcacctgaatccgggaggcagaggttgcagtgagccgagatcaagccactaaactccagcctggacaacagagctagactccatctcaaaaacaaacaaacaaacaaaaagccagatagtagatatttttggtttttcaggccatacagtttttgtcacaactactcagttctagcattttagagcaaaaccagctgcagacattatgtaaaggaattatgccatggctgtgtttcagtaaaactttatttacaaaagctgcagcagctgttgaggatgatgggtggccctgcctctgacccttgggtagccagcactgcctgctacctgctctgaggacttaaaattctcatttcattctcccaacaccatatgagacaagtactattactatccccattttgcagataaggaaaccgaggctcatgccaggcgtggcgactcacgcctataattccagcactttgagaggctgagatgggcggatcacttgagcccaggagttcgagaccagcctgggtctcatagtgagacccggtccccacaaaaaatacaaaaattacccagatgtggtggcgtgcacctgtagtcgagctactgcggcaggctgaggtgggaggatcattagagacctcccagcaagtcgaggctgcagtgagctgagactgcaccactccagcctggctaacacagtgagaccctgtctcaaaagaaggaaaggaaggaaagaaggaaaggaaaaaggaaaggaagaaaagaaggaaaccgaggctcagagaggacggaaatgagtcctccgaggtcacacaggtaggaaatgccagagctgaaactggaccggggcgcgctggactccaaagcctggcttcgggactccgctgtgcttgctcccgcagctctgctgaggacacgcccccctcccaggagccagacctcctccagcggggaagaggccccagaatggaaataggattgggagggggacctgccaagctagaaagatagccctgggtggtggccgagaaaaatcaagcccaacctcttttggcttcagggtgttgcagccccagcccctgtgggcccccttggggcctgcggaccgcgacagttcccaggcagctcagctgcgccccctcccggctgggcctggtggggctgatccatgacgttgacttggagtccagccaagcagtgtttcttgtggtaaaagaaacagacctccccctggatgattggggatgggatggccaggctgagacccacaatctcaggagccttcagcggacagctcctgacaagtccagtttgtcacctgcgaccaagggtgacattcctgatgtttaagcaatggcacagcagcaaatggaggctgggtgctggagcagggtcttgaagaccctgtcccctcccaccatgtgtcaccacccctgctggggctggcattaaccctttagctactggattgtgggcaggtctaggaggtccctggggaggcatcaggaagagaggaggaatgctcgggtggcttagggcagccctgggtaaccagcactctgcaggcatgagagacagtgcagagaccctgctgggccccagggcagagaagggaggcacagagtcatgcagttcccaaacctttggtggcagacaggacagcctcttctactccgtgtccctcgctgcctctctctggcctggcacttagaactgatgccgttcagacccggctggtgtgtgcagggagaggaagccagatgctcccagacactggggactgtcctgggcctccgtccccaaggtgtggctggaggaagcagagtctactcccgctaagtctgtccgctcactgctggccaaagctgccctgcgtctcctccccaccgccagccagagggaacctgcaatttcacctcatttagaggtaaaacatctaaatttaacgttatgggcttttggggctgggtggcttttatgcctgagtccctcacttagggctcctttttatccactcaaatgccagctagggcttagtttgtttataggagtttccaaaatagctcctttggtttcgcatgaaaggaaatggcaaaatagcccaggaagaggaatgtgagtttacacagaagacagacaggcgcccgaggaggcttctctgggaaccagttcgcctgtaccagagggggcccgagaaagtgtggagtccaacagtccaactcgctcattttaccgatgtcaagactaagcccaggatggtcacacaacttgcctggaccacccaaaggcgactggaagagccagaagaacccaaactactcctccctgccaaagcacaggcctcagctggagcccccctccagcctttgccctggctgtgtcctctgcctggcacacgcctcccttcccccaggtcttccctatcactctctcccagcttgcaggcctcacaaccaaggccacctcctcagagaggtcctccctgacccccttggctaacgtggacttgcccctcacccgtatccttcaaataactcatggttctgattgtcttattcatcagttaatcaagtatgcttctcaagaatcttcgctccagggagcagggggtttttctgtctttttaaaaaaaagtttttggggccaggcacactcctataattatgggctcacgcctgtaatcccagcactttgggaggctgagtctggtggatcacctgaggtcaggagttcaagatcagcctggccaacatggtgaaaccccatctgtactaaaaatacaaaaattagccaggcgtagtggcgggctcctgtaatcccagctactcaggaggctgaagcgggagaattgcttgaacccaggaagcggaggttgcagttagctgagatcacaccgttgcactccagcctgggtgataaagcaagacttcgtctcaaaaaaaaatttttttaaataaataaatacataaattattattattattatttttatagagatgggtcttgttatgtcacccaggctggtctcaaactcctggcctcaagcgatccacatggtgtgagccaccatgcccagcctgctggtttttaaagagcatatttaaatgaaaagagacaaatttaaaggacccttggtttaaatagagcaggttggaaccagcttcagggcagcccatggtcctggctctgccatcctccagaaccacctggagccaggaggggacacccaaggtgtctctgcagaggacagcggcctgacggatagacacacaatgagtgccctgatttgtgatttaagagaagaacaagcagctccttgggaagccccagtgtcccctgcgctccactgtcccaggactgcaggcaaagggacgcctcctgaccgcagaatagtcaacagcaggcacgggagtgaggaccgggatccagggaggccgcttccctctgtctatcagtctgcagccctgggtcccagctcactccattggggttttcccagataaagatgactcatgaatttctttgaattatccagagggcattttaattcaaatggccccatcactgcctcgtcaccactcccaccagccatgccaggggtcagccaggggtcaccttgaagacaaagcctctctttgggaaagaagcctttgaggccactgtggggtggctgtgtgtcggggcgccaggtgggaaggcagctggggcctgcccaggctagggaagagagcgtgggggtgtgggggatgaaggaagataaagatggggtagcgggagggagcgggggaagctgggcctccaagaagcacaggctgacttgggaatcccatctcaaacgtgcctcaccctggtctccagctgtaggagcagtgataaggatgaggacaggcaggagggactgagatgagatgagagcatttggagcctggagagagacccctgacactgagggagtgaggtgacctggtggtggctattccaggccaagcacctttgttcaactgtcaaaaactagaaacaggccgggcgcagtggctcatgcctataatcccagcactttgggaggccaaggtgggtggataacttgaggtcaggagtttgaaaccagcctggtcaacatggtgaaaccccatctctactaaaaacacagaaattagccaggtgtcgtggtgcatgcctgtaatcccagctactcgggaagctgaggcaggagaattgcttgaacccaggtggcagaggttgcagtgagccaagattatgccactgcactctagcctgggtgacagagcgagactccaactcaaaaaaaaaaaacctgaaaacagcaataatgccagtggtacccaacacggctacaggacttgtgcagtgccaggcattgctcaaagaattcactcattgaattcctggtacccaggaagtctggcctcaagcctgagctctgcccccgctgagcagaacccctctttctaaaccccggacactgggctggacacacagcctctcactcacccccacagctccccgggggcagggcactgttaccattgtacagaccaggaggctgaggctcgagatggagccacctgaagtggcaagttggtagcattgtacctccaatgactcaccaaaacgcctgcataaaaatccaggtgcagtggcactcacctgtagtcccagctacctgggaggctgaggcaggaggagcacttgagcccaggaagtttgaggccagcttgggcaacaccatgagccaaaaataaataaatacaatacaggtaaagtgcttgtgtataggcagtgtggtgcaggtgtgtgtacgcctgacctaggtcccaggagtgactaagggacacgcagagtggggctctccaatcagaagccctcactctggaattggttatgggagggtccctggacctcagcatgtaatgctttgctgtggaggctgtcctgtgtgttcagcagtgtccccacaccaggagcacagcctgtgacagcctggcacatctccagacactgccacacgtccctgtgctaaaggcatctcttcggacctgggctggagggttcattaacccgtatggtggcttaggcataattttccatgggcgaaatgagtagtattgaagacactatggtatttggttggctacagtattgctcgggcaccttcccctcacacgaacaggtgtgggactctggcagaccacacagcctgacacctacggagcggccctgggtctgcggctcccacagggcctctgccactccagccgtgacacacacacatgctgtcaccagcctaggatgtgcacaaagcctgtaggtctgagaccaaggacatgagcttggaggagctggctggattcctcagacctctggggcccgtttgccagagcagagccgagacctgcctgagacctgccttcattgcaaccctggccctgtggcacggctgcaagggaggagagaccgggtgaagggctgcttcccctttccatccaaaaacacacaaacaaaagacgcctgagcttggtgaacacacgcactggtcaggcttagctccatgcggggaggatgtaaattcaaacccaggtgggctgaactccaaagcactcttcggccaaccactggtcactggaatgaactgcccccaaccctctgtcatctcggggacacagaccctgcccccctccgcagggctggacagcagagcagcttccctctacaaaacggtcaaaaaggcaaagaaagacttccacaccctgccgctgcctgggagaaccctgagcttcctttctgcagtgacctctccattagacgcacaggcccacgcatgcgcccacgaacacatgtgaattacttctaggatcagaaggcaaaaaaatgttctttaggtcaaagaaaatgtgttattatataagagtaatgtattcattgttatagcaagttgtaatatgcacttcttttttttttttttttttgagacggagctttgctctttttgcccaggctggagtgcaatggcgcaatctcggctcaccgcaacatctgcctcctgggttcaagcaattctcctgcctcagcctcctgagtagctgggattacaggcatgcaccaccacgcctgggtaattttttgtatttttagtagagacagattttctccatgttggtcaggctggtctcaaactcctgacctcaggtaatccgcctgcctcggcctcccaaagtgctgggattacaggcgtgagccaccacgtccggctcacttctttttttttctgagagagagacagggctcactctatcacccaggctggagtgcagtggtgggatcacagctcactgcagccttgacctcctgggttcaagcgatcctcccatctccgcctcctgagtagctgggactacaggtatgtgccactgcacccggctaatttttttaaaatttctttagagacagggtctctctatgtggcccaggccagggtgcagtggtgtgatcatagctcactgcagcctccaactcctggactgaagcagtcctcccacctcagcctcatgagtagctgggactacaggtgtgagccccacacctggtcctatttcttattggaagggccactgcaggccaagatgctcggtgcccagggaagtcatagcacggccctgcccttgaccccttccagccatcctgggaatctgtccccagccacctccaggccacaggctccttccccatctcttccccacccaggagctgtgttacacagatcactttagcgtttcccacgctaaaccactgaaactgagggttttatctcccactctccttgctcccagttccgcatggccactggcctggctgagacagagccgctggaagtgcccggcaggacctctcacttgaccttcagcctcttacttaaccctcccaatgcagggcggtagcctccactgggtcctgttcagcccctactcctagaaatggtctggggggtaagggtggggccccctgcagagcccttggatgtgtgtttgcgccatggcagtagtacacccacccccttctgcttccgcagaacctggccggctcctccccatcccagggggagaggctcctcctgctcccacttccctcctttacatttggctactactcttaggggtggggctttgtttactttgcattttcctgattcctaatgagattaagcacatttccttaggtttaccggcttcccaatctcctcttctgggaattttcagtgcctattcctttgtccagttatctgccttcttcttcttgattgctgggagcggctgataaatcctagatgaccatcctttctcagttttctcccagcccctcaccttttaatcttaaattgttctctgtgggtgtgtgtgtgtgtgtgtgtgtgtgtgtgtgtgagagagagagaaacagggagagagatacaagcttcaaatacttatgtggccaaatccgtcacaggttttccttctgtgttttacaatttggaaactgctttctggtctctaagattaagttgctaatctgttaaccactggtgtttaattccctgggggagaagcttgtcttcccagccaggctgcaaagtcccccagggtatgtctccatctcccccctccccccatgaggccttagtcaagagattccactgctcctcagaaataccgtgaatttgtcccctccttcctgcctccacccctgctcgccaggaccaatctctgcctccctctcaccctctgccccacatgattaccacagtgatttaaaaaaaaaaaatccatcagaggccaggtgctgtggctcacgcctgtaaccccagcactttgggaggtcaaagcgggcagatcacctgaggtcaggagttcgagaccagcctgaccaacatggagaaaccctgtctctactaaaaatacaaaattagccgggcgtagtggcacatgcctgtaatcccagctactcaggaggctgaggcaggagaattgcttgaacccgggaggcagaggttgcggtgagccgagattgcaccattgcactccagactgggcaacaagaccgaaactccatctcaaaaaaaaaacccaaaaagaatagaaaaatgtccatcagggctggatgcagtggctcaagcctgtaatctcagcacttttttttttttttttgagatggagtctcgctctgtcgcccaggcggaagtgcagtggctggatcttggctcactgcaacctttgcctccaggctccagtgattctcccgcctcagcctcccaagtagctaggactacaagtgtgcaccactaggccaggctaatttttgtgtgtgttttttttttttttttagtagagttggggtttcaccatcttggccaggctggtctcaaactcctgacctcaagtgatcctcccgccttggcctcccaaagtgtggggattacagctgtgagccaccatgtctggccaaatcccaccactttgggaggccgaggtgggaagaccgcttaaagccaggagtttgagaccagcctgggcaacacagagagacccatctctacaagaagtgaaaaaaaaagttgggcacatgttctcaggaccccctgaggctatgtcttgggtcatggtcactcataaataaattgttttaattaaaaataaatgttgaaaaattagctgagcatggtggtatgtacttgtagtcccagctaaggcaggaggatttctttctttctttctttttttttttttttttgagatggagcctcactctgtcgcccaggctggagtgcagtggtgcaatctcggctcactgcaagctccgcctcccgggttcacgccattctgccattctcctgcctcagcctcccaagtagctgggactacaggcgcccgccactgtgcccggctaattttttgtatttttagtagagatggggtttcaccgtgttagccaggatggtctcgatctcctgacctcgtgatccgcccacctcagcctcccaaggtgctgggattacaggcgtgagccaccgcacccagctgggaggctaaggcaggaggatttcttgagcccaggagttcaaggctgcagtgagccaggattgtgccactgcactccagcctgggcaacagaatgagaccacatctctaaaaaaatttaaaaataaataaaaaataaaaatgtcccttagtttgtcactatattgcccaggctggtttggaactcctggcctcatgtaatcctcccagctcagcctcccaaagcgccgggattacaggcataagccactgcacctgaccccaaccgaaaattcttaaggcacatttttgacactaaaaacagtattttataactgctaaaatagatatgttaattctagtcttttcttgtgcacagaaacaaattaccactttagtttctcaaggagcacatgtatacatgttaaatgcattaatttgtttaatataaacatgaaatactttttataacctggattggtacatatttcttttcttttcttttttttctttgaggcaagatctggccctatcaccaggctggagtgcagtggtgcaatcttggctcactgctacctctgcctcccgggctcaagcaatcctcccacctcagtctcctgagtagctgggactacaggcatgtgccaccatgaccggctaatttctgttttgtttttttttgtttgcttttttttttttttttttttttttttgtagagacggagtttcaccatgttgcccagactattctcaaactcctgggctcaagcgatcctcaacctcaacctcccgaagtgttgggattccaggtgtgagccactgcacccagcccttttatttattttttattttttattttttttttttgagacggagtttcgctcttgtcacccagactggagtgcaggggcgcaatcttggctcattgcaacctccacctccgtggttcaagcagttccccggcctcagcctcccgagtagctgagattacaagcacacgccaccaagcccagctaatttttttgtatttttagtagagacagggtttcatcatgttgaccagcctggtctcaaactcctgacctcaggcaatccgcctgcctcagccttccaaagtgctgggattacaggcatgagccactgcgcctggcccccatttcttattatacagtagtttacaaaaaaatcccagcagccagctccagagaggccttgttctgtggtgtctaaggatggagcccaggcagggacggccaaaagctcgctacccctgcccaggaaggcaggagcaccgttgtgtcccgttcctatccctcaaaaataaatcacagccagctcatgtcataggacagagcctgttcgcaatccatcctgtgtctgcggattctcccaggtctgtaaggcagcagggagatgcggcctctcccactccacccaacacgtagccagggcgaggtggggccgggggagaggctgacattcaaaggcatctgagtggtaagaggtgagcgagtgaggtgaatggggactacgttagaaggaccctacgttagaagggtgaggcgctagggccatagcctaagggcactgggaaccctgtgggcatgcgcagttcaagcccatccccgctccctccagctgctgtccatccctgccacacctgaccatttgcctaacctagatccttcctgtcttgcatttcctcaagcatccggagcccaggactgctgagtcaaccctctggaatgcccacaactccccacaggccagccggccttgggactcccgcacagccacgtgagccggtggagccgggtctgtttgctagtggaggctgttaacagcacgggaagtggtcaagggttcaacaagagatgagccatctggtcctccagaggtaaacaatttacaagagacacatcaagccggcctgctgttctggtttttcttttgacagtgaaatatgcagtttctttttcatcctggtgcctattggagagggagactgttccaggcactctgaccccagctaaagcgcctccctggggcaggatctatgcagggaggcagaaaagtcagattttttttcacatcttctttgttccattcccaggactgagcaacttcatgtatttatgtatttatttatttatttatagacaggttctcactatgtcgcccaggctggagtgcagtggtgcggtcacagctcactgcaacctcaatgtcctaggctccagtgatcctcctgcctcagcctcctgagtagccgggaccataggtgtgtaccaccatgccaggggaatttttgtatctttggttagagaaagggttttgctgtgatgttgcccagtctggtctcaaactcctgagctcaagcgattcaccctcctgggcctcccaaagtgctgggattacaggtgggagccactgtgcgtggcccaggactgagcaactttaagtcagatggttaacctacatcatgaggaaagtggatttcctcccaaaggaacagacttattttctagaacccaaagccttgaatttcaagaacctttagccttaaatccatttcctgttggaagcagaccccctcctggtctccccaggtattgcaaccctgctctaccagccactataaatgcccacacaaaaggaacaggggctccattcctgatgctcagagtggagtcactagattctcaagcatatgagatatgtatgtggcctaacagcctgaaataaacagcttgtgctgggattgtaattctagagtttccaaaagtgttgcaaaatattccaggcagacactaaagtttgtgtactacaagctgtttattaattaatttcttatttgccaaacattcttgcttgggtctactgggagatgatttcgttgggaatgcatctccaattttgtaataaagatcaccagggaaagaaggcttgcttcataggggctcatattacaggaaatgtggctagcataggtagttcccataagaaaaaggacagtactaagttttgagctcatgtgaaaaagaaaagggggccgggcgtggtggctcacacctgtaatccgcactttcggaggccgaggcggacggatcacttgaggtcaggagttcaagaccagcttggccaacatggcaaaaccctgtctctattaaaaatacaaaaattagccaggcgtggtggcggacgcctgtaatcccagctacttgggaggctgaggcaggagaatcacttgaacccgggaggcggaggttgtagtgagccaggatcgcatcactgtactccagcctaggcgacaagagcgaaactccatctcaaaagaaaaaaaagaaaagtgacgtctggggaccaggattttgggactctttgcagacatgccaataacctgtgagatacacacccacagactgacacagaggtgagcagaggccttggagtcaacagaagttcaagtctagcctttgcctcattctcttttttttttttttttttttttgagatggagtcttgctctgtcacccaggctggagtgcagtggcgcaatctcggctcactgcaagctccgcctcccgggttcacgccattctcctgcctcagcctcccaagtagctgggactacaggcgcccgctaccatgcccggctaattttttgtatttttagtagagacagggtttcaccgtgttagccaggatggtctcgatctcctgacctcgtgatccacccgcctcggcctcccaaagtgctgggattacaggcatgagccaccgcacccggccacctcattctcactatgtgaccttggacaagtcacttatcctctttgagcctccagttcctcatctatacattggaagccactgaaattatcttacagttactttcagttgcgaggacttgctctttttctcttcattaaaaggaaaacacaaaatataatttatagcttatcctaccactttgtgctgctaaggttaaaatccagggtagtggccaggcgcggtggctcatgcctacaatcctagcattttgggaggccaagacgggtggatcacctgaggtcaggagttttagaccagcctcaccaacatggagaaaccccatctctactaaaaatataaaaattagctgggtgtgttggtgggcacctgtaatcccagctactcgggagactgaggcaggagaattgcctgaacctgggaggcagaggttgcagtgagctgagatcatgccactgcactccagcctgggtgacaatagcaacactccatctctaaaaaaaagaaagaaaaaaatccagggtactctatggtttcctggggctgctgtaaaaaaaaaaaaaaatactacaaactgggtggcttcaaacaagggaaacttattgtcttgcagttctggaggttagaagtccaaatcaaggtgtcggcagggccatgctccctctaaagcagcggtccccaacctttttggcaccagggacaagttttgtggaagacaatttttccactgaccagggtgggggttgggtgggtggttttggggtgattcaagtgcattacattcattgtgcactttatttctactactacattgtaatatataatgaaataattatacaactcacattccataatgtggaatcagtgggagccctgagcttgttttcctgcaactagatggtcccatctgggggtgatggagacagtgacagatcatcaggcattagattctcataaggagcatgcagccgagatccctcgcatgcgtagttcacaatagggttcgtgctcctgtgagaattgaacgcctccactgatcagacaggaggtaacacaagcaatggggagtggttctaaatacagatgaagcttcgcttacctgcctgctgctaacctcctgctatgtggcccagttcctaacagaccatggacaagtacaagtccatggcccaggggttgggaacccttgctctaaaggatctaggaaagaatccttccttgcctcttcctagcttctggtgatggctaccaatccttggcatttctggtcttgtagctgcatgacgccaatctctgcttctgttgtcccatggtgcttcccagtgtctctgtcttcacagctcttcctcttcttataaggatatgacggtattgaattagaggcagggcacaatggctcacacctgtaatcccaacactttgggaggccaagatggacagatcacctgaggttaggagttggagaccagcctggccaacatggcgaaaccccgtctctactaaaaatacaaacattagccaggggtggtggtgggtacctgtaatcccagctactcgggaggctgaggcaggagaatcgctcaaacccaggaggcagagtttgtagtgggctgagatcatgccattgcactccaacctggctacagagcaagccatctcaaaaaaaagaaaagatgtaggcccctgggaaggatgcctggcacactcagaggtgggacaatggtgacagtcctgacactttccctctggagagcatggacccctggtcttggccatgcagccaggcctggcccagcctgcctgtcccccacccccacccacagggactgggcccttcctgcccaggaactattctgattctgcagccctcaggctttattgcttttcctgcttaatgaggtgaggttcgctcagcccagtggcaattcctgtaaaagccatttggatgacccaatggggtgagtttacagggaagcgctccctgagggctaggctggctggaagcagatactttgcccccttccaaggacggtaatgaggtttgaggcttccaatgggggcaggggatgatctgggcgccaaaagccctggcctggggtcaaaagatctgggctccagccccagccgggctaactaggcagtctcctgtctgagctgtactcccctcatgggtgaaatggggatacaggcacatactttatagcctgcagtctgtgtgtaacagagaaccataagctggtgggtcggggagtggggctgggggttatttctcatggttctggaagcgaggagttggagatcagggtgactgcatggtaaggttctggtgagggccctcttccaggttgcagactatggtcctctcatttcatccccacgtggagggaagaggcaagagaacactctggaagttataagagcactaatcccattaatcccattcaccaggcctccaccctcgagatctattcacctcccaaaggccccacctcctaatcacagcacgggggtttgggttcaacataggaatttttttttgagatggcatctcacacagtgttgccaggctgtagtgcagtggcgcaatctctgctcactgcaacctccacctcccgggctcaaacaatcctcccacctcagcctcctgagtagctgggaccacaggtgtgcaccaccacacctggctaactttttgtacttttagtagagacgagatttcaccgtgttgctcagattggtctcaaactcctgagcttaagtgatttgcccacgttggcctcccaaagtgttgggattacagatgtgagacaccacatccaacctcaacatatgaattttagagtgacccaaacattcagtccatcacagtctcctgcaggattacagatgtataatcagtctgtaatccacaaagttctaagcaaaggaaaagaagtaccaatatcactattgctattgttattatcagcacccaagaccttccccagtctcagtgagtggaatattaagatgatcccaaagacgctcgacttctctaacccgtaatactcacaggttccacccctttgattgtgggcagaacctgtgaatattatgatctgactgccatggttaccttatatgacaaaagggagattagcctgggtgggcctgacctaatcagagagccctcaaacaggactggctttttgttttttgttttttttttgagaccgagtctcgctctgtcacctagactggagtgcagtggcatgatctcagctcactgcaacctctgcctcccgggttcaagcagttctcctgcctcagcctcctgttggctcttactggccaaggagattcaaagtatgagagggattcagcgtgagagagattttccattgcaggctttggaggtgaaggtggccaatacaggaggcctctagaagctgaaagcagccccccagtgacagctaggaaggaaatggggacctcagtcctatggccacacggaactgaatgaccttggaagtggatttgtccccagatcctccagacaagaactcagccggaccaacatcttgcatttcagccttgcgatgagcagagaactcagccatgccagactctggaccaacacagctgtgagctcatacatggatattgttttaagctgctgggtctgggatcatttgttacacagcaatagaaaaccaatacatactctgtcaaggaagcctgagaatggaaggcccctactcaatctaccctcccactatagtctggtggttagagacaagggctctggagtcagatggaacggtgttcgaatcttggcaattccatccactggctgtggaacgttgtccacactcccttcctcacctcttatatcctcggtttcctcatctgtgaaatagcagtggaaataaaatgcatggaaatcatcagagcagggtatgcatcgtaaggacacatagtaatagctcaagaaacactgtatatgttaaacattagaaacgagctgagaactaacaccaatgcacctgtgttcttcaatgcactgcctacacaccagagaagagggggaggagcccagatctgctgctctgggagctacaggccaattagggaatcaggaacctgaacagaaaaccacaacttcagggaaagtagatggtacatgtgttatgttattgtggacatggcggggcaaagacaagaccttaggtctttgaactccccctcggagtctggcacaggcctgagtggagctccctcctcaagacttcctttgcactggcttccctgaggaagcatttgcatttagggttctgctgtggaacctctttctcttgatctacatgaagcctgaggccaaggccttacacctgtaagggaggaggtggccctgggcccaggaaaagggtgggtccagtctcccagttctgtccctggcatgtactctccccaggcctatcccacccccaagtcctcccaggctccaaaccctgaggccccaccgactgtcactcaagaaatcaccgaggctgggcatggtggttcatgcctataatcccagcactttgggaggctggggcaggtgaatcacttgaggtcaggaattcaagaccagcctggccaacatggtgaaaccccatctctactgaaaatacacaaattagccaggcgtggtggcgggcgcctgtaatcccagctacttgggaggctgagacaatagaatcgcttgagcctgggaggtggaggttgcagtgagccgagattgcaacacggcactccagcctgggcgacagagagagattctgtctcaaaaaaaaaaaaaaaaaaaaaaagaaaagaaaagaaaagaaagaaagaaaagaaaagaaaaaagaaatcaccaagccctctctatacccataagcacagccttgaccgaagtcctgacggctgagcatgcagctcaaatccctggggagataccactgcacatacacgagggcggtcgtcatccaaaagacagaccacagcaagcgctggagaggatgtggggcaactgaaaccctggtacgctactggtgacatgtaagagggcgcagctgctttggaaaacagcttggcagttcctcaaatggttaagtatgaaattaagcaaatggttaaatgtaaatacgacccagcagttccactcctaagtatacatccaagaggaatgttaacatatgtccacgcaaaacccatacacagacattcagagcagcattactcatagtagccaagaagtaaaaacagctcaaatatttatcagctgatgagtgggtaaacaaaatgtgggacagccatacaaagggacactatagggccatataaaagggaccaagccctgatgcatgctgtgagacagatgaaccttgaaaacggtatgctaagtgaaagaagccagaaccagcaggccacagaaggtatggctccacggacaagcaagtccagagcaggcaaatccagagagacagaaagtggatcagtcattgccagaagttgagggaggggagaatgggagtcattactaataggtatggggtttctttctggggtaaaactgctctggatttagatagtggtgatggttgcacagctttgtgaatatgccaaaaaaaagaaaaaaacactaaattgtatacttttttttttttttgagacagcctccctctattacccaggctggagtgcagtggcacaatcttggctcactgcaacctctgcctcctgggttcaagcgattttcctgcctcagcctcctgagattacaggcgtatgccaccacacttggctaatttttttttttttcgagacagggtctccctctgttgtccatgctagagtgcagtggtgccatcttggctcactgaaacctctgcctcccgggtttgagcaattctcccacctcagcctcccaagtaggtgggactacaggcatgcaccacccacttggctactttttgaatttttttgtagagacagggttttgccatgttgcccaggctggtctcaaactcctggagtcaaacaatccatccgcctcggatatttctcactggcataatcagtgctgaaagatcattgaaaaggggcttgcatgatccagaggtcactgttatttaatgcgctctcctcatctcctgcaggttgtgacttcaatataccctctcatgagacctttcctggccccttatctgtaagtgtgaccaccgggaccccgtctcccaattccctcttcctgctttatttttctctttagcatttaataccatctgacattccaaacattaccttgtccttgtctggtgtttgtcaccccaactagaagtgctagggggcaagtactaggaagcagggttttttctttttctttcctttttttttgagatgaaatttctctcttgtctcccaggctggagtacagtggcgcaatatcggttcactgcaacctccacctcccgggttcaagtgattctcctgcctcagcctcccaagtagttggcactacaggtgcctgccatcatgcccaactaatttttgtatttttagtagagatggggtttcactccgttggccaggctggtctcgaactcctgacctcatgactctcccaggaggcagagttttttttttttttttttttttttttgagatggaggcttgctctgtcacccaggctggagtgcagtggcgcaatctcggctcactgcaagctcccaggaggcagagtttttgtctgcgtggttcactgctaagtccccagcactaagaggacaggacctaacacataggaggcactcagcaaacatccatgaacgaatgaatgaatctgtatctccagtggctttgtcctctcttatatcagctttgttccacatcttacacttaggaggtgattcaattaaacattgctttttgtgtgtgtttggtggggtttttttgtttttttgtctttttgttttttgagacagcatctcaatcttttgcctaggctggtgtacagtggtgccatcaccactcactgcagcctcaacttcccaaactcaggtgattctcccacctcaactccccgagtagctgggactacaggcacgcaccaccacaccagctactttttgtatttttagtagagatggggtttcaccatgttgccatggctggtctcaaactcctggcctcaagccatccatttgcccaactaggccccccagagtgctgggattatgggcgtgagccactgcacccggccaaccattgttgagtgagcaagtaaatgaactcatcaacgaatttctccctgggtagcgaagctcccgctttttgaggtaacccagacttttcatttgtatgtttcctgagtccctgactgtacattcttcctgcatattgtctccaggactaagagaatggagccttttgccaagaagaggttgcgttctcaggcctccagtttagattaaatagtggttttacaaggggaaacaaagtgacaaacccccagaagttgtggggagctgagacggggtgggaatagggttccccagggacaaactccttcttgtccccctgcccagcgccccagggcacactgcccagtgggtgaaagcagccagacacacgggctcactgcagttaccaagcaatcctaagtgtatcaagatgggcgcgctgacgacagctgcaccgagtgaatctggaatgaattggggggttgttaagatcgccgacttaagcctcttctcacaggaaaaatatttatttcactgagctctgctttttggaaagctgcgcctgtaacagtctgcatcgatctagcagagttttggctactgctgggttttgtctccctcctgtctcggttagcgtgggctgtgtgtgtgtgtgtgtgtgtgtgtgtgtgtgtgtccacagcacaccctcccattattaagcaatcagccaaaagactcagccaggacactctgctcttctttaaccagaaaaaagatgaacacgaactgagttcgtggcatgaggcacgagaccttcatgttttatcttcaaagatgcctgcttgaaactagcaattgtttctttccctttttatctagtaaattgctccctctacctcctcccagtttgggaaaaaaatacaagtatgaagaagaaagtaaaaatcactcctatggttaggttaggcttgactgcagaattgtttcctttttttttctttttttctttttttttgagacggagtctcactgttgcccaggctggagtgcagtagcatgcgatcttggctcactgcaacctccacctcctgggttcaagcgattctcctgcctcagcctcctgagcagctgggactacaggcccgcatcaccatgcccggctaatttttgtatttttagtagagacggggtttcaccatgttggccaggctgtcctttccttttctactctttggagtcgaggggcctgggtttgaatcctagctctgccacttgctggctgtgtaaccctgagaatcatgtcaccacttggaacctctgtttcctcatctgtcaaatggtgtaataatagtgcctgcctctttgattgttgggaggagtaaacaagagaaagtatatatagagcttagtgaaggttcagcaacaataaatgcaaatggtacctattttataggagaaatatatacaattttttatcttctttttcataaaacagtatagcgtatacagtttcctgtttcactgcctcattttagaggcagagtcttgctgtgttgcccaggctggcctcaaactcctgtctgtgctcaaatgatcctcctgcctcagcctcctgagtagctgggattacaggcacccaccaccatgcccggctaatttttgtacttttagtagagacggggtttcatcgtgttggtcaggctggtctcgaactcctgacctcatgatccgcccacctcggcctcccaaagtgctgggattacaggcgtgagctaccgtgcccggccaactcacaagcatttctaatgactgcaaaatagtccattgggggcccatccctacattagtttttaaaccgtttcctatggatggaaatagaagttgcttccgaaatattataaagattgctgtgataaacacctccagactcaaatcttggtctatgtttgaggctctgtctttaggatgaattctcagaagttaaattgttggtcaaatagtatgatcacttccaagagccttgatataccatacttagcactgcaagttgctttcgagaaagattacctacagatgtgtatgagagaaccctgtggccaagcgaggtggctcatgcctatagtcccaacactttgggaggccaaggtgggaggatcacttgagcccaggagttcaagaccagccttggcaacatagtgagaccccatctctccacacacacaaagaaagagagaggctgggcatggtggttcacacctataatcctagcactttaggagacctaggcaggtggatcacttgaggtcaggagttcgagaccagcctggccaacatggcaaaacctcatctctactacagatacaaaaattagccaggcctggtggcatgtgcctgtaatcccagctacttgggaggctgaggcaggagaattgcttgaacctgggaggtggaggttgcagtgagctgagatcacaccactgcactttagtctgggcaacagagtgagactcttctcagaaaaaaaaagagagaaagagagagagaacccatcttaccacatcccagctgcattgaggattattaccttttagttgttgttaatttgacaggcaaaatgacatctcattcttaaatctgcattaaaaaaatttcacccagtatttttctaatactagttttattggctacaatgtgttgagtgtttactacatgccaagcatagggtaaatgtcttttacttttttttttttttttttttttgagagacagggtcttgcctgttgcccaggctggagtgcagtgatgcgatcatacctcactacagccttgaactcctgggctcaagggatctttctgcatcagcctctctcttaagtagctaggactccaggtgcaccaccatgcctggctaatttttttttatttttatttttttgtagtaacagggcctcggtatattgcccaggctggtctcaaactcctgggctcaagtgctcctcctacctcggcctcccaaagtactgggattacaggtgtgagacaccatacccagcctccataaaatgttttaatcaaacttctgggtatgtacccaaaagaagcgaaagcagggactctaacaaatatttgtacatctatattcatagccacattattcatcatagcaaaaaggtgcaagagacccaaatgtccattgacagatgaatggataaacaaaatgtggtgtgtatatatgcaatgagatattattcagccttaaaaaggaagaaaatgttgacccatacgacaacatggatgaaccatgaagacattatgataagaaaatatgcccatcacacaaggacaagtgatgtatgatccttttatatgaggttcctagaatactcaaattcagagacagaaatcagaatggttggactgagcgaggtggctcacacctgtaatcccagcactttgggagaccacggtgggaggatcgctcgagcccaggagtttgagaccagcctggacaacagagtgagatcctatctctacaaaataaaactttttaaaaattagccaggtgtggtggtatatgcctactactcaggaggctgaggtgggaggattgattagactcaagagatcaaggttacagtgagctgtaattgccccactgcactgcagcctgggaaacagagcgagattctgtctcaaaaaaaaaaaattataatcaatctttacatgttattttataagatgttattattaccctccagccttctttaaaagatgatgaaactaaggctcagagagatgaaacacgttcctagagtcacatagcttataaatggtgaagacaggtttagatgccaggtcttactctccaaggctacgttatcctgcaaattctggtggacctgggaggtaaaggggaaatacaatcaagctctaggtggcagatggagttagcaagtacccagtgccaaacggaactgttggccctgagagctcagagttcagttctaaatttattctctctgaccttattgtggattctaaatttggccaaacaagttcttccagaccgattagtgacgtggatacattgttttgaagataaaacccttttcctggtatgaaagaaaaaaaataataatgcagcatttgcttgcagaagccaccctgccgctggttaacagccatctgaaaatgattctgggttttccacctgatggtggacacatgtgcttgttggccgatttcttggggacagtgattaaaaccatttcctgaaccgaagtctgaatttctcctcctgaatgaaccagaaacaaaacatcaggaaatatccaggcatgaaaattctaaccagggccgggtgcggtggctcacaactgtaatcccagcactttgggaggctgaagggggtggatcacctgaggtcaggagtttgagaccagactggttaacatgacaaaaccccatctctactaaaaatacaaaaattagccaggcgtggtggcaggcacctgtaatcccagctactcgggaggctgaggcggaagaatcacttgaaccctggaagcagaggttgcagtgagccgagatcacaccattgcactccagcctaggtgacaagagcgaaactccgtctcaaaaaaaaaaaaaaaaaaaaattctaaccagaaccaaacttaaaattctcccaaaattacaaattaagaccaaaataggcctctttgggtggtttccaggccagggaggagttatggggtcctcggataatcaccagctgagtcctgacttctctgggaaggtattggggtgactgacctgtttgtgaccccttctcagacaccctatctccaatcaggtggaggaggcacgtgacccacatggtctggccactgatgactgaacaagctatggacaccggaccccggagagaccattcactcactggccacgaacatgagttcagatacatgccccaaaaggatgagcctgggtactggattccctccctcagaaacgtgaatcaagagacacaggatgttcctgttggtccagatacttgagctaaaaggtgatggatacctggatgtggggtggtcattctggggagtacgtccatatagaaagaggagcaggtgctgtgggattctggatcccagtgatagagctaagtggctggatcaagcttcacctgaaacccactctacttgtcttagtccattttgtgttgctataaaagaatacctgcaactgggtaatgtataaagaaaagagacttatttcattttatagctctgcagtctgagacatttaaagggatggccttgacttctggcaagggcttgcacgttgcatcaccacacgcaggaaaagggaaaacagaagggagactgcaaaaaaggggaaaacctgaaggtcatcatagctttataataacccactctcacagcaatgagttagatggagaaccaattcagtctcacgagagtgacagcaagaactcactcactggtgagagggtagcacttccaagccattcatgagggattcacctccatagcccaacacctcccactaggccccgcctcccaacactgccacagtggagatcaaattgcaacatgagttttggtggagacaaacaccccctatccaaatcacagcactacccccgaactttccattacacgaaccaaagaatcttcctttaaattagtgtcattgggttttctgggatttacctttttttttttttttttttttttgagacagggtcttgctctgtcacccaggctagagtgtagtggcacaaacacagctcactgtaggctcgacctcctgggctcaagggatcctcccacctcagcaccccccacctatgccctcaaagtagctggaactacaggaaccaccacacctggctaattaaaaaaaaaaaattttgtagaggccgggtgcggtgtctcatgcctgtaatcccagcactttgggaggccgaggtgagtggatcacctgaggtcaggagtacaagaccaacctgaccaacatggtgaaaacctgtctctattaaaaatacaaaaattagctgggcgtggtggcacatgcctgtaatcccagctactcgggaggctgaggcaggagaatcgcttgaacgcgggaggcggaggttgcagtgagccaaaatcacgccactgcactccagcctgggcaacagaacgagactgtcttaaaaaataaaaaaaaaaattgtagggacacagtttcactatgttgcccaggccagtcttcaactcctgggcttaagcaatcctcctgtctttacctcccaaagtgctgggattacaggcatgagccaccacacccagcctaacatgtttttaataaccaaaagaaaataaacatctccatagccttgtgacacatccttttttaacctgaaattggtagagtaggtttctgattcatagaacccaaacaaacattcctccctaaagcaggcagaggctctttgggagagtttactttcaaaacagtgtccctgcaagtcccaagtgacaagttcccctttcctggtctgaagcattctgaggagaggcaaagaggttaaggcttagctgcaggaacacgctgtccccacgacaaccccttctcagcctcggaactgctgttatcagagttgtgttgtctcttgaaggtccggacaaagggtgaagccaggactcctgagtctaaagggcaggtcggggcctgggagagaaggggaagctggcctggggcagagatatgttttccatttcccctggggtcccgcgcaggacagctgctgtccatagccagttcagccaaccgtgggaggaatcacattcactcgacaggcaggcagtggcggcactgggatttgaacatggggctgatgggctccaggcgtggcacctgtgccactacagtgcggggaagctctgactgctgcagcctccgttgtcgccacatctgccctcgaatttcccttccagctggttctgcaatcagcagcccctccagacatccctgggaggctccaaggagctgactcctgatgaggaaaaggatcagccgcagactcactcctcctccagccacttccagacccacgggaggcgtgggtgtgacccagactacctgggagcatctgcttttgtctttatccagggcaggaccctctgaaggaggagccctcagagaagaagggaaaagccaaaaggaattaaaagccaatttccaaatggaaataaataagcagtcataattggccacattggtgaaattgggcactaagggccaggcaatgggctgtgggataatgtcacttgaggcttacggccacgctcagaggaaggtgctgaggtccagggaggttcaggcaggtgacatgactcacccagagccactggctggagagtggcagggctagcttggagcccaggtggtggacttcataggtgcagccgtgactgctgtcatccctcccatggggagttgttgcagagagtgagacacagcgccctaagctatttctccatctggtgtgcctggatgcctctgcccacccaggaccctcctctcgggtggcccaccactgcccttccatcacagttcataccttctctcgtggacccacaggggtgtccactggaccaggaccccagggaggatcccagtcctggccccaccactctctggctggtgacacgggggaagtcaattctttccaagcctcagctttctcacctgtacaatgggagtttgtgctagacaagaagttttcaggacggacacagtggctcacgcctgtaatcccaacacttggggaggccgaggcgggtgggtcacttgaggtcaggagttcgagaccagcctggccaacatggtgaaacctcatctctactaaaaatataaaaattagccagattgtcatagcgcatgcctgtagtcccagttactcgggagactgaagccaggagaatcacttgaaccggggaggtggaggttgcagtgggccaagatctcaccactgtactccagcccgggtgacagagagagactgtctcaaaaaaaaaaaaaaaaaaaaaagttttcaaactgctgactgcacctgtttgtaggccctgagctcaactgagtgagtcacacaggtattcactgacgtgattctgcctccaaggccacattttctccagtgcacctctgtccttcagcaagtgacctgaggctgggactgccacatgctgcctcccatcagcaccttacccagacctactgctggctacaacgtgcttaatgattgtcaggtggctcacataccctgttctgttcggcttcacagtgtctgccagcccgtggagtatggcacagtggctactagccagggtcaggcagtgcgactccacctctccaagcgccccagtgcagcgtctgatcatagggaataactctacctgcttcacaggactattctgtgaatactaattaggataacatatgtgaaggtcttggcgttgtgagcacatgagcccaaggtcagctccctaaaatgatgaagttcatatcacaagaggcccctagaggtcaggacagcctttcggtacctttctatggcaaacaagcggctggttctctgccacctattttctccccttcattgggcaagactgagctggcagaataatggccacatcagtatttactctctactttctggcttccctagattggtgccagccatttgtccccccatccaagacaaccctgatccccatccccagaaggaaaacacctcacctctctcacaatttgagaatggctgtcaactcgagcacattattagatttaattgtatctaatacttgaatacctaatatattactttctctctctctctcttttttttttttttttttgagatggagtttcattcttattccccaggctggagtgcaatggcgtggtctcggctcactgcaaactccgcctcccggcttcaagcgattctcctgcctcagcctcccgacttgctgggattacaggctctcgccactacacctggctaatttctgtatttttagtagagacggggtttcaccatgttggtcaggctggtctcgaactcctggcttcaggtgatccacctgcctcgacctcccaaagtgctggcactataggcgtgagccaccgcacctggccccaatatattactttcaaggacaaaaactgcaattacttttgcaccaacctaatatttaaggtaggacaccgtgaagcccagtgagatctctgtggtggacaaacagcgtggctgctaggctgtgagccctcccgcctgtctgctctccatgctgtgcgccactcctgagagctgggtcagcagtcgcactgttggctttgctctgacctcgagtctatttcttttccatgtgtgtgtgcagccgctgcctggagtggaaatctccactgagggacagaccttctttcaaggctcacatcaggcaggatcccacgggaaagggggctggctgagtcaggagggttctctgcctgagttcccgtgtgacattgggcaaatcactcaacgctcagagctgcgcatttttatctccatctgcttggagcaggaattgcaaagagtttcagctcaggttccaacgccaaggtgggagatattggttgctttgggtatagtgttgagatagattctgaggctgtgtatgggctcactaggaaagggtgctgtgtctgccatgaagtgggaaatcggtgggggaagaaaaaagaaacccggctgggcacggtggctcacacctgtaatcccaacactttgggaggccggggcaggaggatcacctgaggttaggagtttgagaccagcctggccaacatggcaaaaacttgtctctactaaaaatacaaaaattagccaggcatggtggcgcatgcctgtaatcccagctacctgggaggctgaaacaggagaatcacttgaacccaggaggcagagattgcagtgagccaagatcatgccactgcattccagcctgggtgacagagcgagaccctgtctcaaaaataaaacccaaaaaacaaaacaaaacaaacaaaaaaaccccatggtgctggccgggtgcggtggctcacgcctgtaatcccagccttttgggaggctgaggcaggaggatcacttgagccctagagttcaagaccagcttgagcaacatggcaaaaccctgactctactaaaaataccaaaaaaaaaaaaaaaatgctggacgtggtgatgtgtgactgtggtcccagctactcaggaggctgaggcaagaagattgctggagcctaggcctgtctggctaacttggctctaccttggctcagagccccctcttcctttttttttttttttttgagatggagtctcgccctgtcgcccaggctggagggcagtggtgtgatctcggctctccgcaacctccgcctcccgggttcaagcaattctcctgcctcagcctcctgagtagctgggattacaggcacgcaccacgacactgggctaattttttgtatctttagtagagatggggtttcctcatgttggccaggctggtctcgaactcctgaccttgtgatccacccgccttggcctcccaaagtgctgggactacaggcatgagccactgtgcctgccgggtgggtttcttatggcgaatgatcccacaccacttttcagtgcacgtagttacatggagtagaacacagacagtcacacctccacgttcttttggccaccatgactggttcaaccaaggaatctgctccaagctaggtgaaggaaaatcagccctaaggccccccgtcgccacgcacctcactgtatccagctatacctaaagccaaaccgaatcccagacatctaaattttatgagccagtaaattcccttctttgcttaagccagtataaatcagggtcccatcccttgtaatgctgagtcccaaggcccagactctgggcctttcgtttttccattcaggccccagaatgaagaccaaggaagggtttattaacatgacagtgaacatctagggccttatatacatttttcttcaattcagccaagacaagaggcctagaatacagagaaaggacacccatttcttttcttttcttttcttttcttttcttttcttttcttttttgagacggagtcacgctctgttgcccagagtcacgctctgttgctggagtgcagtggcgcaatctcggctcaccgcaatctccgtctcccgggttcaagcaattctcctgcttcagcctcccaagtagctgggactacaggtgcctgccactacacccagctaaattttgtatttttagtagagatggggtttcgcctgttgctcaggctggtctcgaattcctgacctcaggtgatccacctgccccgacctctgaaagtgctgggattacaggcgtgagtcaccacgcccagcctaggacacccatttctttgtttgttttgtttgttttttgttgttgtttgttgtttttttgagacggcgtctcactctgttgcctaggctggagtgcggtggtgccatctcagctcactgcaacctctgctgggttcaagcgattctccctcctcagcctcccaagtagctgggactacaggcgtgtgccaccatgcccagctaatttttgtattttttgtagcaatggggtttcaccatgttggccaggctggtctctaactcctggcctcaagtgatccactgccttagtctcccaaagtgctgggattacaggcgtgagtcaccgcgaccagcctaggacacctatttcttcaaacataggaagaaacatcttatctgcatgatccctcctggggagctatcttacggtcttatgtgataaggtgacctctcctttgggtgagtggagtaaaaaagcgggcgaggtatctggaatatgcttctatccctcctaacctcccctacaaacaaagctttcctgaatgccttgtccttagcttcctggactctgaacctagcttggctaaattttctttttaaagcatcagcataaatcatgaataccttgggaagggaacattaaccatcagccggcaaagttaaaattcccctccagcccacttaaatggactgaaacctccaccccatccccaaacgagcctccctggccttgtccttgaacatttcaaaatcgggtttctgctttaagttctttaaactactttttttttttttttttttttttttgagagggagtctcgctctgttacccaggctggagtgcaatggcacgatcttggctcactgcaacctccacctcctgggttcaagtgattctcctgcctcaacctcccaagtagctgtgattacaggcacccgccacaacgcccagctaatttttgtattttcagtagagacggggtttcaccatgttggtcaggctggtctcaaattcctgacctcgtgatccgcctgcctcggcctcccaaagtgctgggattacaggcgtgagccaccgcgcccggccgctgctttaagttcttaacacgtcctcaccgcggctcagctcactgtacggcccctgcatagagaggggagggcaggggccatgatactgaactcacagctctgggcatttctgaagtctggcccttgaagcttttcctagagcaaattgttttcttctccccttcaggaaactgaaaattcacacagcagccgtgggagtcagagtagcaaaggctttgagatcacatagacctgggacacgtgcttgctttgtgattacgggtaagttccgtagcctctctgagcttccatttctctttctgtggaaaggggatactaatactgcccactgtgagggctgtttctgagattccatgctgctgtgactagcatctgcctgactagcagaggatagttacagtgtcagcgtccatggccctctcatgagactggggcagggggtgggaagggtgtttgggacaccctgggaaccttagcatcattctgccatgggagtatgacctgctgttccctggctgccctcagtctgaggcttgctctgccagtcccgggagaactgtccactcatcatcccggcagagacagatctctttttcgctgtgcacaggtggggaaactgactctgcccagtcacctgagtctgttgacagaggcacctggccatctgtatcctggtcctcagctctgagaccagcaggaaagagaggaggacattcctaatgggggacacagtgaccttcctcctcctgcccctgatgctcccaccactgtggtcccctggtggtgccaaccactcagactccagaagctgagtcccctctcagtcccatgaacaggcaaagctgcttcctgcagaggccccaacctttgcgcctcctcctcagagaatctgattctccctcctcagtgcggttcctttcccacacacacccacacaacaatccagactcagagaatggagtgcctcaaattaggggaaatggccgggcacagtggctcacacctgtaatcccagcactttgggaggtcaaggcgggcggatcacttgaggtcaggagttcaaggccagactggccaacatggcaaaactccatctcccctaaaaatacaaaaattagctgggcgtggtggcgggtgcctgtaatcccagctacttgggaggctgaggcgcgagaatcacttgaacctggatggcagaggttgcagtgagccaagatcaagccactgccctccagcctgggcaacagagcgagactccatctcaaaaaaaaaaaaaaattcaggaaatgatgaggctgcacacacacatgcacacaccacacatggctcatgcatgcacatgacatgcattcacatgcacacatgcccatgcatgtatgtgttcacatatgcacacacagcacctgctcacatgcacacacaggacagtcacacacatgcacacacacgcatgcacatgcacactcacatatacattatacacatatgtacacatggcgcgcacacacccatacatgcatgctcacacatgtgcacacatggtgcacactcacacacccacacatgcatgctcacacatgcacatacacactcccctcttcacccagccaccccgctattcattctggatccccttacccaaccctggttcaggaccacctctcctgacccctaggctaggtcaggccactctcctgacaccctcattactcataggctttctcttcagcacttttaccacgattgtgactaattacatgtgtgatcattttggtaccaggtaatattaatagctcgaaaaatattactgggctttcctctttggcctttctgggtaagtagcccttcccctctcaggcctgaggcacctggctacctggaaaatatttgaatttgtacagaaggaataaagaggaccatggcagatctagttcctgatcatcaacacagactttgctatgagacagtaggtttagaagtcaccgctgcgcactgaccctctgggggcttgggaggaaagtgctctgagaccaccctccagcagcttaggggtggggcccctggaggtgcagcaagttgagaggaaggaagccctggtgtttcgctttccttggccacactggtcccctccgggccaggatgggctcaatgtgttcttgctcaggtaggtgacacctcattaagacaaggggcagagggacctgcagaagggtctgggagtagatggctcaggtaggggtcagatgcagtagagaagagggggctctgaaaaaaaaaaaaaggctgggcgcagtggctcatgcctgtaatcccagtactttgggaggccaaggcgggtggatcacctgaggtcaggagttcaagaccagcctggccaacctggcgaaaccccatctctactaaaaatacaaaaattagccgggcgtggtggcaggcgcctgtaatcccagctactcaggaggccgaagcaagagaatcacttgaacccgggaggcggaggttgcagtgagctgagatcgcaccattgcactccagcctgggcaacaagagcagaaactccatctcaaaaaaaaaaaaaaaaaaaaaagaagagggcgctctgagccaaggtagagccaagtcaaccaggcaggcggaggcccagagcatgagttttacctactgccggtttcggccaaaatgccagaaagccaagcagcaatcttgggcatcggacttggctagaaatccctctcctcctgggaagtctcgtcatcaacacccagacacccccacccccacccacccacccagtgccagcttagagaaaagctgggaggagcagaaggaaatttgagaccaagcattacctaaaggcctaaagtactggatcagattaaattaaatctagttctttttccttgttgccctatgagtaggctttgtgagaaatatcagatccattgcaggaaataaagaaaagacatgttctctgcagacccacatggcaggtgagtaaatgtacagcatggatatccgtcaccagcggtgcattcccctaggcaggcaccacaccgtcctgtggagcattgtgttagtaggcgctcagtaaacacccatgaagtgagtcaatgtatgattaaattccaaccctgcgtggcagcagacaggtggctggtctctgggactggggaagaacaaggctctgtatgtagttggcttcctctggaaggacaggttttaagtcagatccgactgggttcaaaccttggcttggccaaggcagcttctagcatggcctccagtgatcccacctcctggcctgagtgtgggtggacacttctgctatttatttatttatttgatttattttgagacagagtcttgccctgacacccaggctggagtgcagtggtgtgatctccgctcactgcaacctccacctcccggcttcaagtgattctcctgcctcagcctcccaagtagctggaaccaaaggcatgcaccaccatgcctggctaatttttgtatttttagttgtttaggatttgttttagttgtattttagttgtttagggttatttttagttgtattttagttgtttagggtttcaccatgttggccaggctgatctcaaactcctgacctcaagtgatccacccacctcagcctcccacagttctgggattacaggcatgagccaccatgcccagccatgggtaggcacttctaatgagcagaatatggcaaaagggacaggatgtcacctccaagattacgttataaaagacagtgacttccacctttctctctccctctctctcacatgagcacgcgcgcgtgcacgcgcgcgcgcacacacacacacacacacacacacacacacataacttcttggctttcttgctttgacacagcaagctgccatgctggagatacccacatagaaaagaattgaaatcagcctccagccactaatagatagataactaatagcaatagataactaatacagccactcattagctctgccctcagaccagttcttaacctgtatgtgtgtaccctcatctgaaaatgggcataatagcacatccccaccatacaggctaattgtgaggaccacgaaaatgaatggatgggtggatgggtagatgggtagatggatggaaggatggttgggtgggtgaatagatgaatgggtgggcgaagggatggatggatggatggatggatagatggatggatggatggatgagtgggtagatgggtggatgagtgagtgggtggggatgggtaaattggtggatggatagatggatgggtggaaggaagaaaagatggatggatggatggatggatggataggtggatgagtagatggttaggtggataggtggatgggtgcgtggatggatggatggatggatggatggatggatggatgggtaggtgggtgggtgggtgattggatggatggaaggatggatgggtggatagatggaaccactggctggagccgtgtgaatatcacagaatcaggaagtgaagcccaatcagtaagtcccgatggggctgtgaataaccctcactgagcagctgtcacaaggctaagcacctggctgtccccctaggaaagcccctcaccaaggtgaagctctacctccgtcctcacttcacccgcagcccgttcctcctggggctgcaaaggaccagtgcttagagctacctctggtcagaagccagcagagtcctacagtaaaaggggttggggccccctgctgagctcagtactgaagaggaggcatggccaggcagcccagcaccctctgccctccacccagcaacccccaggctagattctgccatccttttctcattgattatgttccccatgaggctgctcttgcccaaaagctggcccagcttgacaccacagagacaaaccaaccaacagccacagcatctgatagccccagtaactgactcttgtgggaaggccgcggccagctggcctgagtcccacagcctgaccatggctccgtcagtggccagtcctgggggcaacagggacacaggggaagggtcaggccccagctctcatggtgtaacagataccagctccaagtcaggagaatggactttccacctgcatgcacgtcacagtgcccagaaccctaacagacatgcatcagacttgacaccttccccacccaccaccccaggtttggctgcccatagaggcgcacacctgtcctcccatctcctcctgggctggagccatgtgagcagaaggggatgtgaagacagatccttgatgtccacaggtgcggggactgcttgccaacccatggcaactcacctgtagcgcatggagatttgggggtaaacccagtgatgctgagtatgtgggctgcccaagctgggtggcaaccaaccccagcattgaatgtgctgagagctggaccggtggatgtccccactgccctgaggcaggtccaggagaaggctcctacctgctgtgcagagggcaaggtaagtcttgtccttgatctacagccagaagacagcagggctgcttctgtctgcgtcccagcctccactcttctctagataccatcaccaccatgacaactgcagttaacacttagtgaagacccctaggtgccacgcgcccatgcaatacccacagacccctctgaggtaggccctgtaatggtccctacgttagggaggggcggtcccaggcccagctggtcagtgagggaggtaggatttgaacccaggcagcctggctttggggcattgctctctgccacgtggccggttcctacacccttgccagctaccccctgcaaaggtggccagcctggctttgtccttcaggatcctctgatctctctcaggggcctccaacccacccactggctctccctgctgacaaagcaactatttcctggccccaaagccccagtctctggaaatgaccctccttccttctccagctcatggctgtcagaggctcctaactgcacccacccccgatcagccaaagccaattactccacgtccaagacacaggcgtgttaaaatgtaaacggccattcatcatctggcagcgtgaccgtttgcccctatcaggccccagtccggcctcaccacgcctccatgctctcctcagcgtcaaagaaacagccttgtcctgccagggccttcgccaggcccggcctgggaagtcagggctctctgacccaatggagcgaatcgttgcgagcatttatttaatagcattatcttgatgatgaataaatcacaccttcatgaaccatttttccatttgtagccccaagtcaagactcccaaaatgttaccagatgtcaggcatgaaatatacacccctggcaagctctgccatgcacaggagtcatgctgctttcaatccacgggagagccggacacgagcgttccaaggcaggagcagaactccatgttggccattataaagacaagattccttttcccccgctttttatagcctctctgggtaagatgtgtctggaaggaggtgtgggccaggaaagagcatatgtctgatgcctgggattccaggacagaaagtacagccgactccaagctcttgggatgggattggagggtctcaaaaccttgccagctttggagtcagagagaaaagagtcccaaccccagatttatcatttgctaacaaatgtgaccttgggcaaatcagctcacctctctggccctcaacagagataatcctgggacctatttcacagtcatggagaggattaaatgggaagaggaataaaaggagtttggagaagtgcccaggacatagtatgcactgagcaaattccgtagtaagaggagtattagtaatagctactattgattaggtggtgttgtgagttgaacagtgttcccccaaaattcatgtccacccagagacacagaatgtgaccatttgtggaaatagggtctttgcaaatgtaattagttaagatgaggtcatactagattagggtaggccctaagtctaatgactggtgtccttattagaagaggataggggctgggcacagtggttcacacttgtaatcccagggctttaggaggctgagacaggaggatggcttgagcccagaagttcaagaccagcctggacaacatactgaaacccatccagtctctaaaaaagaaactaaaagccaggtgcagtggcttacatctgtaatcctagaactttgggaggctgagacaggaggatcacttgaggccaggagttcaagaccggcctgggcaacatagcaagaccccatctctattaaaaaataaataaataataaataaaattaaacttaaaaaatttaaaaattagccagccatggtgggaggattgcttgagcccaggagtttgaggctgcagtgagctatgatcaggccactgcactccagcttgagcaacagagtgagatcctgtctaaaacaagatgggacacacaggaagtggtggtgcaaagatggtgcagccgcaagctgaggaacaacaaggatccctgaccaccagcagaagctaagaggcaagaaaggattcttccttagagtcttcagagggaaagtggccctgctgacaccctgatttcaaacttctagcatccagaactgtgaaagaacgagtttctgttgtttgaagccacctagttggaggtgcttcattacagcagtcctaggaagctaatacaggtggttaccatgcactaggttctctgcattataaatgcatcatatgtgtgaattcacctaaatcctcacaacaactgtaagaggttactataccatgttacagagagagtgatcaagactcagagaagttaagggactcatagctggatctagatttgaatttggtctatctggtctaaagcccacttttaaggaccaactgtgtatgccaagtgctagagccacttccttgctcagtcctcaccgaccacactgtgctatctttattcacattttgcagttgaggaaacaggttcaggcaggtctgtccaaggccaccctatttagggtctgacaaagccaggttcatctgactccaatgccaacgcccatgctgttgaccagaccatccctgccaacttcccaatatgaatgctttgagtggctctcggcccccgggctgtggaacaccagcacaccagtgcctggagggcagcagctgtccagcaaatgtctgctcattcatttgttcacgcattcagggcaagtcgaacggcagacggggcaggagatccagggccagcatcgatggtgcagggagccctctggccccttaaatatcctcagacaatcccagaaatcattcctgggtctaccttatgtgggttttctagaatagctactgctgaactgtagggtagtgagggagagacacaaatacaggcaaaaccacacaacagcgaagattccataagggcaggagtgtttttccaccaggaaacaagaaccagttcatctgagttccttcgccttttgtcccaagacacaaaaatgtcaaaaatgtgcaagggctctggccacagatggatactgtgtctcaccccgacgggctgcttcctgcatggggctgaaggtcagggtgaaggttgctgccctgagagtcaggggaagttggtctgcctcctgctgtgtcacgtacttgccatggtgtgtgggatgaggcccatcatctcccagaattaagctgtagctgactataaaaccacaggtgagggctgggtgtgggggctcatgcctgtaatcccagaattttgggatgctaaggcaggaggttcgctggagcccaagagttcaagaccatcctgggcaacatagagagaccccgtctctactaaaaatacaaaaattagccagacatggtggtgcatgcctgtggtcccagctactctggaggctgaggtaggaggatcacttgagcccaggaggtcaaggctgcagtgagccgtgatcatgccactgtactccagctggggccacagagcaatactcagtctcaaaacaaaataaaaaaacccacaggtgaggtcacatcaggcttttcccctttattctccctaagggctccacaacatcagaaatgggtttctctatcagtgcatcctccagattctactttttttttttttttttttttagtagaggtcttgcttatacccaggctggtttcaaactcctgggctcaagcaatcctcctgcctcattctcccaaagtgctaggattacaggtgtgagccacccacacccagcccagatactgggccctgtccgtcaatccatttgcagtcaccaagcaccagctgcgtgcaccgcattgtgccaatgtctggaaggatgctcacaactatcaacacagataactaataatatcaacaataccacagtgtaaatgcaaagcaatatgtgcctggtctcatttaattctcacatcaaacccttgggatacatatcattagcaccctcatttgctagagaaaactcagttctgagggtggatgatttgccaaggccctgggaattcggactaactcccttcataggtggctgcaagtgcctgtggcccagacattcccatagataggtcattttctcacaccagcttatatttcttgtgccagacacagggtccaaacccatttgcattacgtctctgagacttcacaagaccttcctgtgaatttgttaccattattattattattttcttgttttacttttttttttttttttttttttagaccgagttctgctcttgttgcccaggcctgggtgcagtggcgcaatctcggctcaatgcaacctccacctcccgggttcgagcgattcttctgcctcagcctcctgagtagctgggatgacagacacacaccaccacacctggctaatttttgtatttttagtaaaaatgggttttcaccatgttggttaggctggtctcaaacgcctgacctcaggtgatccacctgcctcggcctcccaaagtgctgggattacaggcgtgagccaccgcgcctggccttttttttttttttttgagacaagatctcactctgtcacccaggctggagtgcagtggggcgatctcagctcactgcaacctctgcctccctggttcaagtgattctcctgcctcagcctctcaagtacctggaattacaggcacgtgccaccacgcccagctaatttttgtatttttagtagagacggggtttcaccatgttggccaggcttgtcttgaactcctaacctcaactgatgcacctgtcccgacctcccaaagtgctgggattataggcatgagccaccgcgcctggccttattattcgtcttcttagtctgctttcagatgaggacacggagccacaggagttacaagggtggaggtgctgggaagtcaggaccaaaatcagcctcctgactccccatctcattttctcaatcatcatgggttttgccccccaagaagagaaccagccaccatgggtgctaaaccggtggctgatatagcaaagtgtgttggagcagagatcccagcgggcttcctagaggaagatcacaaaaaatgggcagagcccacaaggcacagtacaggtttcctctctccagtccacataagggcattcaccaggaacctgggggctgggtggccttggcccaaccctctgaaactcaccctgtggctcctcactcaccagatggacacagcaggaatctgtcccacctggctcagggagggcctacccccacctcccaccccaccccacaggcctgtttgcatgccaggccagagttggccacatgcccacatatctctcctcgttctgcaggctccgggaaggaaagtccaaggccaaggttatcatggggttcacataccttaaggggtgggtggaggggttggctcacaagctacaggttattcaatgcagtgctattcgtgatcacaaaagatcggaactgatctcacgtccatcatcaggggactggactaagatattttggcacaaccacatacgactcaagtgtcaagggaggaagagtgggggagtcttgaataaataacaaaagctggcgttgacacaggactttgcacatggtggaggctccttgcaagtgctttgcatgttattaactcattcaatccttacatcaccccaggaggaggaaccattattatctgtttaaaatgcctgtaatcccagcactttgggaggcccaggtgggaggatcacttgagttcaggagttcaagaccagcctgggcaacaaagtgagacttcatctctacaaaaaattcaaaaaattagccaagcctggtgatccacacctgtagtactagctattcaggaagccaaggtgggaggattgcttgagcccaggaattcgaggctgcagtgagccaggattgcaccactacactccagcctgggtgacaaagcaagatcctgtctcaaaaaaaaaaaaaaaaaaaaaaaaaaaaaaaatatatatatatatacacacacacacacacagagagagagagatacacatataatattttaaaatatatatacctcaaaaaatatatatataatatttaaaaatatataaaatgagggtgccacttggttgagtttatggttaaaaaaaagaataaaatttaaaaaagaggcaccaagaagtgaagtttcttgccccaggttcatggccaacaaacagtggactgggccgggcacggtggctcacacctgtaatcccagcaatttgggaggctgaggtgggaggatcgcttgagttcaggagttcgagaccagcctgggcaacatggcaagatcccatctccactggtgcagtggctcacgcctgtaatcccagcactttgggaggctgaggtgggcagatcacaaggtcaggagatcgagaccattctggcaaacatggtgaaaccctgtctctactaaaaatacaaaaaaaaaaaaaaaaaattagccgggcgtggtggcgctcacctgtagtcctagctactcgggaggctgaggcaggagaatcacttgaacctgggaggcagaggttgcagtgagccgagatcgcgccactgcactcgggcctgggcaatagagtgagactctgtctcaaaaaaaaaaaaaccgtctctactaaaaaactgcaaaaattagccaggcatggcggcatacacctgtagtcccagctacccaggaggctgaagtgggaggatcacccgagctcaggaggttgaggctgcagtgagccaagatcacaccactgcgctccagcctggcaaccagagtaagatcctgtctcaaaacaaacagacaaacaaacagtagacctggaatttggtgcaagcagcagggccagatccatgcttggtcctctcctcccagcatctctgttttgtcctgggtccctctgccaggagctccctggaacccgggaaggaagagaagcatgcagacaagcgtcagcacccccaccctccgctggccttggggagcaggtgtcattagacgaggctcaactgaagcctgcatctgaaacagattctcataactcaggcgctgtggtagccaacttctctctacagtcaattacggccagcggggcccactgattatttttatagcctttcctggagtcatgacaaggaaaactaaatgatccacaggacccctttcatcactaacagccgggtttcacggttgggggtggaggaaggaaagaaaaatcacttttttccccactttgagttaatcaacagaactcaagtctgggcagccaatatgttggtgttgagaaaagctgttctcaagcagaactaattggcaacagaaatggaattttcttccccacctccctaaaaaacataaacatgctttatcccagagaattacccttcagaataggtattgcagacgtgtcgtgtgcagccgcctgtgcgaggctggtaatctcacgccgacctggccaaaggctgcctgtgctggttacagctcaaccagagcagcctcttacgcaatacaatggtcatgaaggtcagtggcccaaaccagcccagccagaccagagtggacacttgctttcctgatgcccagcacctatctcctgctcctacgaacagcctttggcttttgttgggtcggggggtgggagggcaccctgatttgcaggtacaagtgtgagtggcattgataccccctcaactccagagatgagctctgacaggcgtaagccaaagaacatattccggggaaatttcaagaaggggcatatgacctaacaagaaacaatagctggcacagtggctcacaccttaatcccaggactttggggggctaaggcgggtgaatctatttcagctcaggagtttgagaccagcctgatcaacaaagtgagaccccagtctctgcaaaaaataaaaactggctgggcgtggtggtgcatgcctgtagtcccagctacttgggaggctgagatgggaggatctgcttgagcccaggaggtggaggctgcagtgagccaagattgcaccactgcactccagcctgggcgacaaagccaaactctgtctcaaaaaaaaaaaaaaaaaagaaaagaaaagaaaagaaacaataaggcactagaaaaaaaaaaacatataatgaggcttctggaaaagatagctctttttattttagagaaattgaggttcaaagacgtgatgtaactgctcagaggtatacagtgaggtggtaggggatccagggtttgaacccaagtctgtaggactgaggagtttgcaacccttcactgcaggcgctactgaaaggcagcataaaccttggacgaagccagaagagctggattcaaacccagttctgccatttactccctgtgtaatttcaggcaagtcacttaacctctctgttcctcagtttccatatctgtaaagtggggcttctaagggcacctccatttgcaggatctgagaggtaaataattaacagcaatatctgtaaagtgcatgcaacagtgcctgtcacggaccaaggattcgccccttcctcaccacaccgttccccacacgcgttgagaaccagaccaaataaaaggaggtggaggggaggaaggaaaagttgatgttatcagatctgtaatccctacgccatgtgttcctttctgtagcacagctgccaggcttgcctctcctgcaggagaagctgtgccacaaagatgtgggacgtcacaaacacgcagcccattcaaggcttttggacattccagccccgtccttgatgtcagtcaagatctagggcaaattccacccattcaggtggcaaaaagtaagatgtttgacaaatgccatgtgtagagggtggcttgtgaattggtacaaccattagaagataattcagcatacgatttcatattccagcaattctacttccaggtacaaacttgagacacactcctgtatgtgtgcaataggacagtacaagaaccctcaaagcagaaccgtgtatgtagcaaacaaaactgacaaatggaaacaacccacatggtcatagacaggaaaaataaatgctggtatgccatggaatagtacacagcagagaaaagtgaatgaacaagtgataagcaattatatcaacgaattttagcaataaaatgatttataaaaatgtaagtgcaggggctgggcaccatggctcacacctgtaatcccagccctttgggatgctaaggcaggctgattgcttgaggccaggagtttgaggccagcctgggcaatatgcaacatggcaaagccccgtctctacaaaaacgtagccaagtgtggtggtgcacgcctgtagtcccagctaccagggaggctgaggtgggaggatcacctgagcccaagaagtcaaggctacagtgagctgagattgcaccactgcactccagcctgggtgacagagtgagaccctgtctcaaaatatgtgtgtgtgtgtgtgtctgcatgtatgtgccagaagacaagatatatctttacgtaaagttcaaacagcagtaaaagtaaatgatatattattttggcatacatgtaagtgataaaactttttttttttttgagacggagtttcgcactcatcgcccaggctggagtgcagtggagcaatcttggctcaccgcaacctctgcctgccatgttcaagtaattctcctgcctcggcctccctagtagctgggtttacaggcatgcaccaccacacctggctaattttatatttttagtagagacggggtttctccatgttggtcaggctggtctcgaactcctgacctcaggtgatctgcccgccttggcctcccaaagtactgggattacaggcatgagccaccgtgcctggtgataaaactatttttttaagcaagggaaattgagaagatgaaaatgttctggaaatggatggcgctgataattgcccaacattgtgaatgtgcgtaatgccgctgaattgtacccttaaaatggctaaagtgataaattctatccacaatttttagaaaggcaagggaaagctaaacacagaattcaagatagcagctactaccacagtgagagagggagggaggcaagatgggaaaagagttcggaagttgatataaaatggttgtctgagttccagcacgcaggtgggaccatgggttcacaggcatttatcattttataaatagctataaagagattgttagatggaagtgtaggtggggctgttcaggtgccctttcagtatgtcattatggaattatgaatcattcaatcctacttactttttcttttttttttttctttgagacggagtctcactccattggccaggttggagtgcagtggtgagatctcagcttgctgcaacctccagctcctgggttcaagtgattcttgtgcctcagcctcccgagtagctgggattacaggttcatgtcaccacacctgtcttatttttgtatttttagtagagacgggctgtctctactcctgttggtcagactggtatcaaactcctgactgcaggtgatccacctgcctcggcctcccaaagtactgggattacaggtgtgagccaccacacctggtcaattctacttacttgaggtccatttttaaaattctagaggaggccgggtgcagtgtctcacacctctaatctcagaaactttgggaggccaaggcaggtggatcacctgaagtcagtagttcaagaccaacgtggccaacatagcaaaaccctgtctctactaaaaatacaaaaattagttaagcgtggtagtgtgcacctataatcccagctactcgggaggctgaggcaggagaatcacctgaacccgggaggcagaggttgcagtgagccgagattacaccattgtactccatcctgggcgacagtgtgaggctccatctcaaaaaaaaaaataaatccctaccttcaggaaactcacattccagcaggggacacttaagaaaataagaatatatatgtatgttagatgatgagacgtgtgtggtaaggagaaaaataaaccagaaaagggatgaagaagaccagggagtgaaggaagctgtaattttaaatggagagtcaggatggcctcgctgcaaaggtgatgtttgtgtaaagacctgcgcgatatgaacaagtgggccacttggatatctgaagaatgagcacccaggcatagagaaaagcgattgcaaaggtcctggggcaggactgtgcccgacctcaagaacagcaattgtggggagtgggaaggaggagagataaggttagagaggcctgggccctgcaggccttgtgggccgtgatgaggactttgcctgtgctctgggcaaggtgggaccagggctggagataggaggtgtcctgaacagagaaggaactggatctaatttcattttaacaggaccctgctggctgcacacggagagtagaccaggagggaggcaagaggagaagcagagacactggtggggaggcaactgcaatagcccagagagagacaccatggccgctgggaccagggtggagggagcggaggtgacagagctgtcagcttctgggtgcaggttgacaggggagccaacggaatttcttttctgttcctctttgtttttgagacagcgtctcattctgtcactcaggctggattgcagtggcacaaacatggctcattgcagcttcaacttcctgggctcaagtgagcctcctacctcagtttcccgagtacctgggaccacaggtgcatgcaaccacacccagctaatttttaaaaatatgtttgtagagacaaaggtcttgctatgttgaccaggctggtcttgaactcctggtctcaagcgatcctcctgccttggcctcccaaagtgctgagattgtaggtgtgagccaccacgtccagtgtagaatttcttttttgctggaaagtgggagaggctgggctctatttaagtgtttaagggtcaagaaagtttgagacctggttataaagtagagacatggccatccaaactgagcccttcttgagagctgatgatgggatagaatttttttttttttttttttgagatggagtcttgctctatcacccagtgcagtggcgcgatctcggctcactgcaacctccgcctcccaagttcaagcgattctcctgcctcagcctcccgagtaactgagattacaggagcccgccactgcgcctggctaatttttgtatttttagtagagacggggtttcaccatcttggccaggcttgtcttgaactcctgacctcgtgatccacgtgcctcggcctcccaaagtgctgggattacaggcatgagccactgcgcctggcctgatgggataaaattttttaaaaaataggctgggcacggtggcccacacctgtaatcccagcattttgggaggccaaggtgggcggatcacctgaggtcaggagtttgagaccagcctggccaacatggcgaaacccccgtctctactaaaaatacagaaatcaggcatggtggcatgtgcctgtaatcctagctactcgggaggctgaggcaggagaatttcttgaacctgggaggcagaggttgaagtgagcaggatcacgccactgcactctagcctgggtgacagaacgagactctgtctcaaaaaaaaaaaaaaaaaccctcctcttctaggacttctgtgatgtgggcattaaaatgaatgttttaggtcttcatgggctcacatggaaaaatgtccaggacacctgttggttgaaagaaggaagatgcagaataaaatgtatagaatgatcccatttttgaaaattaaaattacgtgacaaagaaaaaaataggaatgaagtgaatgaatggcctgaaagcatagacgcctggctctttctttgttttttttaagaaggagtctcgctctgttgccccaggctggagtgcagtggcgcaatctcggctcactgcaagctccgcctcccgggttcacgccattctcctgcctcagcctcccgagtggctgggattacaggcgcccaccaccatgcccagctaattttttgtatttttagtagagacggggtttcactgtgttagccaggatggtctcgatctcctgacctcgtgatccacccgcctcggcctcccagagtgctgggattacaggtgtgagccactgcgcccggccaagacaacgggctcttaacaggggtggcccagggtgagtataggagctactgaggtttaaactcaggcgcgcccttcctacctcgcaaaacaataaaacacgcatgcttggggccgggcatggtggttcatgcctgtaattccagcacttttggaggcctacgcgggtagatcacctgaggtcaggagttcgagaccagcctgaccaatatggtgaaaccccatctctactaaaaatacaacaattagccaggtgtggtggtgggcacctgtagtcccagctactcaggagctcctgagacaggagaatcacttgaacccaggaggcggaggttgcagtgagctgagatcgcaccactgcactccagcctgggcgacagagcgagactctgttaaaaaaaaaaaaaagaaagaaagaaaaaaaagtgcacgttttttagttctcttttaaactgctgtgggcatggttctcctcttgtattgcagttgcgtttctcttagctaatgtcccacctgggccttgtccttggcctggactataagagagcggaacttgagaatgagaaatattttcctctcgtgcttgctggcttaggcccttaaaactggaaagggcccacctggctcctagagtttctaaaaatagcaagctatctgaacttttcctataggtttggcttagccgttcctctgacttgttccaagatgccatttctgttagggcatcattcgctgtgcatgaaaacaaaggatggcaggagacagactagaaacggaagaggactaatcgggagccgctgcggacagaatccagaaatgccctgcaaatgcagacacagtggacgcaatggagagtgcataccagggcctgtgtcgctggtggagctggggcccacatgccgctagtgtagacaggatctctactccacccgatctctctgcatccactccagtgcttcgggcaagagagaggagtttggctgctggtttaagtgagatggggaaattgaaagatttgcatccaaggcccatgcctggggtctgaattcctttggaagaggactgaggactgttcaggagacgatttttgacaatccagacacaaccatagatgctgtggaaccagtggtatgctggcaaaggtttagcaaccagctctccaagggaaagtgagtgtgtggtgcacaagtgtgcatgggcgagtacatatatttgcatatccacacacacatatacttcggtttattataaattgtattgacgtaggctgggcgcagtggctcgtgcctgtaatcccaacactttgggaggctgaggtaggtggatcacttgaggttaggagttcgagaccagcctggccagcatggtgaaaccctgtctctgccaaaaatataaaaaactagccaggcatggtagcgggtgcctgtaatcctagctatttgggaaggtgagacaggagaattgcttgaacctgggaggcggaagttgcagtgagccatgattacgccactgtcctccagcctaggcaacagagtgtgactccatctcaaaaaaaaaaaaaaattgttttgatgtaaatgatgtgcagcacacaatttacaaataaaaataaaacttacaataccttttcttttataaatgtaatataatcatttcactcacaggtagcagttttgttgatttttgcccccagcaaaatctgtaatcaacctatggttacaattgatgaaggagtgtaattcttcagaaataccagttaatattttcctttctaaaaaatttctaattatttgtttatgtatttttatttttttgagacagtgtcttgctctgtggcccaggctggagtgcaatggtgagatcacagctcatggcagcctcaacctcttgggctcaagcaatcctcccacctcagcctcctgagtagctaggactacaggcacgcaccaccacactcggctagttttttctcaaattatttgtagaaatggggtcttgttatgttgcccaggctggtctcaaactcttgggctcaaatgatcctcctgtctcagcctcccaaaatgctggaattacatgcatgagtcaccacactcagccttgactacctttgtttttcatagaatttattgaattgtaagttcatataatttaatttttaacaatggtgtgtttagcaacccgttcacctaattcctgaaaatctgacaatcagctttcacaagctggcacaagctgcctccagcacacctctgtctggggacaacatggcaaagaatatcaccgaactgaggaggaagccattctcttcacatctgcccagaacccagggtatctcaagcactaaacagcgggaagctttgggagtttacagagatggcaccatggacgctgctgtgctgggcagggaagcatctccaaatggcctcagaggaaagaggaagcaggaaggatgaataaaactagagactggttcacaaaggcactcgaaatacccctttggagctccccaggatgaactgggggaccctggggggagcactgaggttctgatggggaaaacctcagcaaccagtgggcatctgggccaaatcacttagcacaaacgctgagccacaaaatcatgatacgctttcaacaacccattaggcacctcagaagctgcagggccatagggttgttacaaccgacacccatgcaggtggtaccacaggccatcagtccttttccccaagtccaccttcattcccgcctcctccagtaccatcctggacttctctagggaagtggtggggagatttactcctctccaaacttcttttttttttttttttttttttgagacgaagtctcgttctgttggccaggctggagtgcagtggcacgatctcagctcactgcacctctccctccccagttcaagtgattctcctgcctcagcctcccaagtagctgggactataggcacgcaccaccacggccagctaatttttgtatttttaatagagatgaggtttcaccatgttggccaggctagtctcaaacacctgacttcaggtgatccgcctgccttggcctctcaaagtgctgagattacaggcgtgagccaccgcgcccagcctcactccaaacttgatcttactctcagacatcttactctcagacgcttaagttctcagttcggaacttaagcgctcttcaaccagactcatggaatctcagggttgcggagagattttagaggtttcctgaaatagtccctttgttttaaagacgaggaagctgagagaagggaagtgacttgctcaaggtcacagagaaaatccagggtggaactgggtagggtgaccaattgtcccagtttacctgggactgggggggcttctggacaagttgctcccagatctgagaccacaactcagggctgacttgtccccaaggtgggtggtggggggttcacagccagtccctgaaaggaaaggaggcctggatcagtacagagctgctttgggatggaattttccaagggaagaaaaaagaaaaggccacatctgggtgctgggacctttcactctggcccgatgcctggaaccccagggattccccacctgctgtgtgtttcctggcccttgagtgtaacctgcaccctccctttccgtgccagggagacacgagctgactttatctgtctcttatctcttggctgctgccagccacagctgcagggatatatatgcaaatagcttacgataatattaatatgtgattccccccaggggaggcatgtggaaagcgctgtatgcgtttcacaattctgtttcatccaaaaacactgcacgggcccagagtgattcgaaacagattttctgcaaaggcaaaataaaattggaacaaaacctagttgaggcatatgccagtctccctgcccccagctctctccacctctgacatattaaccctcttctctcatgcccccaggagcctcctacgacaaggcagcaaatctaggaaattgcactgggcactcttcgttcaagcctggagcccctgccccatcccagggcagccagcctccaccctgggctgacacatcctttagagagtaaaaggctgcttgccagtgtcagcctgtcactccttctgtcccacctctgcctccttcctgcaggtaatgctccatccatacctccttctccttcacccctgctgcttgggataattgcagaaccatggagcacagaatacagaatactgggggtgtacaggagagtttagccaaagcaagtcagaaaaacctaggtcttttttcgtttctgccactgaccggctgtgtgacctcagcaagtcacttcacttcgcccagctgctgtttcctcagggtacaatgaggacaccaataatacttaccctgcagggggcttaccgggatttcccgagccgacgcttcaaaaatgcctacacagtgcctggcgtatagcagatacacaaggaatagctgtcatcatgagaatctcttaaagatcacctagttcaaccactgttttagaaacggacaaatggaggcctgcagagggcaaataacttgcctaaggccacacagcacgataccgccatcccatctcagcctcaacccaggttccctcctccggccttgggagctccaggtggcctgtgaggaacggctgcctcctcctgtcacccccagctccagaagtctgtccacacaaggcggcgtcacggcacacatggggagcagtcacttcacactcaccatcgagcaggtctggacactcgagtgcagtcccgccgccctccttgcagctgcctcactttccctattgccgccagcaagcgtctgctcccatctggcccgggactcccggactcgagctagggctctgcaaattccatccacactggccaccagccgctggtcccgctctctgggaagatcgccttgaggacctgctgcgccccgagtcttccttctggtgcagggaggccggtgccctgccgggctctgataatgcagccgggactcttatctggcctgtgtcagggtgcaggcggccatggagctggggttccaggaagccctcctggggccccccagccggccccgctccccccggatgccgcctgctgctctggacgcggccgattgcttgtcagtgtcactcccagctctgccggggggaattccatgctggcccccagcaggcggggcccccaccccttcacgtcccaccccccactcccattttggcaaggggactgggaaaaggcagctaatttcaagtccgcacagcgtttgtggtcgtgtcctgaatcctccacgattaatcacagagcatctgatttctgctttgcctcagagaggggcggaggggacgcctggaagtttctgtttactccattctgcaccaggctgcgtgctaatcacaaacagactgggacgcagcctacccctcctaaactgctcttggccaccccctccctcctccagccctctccttcctcttcttaccttgtcactttcctccagccccttcctcactctttctcctttccctcctttctttcccctttcccatctgtccgcctcttcagtccagatctgatccattgcacaccccttccttccgtcctgggtttcccccaagcccctttccccctttgcgcctcccacttctcctagattgagagtcagcttggttctttcctttacatccattagtgagggtcaggctcttttgttatgtttttttttcttttgtataacttaattatttcagggttcggggtgggcgctcgccccttgcccagtcacactggtgtgtgtgcgactcctacaaagttaacagtttctccaggtcaaggggtgggatccaggcttggtgatgtgcacaatttcttttgtccacttgacacatctctgcgtcctgattctgctcagggacggacccaagaacaaagcagccatttaccgcctccggaggggaggccagccctgtggcacatccagggccttggaacacctagagacagatttctctccctcgccttggctcctttccactctgcagctagtgtggaaaagaaaccagaaataaacagcaccaaagaacaggaacggacacccctccccattaaagcacacacacagactctgaagggtaatttggcaaagacctctgaaaaccagagatgagggtctcctactacttatgcctgtgcacaggagacaggcacagagatgcttgctgagagctgcttatcataggaaatgatgggaaataactgaaatgctcatccaataatgactgcttgaacaagatgtgaaagatatggtacatcaggc

SEQ ID NO: 9 - DNA, 403 bp, Homo sapiens
tcaaaccctaggttctctctgcactattaacacagacatctcaggacatggtttgcttttttttaagacttaaataggaaactaatttttctttctttaaagcaattgcgttcttcagtgaactctttctttaggccagttgatggcttcttagcagtttattgacgagatcctagggtagcttccgaagctgggttgattgattgcatttgggtgcggatggccaaagtgagtggccctactgcctgtgctgctcagggctcctgggctgatgtggtggcttcttccctttgtgctgctgaacatagggaaagtgaggttcacagtccaccatccaccagccgccgtctgtgagctccaccagcactcgcagcaagtcagtcgggctgagagtgtggttgta

SEQ ID NO: 10 - DNA, 399 bp, Homo sapiens
tcaaaccctaggttctctctgcactattaacacagacatctcaggacatggtttgcttttttttaagacttaaataggaaactaatttttctttctttaaagcaattgcgttcttcagtgaactctttctttaggccagttgatggcttcttagcagtttattgacgagatcctagggtagcttccgaagctgggttgattgcatttgggtgcggatggccaaagtgagtggccctactgcctgtgctgctcagggctcctgggctgatgtggtggcttcttccctttgtgctgctgaacatagggaaagtgaggttcacagtccaccatccaccagccgccgtctgtgagctccaccagcactcgcagcaagtcagtcgggctgagagtgtggttgta

SEQ ID NO: 11 - DNA, 401 bp, Homo sapiens
cctaaccagcttctcctcttagaatttcctgctgatccatcccagaatgaatgggagttcaatctgtactgaattatctttcatctagcaattgtgcaattccaaatgcaggtgaggttgagggaaagcgggcatcccctcacatccatgggatctatgtgtgggttgtatcaagagtctcaaaaatgctcatattctccagtcctagaattgggtctagcctaaggaaataattcagaactccatgtttttttaaagctttatgcacaaacatgatcataagacatgatttatgataaaaattggatgaagtaaactttcctatgaaagcagctgagtaggttaaattaaggtatacacttgatagccccttcataaagaattctcaagtgaaaaaaaaa

SEQ ID NO: 12 - DNA, 401 bp, Homo sapiens
cctaaccagcttctcctcttagaatttcctgctgatccatcccagaatgaatgggagttcaatctgtactgaattatctttcatctagcaattgtgcaattccaaatgcaggtgaggttgagggaaagcgggcatcccctcacatccatgggatctatgtgtgggttgtatcaagagtctcaaaaatgctcatattctccggtcctagaattgggtctagcctaaggaaataattcagaactccatgtttttttaaagctttatgcacaaacatgatcataagacatgatttatgataaaaattggatgaagtaaactttcctatgaaagcagctgagtaggttaaattaaggtatacacttgatagccccttcataaagaattctcaagtgaaaaaaaaa

SEQ ID NO: 13 - DNA, 401 bp, Homo sapiens
cacctgcagtccccacaacaacctgggaggggctgctgtcaccagcctctccttacagacaaggaacctggccttctgaggggaggtcccacggggcagaggcacagctgggatcacagctactgtttgacggcacattctgcaccttgaatgtggcctggggttacctcactgaaccccgtgcagtgccctcctcctatacagatagggaagcagaggctcagagatgtgaatcatttgcctagagtcacacagctgactgaagagtgtgctgcaactccaggacttgtctcccttacctccccacaaagagtgtgtatctctgagcccagcccagccacagcctccactctgggccccgattaactctggctattaggaaggcagaagaggctccccga

SEQ ID NO: 14 - DNA, 401 bp, Homo sapiens
cacctgcagtccccacaacaacctgggaggggctgctgtcaccagcctctccttacagacaaggaacctggccttctgaggggaggtcccacggggcagaggcacagctgggatcacagctactgtttgacggcacattctgcaccttgaatgtggcctggggttacctcactgaaccccgtgcagtgccctcctcctatgcagatagggaagcagaggctcagagatgtgaatcatttgcctagagtcacacagctgactgaagagtgtgctgcaactccaggacttgtctcccttacctccccacaaagagtgtgtatctctgagcccagcccagccacagcctccactctgggccccgattaactctggctattaggaaggcagaagaggctccccga

SEQ ID NO: 15 - DNA, 401 bp, Homo sapiens
atgtgcggggatggcatggggaagggtgcacgatagagtgacaagagctgagccaaggacagtgggagaaacagacggggaggctggcaggaaacgtggagctcgggtcacccggtgggagtggtggccactgggtcactgctggaaggaggtgcactcaccggagaccctgggagcccccaaacagggacagctcatccagggcgaagtcggcattgaggaaggcgaagctctccaggatgcactccatcaggctctcggccgaggtgtgctcctgccgtgctctgcagggctgtggacgaagtggccagacctgagggcaacaccgggccccacccacccgactgggacactggccaggggcctcacggcagacttgggcaatgtcccggtcccaagcc

SEQ ID NO: 16 - DNA, 401 bp, Homo sapiens
atgtgcggggatggcatggggaagggtgcacgatagagtgacaagagctgagccaaggacagtgggagaaacagacggggaggctggcaggaaacgtggagctcgggtcacccggtgggagtggtggccactgggtcactgctggaaggaggtgcactcaccggagaccctgggagcccccaaacagggacagctcatccggggcgaagtcggcattgaggaaggcgaagctctccaggatgcactccatcaggctctcggccgaggtgtgctcctgccgtgctctgcagggctgtggacgaagtggccagacctgagggcaacaccgggccccacccacccgactgggacactggccaggggcctcacggcagacttgggcaatgtcccggtcccaagcc

SEQ ID NO: 17 - DNA, 401 bp, Homo sapiens
gccaattcccgtgcccctcagcagaagtctcagggcctccagaaaggcctccgcccaccccctctcagccctgttacctttcatcctgatgtggaactcgcccaggtgaacctccagggccccctcgatgagccacatgtcctgcaaagccccggaggtggctcagctggctgcctggggctaggccacgagggcctctaaccatccctgcagccagacagaggccacaggcagagagacgcctccttggggcccagaacacctcctccagcccccactggcccagctctcgatgtccccactgcccggcccagctcttgctgcccctgctgcccagcccagcttggcccggcccacctcggcgcactcgtgcaggctgcggcccagctcctgcaggctct

SEQ ID NO: 18 - DNA, 401 bp, Homo sapiens
gccaattcccgtgcccctcagcagaagtctcagggcctccagaaaggcctccgcccaccccctctcagccctgttacctttcatcctgatgtggaactcgcccaggtgaacctccagggccccctcgatgagccacatgtcctgcaaagccccggaggtggctcagctggctgcctggggctaggccacgagggcctctacccatccctgcagccagacagaggccacaggcagagagacgcctccttggggcccagaacacctcctccagcccccactggcccagctctcgatgtccccactgcccggcccagctcttgctgcccctgctgcccagcccagcttggcccggcccacctcggcgcactcgtgcaggctgcggcccagctcctgcaggctct

SEQ ID NO: 19 - DNA, 416 bp, Homo sapiens
cactgccccaccccaccctgcaacatccacgagccagctgaccttgctgatgtgaaactccatcttccgaatgtgcctttccacacagcgcgtttgcttctcccggaaaaagggaagatgtttgcaaagttgcctgggccacccacctgccccgcttgcccctgccaccctcctacaggtcctaactcagagaatggggcttagtgccgggccggcccctcaccatccctgaggaaggctcatcgcagagactcagccttcccattcctaaaatggggaggagacccaggttttctgcccatcaggcagccaggaagatgcaatgaggcacagtcattctcatccagccaggcccagcccacctcactcaccgtatgcagactcaccttgtccaggtcataatagaaagcctgtga

SEQ ID NO: 20 - DNA, 400 bp, Homo sapiens
cactgccccaccccaccctgcaacatccacgagccagctgaccttgctgatgtgaaactccatcttccgaatgtgcctttccacacagcgcgtttgcttctcccggaaaaagggaagatgtttgcaaagttgcctgggccacccacctgccccgcttgcccctgccaccctcctacaggtcctaactcagagaatggggcccctcaccatccctgaggaaggctcatcgcagagactcagccttcccattcctaaaatggggaggagacccaggttttctgcccatcaggcagccaggaagatgcaatgaggcacagtcattctcatccagccaggcccagcccacctcactcaccgtatgcagactcaccttgtccaggtcataatagaaagcctgtga

SEQ ID NO: 21 - DNA, 401 bp, Homo sapiens
cagcggcagaggccactgtgacatacccaagatgtgacacctgacccactttcctggcattacagaagccatcccaagtccaggtcacctgatggccaaggtctataaaataggaccacctaaaagaaatgcacctccatacactgcccaccttagcattacttctagaaccgagagacagtgtgacatgggcctaaaacatgtgaactgctgtacgtgccaaagtgaagttaactcagtgcaacgtgaagaggctattccataaacctctagttctgagaaagagtcacaccgtgacataggctagaaggaacgcagggttcatcttttactcctggccaaggctatctgggtgggaagcaggcagggaggggtctcaccagcctggaattcctcctggt

SEQ ID NO: 22 - DNA, 401 bp, Homo sapiens
cagcggcagaggccactgtgacatacccaagatgtgacacctgacccactttcctggcattacagaagccatcccaagtccaggtcacctgatggccaaggtctataaaataggaccacctaaaagaaatgcacctccatacactgcccaccttagcattacttctagaaccgagagacagtgtgacatgggcctaaaacgtgtgaactgctgtacgtgccaaagtgaagttaactcagtgcaacgtgaagaggctattccataaacctctagttctgagaaagagtcacaccgtgacataggctagaaggaacgcagggttcatcttttactcctggccaaggctatctgggtgggaagcaggcagggaggggtctcaccagcctggaattcctcctggt

SEQ ID NO: 23 - DNA, 401 bp, Homo sapiens
ctctgcagtgcgtgctccacaagatcagagtcctcctgccttagtcactgccaggtttccagtgcccaaggaccgggctgagcacgcggctgcaccctgacatacttgcttactaaacgaatgaccaggaacttaacctgtcacctcttgtagacaagacccatccacgcttccccaggaagagacagagaggaggcgagatagaggaatgcacttcttaaaggcagcacacagcccagccttacttgaggcctcttttcaatgcttcgaagatcttcttcacctgctggggcttcgggtctgcacagaccgaccccttccgcagcgtgccgtacatcttggaggattttgcaggcattcgcgatctcacggagttcctgttgatggactttctgtgagaa

SEQ ID NO: 24 - DNA, 401 bp, Homo sapiens
ctctgcagtgcgtgctccacaagatcagagtcctcctgccttagtcactgccaggtttccagtgcccaaggaccgggctgagcacgcggctgcaccctgacatacttgcttactaaacgaatgaccaggaacttaacctgtcacctcttgtagacaagacccatccacgcttccccaggaagagacagagaggaggcgaggtagaggaatgcacttcttaaaggcagcacacagcccagccttacttgaggcctcttttcaatgcttcgaagatcttcttcacctgctggggcttcgggtctgcacagaccgaccccttccgcagcgtgccgtacatcttggaggattttgcaggcattcgcgatctcacggagttcctgttgatggactttctgtgagaa

SEQ ID NO: 25 - DNA, 401 bp, Homo sapiens
aggaacaaacagagtcagaccaaatctccatgacagtgagttcctggatctagctatgtctaaagctgaacctgcccgtggactttgcagttacatgagccaactggctctcttttttagcttaagccagctggagttgggagtgtggactggatgatcctaaaaactgcctttcagtggtgatggctgggtccctcaacatttagagatgtagcagcatctcaagactgattataggagtacgaggccagggcaccctcatcacagcacagagctggtttccctggcatctaagcctcttctcaggatcccataacttatccatgaggctggctgatgcagcctttgctcaccaacagatgtgttgaattctgctcttagccctctaaagccatcagcca

SEQ ID NO: 26 - DNA, 401 bp, Homo sapiens
aggaacaaacagagtcagaccaaatctccatgacagtgagttcctggatctagctatgtctaaagctgaacctgcccgtggactttgcagttacatgagccaactggctctcttttttagcttaagccagctggagttgggagtgtggactggatgatcctaaaaactgcctttcagtggtgatggctgggtccctcaacctttagagatgtagcagcatctcaagactgattataggagtacgaggccagggcaccctcatcacagcacagagctggtttccctggcatctaagcctcttctcaggatcccataacttatccatgaggctggctgatgcagcctttgctcaccaacagatgtgttgaattctgctcttagccctctaaagccatcagcca

SEQ ID NO: 27 - DNA, 401 bp, Homo sapiens
tcttggggcatcaacttaaacccttcccaggctcccctccactgagaatgtgtctcaaggcctcactgcagcccatgaggctccgcagggtcctcctccctccctgactgctgtcacgcatgccagccgcacacctgctttctgtcccttaaagctcattcccacccaggacatctgcactcgcagctgcctcccgccgccgaaggcttcccggcccacccccatctgcacacgcgcagatccacttcttctgtcccttcctgcctccactccccatgcccctgtctcgtcaggctctcccaggagaccatgggtgccctcccccacccccagttcagttccctcacagcactgccaccagctggatctgtctcaattatcactggcttattgtttgctgc

SEQ ID NO: 28 - DNA, 401 bp, Homo sapiens
tcttggggcatcaacttaaacccttcccaggctcccctccactgagaatgtgtctcaaggcctcactgcagcccatgaggctccgcagggtcctcctccctccctgactgctgtcacgcatgccagccgcacacctgctttctgtcccttaaagctcattcccacccaggacatctgcactcgcagctgcctcccgccgctgaaggcttcccggcccacccccatctgcacacgcgcagatccacttcttctgtcccttcctgcctccactccccatgcccctgtctcgtcaggctctcccaggagaccatgggtgccctcccccacccccagttcagttccctcacagcactgccaccagctggatctgtctcaattatcactggcttattgtttgctgc

SEQ ID NO: 29 - DNA, 401 bp, Homo sapiens
gtttctgtctgctggttgttaaacacgtatgagctcctcactgctgttacccctatcagcacctatgcagggcctgagaagctgctcaaactgcttgatccccccagccaagccaggcaagagaataaggacggagtagggagggattcccaaaggtgagtagttgagacgtactccggagccagcctgggcactggagccggaaggggcttccccggcccctccctctgcaccttcccatcagaagccttctgggccgttcctggagcttcaccccagtcactccacttcaaggtcagagagaaggacaattgctaagcagttcctcccgatgcaaagctcaaaacaagccccaggtcctcctgctcagtgtgagagagaggacgacgaaggagggaaac

SEQ ID NO: 30 - DNA, 401 bp, Homo sapiens
gtttctgtctgctggttgttaaacacgtatgagctcctcactgctgttacccctatcagcacctatgcagggcctgagaagctgctcaaactgcttgatccccccagccaagccaggcaagagaataaggacggagtagggagggattcccaaaggtgagtagttgagacgtactccggagccagcctgggcactggagctggaaggggcttccccggcccctccctctgcaccttcccatcagaagccttctgggccgttcctggagcttcaccccagtcactccacttcaaggtcagagagaaggacaattgctaagcagttcctcccgatgcaaagctcaaaacaagccccaggtcctcctgctcagtgtgagagagaggacgacgaaggagggaaac

SEQ ID NO: 31 - DNA, 401 bp, Homo sapiens
ccaaggtgtggctggaggaagcagagtctactcccgctaagtctgtccgctcactgctggccaaagctgccctgcgtctcctccccaccgccagccagagggaacctgcaatttcacctcatttagaggtaaaacatctaaatttaacgttatgggcttttggggctgggtggcttttatgcctgagtccctcacttagggctcctttttatccactcaaatgccagctagggcttagtttgtttataggagtttccaaaatagctcctttggtttcgcatgaaaggaaatggcaaaatagcccaggaagaggaatgtgagtttacacagaagacagacaggcgcccgaggaggcttctctgggaaccagttcgcctgtaccagagggggcccgagaaagt

SEQ ID NO: 32 - DNA, 401 bp, Homo sapiens
ccaaggtgtggctggaggaagcagagtctactcccgctaagtctgtccgctcactgctggccaaagctgccctgcgtctcctccccaccgccagccagagggaacctgcaatttcacctcatttagaggtaaaacatctaaatttaacgttatgggcttttggggctgggtggcttttatgcctgagtccctcacttaggactcctttttatccactcaaatgccagctagggcttagtttgtttataggagtttccaaaatagctcctttggtttcgcatgaaaggaaatggcaaaatagcccaggaagaggaatgtgagtttacacagaagacagacaggcgcccgaggaggcttctctgggaaccagttcgcctgtaccagagggggcccgagaaagt

SEQ ID NO: 33 - DNA, 401 bp, Homo sapiens
tacgttagaaggaccctacgttagaagggtgaggcgctagggccatagcctaagggcactgggaaccctgtgggcatgcgcagttcaagcccatccccgctccctccagctgctgtccatccctgccacacctgaccatttgcctaacctagatccttcctgtcttgcatttcctcaagcatccggagcccaggactgctgagtcaaccctctggaatgcccacaactccccacaggccagccggccttgggactcccgcacagccacgtgagccggtggagccgggtctgtttgctagtggaggctgttaacagcacgggaagtggtcaagggttcaacaagagatgagccatctggtcctccagaggtaaacaatttacaagagacacatcaagccggc

SEQ ID NO: 34 - DNA, 401 bp, Homo sapiens
tacgttagaaggaccctacgttagaagggtgaggcgctagggccatagcctaagggcactgggaaccctgtgggcatgcgcagttcaagcccatccccgctccctccagctgctgtccatccctgccacacctgaccatttgcctaacctagatccttcctgtcttgcatttcctcaagcatccggagcccaggactgctcagtcaaccctctggaatgcccacaactccccacaggccagccggccttgggactcccgcacagccacgtgagccggtggagccgggtctgtttgctagtggaggctgttaacagcacgggaagtggtcaagggttcaacaagagatgagccatctggtcctccagaggtaaacaatttacaagagacacatcaagccggc

SEQ ID NO: 35 - DNA, 401 bp, Homo sapiens
gggtttcccccaagcccctttccccctttgcgcctcccacttctcctagattgagagtcagcttggttctttcctttacatccattagtgagggtcaggctcttttgttatgtttttttttcttttgtataacttaattatttcagggttcggggtgggcgctcgccccttgcccagtcacactggtgtgtgtgcgactcctacaaagttaacagtttctccaggtcaaggggtgggatccaggcttggtgatgtgcacaatttcttttgtccacttgacacatctctgcgtcctgattctgctcagggacggacccaagaacaaagcagccatttaccgcctccggaggggaggccagccctgtggcacatccagggccttggaacacctagagacagat

SEQ ID NO: 36 - DNA, 401 bp, Homo sapiens
gggtttcccccaagcccctttccccctttgcgcctcccacttctcctagattgagagtcagcttggttctttcctttacatccattagtgagggtcaggctcttttgttatgtttttttttcttttgtataacttaattatttcagggttcggggtgggcgctcgccccttgcccagtcacactggtgtgtgtgcgactcttacaaagttaacagtttctccaggtcaaggggtgggatccaggcttggtgatgtgcacaatttcttttgtccacttgacacatctctgcgtcctgattctgctcagggacggacccaagaacaaagcagccatttaccgcctccggaggggaggccagccctgtggcacatccagggccttggaacacctagagacagat

SEQ ID NO: 37 - protein, 1068 aa, Homo sapiens
MLVGSQSFSPGGPNGIIRSQSFAGFSGLQERRSRCNSFIENSSALKKPQAKLKKMHNLGHKNNNPPKEPQPKRVEEVYRALKNGLDEYLEVHQTELDKLTAQLKDMKRNSRLGVLYDLDKQIKTIERYMRRLEFHISKVDELYEAYCIQRRLQDGASKMKQAFATSPASKAARESLTEINRSFKEYTENMCTIEVELENLLGEFSIKMKGLAGFARLCPGDQYEIFMKYGRQRWKLKGKIEVNGKQSWDGEETVFLPLIVGFISIKVTELKGLATHILVGSVTCETKELFAARPQVVAVDINDLGTIKLNLEITWYPFDMEDMTASSGAGNKAAALQRRMSMYSQGTPETPTFKDHSFFRWLHPSPDKPRRLSVLSALQDTFFAKLHRSRSFSDLPSLRPSPKAVLELYSNLPDDIFENGKAAEEKMPLSLSFSDLPNGDCALTSHSTGSPSNSTNPEITITPAEFNLSSLASQNEGMDDTSSASSRNSLGEGQEPKSHLKEEDPEEPRKPASAPSEACRRQSSGAGAEHLFLENDVAEALLQESEEASELKPVELDTSEGNITKQLVKRLTSAEVPMATDRLLSEGSVGGESEGCRSFLDGSLEDAFNGLLLALEPHKEQYKEFQDLNQEVMNLDDILKCKPAVSRSRSSSLSLTVESALESFDFLNTSDFDEEEDGDEVCNVGGGADSVFSDTETEKHSYRSVHPEARGHLSEALTEDTGVGTSVAGSPLPLTTGNESLDITIVRHLQYCTQLVQQIVFSSKTPFVARSLLEKLSRQIQVMEKLAAVSDENIGNISSVVEAIPEFHKKLSLLSFWTKCCSPVGVYHSPADRVMKQLEASFARTVNKEYPGLADPVFRTLVSQILDQAEPLLSSSLSSEVVTVFQYYSYFTSHGVSDLESYLSQLARQVSMVQTLQSLRDEKLLQTMSDLAPSNLLAQQEVLRTLALLLTREDNEVSEAVTLYLAAASKNQHFREKALLYYCEALTKTNLQLQKAACLALKILEATESIKMLVTLCQSDTEEIRNVASETLLSLGEDGRLAYEQLDKFPRDCVKVGGRHGTEVATAF

SEQ ID NO: 38 - protein, 591 aa, Homo sapiens
MLVGSQSFSPGGPNGIIRSQSFAGFSGLQERRSRCNSFIENSSALKKPQAKLKKMHNLGHKNNNPPKEPQPKRVEEVYRALKNGLDEYLEVHQTELDKLTAQLKDMKRNSRLGVLYDLDKQIKTIERYMRRLEFHISKVDELYEAYCIQRRLQDGASKMKQAFATSPASKAARESLTEINRSFKEYTENMCTIEVELENLLGEFSIKMKGLAGFARLCPGDQYEIFMKYGRQRWKLKGKIEVNGKQSWDGEETVFLPLIVGFISIKVTELKGLATHILVGSVTCETKELFAARPQVVAVDINDLGTIKLNLEITWYPFDMEDMTASSGAGNKAAALQRRMSMYSQGTPETPTFKDHSFFSNLPDDIFENGKAAEEKMPLSLSFSDLPNGDCALTSHSTGSPSNSTNPEITITPAEFNLSSLASQNEGMDDTSSASSRNSLGEGQEPKSHLKEEDPEEPRKPASAPSEACRRQSSGAGAEHLFLENDVAEALLQESEEASELKPVELDTSEGNITKQLVKRLTSAEVPMATDRLLSEGSVGGESEGCRSFLDGSLEDAFNGLLLALEPHKEQYKEFQDLNQEVMNLDDILKK

SEQ ID NO: 39 - protein, 1048 aa, Homo sapiens
MLVGSQSFSPGGPNGIIRSQSFAGFSGLQERRSRCNSFIENSSALKKPQAKLKKMHNLGHKNNNPPKEPQPKRVEEVYRALKNGLDEYLEVHQTELDKLTAQLKDMKRNSRLGVLYDLDKQIKTIERYMRRLEFHISKVDELYEAYCIQRRLQDGASKMKQAFATSPASKAARESLTEINRSFKEYTENMCTIEVELENLLGEFSIKMKGLAGFARLCPGDQYEIFMKYGRQRWKLKGKIEVNGKQSWDGEETVFLPLIVGFISIKVTELKGLATHILVGSVTCETKELFAARPQVVAVDINDLGTIKLNLEITWYPFDMEDMTASSGAGNKAAALQRRMSMYSQGTPETPTFKDHSFFRWLHPSPDKPRRLSVLSALQDTFFAKLHRSRSFSDLPSLRPSPKAVLELYSNLPDDIFENGKAAEEKMPLSLSFSDLPNGDCALTSHSTGSPSNSTNPEITITPAEFNLSSLASQNEGMDDTSSASSRNSLGEGQEPKSHLKEEDPEEPRKPASAPSEACRRQSSGAGAEHLFLENDVAEALLQESEEASELKPVELDTSEGNITKQLVKRLTSAEVPMATDRLLSEGSVGGESEGCRSFLDGSLEDAFNGLLLALEPHKEQYKEFQDLNQEVMNLDDILKCKPAVSRSRSSSLSLTVESALESFDFLNTSDFDEEEDGDEVCNVGGGADSVFSDTETEKHSYRSVHPEARGHLSEALTEDTGVGTSVAGSPLPLTTGNESLDITIVRHLQYCTQLVQQIVFSSKTPFVARSLLEKLSRQIQVMEKLAAVSDENIGNISSVVEAIPEFHKKLSLLSFWTKCCSPVGVYHSPADRVMKQLEASFARTVNKEYPGLADPVFRTLVSQILDQAEPLLSSSLSSEVVTVFQYYSYFTSHGVSDLESYLSQLARQVSMVQTLQSLRDEKLLQTMSDLAPSNLLAQQEVLRTLALLLTREDNEVSEAVTLYLAAASKNQHFREKALLYYCEALTKTNLQLQKAACLALKILEATESIKMLVTLCQSDTEEIRNVASETLLSLGEDGRLAYEQLDK

SEQ ID NO: 40 - protein, 479 aa, Homo sapiens
MLVGSQSFSPGGPNGIIRSQSFAGFSGLQERRSRCNSFIENSSALKKPQAKLKKMHNLGHKNNNPPKEPQPKRVEEVYRALKNGLDEYLEVHQTELDKLTAQLKDMKRNSRLGVLYDLDKQIKTIERYMRRLEFHISKVDELYEAYCIQRRLQDGASKMKQAFATSPASKAARESLTEINRSFKEYTENMCTIEVELENLLGEFSIKMKGLAGFARLCPGDQYEIFMKYGRQRWKLKGKIEVNGKQSWDGEETVFLPLIVGFISIKVTELKGLATHILVGSVTCETKELFAARPQVVAVDINDLGTIKLNLEITWYPFDMEDMTASSGAGNKAAALQRRMSMYSQGTPETPTFKDHSFFSNLPDDIFENGKAAEEKMPLSLSFSDLPNGDCALTSHSTGSPSNSTNPEITITPAEFNLSSLASQNEGMDDTSSASSRNSLGEGQEPKSHLKEEDPEEPRKPASAPSEACRRQSSGAGAE

(19) United States
(12) Patent Application Publication
(10) Pub. No.: US 2008/0227737 A1
(43) Pub. Date: Sep. 18, 2008

(54) Type 2 diabetes mellitus genes

(76) Inventors: Andrzej S. Krolewski, Needham, MA (US); Marcus G. Pezzolesi, Somerville, MA (US); Terumasa Nagase, Matsuyama-Shi (JP)

Correspondence Address: FISH & RICHARDSON PC, P.O. BOX 1022, MINNEAPOLIS, MN, 55440-1022, US

(21) Appl. No.: 11/805,817

(22) Filed: May 24, 2007

Related U.S. Application Data
(63) Continuation of application No. 10/694,685, filed on Oct. 28, 2003, now abandoned.
(60) Provisional application No. 60/421,844, filed on Oct. 28, 2002.

(51) Int. Cl. A61K 31/70; C12N 15/11; C12N 15/00; C12N 5/06; C07K 14/00; C12Q 1/02; A61P 1/00; A01K 67/027; C07K 16/18; C12P 21/04; C12Q 1/68; C40B 40/08

(52) U.S. Cl. 514/44; 536/23.5; 435/320.1; 435/325; 435/366; 530/350; 530/387.9; 435/69.1; 435/6; 506/17; 435/29; 800/3; 800/13

(57) ABSTRACT

Methods and compositions related to novel genes associated with type 2 diabetes mellitus.

### RELATED APPLICATIONS

This application is a continuation of U.S. application Ser. No. 10/694,685, filed Oct. 28, 2004, which claims the benefit of U.S. Provisional Application Ser. No. 60/421,844, filed Oct. 28, 2003. The contents of the foregoing applications are incorporated herein by reference in their entirety.

### GOVERNMENT FUNDING

This invention was made with support from the United States Government under grant number DK 47475 awarded by the National Institute of Health. The Government has certain rights in the invention.

### BACKGROUND

Type 2 diabetes mellitus is a metabolic disease of impaired glucose homeostasis characterized by hyperglycemia, or high blood sugar, as a result of defective insulin action which manifests as insulin resistance, defective insulin secretion, or both. A patient with Type 2 diabetes mellitus has abnormal carbohydrate, lipid, and protein metabolism associated with insulin resistance and impaired insulin secretion. The disease leads to pancreatic beta cell destruction and eventually absolute insulin deficiency. Without insulin, high glucose levels remain in the blood. The long term effects of high blood glucose include blindness, renal failure, and poor blood circulation to these areas, which can lead to foot and ankle amputations. Early detection is critical in preventing patients from reaching this severity. The majority of patients with diabetes have the non-insulin dependent form of diabetes, currently referred to as Type 2 diabetes mellitus.

### SUMMARY

The invention is based, in part, on the identification and cloning of two genes associated with susceptibility to Type 2 diabetes mellitus, referred to herein as T2DM genes, e.g., T2DM-1 and T2DM-2, each having a long form (T2DM-1a and T2DM-2a, respectively) and a short form (T2DM-1b and T2DSM-2b, respectively). Numerous polymorphisms associated with diabetes, e.g., SNPs, of each gene have also been discovered. The nucleotide sequence of T2DM-1a is shown as SEQ ID NO:1 and its amino acid sequence as SEQ ID NO:2. The nucleotide sequence of T2DM-1b is shown as SEQ ID NO: 3 and its amino acid sequence as SEQ ID NO:4. The nucleotide sequence of T2DM-2a is shown as SEQ ID NO:5. The nucleotide sequence of T2DM-2b is shown as SEQ ID NO: 6. Fourteen polymorphisms of the genes associated with diabetes are shown as SEQ ID NOs: 9-36. The sequences described herein are useful for, inter alia, genetic screening for susceptibility to type 2 diabetes mellitus, diagnosis, therapy, and pharmacogenomics applications.

Accordingly, in one aspect, the invention features T2DM-1 and T2DM-2 nucleic acid molecules that encode a mammalian T2DM-1 or T2DM-2 protein or polypeptide, e.g., a biologically active portion of a T2DM-1 or T2DM-2 protein. In one embodiment, the invention provides isolated T2DM-1 nucleic acid molecules having the nucleotide sequence shown in SEQ ID NO:1 or 3 or isolated T2DM-2 nucleic acid molecules having the nucleotide sequence shown in SEQ ID NO:5 or 6. In one embodiment the isolated nucleic acid molecule encodes a polypeptide having the amino acid sequence of SEQ ID NO:2 or 4. In still other embodiments, the invention provides nucleic acid molecules that are substantially identical (e.g., naturally occurring allelic variants) to the nucleotide sequence shown in SEQ ID NO:1, 3, 5 or 6, e.g., naturally occurring variants, e.g., having single nucleotide polymorphisms (SNPs) described herein, e.g., isolated nucleic acid molecules including a nucleotide sequence shown in FIG. 4A-D, e.g., any one of SEQ ID NOs:9-22. In other embodiments, the invention provides a nucleic acid molecule that hybridizes under a stringency condition described herein to a nucleic acid molecule comprising the nucleotide sequence of SEQ ID NO:1, 3, 5 or 6, wherein the nucleic acid encodes a full length T2DM-1 or T2DM-2 protein, or a fragment thereof.

In a related aspect, the invention further provides nucleic acid constructs that include a T2DM-1 or T2DM-2 nucleic acid molecule described herein. In certain embodiments, the nucleic acid molecules are operatively linked to native or heterologous regulatory sequences. In some embodiments, the construct includes a nucleic acid sequence encoding a fragment, e.g., a biologically active or functional fragment, of T2DM-1 or T2DM-2 linked to a heterologous nucleic acid sequence, e.g., a sequence encoding a peptide tag or other fusion protein. Also included are vectors and host cells containing the T2DM-1 or T2DM-2 nucleic acid molecules of the invention e.g., vectors and host cells (e.g., bacterial, or eukaryotic, e.g., mammalian, e.g., human cells) suitable for producing T2DM-1 or T2DM-2 nucleic acid molecules and polypeptides.

In another related aspect, the invention provides nucleic acid (e.g., RNA or DNA) fragments, e.g., single stranded or double stranded nucleic acid fragments. Such fragments are suitable as primers or hybridization probes, e.g., for the detection of T2DM-1 or T2DM-2-encoding nucleic acids; or as antisense reagents, e.g., siRNA, ssRNA, dsRNA, or mRNA-cDNA hybrid fragments. A probe or primer can include a sequence at least 80%, preferably 85%, 90%, 95%, 98%, 99% or 100% identical to a sequence consisting of at least 20 contiguous nucleotides of SEQ ID NO:1, 3, 5 or 6. In some embodiments, a probe or primer is between about 20 and 500 nucleotides in length, preferably between about 20 and 200 nucleotides in length, or between about 25 and 100 nucleotides in length. The probes of primers described herein can be used, e.g., to detect the presence of a T2DM-1 or T2DM-2 nucleic acid, e.g., to detect a T2DM-1 or T2DM-2 polymorphism, e.g., a polymorphism described herein; or in antisense, RNA interference, or other gene silencing techniques.

In another aspect, the invention features, T2DM-1 or T2DM-2 polypeptides or fragments thereof, e.g., biologically active or antigenic fragments thereof that are useful, e.g., as reagents or targets in assays applicable to treatment and diagnosis of a T2DM-1 or T2DM-2-mediated or T2DM-1 or T2DM-2-related disorder, e.g., Type 2 diabetes or a Type-2 diabetes-associated condition, e.g., obesity, hyperglycemia, hypertension. In another embodiment, the invention provides T2DM, e.g., T2DM-1 or T2DM-2 polypeptides having a T2DM-1 or T2DM-2 activity described herein. Preferred polypeptides are T2DM-1 or T2DM-2 proteins having at least one T2DM-1 or T2DM-2 activity, e.g., modulation of insulin function or beta cell function or another T2DM-1 or T2DM-2 activity as described herein.

In other embodiments, the invention provides T2DM-1 or T2DM-2 polypeptides, e.g., a T2DM-1 or T2DM-2 polypeptide having the amino acid sequence shown in SEQ ID NO:2 or 4; an amino acid sequence that is substantially identical to the amino acid sequence shown in SEQ ID NO: 2 or 4; or an amino acid sequence encoded by a nucleic acid molecule having a nucleotide sequence which hybridizes under a stringency condition described herein to a nucleic acid molecule comprising the nucleotide sequence of SEQ ID NO:1, 3, 5 or 6, wherein the nucleic acid encodes a full length T2DM-1 or T2DM-2 protein or a fragment thereof, e.g., a biologically active and/or immunogenic fragment thereof.

In a related aspect, the invention further provides nucleic acid constructs and host cells, e.g., mammalian, e.g., human, host cells, which include a T2DM, e.g., T2DM-1 or T2DM-2 nucleic acid molecule described herein.

In another aspect, the invention provides an isolated polypeptide that includes an T2DM-1 or T2DM-2, or a functional and/or immunogenic fragment thereof, and a heterologous amino acid sequence, e.g., a T2DM-1 or T2DM-2 polypeptide or fragment operatively linked to a non-T2DM-1 or non-T2DM-2 polypeptide to form a fusion protein.

In another aspect, the invention features antibodies and antigen-binding fragments thereof, that react with, or more preferably specifically bind T2DM-1 or T2DM-2 polypeptides or fragments thereof.

In another aspect, the invention provides a method of determining if a subject is at risk for or has an insulin related disorder, e.g., type 2 diabetes. The method includes: (a) evaluating the level, activity, expression and/or genotype of a T2DM-1 or T2DM-2 molecule in a subject, e.g., in a biological sample of the subject, and (b) correlating an alteration in a T2DM-1 or T2DM-2 molecule, e.g., a non wildtype level, activity, expression, and/or genotype of T2DM-1 or T2DM-2 with a risk for or presence of an insulin related disorder, e.g., type 2 diabetes. Correlating means identifying the alteration as a risk or diagnostic factor of type 2 diabetes, e.g., providing a print material or computer readable medium, e.g., an informational, diagnostic, marketing or instructional print material or computer readable medium, e.g., to the subject or to a health care provider, identifying the alteration as a risk or diagnostic factor for type 2 diabetes.

In a preferred embodiment, the method includes diagnosing a subject as being at risk for or having type 2 diabetes. In another preferred embodiment, the method includes prescribing or beginning a treatment for type 2 diabetes in the subject. In some embodiments, the method includes performing a second diagnostic test for type 2 diabetes, e.g., the evaluation of the level, activity, expression and/or genotype of a T2DM-1 or T2DM-2 molecule in a subject can be repeated, e.g., by performing the same or a different determination as described herein, or by performing another type 2 diabetes diagnostic test known in the art, e.g., evaluating insulin and/or glucose levels in the subject.

The subject is preferably a human, e.g., a human with a family history of diabetes or its associated conditions, e.g., obesity, nephropathy, retinopathy. The biological sample can be a cell sample, tissue sample, or at least partially isolated molecules, e.g., nucleic acids, e.g., genomic DNA, cDNA, mRNA, and/or proteins derived from the subject. Such methods are useful, e.g., for diagnosis of diabetes or diabetes risk, e.g., type 2 diabetes mellitus.

In a preferred embodiment, the method includes one or more of the following:

1) detecting, in a biological sample of the subject, the presence or absence of a mutation that affects the expression of a T2DM-1 or T2DM-2 gene, or detecting the presence or absence of a mutation in a region which controls the expression of the gene, e.g., a mutation in the 5′ control region, the presence of a mutation being indicative of risk;

2) detecting, in a biological sample of the subject, the presence or absence of a mutation that alters the structure of a T2DM-1 or T2DM-2 gene, the presence of a mutation being indicative of risk;

3) detecting, in a biological sample of the subject, the misexpression of a T2DM-1 or T2DM-2 gene, at the mRNA level, e.g., detecting a non-wild type level of a T2DM-1 or T2DM-2 mRNA, non-wildtype levels of T2DM-1 or T2DM-2 mRNA being associated with risk. Detecting misexpression can include ascertaining the existence of at least one of: an alteration in the level of a messenger RNA transcript of a T2DM-1 or T2DM-2 gene compared to a reference, e.g., as compared to a baseline value or to levels in a subject not at risk for an insulin related disorder; the presence of a non-wild type splicing pattern of a messenger RNA transcript of the gene; or a non-wild type level of a T2DM-1 or T2DM-2 protein e.g., as compared a reference, e.g., compared to a baseline value, or to levels in a subject not at risk for an insulin related disorder;

4) detecting, in a biological sample of the subject, the misexpression of a T2DM-1 or T2DM-2 gene, at the protein level, e.g., detecting a non-wildtype level of a T2DM-1 or T2DM-2 polypeptide, decreased or increased levels of T2DM-1 or T2DM-2 protein (e.g., compared to a control) being indicative of a risk. For example, the method can include contacting a sample from the subject with an antibody to a T2DM-1 or T2DM-2 protein;

5) detecting, in a biological sample of the subject, a polymorphism, e.g., a SNP, in a T2DM-1 or T2DM-2 gene, which is associated with type 2 diabetes, e.g., detecting a polymorphism described herein, e.g., detecting one or more polymorphisms described in FIGS. 4A-H and FIG. 10. In preferred embodiments the method includes: ascertaining the existence of at least one of: a deletion of one or more nucleotides from the T T2DM-1 or T2DM-2 gene; an insertion of one or more nucleotides into the gene; a point mutation, e.g., a substitution of one or more nucleotides of the gene; a gross chromosomal rearrangement of the gene, e.g., a translocation, inversion, duplication or deletion. In a preferred embodiment, a SNP or haplotype associated with diabetes risk is detected.

In one embodiment, detecting a mutation or polymorphism can include: (i) providing a probe or primer, e.g., a labeled probe or primer, that includes a region of nucleotide sequence which hybridizes to a sense or antisense sequence from a T2DM-1 or T2DM-2 gene, or naturally occurring mutants thereof, or to the 5′ or 3′ flanking sequences naturally associated with a T2DM-1 or T2DM-2 gene; (ii) exposing the probe/primer to nucleic acid of the subject; and detecting, e.g., by hybridization, e.g., in situ hybridization to the nucleic acid; or amplification of the nucleic acid, the presence or absence of the mutation or polymorphism.

In a preferred embodiment, the method includes performing one or more of the following determinations, for one or both chromosomes of the subject:
(a) determining the identity of the nucleotides of T2DM-1 or T2DM-2 corresponding to nucleotides 201 to 204 of SEQ ID NO:9, e.g., determining whether either the coding or non coding strand of a T2DM gene of the subject includes the nucleotide sequence of SEQ ID NO:9 having a polymorphism, e.g., a deletion, e.g., a deletion of nucleotides TTGA, at nucleotides 201 to 204, e.g., determining if the coding or non coding strand of a T2DM gene of the subject includes the nucleotide sequence of SEQ ID NO:10;
(b) determining the identity of the nucleotide of T2DM-1 or T2DM-2 corresponding to nucleotide 201 of SEQ ID NO:11, e.g., determining whether either the coding or non coding strand of a T2DM gene of the subject includes the nucleotide sequence of SEQ ID NO:11 having a polymorphism, e.g., a substitution, e.g., an A/G substitution, at nucleotide 201, e.g., determining if the coding or non coding strand of a T2DM gene of the subject includes the nucleotide sequence of SEQ ID NO:12;
(c) determining the identity of the nucleotide of T2DM-1 or T2DM-2 corresponding to nucleotide 201 of SEQ ID NO:13, e.g., determining whether either the coding or non coding strand of a T2DM gene of the subject includes the nucleotide sequence of SEQ ID NO:13 having a polymorphism, e.g., a substitution, e.g., an A/G substitution, at nucleotide 201, e.g., determining if the coding or non coding strand of a T2DM gene of the subject includes the nucleotide sequence of SEQ ID NO:14;
(d) determining the identity of the nucleotide of T2DM-1 or T2DM-2 corresponding to nucleotide 201 of SEQ ID NO:15, e.g., determining whether either the coding or non coding strand of a T2DM gene of the subject includes the nucleotide sequence of SEQ ID NO:15 having a polymorphism, e.g., a substitution, e.g., an A/G substitution, at nucleotide 201, e.g., determining if the coding or non coding strand of a T2DM gene of the subject includes the nucleotide sequence of SEQ ID NO:16;
(e) determining the identity of the nucleotide of T2DM-1 or T2DM-2 corresponding to nucleotide 201 of SEQ ID NO:17, e.g., determining whether either the coding or non coding strand of a T2DM gene of the subject includes the nucleotide sequence of SEQ ID NO:17 having a polymorphism, e.g., a substitution, e.g., an A/C substitution, at nucleotide 201, e.g., determining if the coding or non coding strand of a T2DM gene of the subject includes the nucleotide sequence of SEQ ID NO:18;
(f) determining the identity of the nucleotides of T2DM-1 or T2DM-2 corresponding to nucleotides 201-216 of SEQ ID NO:19, e.g., determining whether either the coding or non coding strand of a T2DM gene of the subject includes the nucleotide sequence of SEQ ID NO:19 having a polymorphism, e.g., a deletion, e.g., a deletion of nucleotides TTAGTGCCGGGCCGGC, from nucleotide 201 to 216, e.g., determining if the coding or non coding strand of a T2DM gene of the subject includes the nucleotide sequence of SEQ ID NO:20;
(g) determining the identity of the nucleotide of T2DM-1 or T2DM-2 corresponding to nucleotide 201 of SEQ ID NO:21, e.g., determining whether either the coding or non coding strand of a T2DM gene of the subject includes the nucleotide sequence of SEQ ID NO:21 having a polymorphism, e.g., a substitution, e.g., an A/G substitution, at nucleotide 201, e.g., determining if the coding or non coding strand of a T2DM gene of the subject includes the nucleotide sequence of SEQ ID NO:22;
(h) determining the identity of the nucleotide of T2DM-1 or T2DM-2 corresponding to nucleotide 201 of SEQ ID NO:23, e.g., determining whether either the coding or non coding strand of a T2DM gene of the subject includes the nucleotide sequence of SEQ ID NO:23 having a polymorphism, e.g., a substitution, e.g., an A/G substitution, at nucleotide 201, e.g., determining if the coding or non coding strand of a T2DM gene of the subject includes the nucleotide sequence of SEQ ID NO:24;
(i) determining the identity of the nucleotide of T2DM-1 or T2DM-2 corresponding to nucleotide 201 of SEQ ID NO:25, e.g., determining whether either the coding or non coding strand of a T2DM gene of the subject includes the nucleotide sequence of SEQ ID NO:25 having a polymorphism, e.g., a substitution, e.g., an A/C substitution, at nucleotide 201, e.g., determining if the coding or non coding strand of a T2DM gene of the subject includes the nucleotide sequence of SEQ ID NO:26;
(j) determining the identity of the nucleotide of T2DM-1 or T2DM-2 corresponding to nucleotide 201 of SEQ ID NO:27, e.g., determining whether either the coding or non coding strand of a T2DM gene of the subject includes the nucleotide sequence of SEQ ID NO:27 having a polymorphism, e.g., a substitution, e.g., a C/T substitution, at nucleotide 201, e.g., determining if the coding or non coding strand of a T2DM gene of the subject includes the nucleotide sequence of SEQ ID NO:28;
(k) determining the identity of the nucleotide of T2DM-1 or T2DM-2 corresponding to nucleotide 201 of SEQ ID NO:29, e.g., determining whether either the coding or non coding strand of a T2DM gene of the subject includes the nucleotide sequence of SEQ ID NO:29 having a polymorphism, e.g., a substitution, e.g., C/T substitution, at nucleotide 201, e.g., determining if the coding or non coding strand of a T2DM gene of the subject includes the nucleotide sequence of SEQ ID NO:30;
(l) determining the identity of the nucleotide of T2DM-1 or T2DM-2 corresponding to nucleotide 201 of SEQ ID NO:31, e.g., determining whether either the coding or non coding strand of a T2DM gene of the subject includes the nucleotide sequence of SEQ ID NO:31 having a polymorphism, e.g., a substitution, e.g., an G/A substitution, at nucleotide 201, e.g., determining if the coding or non coding strand of a T2DM gene of the subject includes the nucleotide sequence of SEQ ID NO:32;
(m) determining the identity of the nucleotide of T2DM-1 or T2DM-2 corresponding to nucleotide 201 of SEQ ID NO:33, e.g., determining whether either the coding or non coding strand of a T2DM gene of the subject includes the nucleotide sequence of SEQ ID NO:33 having a polymorphism, e.g., a substitution, e.g., a G/C substitution, at nucleotide 201, e.g., determining if the coding or non coding strand of a T2DM gene of the subject includes the nucleotide sequence of SEQ ID NO:34;
(n) determining the identity of the nucleotide of T2DM-1 or T2DM-2 corresponding to nucleotide 201 of SEQ ID NO:35, e.g., determining whether either the coding or non coding strand of a T2DM gene of the subject includes the nucleotide sequence of SEQ ID NO:35 having a polymorphism, e.g., a substitution, e.g., a C/T substitution, at nucleotide 201, e.g., determining if the coding or non coding strand of a T2DM gene of the subject includes the nucleotide sequence of SEQ ID NO:36.

In a preferred embodiment, the determining step includes amplifying at least a portion of a T2DM-1 or T2DM-2 nucleic acid molecule of the subject, e.g., a portion including a polymorphism described herein.

In a preferred embodiment, the determining step includes sequencing at least a portion of a T2DM-1 or T2DM-2 nucleic acid molecule of the subject, e.g., a portion including a polymorphism described herein.

In a preferred embodiment, the determining step includes hybridizing a T2DM-1 or T2DM-2 nucleic acid molecule of the subject with a probe or primer, e.g., a probe or primer described herein, e.g., a probe or primer including a polymorphism described herein.

In another embodiment, the method includes determining the activity of or the presence or absence of T2DM-1 or T2DM-2 nucleic acid molecules and/or polypeptides or in a biological sample.

Methods of the invention can be used prenatally or to determine if a subject's offspring will be at risk for a disorder.

In another aspect, the invention features an isolated nucleic acid, e.g., a probe or primer, or partial or complete cDNA, or a genomic fragment, or its complement, wherein the nucleic acid includes at least 10, preferably at least 15, more preferably at least 20 contiguous nucleotides of any one of:
(a) SEQ ID NO:10, wherein the nucleic acid includes nucleotides 203 and 204 (CA) of SEQ ID NO:10;
(b) SEQ ID NO:12, wherein the nucleic acid includes nucleotide 201 (G) of SEQ ID NO:12;
(c) SEQ ID NO:14, wherein the nucleic acid includes nucleotide 201 (G) of SEQ ID NO:14;
(d) SEQ ID NO:16, wherein the nucleic acid includes nucleotide 201 (G) of SEQ ID NO:16;
(e) SEQ ID NO:18, wherein the nucleic acid includes nucleotide 201 (C) of SEQ ID NO:10;
(f) SEQ ID NO:20, wherein the nucleic acid includes nucleotides 199 to 202
(GCCC) of SEQ ID NO:20;
(g) SEQ ID NO:22, wherein the nucleic acid includes nucleotide 201 (G) of SEQ ID NO:22;
(h) SEQ ID NO:24, wherein the nucleic acid includes nucleotide 201 (G) of SEQ ID NO:24;
(i) SEQ ID NO:26, wherein the nucleic acid includes nucleotide 201 (C) of SEQ ID NO:26;
(j) SEQ ID NO:28, wherein the nucleic acid includes nucleotide 201 (T) of SEQ ID NO:28;
(k) SEQ ID NO:30, wherein the nucleic acid includes nucleotide 201 (T) of SEQ ID NO:30;
(l) SEQ ID NO:32, wherein the nucleic acid includes nucleotide 201 (A) of SEQ ID NO:32;
(m) SEQ ID NO:34, wherein the nucleic acid includes nucleotide 201 (C) of SEQ ID NO:34;
(n) SEQ ID NO:36, wherein the nucleic acid includes nucleotide 201 (T) of SEQ ID NO:36.

In a preferred embodiment, the isolated nucleic acid or its complement includes a detectable label, e.g., a radioactive, fluorescent or colorimetric label.
In a preferred embodiment, the nucleic acid or its complement includes less than 200 contiguous nucleotides, preferably less than 150 contiguous nucleotides, more preferably less than 100 contiguous nucleotides of the subject sequence.

In one embodiment, the nucleic acid, or its complement, is attached to a solid support, e.g., the nucleic acid is part of an array of nucleic acids, e.g., an array that includes one, preferably 2, more preferably 3, 4, 5, 6, 7, 8, 9, 10, 11, 12, 13 or more of the nucleic acids of (a)-(n) described herein.

In a preferred embodiment, the nucleic acid, or its complement, hybridizes under high stringency conditions to the sequence of SEQ ID NO:10, 12, 14, 16, 18, 20, 22, 24, 26, 28, 30, 32, 34 or 36, but not to the corresponding sequence of SEQ ID NO:9, 11, 13, 15, 17, 19, 21, 23, 25, 27, 29, 31, 33 or 35 (or vice versa).

In another aspect, the invention features an array of nucleic acid molecules, e.g., nucleic acid molecules attached to a solid support. The array includes 2 or more T2DM-1 or T2DM-2 nucleic acids, e.g., probes or primers described herein, that are capable of detecting (e.g., hybridizing to) a T2DM-1 or T2DM-2 polymorphism, e.g., a T2DM-1 or T2DM-2 polymorphism described herein. For example, the array can include one, preferably 2, more preferably 3, 4, 5, 6, 7, 8, 9, 10, 11, 12, 13, 25, 50, 100 or more of the probes or primers described herein.

In another aspect, the invention features a set of oligonucleotides, e.g., primers, for amplifying a genomic sequence that spans a T2DM-1 or T2DM-2 polymorphism, e.g., a T2DM-1 or T2DM-2 polymorphism described herein. FIG. 1, FIGS. 4A-H and FIG. 10 show numerous T2DM-1 or T2DM-2 polymorphisms associated with type 2 diabetes, in the context of the surrounding genomic sequence. One of skill in the art could easily design a set of primers to amplify any one or more of the polymorphisms described herein. For example, the set can include a plurality of oligonucleotides, each of which is at least partially complementary (e.g., at least 50%, 60%, 70%, 80%, 90%, 92%, 95%, 97%, 98%, or 99% complementary) to a T2DM-1 or T2DM-2 nucleic acid.

In a preferred embodiment the set includes a first and a second oligonucleotide. The first and second oligonucleotide can hybridize to the same or to different locations of SEQ ID NO:1, 3, 5, or 6 or the complement of SEQ ID NO:1, 3, 5, or 6. Different locations can be different but overlapping, or non-overlapping on the same strand. The first and second oligonucleotide can hybridize to sites on the same or on different strands. The set can be useful, e.g., for identifying SNP's, or identifying specific polymorphisms or alleles of T2DM-1 or T2DM-2. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tm of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a T2DM-1 or T2DM-2 nucleic acid.

The set described herein may be part of a kit including at least one probe nucleic acid or antibody reagent described herein, and instructions for using the kit to evaluate susceptibility for type 2 diabetes in a subject. The kit may be used, e.g., by a subject or health care provider.

In another aspect, the invention features a method of evaluating, e.g., diagnosing, a subject. The method includes identifying a subject suspected of being at risk for, e.g., a subject having a family history of, type 2 diabetes or an associated condition. The method includes: providing a nucleic acid sample from the subject; evaluating a genotype of the T2DM-1 or T2DM-2 gene of the subject, e.g., evaluating the presence or absence of a polymorphism in the subject's T2DM-1 or T2DM-2 gene, e.g., the presence or absence of a T2DM-1 or T2DM-2 polymorphism described herein (e.g., by determining the identity or sequence of a T2DM allele); and comparing the genotype, e.g., the haplotype, of the subject's T2DM-1 or T2DM-2 gene to a reference. The method optionally includes providing a treatment for type 2 diabetes to the subject.

In another aspect, the invention features a method of treating a subject. The method includes modulating the expression, level, or activity of a T2DM molecule, e.g., a T2DM-1 or T2DM-2 molecule, in a subject (e.g., in a liver, muscle, pancreatic islet, testis, kidney, adipose tissue, brain or placental cell of the subject). The subject can be a human or a non-human animal, e.g., an animal model for an insulin related disorder, e.g., a nod mouse, a Zucker rat, a fructose fed rodent, an Israeli sand rat. In a preferred embodiment, the subject is identified as having or being at risk for type 2 diabetes or an associated condition, e.g., hypertension, retinopathy, nephropathy, persistent hyperinsulinemic hypoglycemia of infancy (PHHI), insulin resistance, hyperglycemia, glucose intolerance, glucotoxicity. The level of the T2DM-1 or T2DM-2 protein can be modulated by modulating any of: T2DM-1 or T2DM-2 expression (e.g., modulating rate of transcription or mRNA stability), protein levels, or protein activity.

In a preferred embodiment, T2DM-1 or T2DM-2 is modulated in-vitro, e.g., in a cell or tissue of a subject. In some embodiments, the cell or tissue can be transplanted into a subject. The transplanted cell or tissue can be autologous, allogeneic, or xenogeneic.

In another preferred embodiment, T2DM-1 or T2DM-2 is modulated in vivo in a subject.

In a preferred embodiment, T2DM-1 or T2DM-2 activity, level or expression is increased, e.g., by administering to the subject an agent that increases T2DM-1 or T2DM-2 activity, level or expression. Increasing T2DM-1 or T2DM-2 expression, levels or activity can, e.g., increase the production of insulin in a subject in need of increased insulin production (e.g., a diabetic subject); or regulate pancreatic β-cell differentiation and/or proliferation in a subject in need of regulating pancreatic β-cell differentiation and/or proliferation (e.g., a subject with β-cell dysfunction). The agent can be, e.g., a T2DM-1 or T2DM-2 polypeptide or a functional fragment or analog thereof; a peptide or protein agonist of T2DM-1 or T2DM-2 that increases the activity of T2DM-1 or T2DM-2; a small molecule that increases expression of a T2DM-1 or T2DM-2; an antibody, e.g., an antibody that binds to and stabilizes or assists the binding of T2DM-1 or T2DM-2 to a binding partner; or a nucleotide sequence encoding a T2DM-1 or T2DM-2 polypeptide or functional fragment or analog thereof. The nucleotide sequence can be a genomic sequence or a cDNA sequence. The nucleotide sequence can include: a T2DM-1 or T2DM-2 coding region; a promoter sequence, e.g., a promoter sequence from a T2DM-1 or T2DM-2 gene or from another gene; an enhancer sequence; untranslated regulatory sequences, e.g., a 5′ untranslated region (UTR), e.g., a 5′UTR from a T2DM-1 or T2DM-2 gene or from another gene, a 3′ UTR, e.g., a 3′UTR from a T2DM-1 or T2DM-2 gene or from another gene; a polyadenylation site; an insulator sequence. In another embodiment, the nucleotide sequence includes a T2DM-1 or T2DM-2 functional domain linked to a functional domain from a heterologous molecule.

In another preferred embodiment, the level of T2DM-1 or T2DM-2 protein is increased by increasing the level of expression of an endogenous T2DM-1 or T2DM-2 gene, e.g., by increasing transcription of the T2DM-1 or T2DM-2 gene or increasing T2DM-1 or T2DM-2 mRNA stability. In a preferred embodiment, transcription of the T2DM-1 or T2DM-2 gene is increased by: altering the regulatory sequence of the endogenous T2DM-1 or T2DM-2 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the T2DM-1 or T2DM-2 gene to be transcribed more efficiently.

In some embodiments, T2DM-1 or T2DM-2 expression, levels or activity is increased in conjunction with another treatment, e.g., administration of insulin.

In another embodiment, T2DM-1 or T2DM-2 can be decreased by administering to the subject an agent that inhibits T2DM-1 or T2DM-2 gene expression, mRNA stability, protein production levels and/or activity. Decreasing T2DM-1 or T2DM-2 expression, levels or activity can, e.g., decrease insulin production in a subject with aberrantly high levels of insulin. An agent that inhibits T2DM-1 or T2DM-2 can be one or more of: a T2DM-1 or T2DM-2 binding protein, e.g., a soluble T2DM-1 or T2DM-2 binding protein that binds and inhibits a T2DM-1 or T2DM-2 activity, or inhibits the ability of a T2DM-1 or T2DM-2 to interact with a binding partner; an antibody that specifically binds to the T2DM-1 or T2DM-2 protein, e.g., an antibody that disrupts a T2DM-1 or T2DM-2's ability to bind to a binding partner; a mutated inactive T2DM-1 or T2DM-2 or fragment thereof which binds to a T2DM-1 or T2DM-2 but disrupts a T2DM-1 or T2DM-2 activity; a T2DM-1 or T2DM-2 nucleic acid molecule that can bind to a cellular T2DM-1 or T2DM-2 nucleic acid sequence, e.g., mRNA, and inhibit expression of the protein, e.g., an antisense, siRNA molecule or T2DM-1 or T2DM-2 ribozyme; an agent which decreases T2DM-1 or T2DM-2 gene expression, e.g., a small molecule which binds the promoter of T2DM-1 or T2DM-2. In another preferred embodiment, T2DM-1 or T2DM-2 is inhibited by decreasing the level of expression of an endogenous T2DM-1 or T2DM-2 gene, e.g., by decreasing transcription of the T2DM-1 or T2DM-2 gene. In a preferred embodiment, transcription of the T2DM-1 or T2DM-2 gene can be decreased by: altering the regulatory sequences of the endogenous T2DM-1 or T2DM-2 gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody which binds the T2DM-1 or T2DM-2 is a monoclonal antibody, e.g., a humanized chimeric or human monoclonal antibody.

In another aspect, the invention features a method of identifying a compound, e.g., a compound that modulates susceptibility t type 2 diabetes in a subject, e.g., regulates insulin synthesis and/or metabolism in a cell, tissue, or subject. The method includes: (1) providing a genetically engineered cell, tissue, or subject, e.g., a transgenic animal, e.g., an experimental rodent, having a nucleic acid which encodes a reporter molecule functionally linked to a control region of a T2DM-1 or T2DM-2 gene; (2) contacting the cell, tissue or subject with a test agent; (3) and evaluating a signal produced by the reporter molecule, the presence or strength of which is correlated with the effect of the test agent on the T2DM-1 or T2DM-2 control region. The cell can be, e.g., an islet, liver, kidney, or brain cell. The cell can be an insulin-expressing or non-insulin expressing cell. In one embodiment, the cell is a stem cell expressing an endodermal marker, e.g., hnF3B.

Examples of reporter molecules, e.g., enzymes detectable by a color signal, include fluorescent proteins, e.g., green fluorescent protein (GFP), or blue fluorescent protein; luciferase; chloramphenicol acetyl transferase (CAT); β-galactosidase; β-lactamase; or secreted placental alkaline phosphatase. Other reporter molecules and other enzymes whose function can be detected by appropriate chromogenic or fluorogenic substrates are known to those skilled in the art.

In a preferred embodiment, the cell, tissue or subject can include a second transgene having a second control sequence from a second gene linked to the same or a different reporter molecule sequence.

In a preferred embodiment, the method further includes administering the test agent to an animal and determining the effect of the test agent on the animal, e.g., determining diabetes susceptibility in the animal, e.g., determining a parameter of insulin function or beta cell function in the animal. In one embodiment, the animal is an animal model of diabetes, e.g., a NOD Mouse and its related strains, BB Rat, Leptin or Leptin Receptor mutant rodents, Zucker Diabetic Fatty (ZDF) Rat, Sprague-Dawley rats, Obese Spontaneously Hypertensive Rat (SHROB, Koletsky Rat), Wistar Fatty Rat, New Zealand Obese Mouse, NSY Mouse, Goto-Kakizaki Rat, OLETF Rat, JCR:LA-cp Rat, Neonatally Streptozotocin-Induced (n-STZ) Diabetic Rats, Rhesus Monkey, Psammomys obesus (fat sand rat), or a C57Bl/6J. Mouse.

In another aspect, the invention provides a method of screening for a compound, e.g., a compound that affects type 2 diabetes susceptibility, e.g., a compound that modulates insulin function, e.g., insulin resistance, insulin secretion or β-cell function in a subject, e.g., a mammal. The methods include screening for compounds that modulate the expression, level or activity of a T2DM-1 or T2DM-2, e.g., T2DM-1a, T2DM-1b, T2DM-2a or T2DM-2b.

In one embodiment, the method includes: providing a T2DM-1 or T2DM-2 protein or nucleic acid, e.g., T2DM-1a, T2DM-1b, T2DM-2a or T2DM-2b protein or nucleic acid or a functional fragment thereof; contacting the T2DM-1 or T2DM-2 protein or nucleic acid with a test compound, and determining if the test compound modulates, e.g., interacts with or binds, the T2DM-1 or T2DM-2 protein or nucleic acid.

In one embodiment, the test compound binds to the T2DM-1 or T2DM-2 protein and modulates a T2DM-1 or T2DM-2 activity. For example, the compound binds to the T2DM-1 or T2DM-2 protein and facilitates or inhibits any binding of T2DM-1 or T2DM-2 with a naturally occurring ligand. In a preferred embodiment, the compound is an antibody, e.g., an inhibitory T2DM-1 or T2DM-2 antibody.

In a preferred embodiment, the T2DM-1 or T2DM-2 is human T2DM-1 or T2DM-2.

In another embodiment, the test compound binds to a T2DM-1 or T2DM-2 nucleic acid or fragment thereof, e.g., the test compound binds to the T2DM-1 or T2DM-2 promoter region and increases T2DM-1 or T2DM-2 transcription; the test compound binds to a T2DM-1 or T2DM-2 nucleic acid and inhibits transcription of the T2DM-1 or T2DM-2 gene; or the test compound binds to a T2DM-1 or T2DM-2 nucleic acid and inhibits translation of the T2DM-1 or T2DM-2 mRNA. In a preferred embodiment, the compound is a small molecule that binds to the T2DM-1 or T2DM-2 promoter region to modulate transcription.

In another embodiment, the test compound competes with the endogenous T2DM-1 or T2DM-2 protein for binding to a T2DM-1 or T2DM-2 binding partner, thereby inhibiting a T2DM-1 or T2DM-2 activity. For example, the test compound can be a dominant negative T2DM-1 or T2DM-2 protein or nucleic acid.

In a preferred embodiment, the test agent is one or more of: a protein or peptide, an antibody, a small molecule, a nucleotide sequence. For example, the agent can be an agent identified through a library screen described herein.

In a preferred embodiment, the contacting step is performed in vitro.

In a preferred embodiment, the method further includes administering the test compound to an experimental animal, e.g., an experimental model of diabetes described herein.

In another preferred embodiment, the contacting step is performed in vivo.

In another embodiment, the method includes: providing a test cell, tissue, or subject; administering a test agent to the cell, tissue, or subject; and determining whether the test agent modulates T2DM-1 or T2DM-2 expression, level or activity in the cell, tissue, or subject. An agent that is found to modulate T2DM-1 or T2DM-2 in the cell, tissue, or subject is identified as an agent that can affect susceptibility to type 2 diabetes, e.g., modulate β-cell function, β cell mass and/or insulin function, e.g., insulin production or metabolism.

In a preferred embodiment, the cell is a pancreatic islet cell, muscle cell, kidney cell, liver cell, or adipose cell. The cell can be an insulin-expressing or non-insulin expressing cell. In another preferred embodiment, the tissue is a pancreatic tissue. In a preferred embodiment, the subject is a non-human animal, e.g., an animal model for a pancreatic or insulin related disorder, e.g., a nod mouse, a Zucker rat, a fructose fed rodent, an Israeli sand rat.

In a preferred embodiment, the test cell, tissue, or subject is a wild-type cell, tissue or subject.

In another preferred embodiment, the cell or tissue is from a transgenic mammal described herein, or the subject is a transgenic mammal described herein.

In a preferred embodiment, the method further includes administering the test agent to an animal and determining the effect of the test agent on the animal, e.g., determining the animal's susceptibility to type 2 diabetes, e.g., a parameter of insulin function or beta cell function in the animal.

The effect of the test agent on a T2DM-1 or T2DM-2 in the cell, tissue or subject can be assayed by numerous methods known in the art. For example, T2DM-1 or T2DM-2 interactions with other proteins can be assayed, e.g., by standard immunodetection and protein separation techniques, e.g., using an anti-T2DM-1 or anti-T2DM-2 antibody described herein. T2DM-1 or T2DM-2 binding to other proteins can be detected, e.g., by standard size exclusion, size separation, or immunoprecipitation techniques. T2DM-1 or T2DM-2 subcellular localization can be detected, e.g., using standard immunofluorescence techniques.

In a preferred embodiment, the subject is further evaluated for one or more of the following parameters of insulin function: (1) insulin metabolism, e.g., insulin responsiveness or resistance; (2) glucose levels; (3) pancreatic β-cell morphology, function or development; or any other symptom of type 2 diabetes.

In a further aspect, the invention provides methods for evaluating the efficacy of a treatment of a disorder, e.g., an insulin or pancreatic α-cell disorder, (e.g., type 2 diabetes mellitus) and its associated disorders, e.g., hypertension, retinopathy, persistent hyperinsulinemic hypoglycemia of infancy (PHHI), insulin resistance, hyperglycemia, glucose intolerance, glucotoxicity. The method includes: treating a subject, e.g., a patient or an animal, with a protocol under evaluation (e.g., treating a subject with one or more of a compound identified using the methods described herein); and evaluating the expression or activity of a T2DM-1 or T2DM-2 nucleic acid or polypeptide before and after treatment. A change, e.g., a decrease or increase, in the level of a T2DM-1 or T2DM-2 nucleic acid (e.g., mRNA) or polypeptide or activity (e.g., transcriptional activation activity) after treatment, relative to the level of expression before treatment, is indicative of the efficacy of the treatment of the disorder.

In a preferred embodiment, the subject is also treated with, e.g., insulin or glucose, before and/or after the subject is treated with the protocol under evaluation. The level of T2DM-1 or T2DM-2 nucleic acid or polypeptide expression or activity can be detected by any method described herein.

In a preferred embodiment, the evaluating step includes obtaining a sample (e.g., a tissue sample, e.g., a biopsy, or a fluid (e.g., blood) sample) from the subject, before and after treatment and comparing the level of expressing of a T2DM-1 or T2DM-2 nucleic acid (e.g., mRNA), polypeptide, or activity before and after treatment. In another aspect, the invention provides methods for evaluating the efficacy of a therapeutic or prophylactic agent. The method includes: contacting a sample with an agent (e.g., a compound identified using the methods described herein, a cytotoxic agent); and evaluating the expression of T2DM-1 or T2DM-2 nucleic acid or polypeptide in the sample before and after the contacting step. A change, e.g., a decrease or increase, in the level of the T2DM-1 or T2DM-2 nucleic acid (e.g., mRNA) or polypeptide in the sample obtained after the contacting step, relative to the level of expression in the sample before the contacting step, is indicative of the efficacy of the agent. The level of T2DM-1 or T2DM-2 nucleic acid or polypeptide expression can be detected by any method described herein.

In a preferred embodiment, the sample is from pancreatic tissue.

In a preferred embodiment, the sample is a pancreatic islet sample.

In another aspect, the invention features a cell which is genetically engineered to express, e.g., constitutively express, transiently express, or overexpress, T2DM-1 or T2DM-2 or a functional fragment thereof. The cell can be a cell type that normally expresses insulin in nature, or a cell type that does not normally express insulin in nature.

In a preferred embodiment, T2DM-1 or T2DM-2 is linked or fused to a heterologous polypeptide, e.g., the cell is genetically engineered to constitutively express, transiently express, or overexpress a T2DM-1 or T2DM-2 fusion protein as described herein.

In a preferred embodiment, the cell is a secretory cell, pancreatic β-cell, β-cell precursor cell, adult or embryonic stem cell, a human neuroendocrine cell, pancreatic ductal cell or cell line, pancreatic acinar cell or cell line, pancreatic endocrine cell or cell line, enteroendocrine cell or cell line, hepatic cell, fibroblast, endothelial cell, or muscle cell, a secretory cell, a pancreatic β-cell precursor cell or a pancreatic β-cell or duct cell or dedifferentiated duct or exocrine cell, liver cell, muscle cell, kidney, or testis cell. In one embodiment, the cell is a stem cell expressing an endodermal marker, e.g., hnF3B.

In a preferred embodiment, the cell is genetically engineered to express or misexpress at least one polypeptide that enhances glucose responsiveness, for example, a glucose processing enzyme and/or a receptor. Examples of such polypeptides include hexokinase, glucokinase, GLUT-2, GLP-1, IPI1, PC2, PC3, PAM, glucagon-like peptide I receptor, glucose-dependent insulinotropic polypeptide receptor, BIR, SUR, GHRFR and GHRHR.

In a preferred embodiment, the cell is a secretory cell that includes a nucleic acid encoding insulin, e.g., human insulin.

In another aspect, the invention features a transgenic non-human mammal, e.g., a primate, a rodent, e.g., a rat, mouse, or guinea pig, that contains a transgene, e.g., a T2DM-1 or T2DM-2 transgene. In one embodiment, the non-human transgenic mammal has a genome being heterozygous or homozygous for an engineered disruption in a T2DM-1 or T2DM-2 gene, wherein the mammal is susceptible to type diabetes, e.g., the animal has disrupted insulin function. For example, the transgenic animal misexpresses T2DM-1 or T2DM-2, e.g., overexpresses, underexpresses, or is null for T2DM-1 or T2DM-2. An T2DM-1 or T2DM-2 transgene refers to an exogenous T2DM-1 or T2DM-2 nucleic acid (e.g., a T2DM-1 or T2DM-2 cDNA, gene or fragment thereof) that is inserted into the animal. The nucleic acid is inserted into the genome of the animal, e.g., in the chromosomal DNA of the animal or in an episome, plasmid, or other non-chromosomal DNA element. In another embodiment, the T2DM-1 or T2DM-2 gene is misexpressed in a tissue specific manner, e.g., the mMafA gene is misexpressed in pancreatic ductal cells and not misexpressed in a non pancreatic tissue.

In a first embodiment, the transgenic animal has a disruption in an T2DM-1 or T2DM-2 gene wherein the disruption causes a reduction in T2DM-1 or T2DM-2 expression, levels or activity. The disruption in the T2DM-1 or T2DM-2 gene can be a deletion, addition, or substitution. In a preferred embodiment, the transgenic animal is a T2DM-1 or T2DM-2 knockout. In another embodiment, the disruption is a disruption that decreases the level of expression of an endogenous T2DM-1 or T2DM-2 gene, e.g., by decreasing transcription of the T2DM-1 or T2DM-2 gene. In another preferred embodiment, the transgenic animal contains a transgene that decreases transcription of the endogenous T2DM-1 or T2DM-2 gene, e.g., by the addition of a negative regulatory sequence (such as a DNA-biding site for a transcriptional repressor), or by the removal of a positive regulatory sequence (such as an enhancer or a DNA-binding site for a transcriptional activator).

The transgenic animal displays one or more of the following phenotypes: (1) it has decreased T2DM-1 or T2DM-2 compared to a wild-type animal; (2) it is susceptible to type 2 diabetes, (3) has high serum insulin levels compared to a wild-type animal; (4) it has aberrant pancreatic cell function compared to a wild-type mammal. The transgenic animals are useful, e.g., as models for insulin related or pancreatic α-cell related disorders described herein, e.g., type 2 diabetes. The transgenic animals are also useful as test subjects in the screening assays described herein.

In a preferred embodiment, the disruption is homozygous.

In another preferred embodiment, the disruption is heterozygous.

In a second embodiment, the transgenic animal overexpresses T2DM-1 or T2DM-2 compared to a wild-type animal. In one embodiment, the animal expresses a heterologous T2DM-1 or T2DM-2 nucleic acid in addition to its endogenous T2DM-1 or T2DM-2 gene. In another embodiment, T2DM-1 or T2DM-2 is overexpressed by increasing the level of expression of an endogenous T2DM-1 or T2DM-2 gene, e.g., by increasing transcription of the T2DM-1 or T2DM-2 gene or increasing T2DM-1 or T2DM-2 mRNA stability. In a preferred embodiment, the transgenic animal contains a transgene that increases transcription of the transgenic animal's endogenous T2DM-1 or T2DM-2 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the T2DM-1 or T2DM-2 gene to be transcribed more efficiently or in a regulated fashion (e.g., through use of a Tet on/off system).

The transgenic animal displays one or more of the following phenotypes: (1) it has increased T2DM-1 or T2DM-2 compared to a wild-type animal. The transgenic animals are useful, e.g., as models for type 2 diabetes. The transgenic animals are also useful as test subjects in the screening assays described herein.

In another preferred embodiment, the transgenic animal, e.g., rodent, expresses T2DM-1 or T2DM-2 or a functional fragment thereof.

In another aspect, the invention features a method of evaluating a subject. The method includes: optionally identifying a subject suspected of being at risk for type2 diabetes, e.g., a subject having a family history of type 2 diabetes; determining the sequence of at least one nucleotide within the T2DM-1 or T2DM-2 gene, or flanking the T2DM-1 or T2DM-2 gene (e.g., within 10, 100, 1000, 3000, 50000, 10,0000, or more base pairs of the gene); and comparing the determined sequence with a reference sequence.

In a preferred embodiment, the subject is at risk for an insulin or β-cell related disorder, e.g., type 2, diabetes, and its associated disorders, e.g., hypertension, retinopathy, persistent hyperinsulinemic hypoglycemia of infancy (PHHI), insulin resistance, hyperglycemia, glucose intolerance, glucotoxicity.

In a preferred embodiment, a difference between the determined sequence and the reference sequence indicates a difference in the subject's response to a therapeutic agent.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a T2DM-1 or T2DM-2 molecule. In one embodiment, the capture probe is a nucleic acid, e.g., a probe complementary to a T2DM-1 or T2DM-2 nucleic acid sequence or a nucleic acid, e.g., a DNA that the T2DM-1 or T2DM-2 specifically binds. In another embodiment, the capture probe is a polypeptide, e.g., an antibody specific for T2DM-1 or T2DM-2 polypeptides. Also featured is a method of analyzing a sample by contacting the sample to the aforementioned array and detecting binding of the sample to the array.

Other features and advantages of the invention will be apparent from the following detailed description, and from the claims.

### BRIEF DESCRIPTION OF THE DRAWINGS

FIG. 1 is a diagram of the gene structure of the long (a) and short (b) forms of T2DM-1 and T2DM2. Specific SNPs are designated by arrows.

FIG. 2A-D are the T2DM-1a, T2DM-1b, T2DM-2a, and T2DM2b cDNA and amino acid sequences.

FIG. 3A-C is a set of tables showing the organization of the T2DM-1 and T2DM-2 gene sequences.

FIG. 4A-His a list of SNP sequences of T2DM-1 and T2DM-2. SEQ ID NOs: 9, 11, 13, 15, 17, 19, 21, 23, 25, 27, 29, 31, 33 and 35 are the reference (standard) T2DM-1 or T2DM-2 sequence. SEQ ID NOs: 10, 12, 14, 16, 18, 20, 22, 24, 26, 28, 30, 32, 34, and 36 are polymorphisms found in type 2 diabetes patients. The polymorphic nucleotides are underlined. Included are 200 bp up stream and downstream of each polymorphism. These sequences are mapped onto the genomic context in FIG. 10.

FIG. 5 is a table of 14 SNPs in T2DM-1 and T2DM-2. Summarized are the source of the SNP sequence, the nucleotide change, and its genomic location.

FIG. 6A-C is a GCG gap alignment of predicted Diff40 long form (BAA20840) (SEQ ID NO:37, top sequence) with the predicted Diff40-short form NCBI RefSeq protein sequence (NP_{—}56948) (SEQ ID NO:38, bottom sequence). A default gap penalty of −8/−2 was used in the alignment.

FIG. 7A-C is a GCG bestfit alignment of predicted Diff40 long form (BAA20840) (SEQ ID NO:39) with the predicted T2DM-1a amino acid sequence (SEQ ID NO:2). A default gap penalty of −8/−2 was used. The amino and carboxyl termini of the Diff40 long form show similarity to T2DM-1a. (Underlined sequence=present in diff40 long form only. Double underlined residues=end of Diff40-short form and end of long/short T2DM-1 common sequence.)

FIG. 8A-B is a GCG bestfit alignment of predicted Diff40 short form (BAA20840) (SEQ ID NO:40) with the predicted T2DM-1b (short form) (SEQ ID NO:4). A default gap penalty of −8/−2 was used.

FIG. 9A-MM is the reference region of chromosome 20 that contains the T2DM-1 and T2DM-2 genes (+/−1000 bp). The reference sequence is 106,707 basepairs in length.

FIG. 10 is a Sequencher document showing the location of the exons and SNP's for T2DM-1 and T2DM-2 mapped to the reference region shown in FIG. 9.

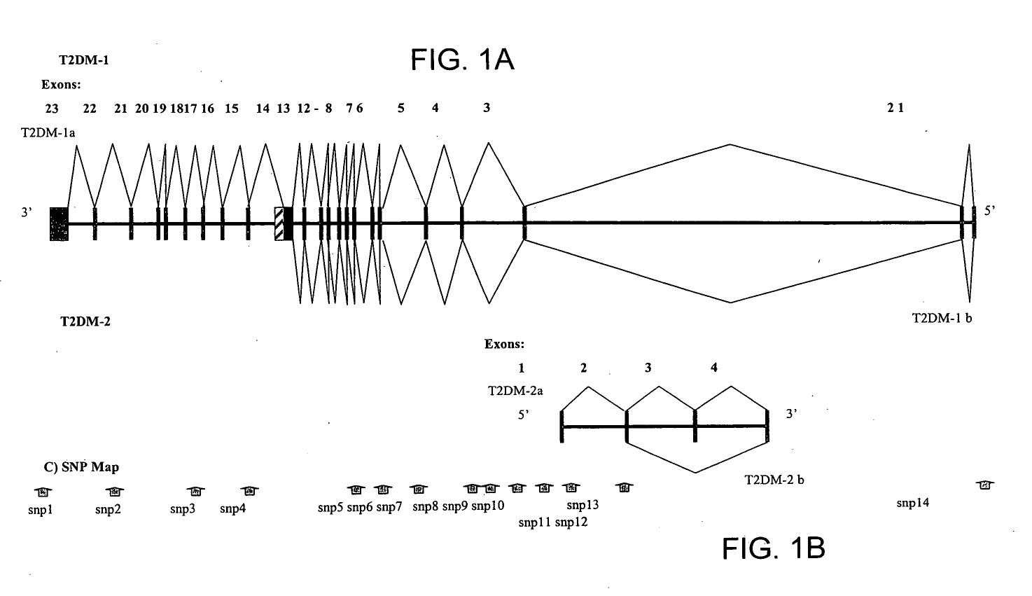

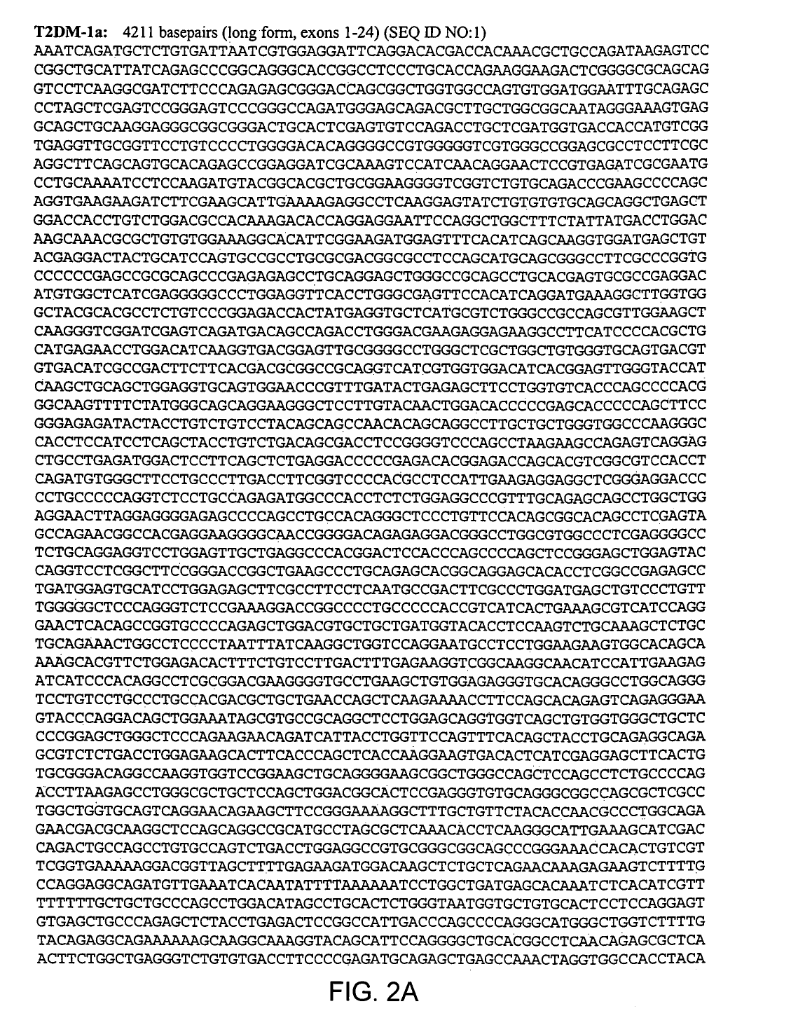

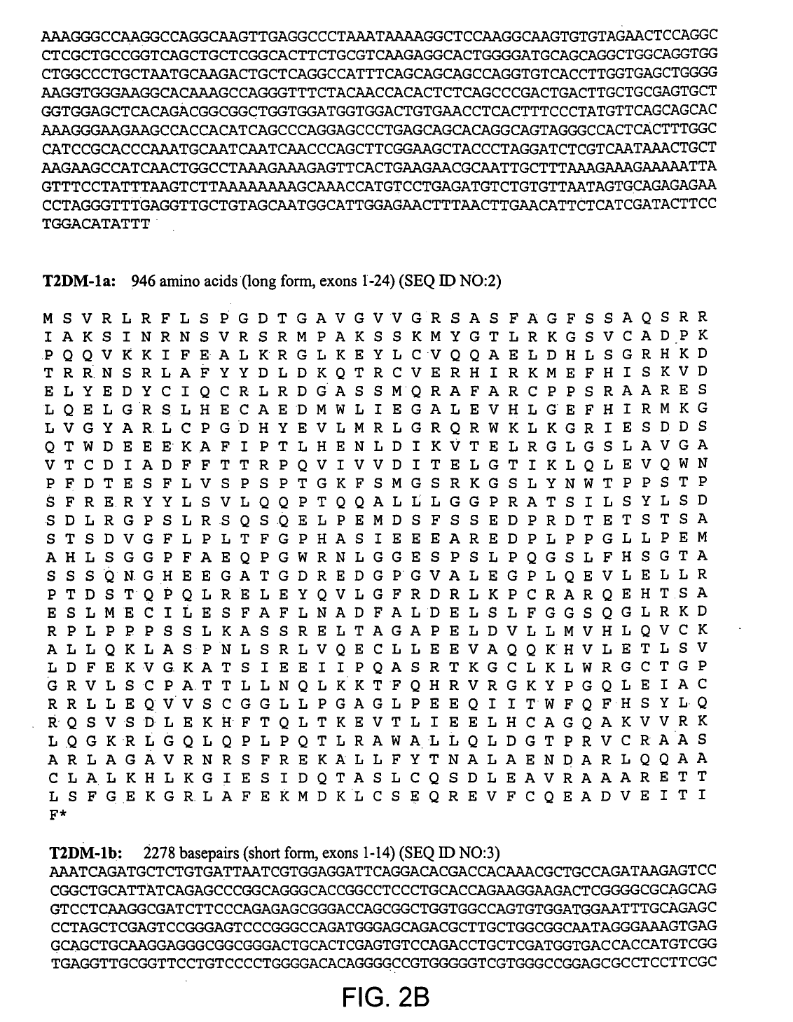

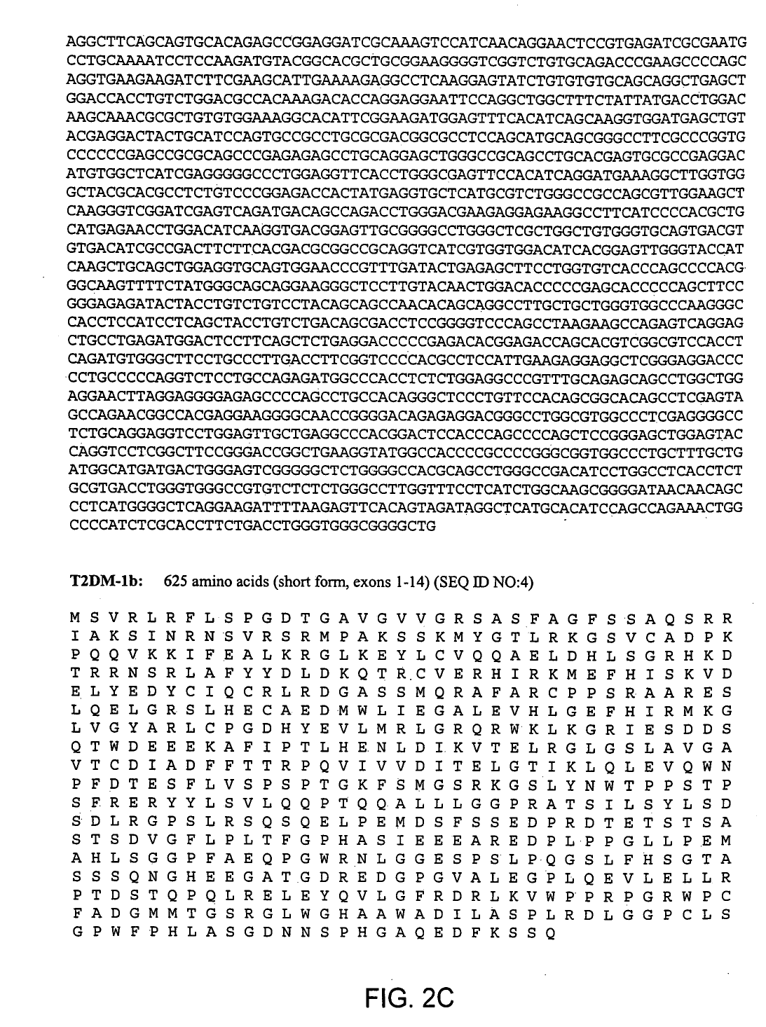

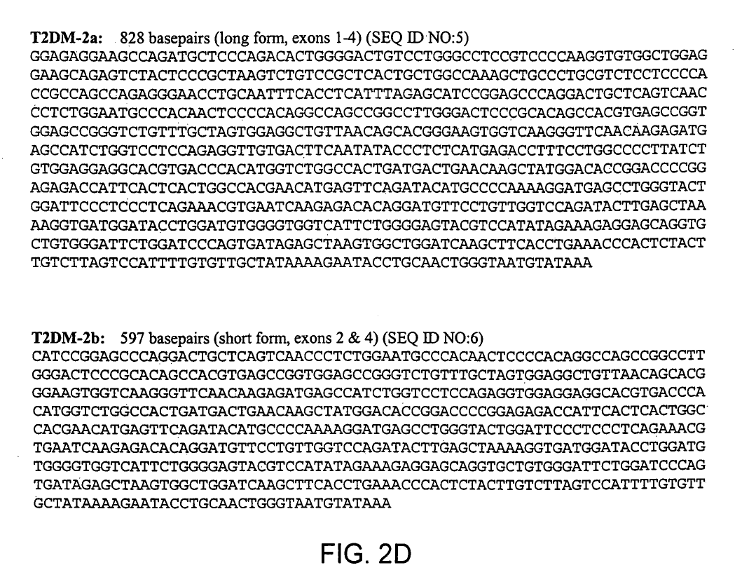

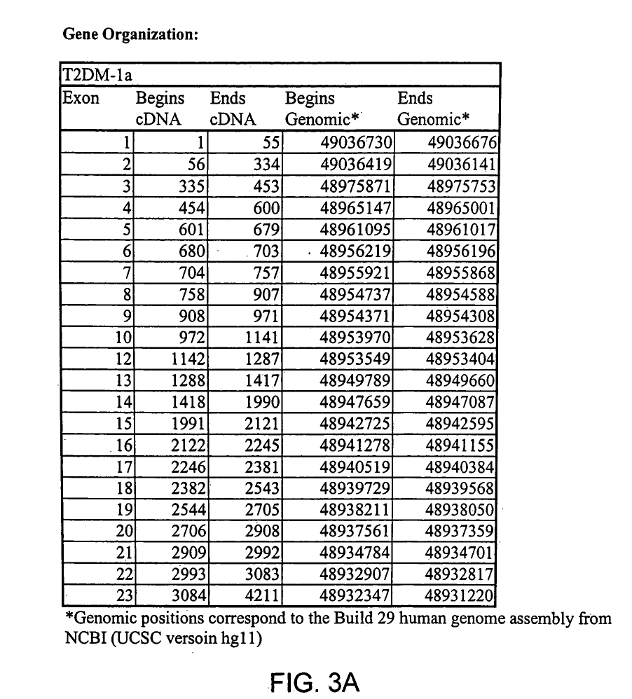

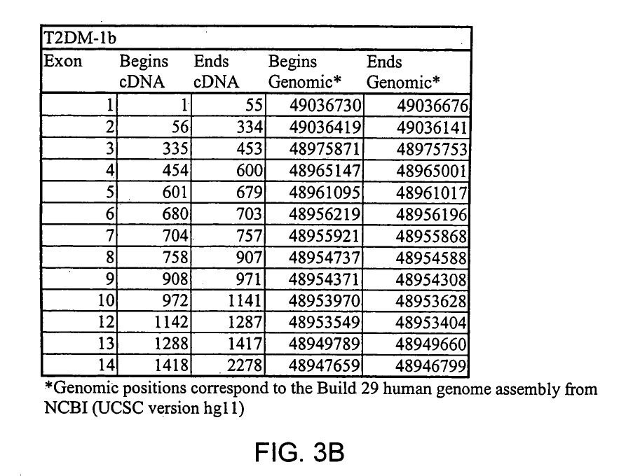

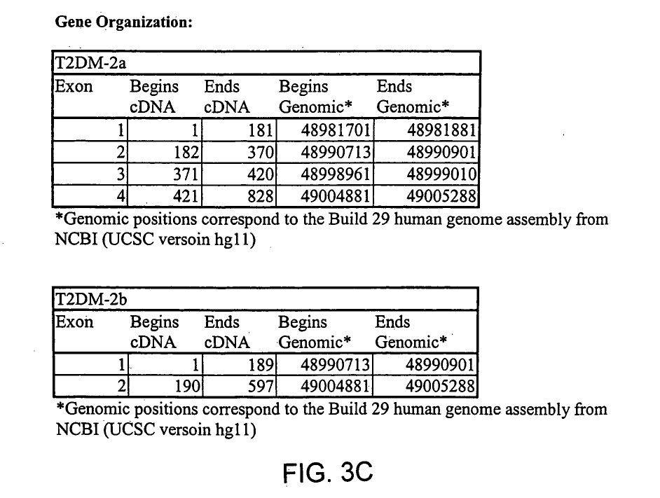

### DETAILED DESCRIPTION

Two novel genes that are associated with susceptibility to Type 2 diabetes mellitus have been discovered, T2DM-1 and T2DM-2, each having a long form (T2DM-1a and T2DM-2a, respectively) and a short form (T2DM-1b and T2DSM-2b, respectively). Numerous polymorphisms of each gene, which are associated with type 2 diabetes patients, have also been identified. The nucleotide sequence of T2DM-1a is shown as SEQ ID NO:1 and its amino acid sequence as SEQ ID NO:2. The nucleotide sequence of T2DM-1b is shown as SEQ ID NO: 3 and its amino acid sequence as SEQ ID NO:4. The nucleotide sequence of T2DM-2a is shown as SEQ ID NO:5. The nucleotide sequence of T2DM-2b is shown as SEQ ID NO: 6. Fourteen polymorphisms of the genes are shown as SEQ ID NOs: 10, 12, 14, 16, 18, 20, 22, 24, 26, 28, 30, 32, 34, and 36. The sequences described herein are useful for, inter alia, genetic screening for susceptibility to type 2 diabetes mellitus, diagnosis, therapy, and pharmacogenomics applications.

### Identification and Cloning of T2DM-1 and T2DM-2

A susceptibility locus associated with Type 2 diabetes mellitus was identified in a 10-cM region located on chromosome 20q13.1-q13.2, between markers D20S110 and D20S428, with the strongest evidence for linkage occurring closest to marker D20S196 (Klupa (2000) Diabetes 49:2212-2216). Preliminary analysis of recombination events within the most strongly linked families contributing to this linkage allowed the narrowing of the critical region to an interval less than 1 MB in length.

To localize the susceptibility genes for Type 2 diabetes within this 1 MB region, both ab initio and homology based methods were employed. The genomic sequence for this region was used in computational gene prediction analyses using GENESCAN (genes.mit.edu/GENESCAN.html), GeneFinder (genome.washington.edu/cgi-bin/Genefinder), FGENE (genomic.sanger.ac.uk/gf/gf.shtml), and GeneMark.hmm (opal.biology.gatech.edu/GeneMark). The sequences of the predicted genes were used to search NCBI's (www.ncbi.nlm.nih.gov) EST and protein databases. Additionally, the critical interval sequence was analyzed for highly conserved regions on the corresponding mouse chromosome (chromosome 2) using the PipMaker program (bio.cse.psu.edu/pipmaker).

The analysis enabled the mapping to the critical interval of six known genes and several new genes. Two of these genes (designated herein as T2DM-1 ad T2DM-2) provided evidence of being true transcripts. The transcripts were validated by RT-PCR experiments performed in a panel of cDNAs from 10 different tissues. The transcripts were characterized using RACE experiments in the appropriate tissues. This strategy revealed that T2DM-1 and T2DM-2 were actually transcribed, each with two isoforms.

The two novel genes are in close proximity on chromosome 20q. Both genes T2DM-1 and T2DM-2 contain a long form (T2DM-1a and T2DM-2a) and a short form (T2DM-2a and T2DM-2b). Each has a number of polymorphisms, e.g., SNPs, which are associated with Type 2 diabetes and likely play a role in the susceptibility to this disease.

### Expression Analysis

The T2DM-1 gene (long form, SEQ ID NO:1; short form, SEQ ID NO:3) is expressed largely in tissues involved in insulin metabolism: liver, muscle, pancreatic islets, testis, kidney, adipose tissue, brain, and less so in the placenta, fibroblasts, and lymphoblasts. T2DM-1a (SEQ ID NO:1), the long form of T2DM-1, is 4211 base pairs in length and consists of exons 1-24. T2DM-1b (SEQ ID NO:3), the short form of T2DM-1, is 2278 basepairs in length and consists of exons 1-14.

The T2DM-2 gene (long form, SEQ ID NO:5; and short form, SEQ ID NO:6) is not as widely expressed as T2DM-1. It is expressed in brain, kidney, placenta, testis, and less so in fibroblasts and pancreatic islets. The long form of T2DM-2 (SEQ ID NO:5), referred to as T2DM-2a, is 828 basepairs in length and consists of 4 exons. The short form of T2DM-2 (SEQ ID NO:6), referred to as T2DM-2b, is 597 basepairs in length and consists of exons 2 and 4, transcribed in the opposite direction as the long form.

### Sequence Analysis of T2DM1

The predicted amino acid sequence of T2DM-1 (and T2DM-2, with which T2DM1 shares exons 1-4) shows homology with a protein known as Diff40 (also known as PL48, Diff48, KIAA 0386, or C6 or f32). Diff40 was originally identified and cloned from cytotrophoblast and HL-60 cells undergoing differentiation (Dakour et al., (1997) Gene 185:153-7). It is thought to stimulate the formation of a non-mitotic multinucleate syncytium from proliferative cytotrophoblasts during trophoblast differentiation. A more recent study found that Diff40 is down-regulated in neutrophils (3-5× fold) when exposed to KIM6 (pCD1-) Y. pestis or E coli K12 bacteria. Diff40 appears to be a late expression gene (Subrahmanyam, et al. (2001), Blood 97:2457-68).

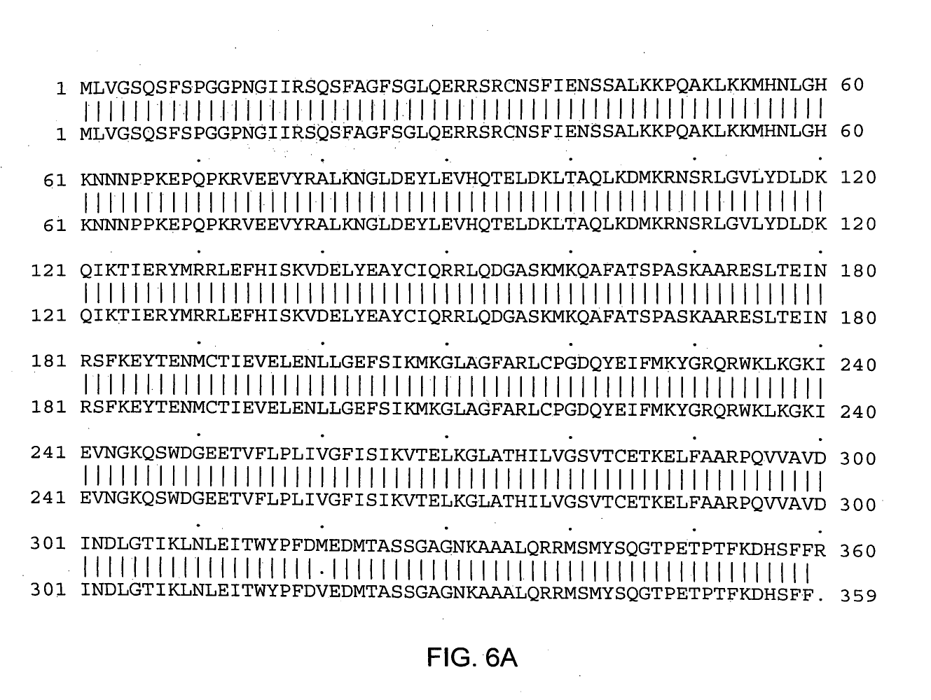

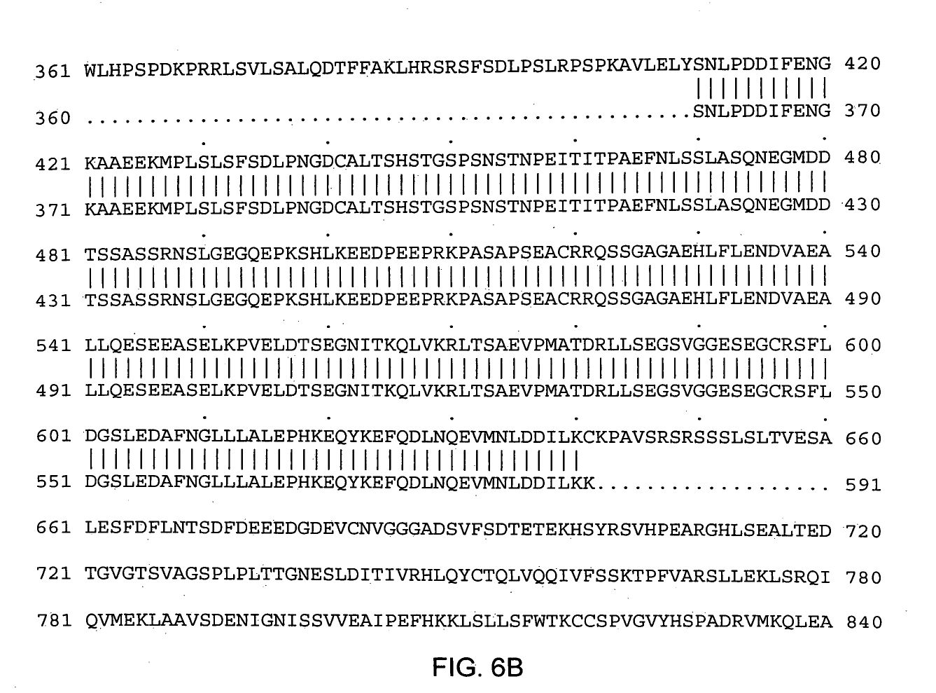

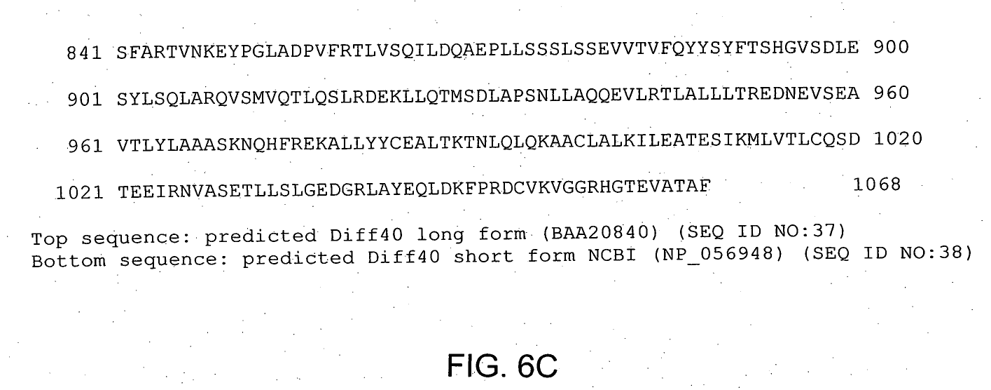

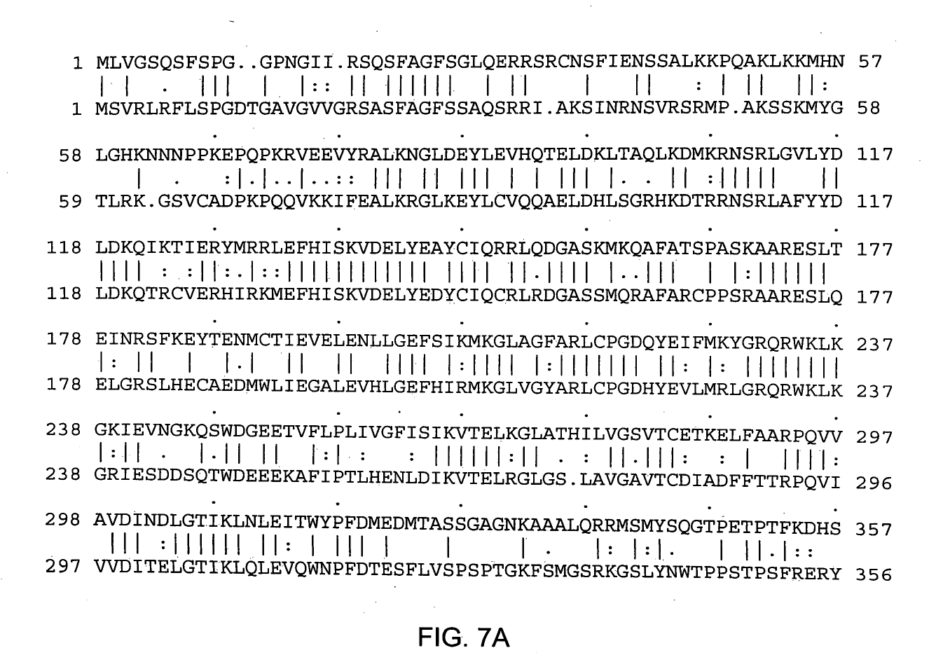

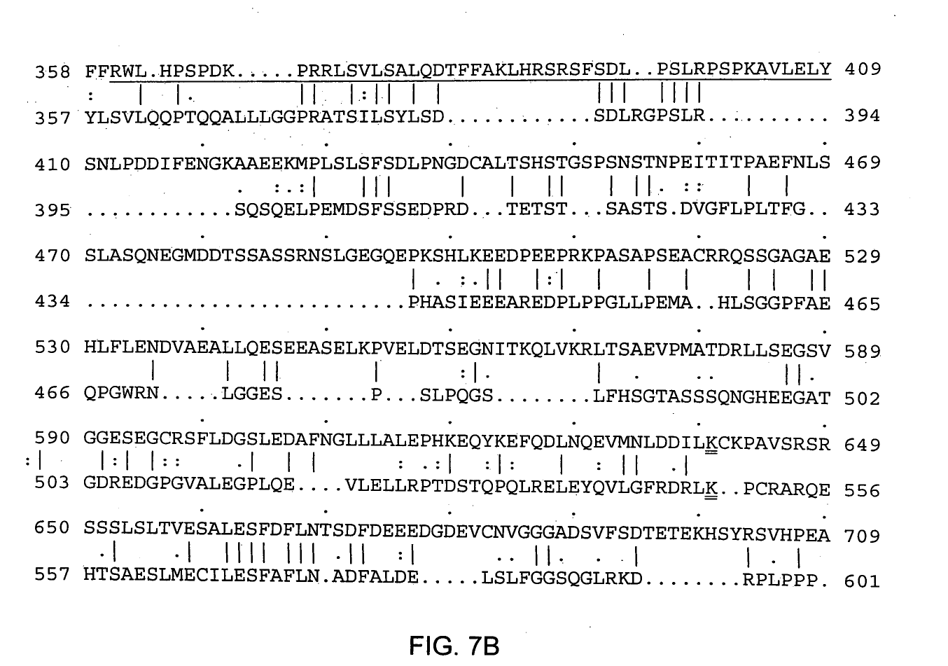

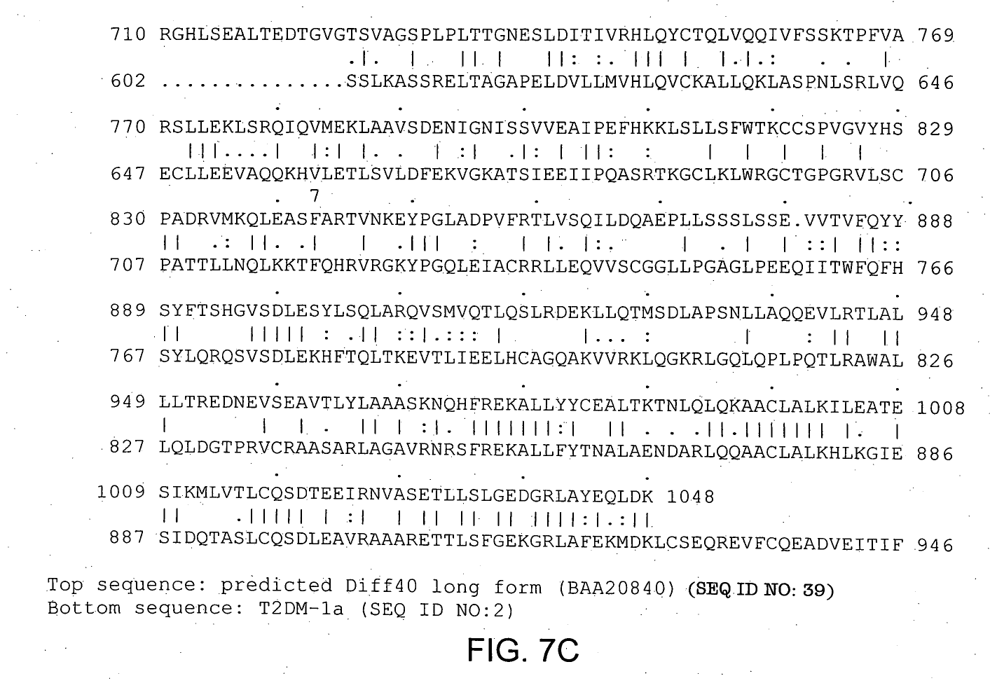

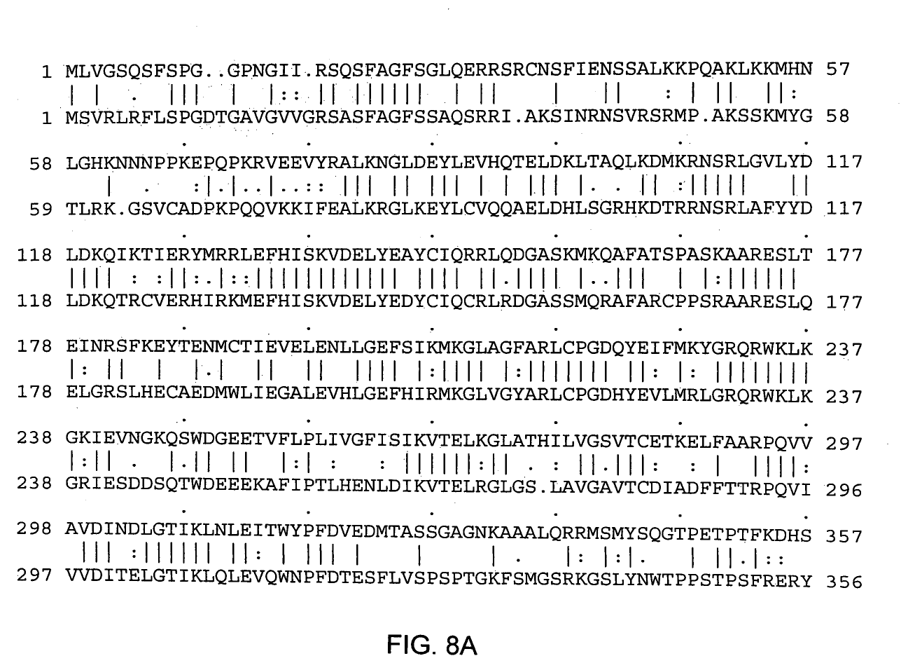

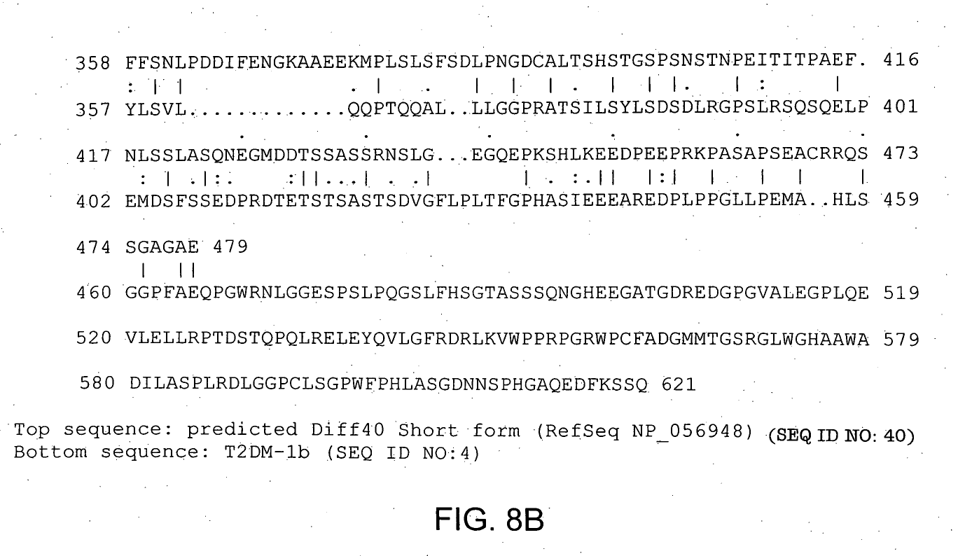

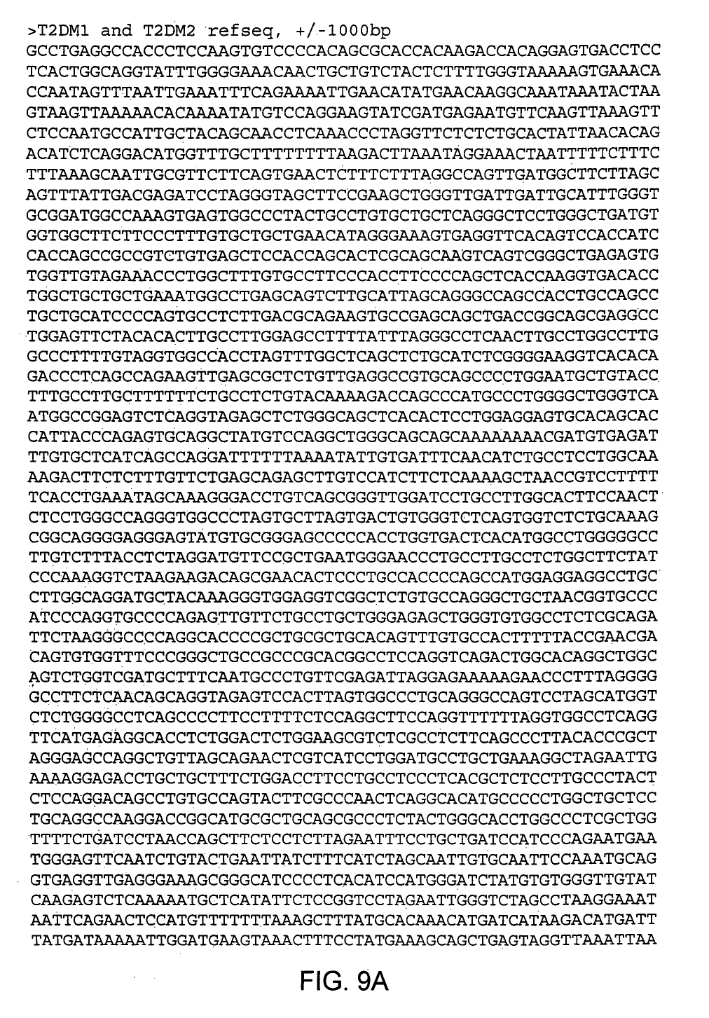

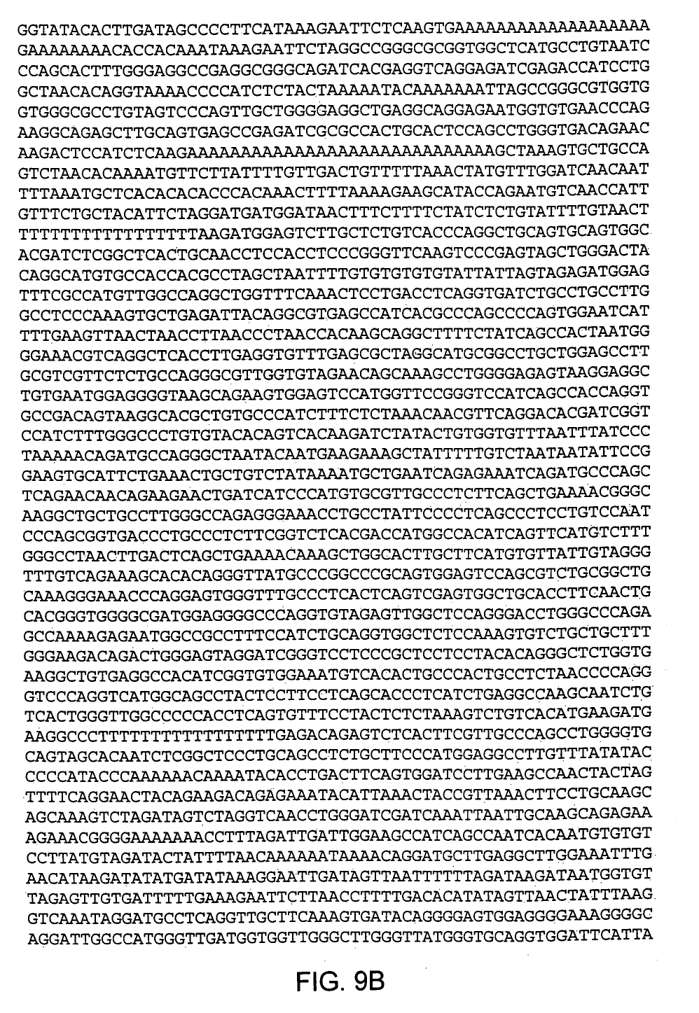

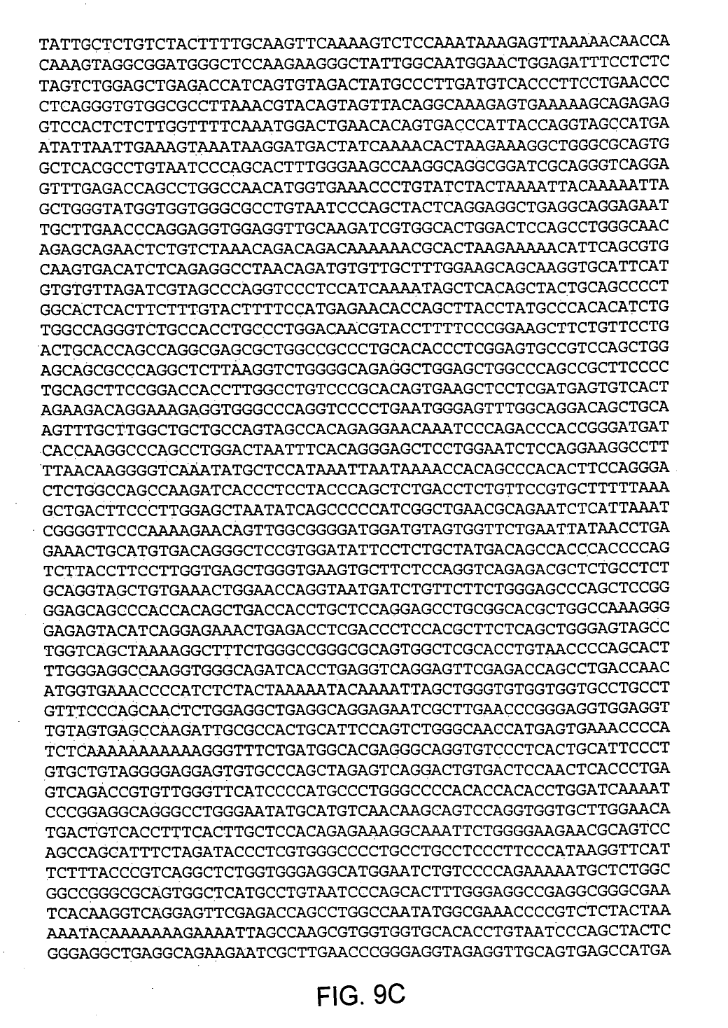

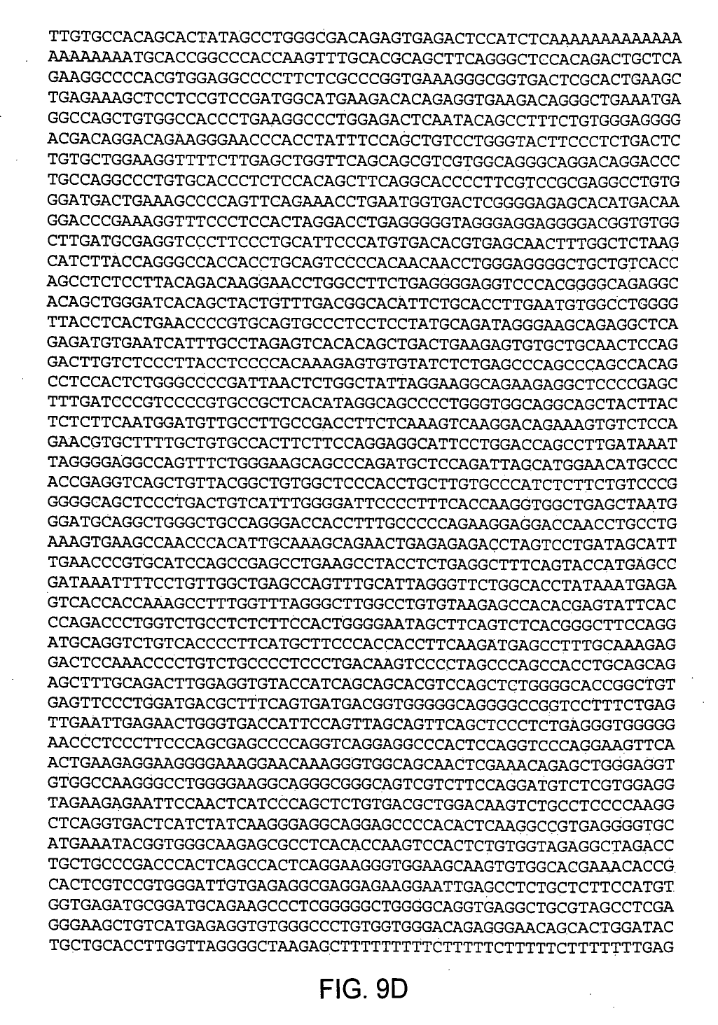

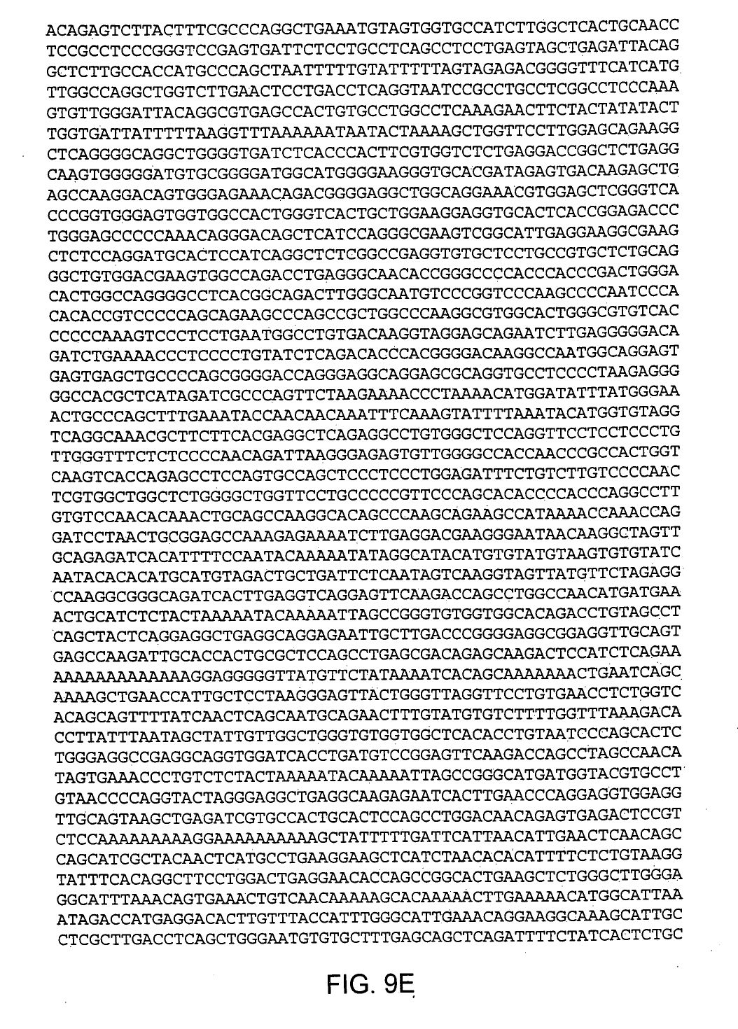

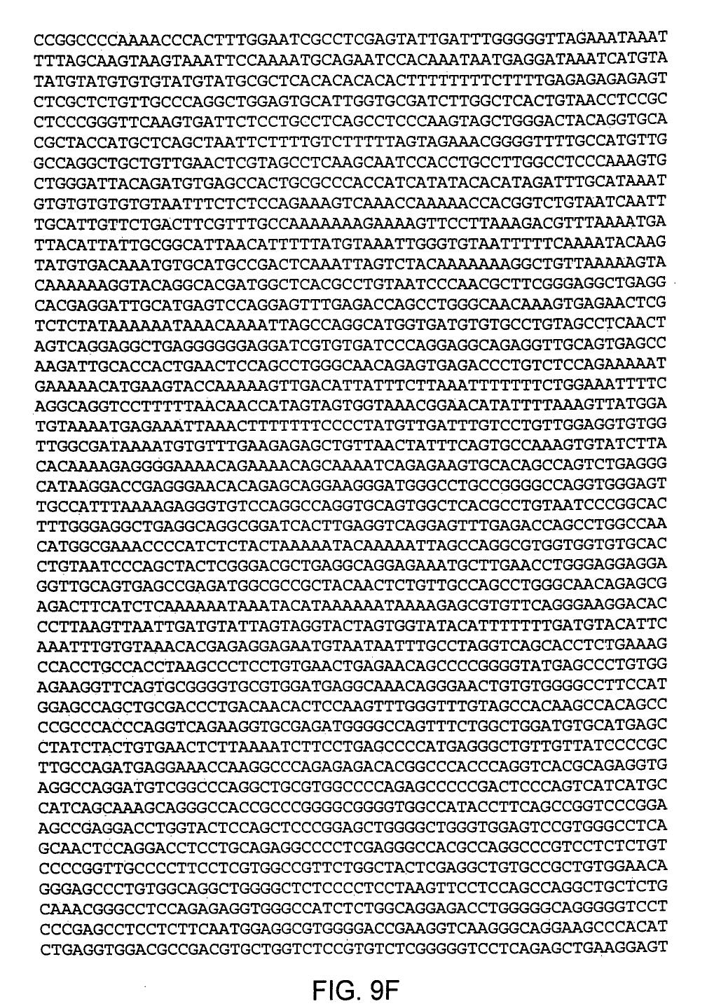

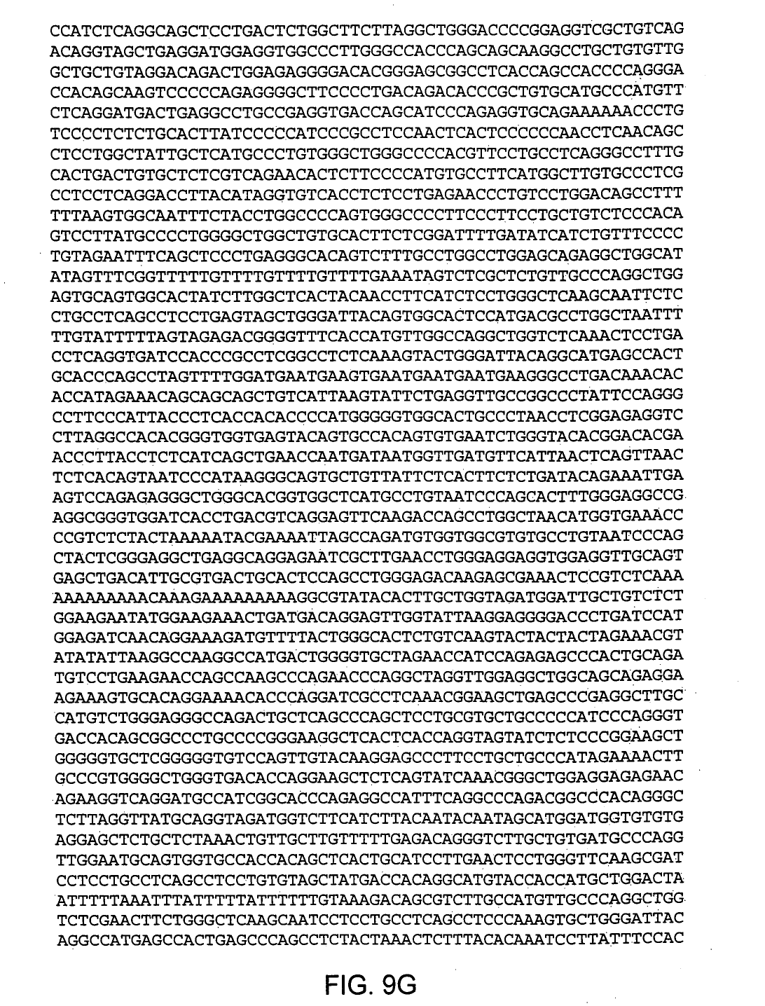

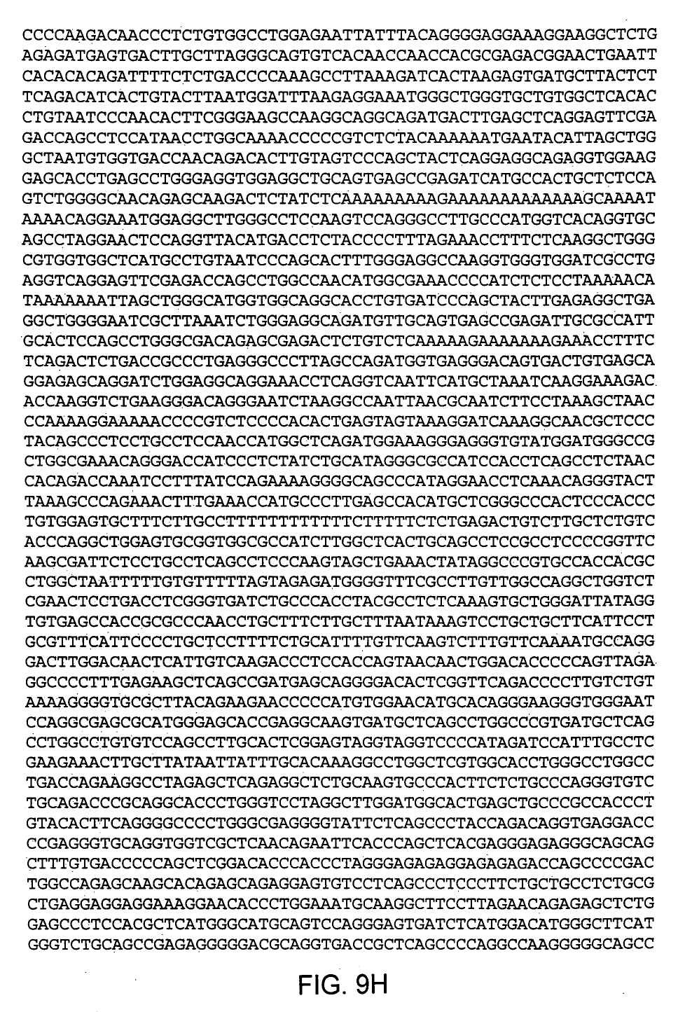

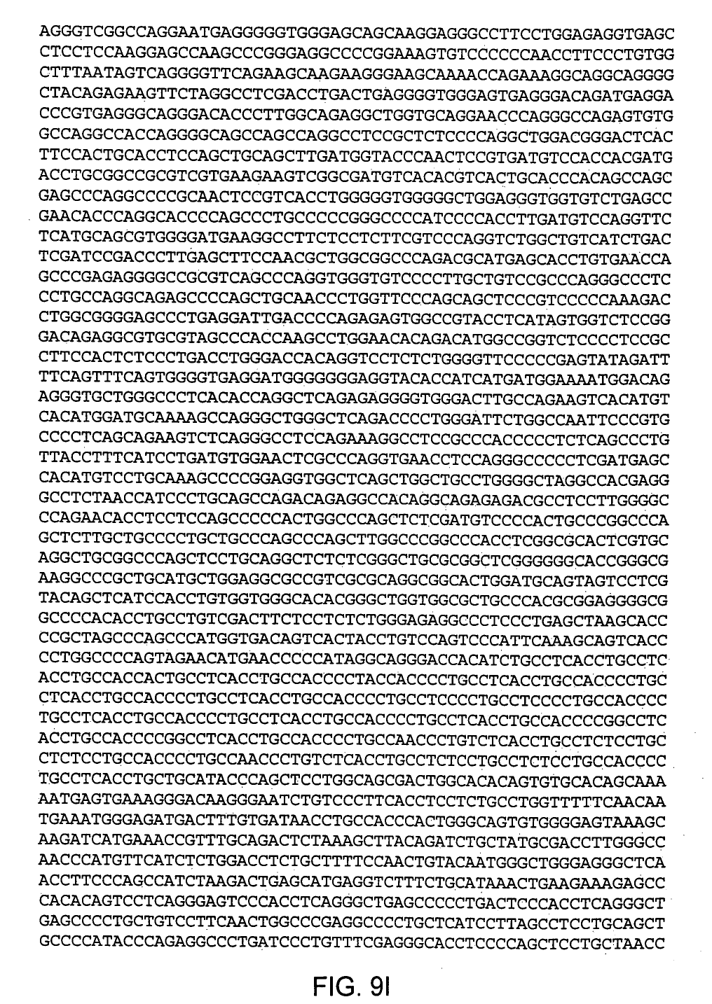

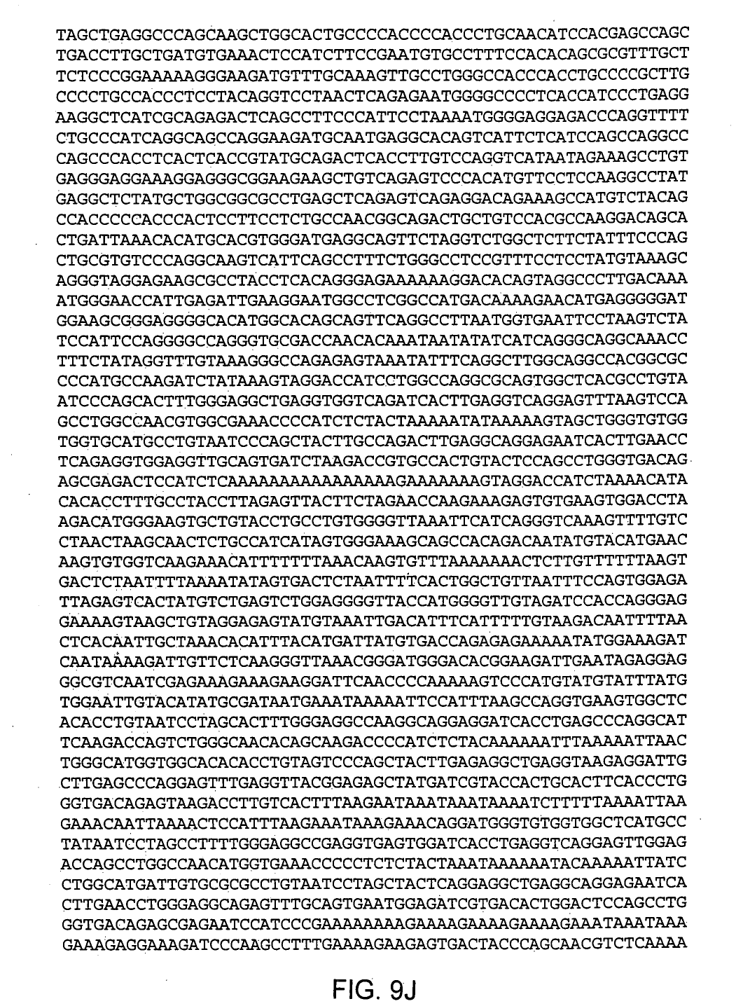

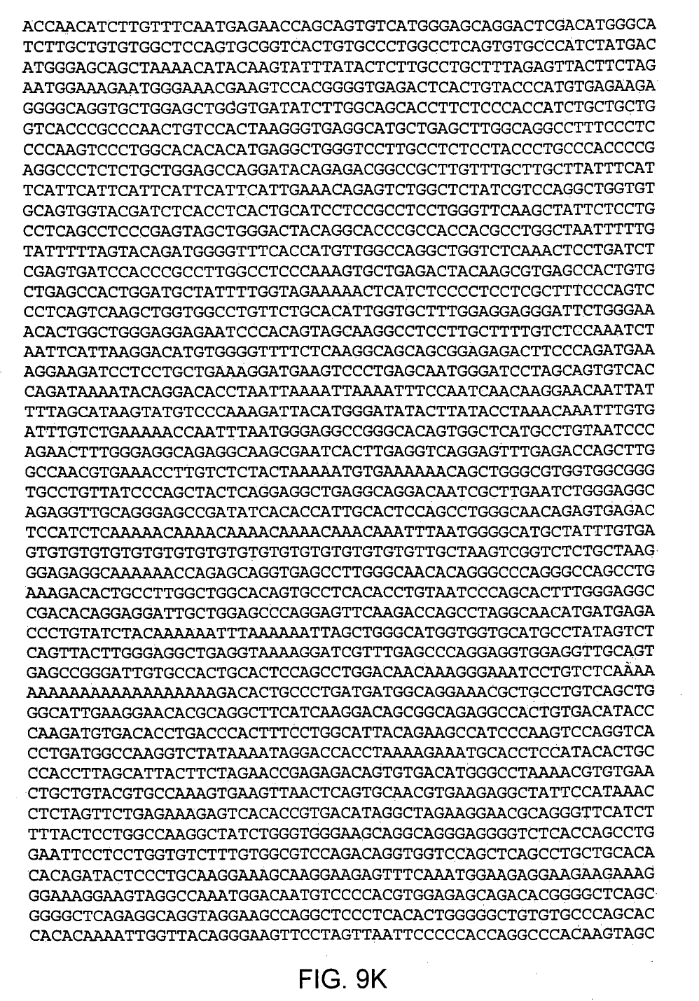

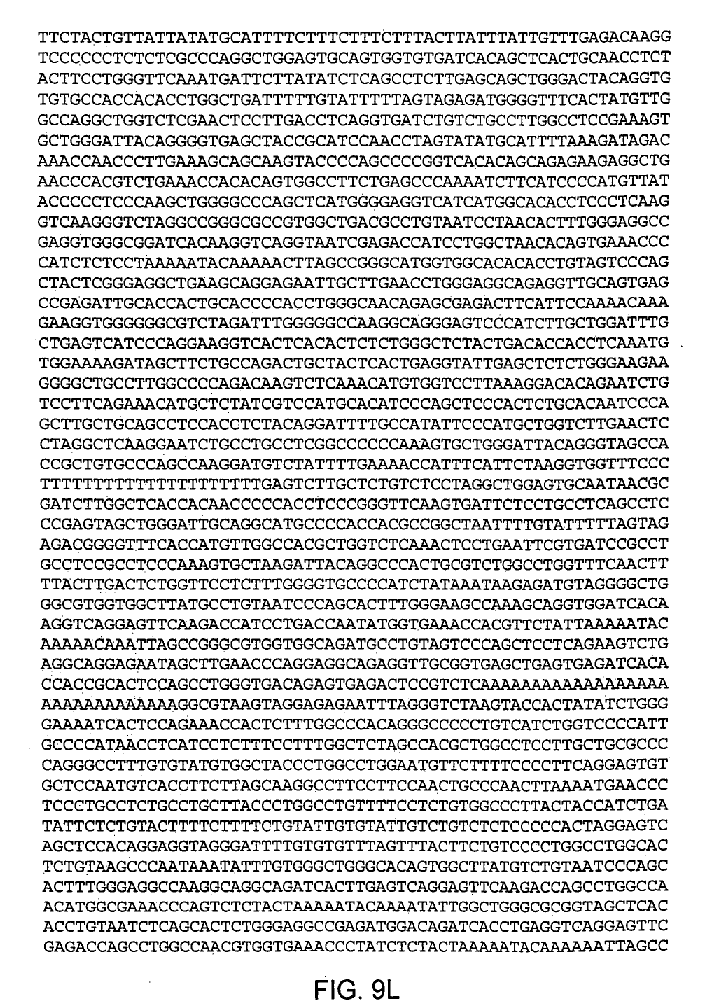

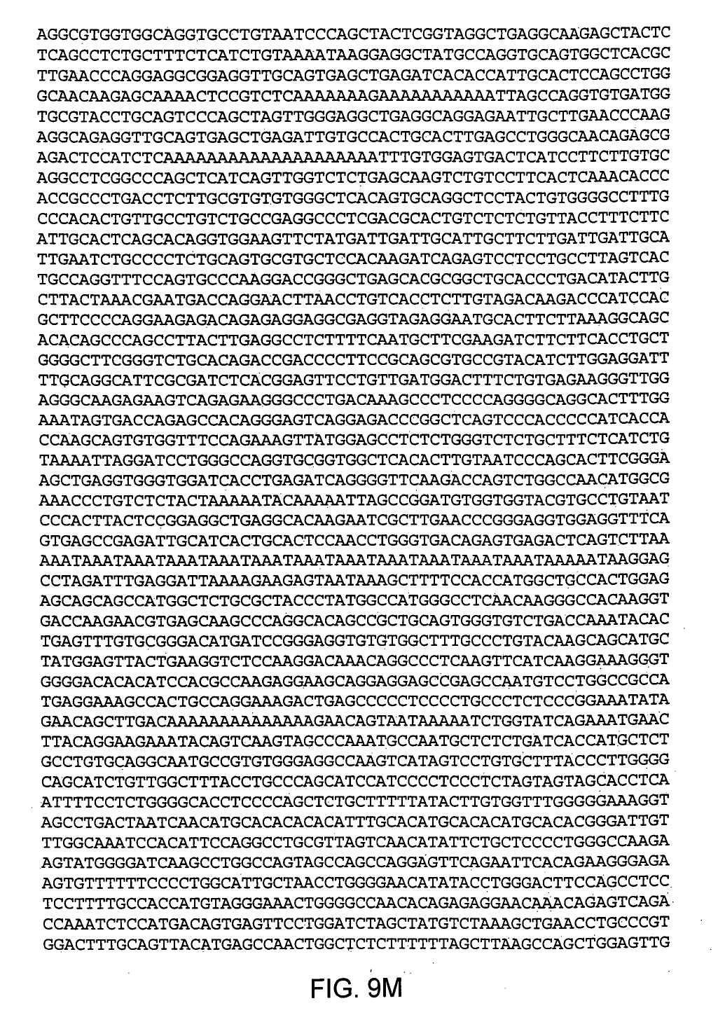

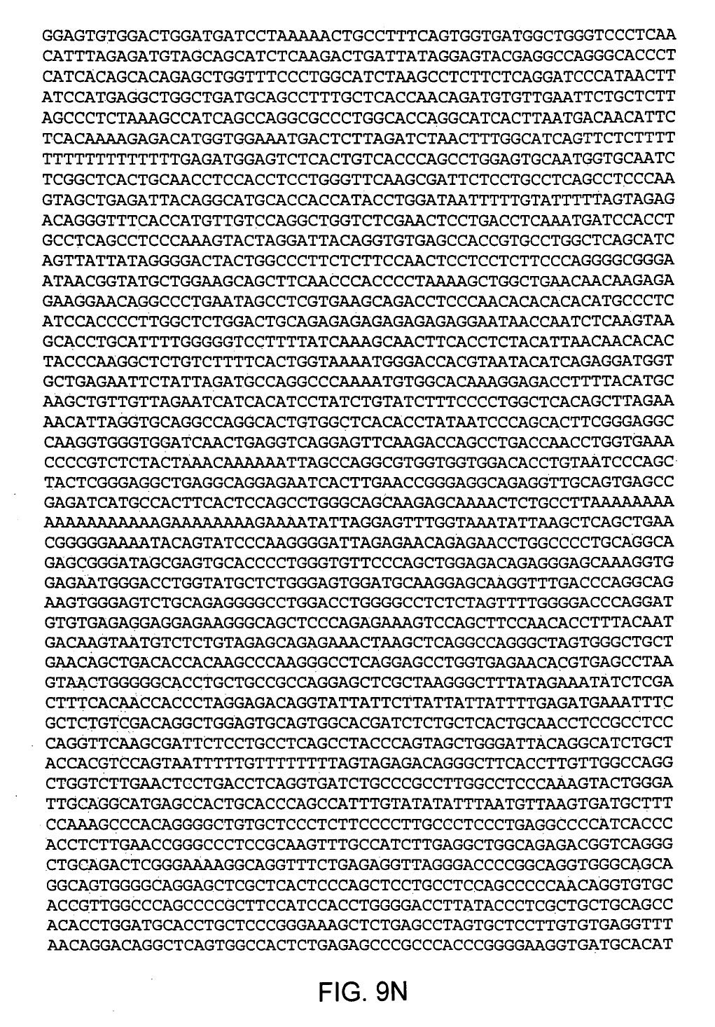

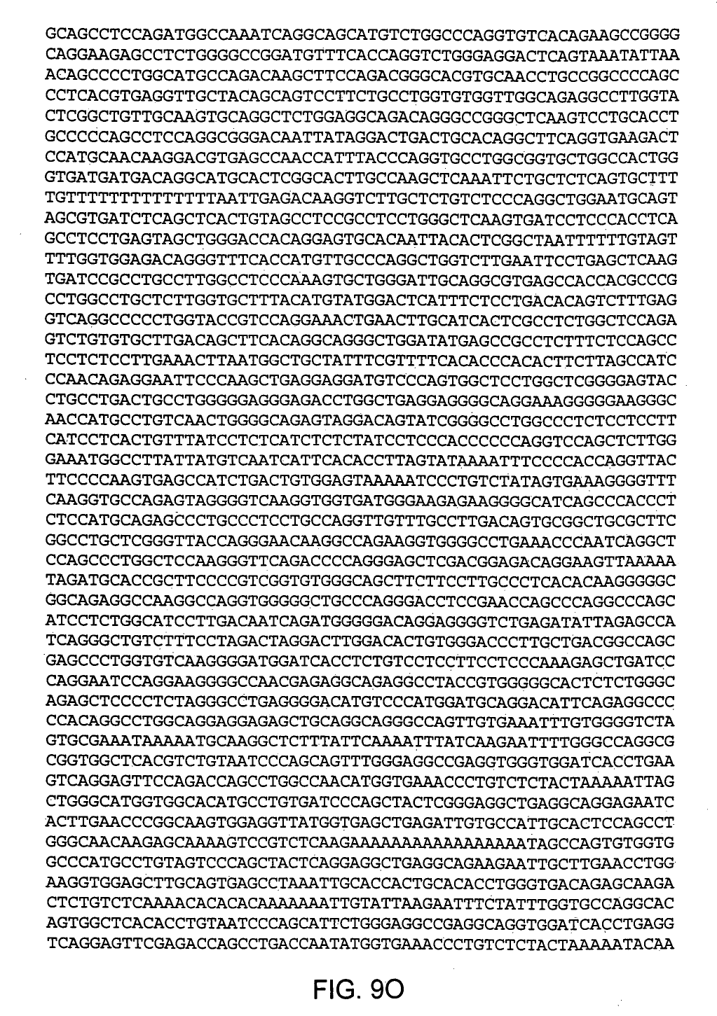

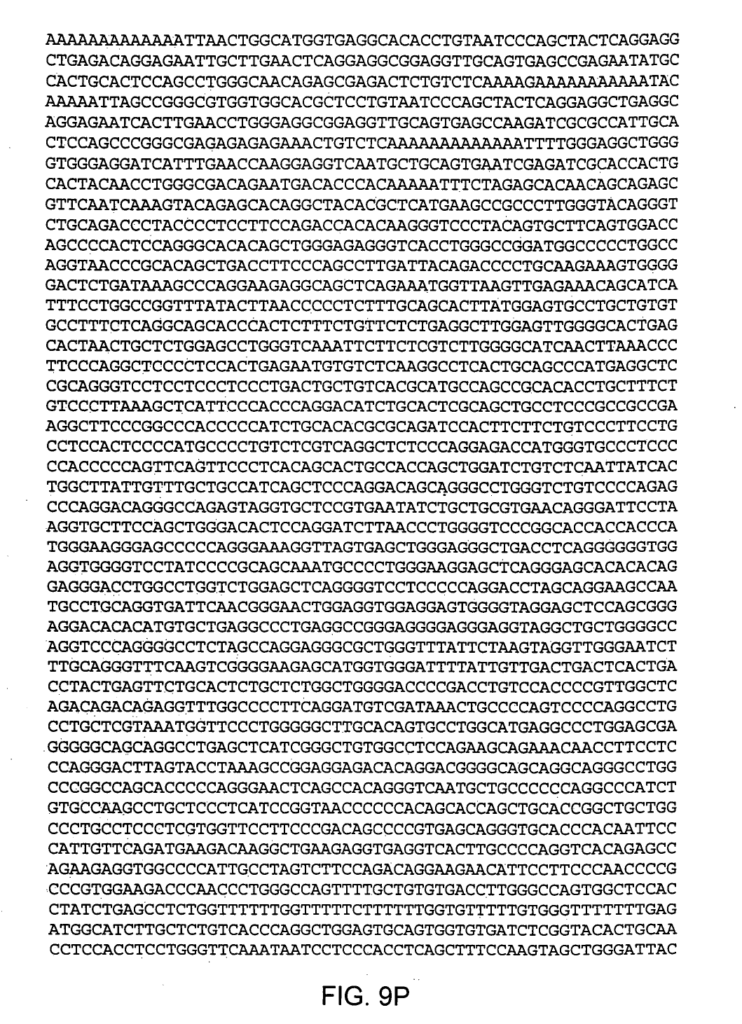

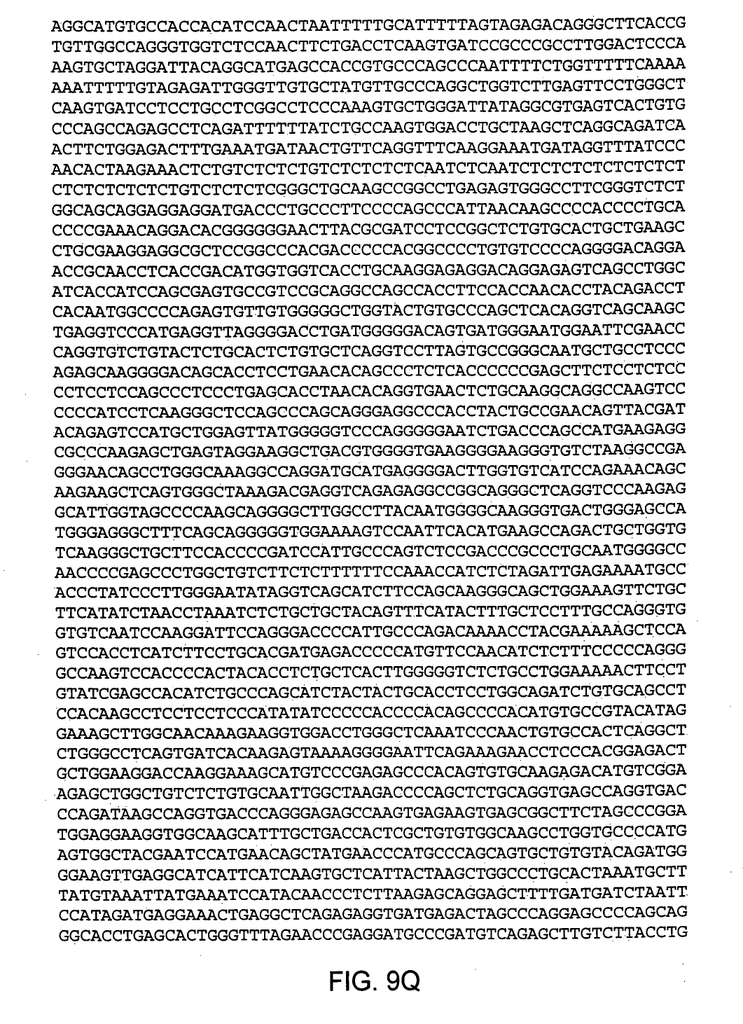

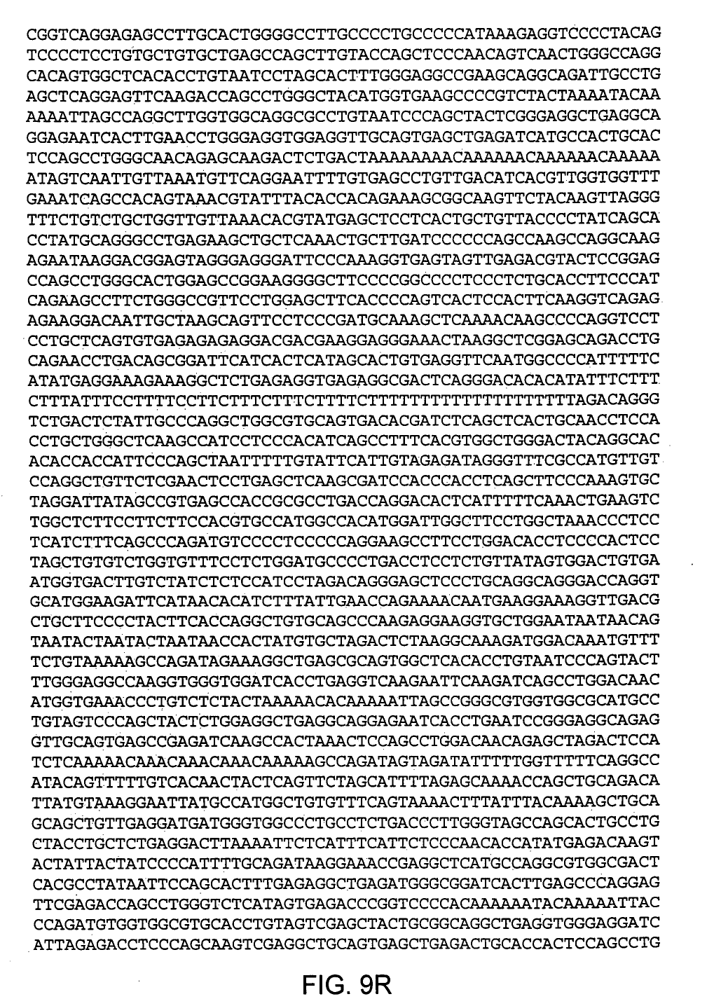

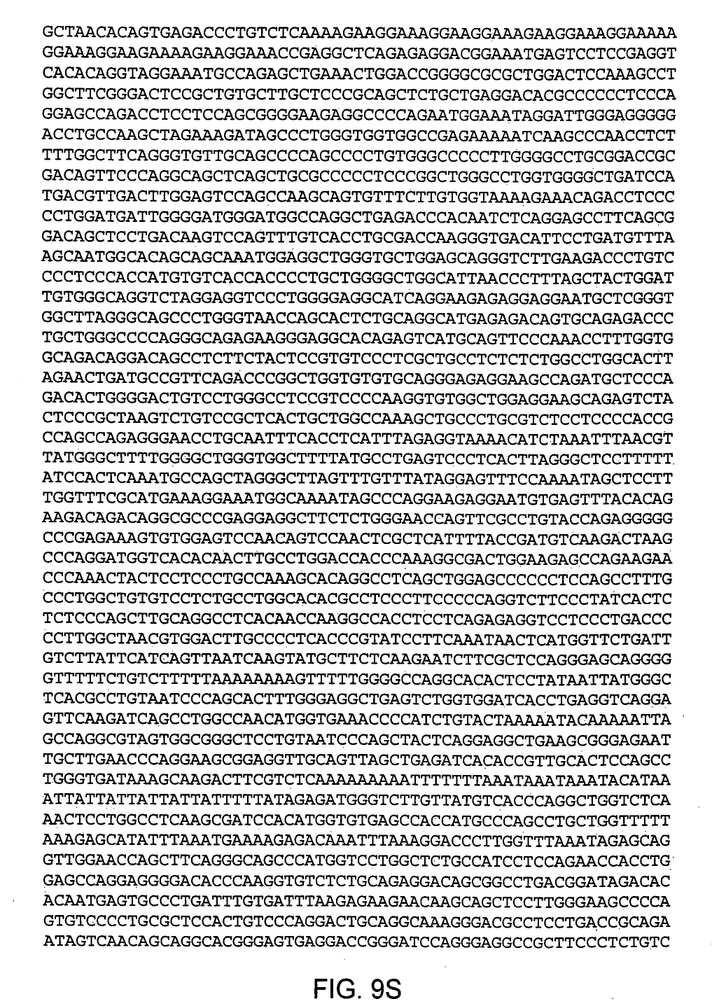

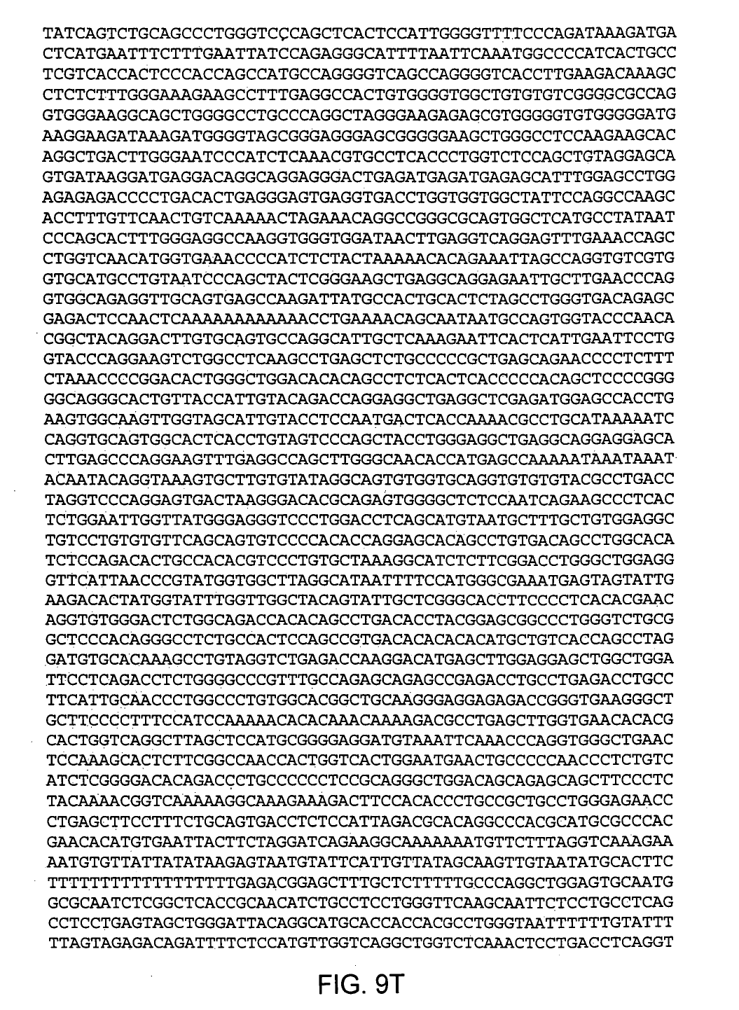

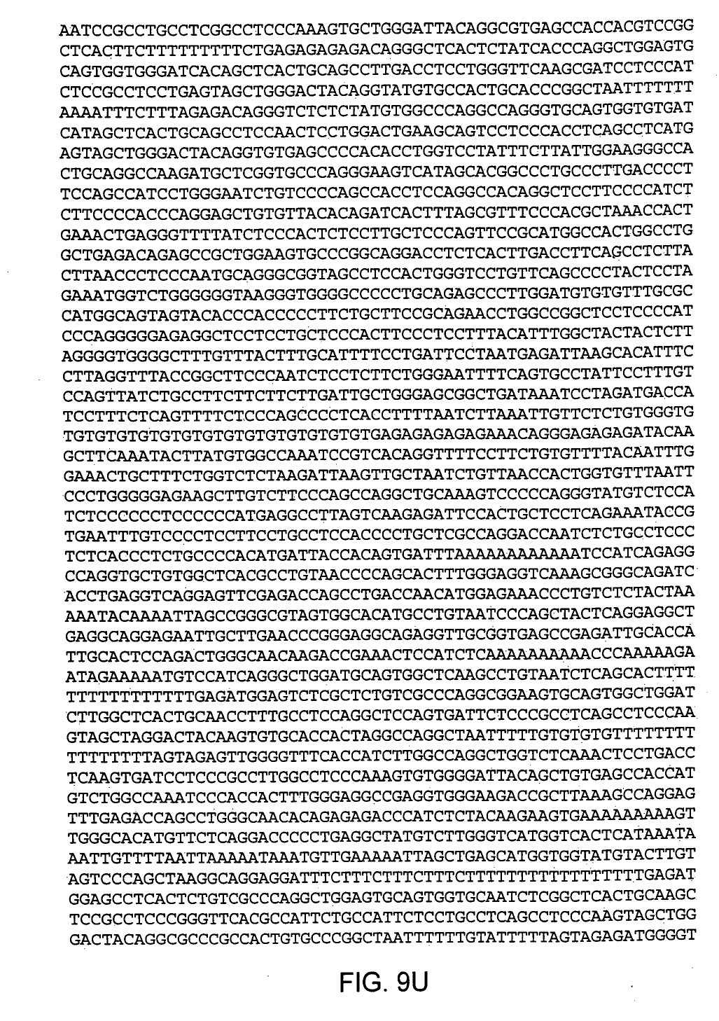

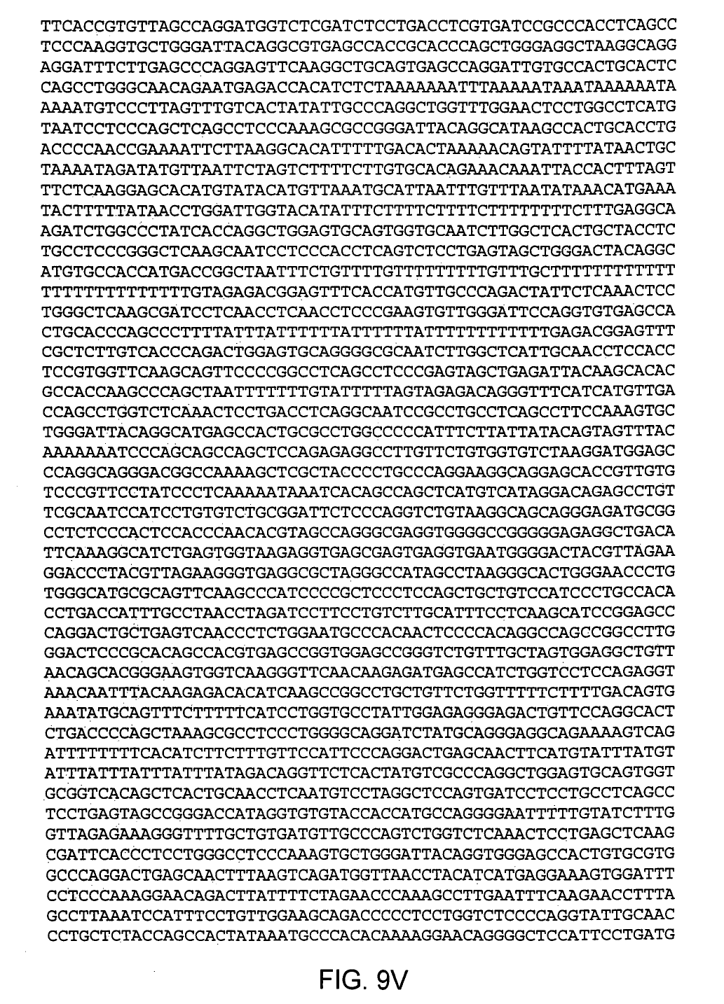

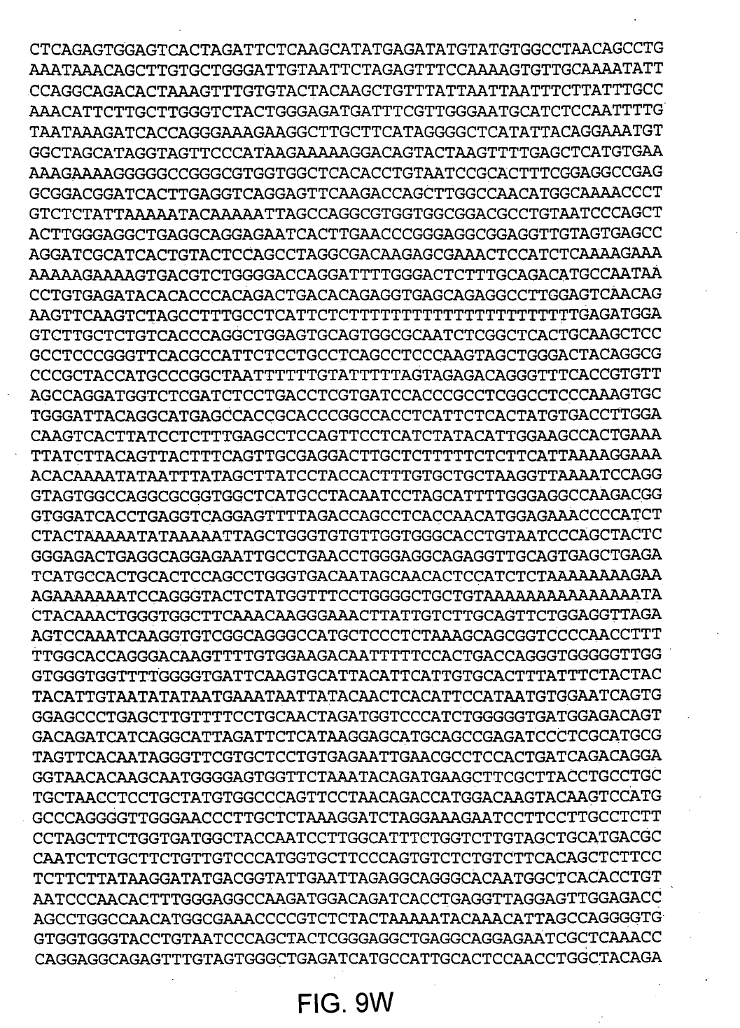

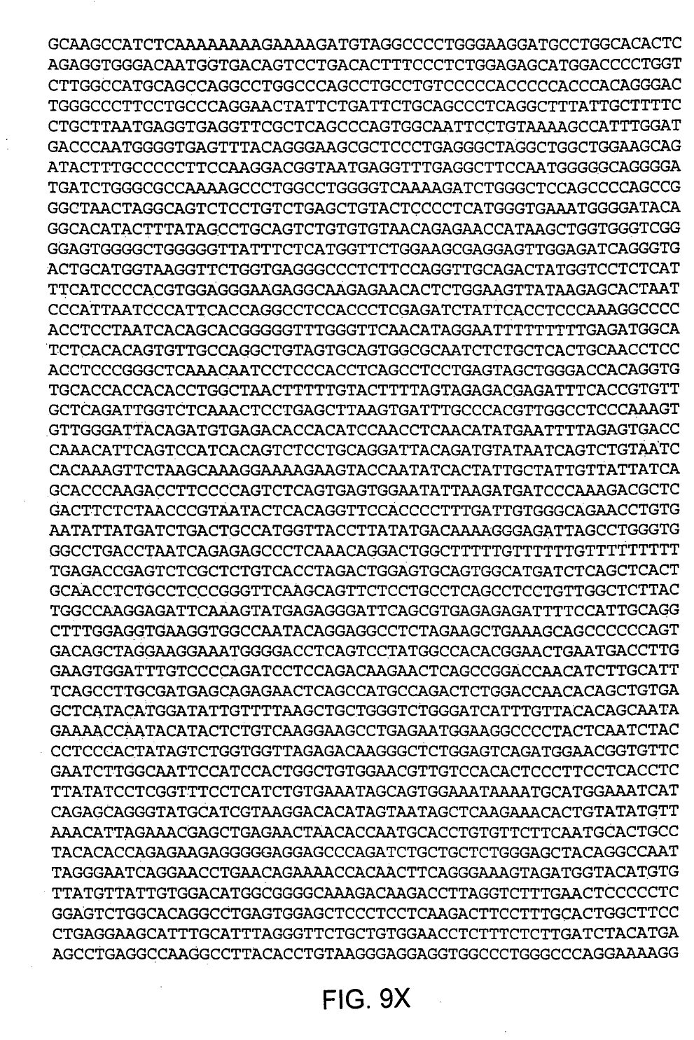

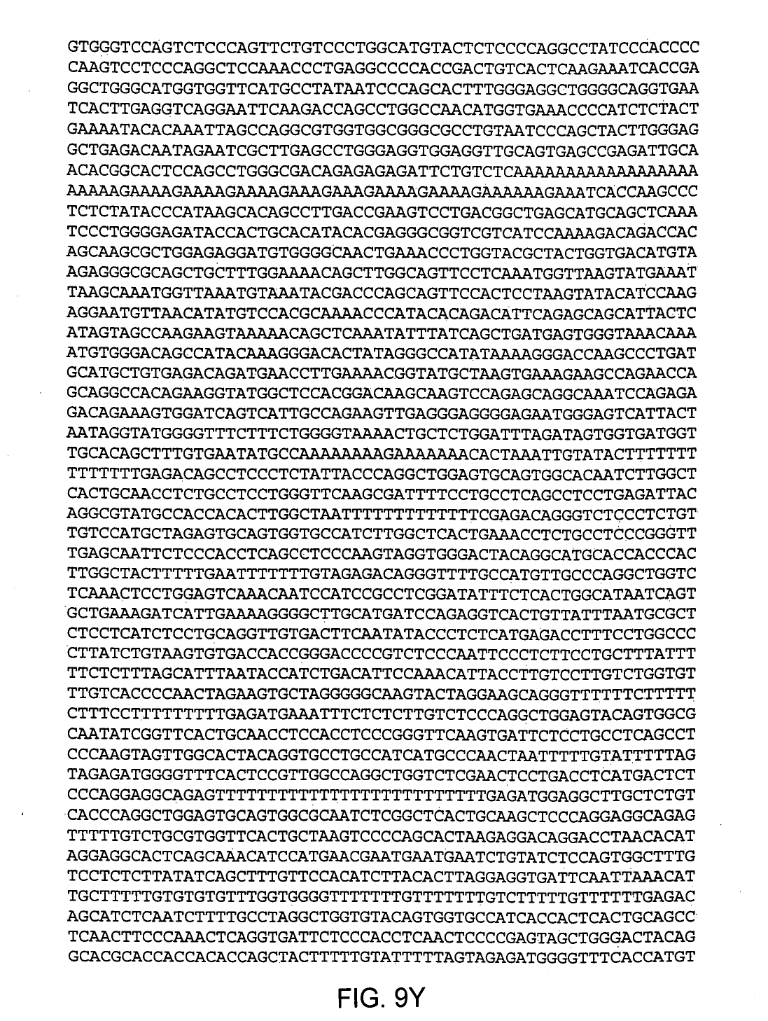

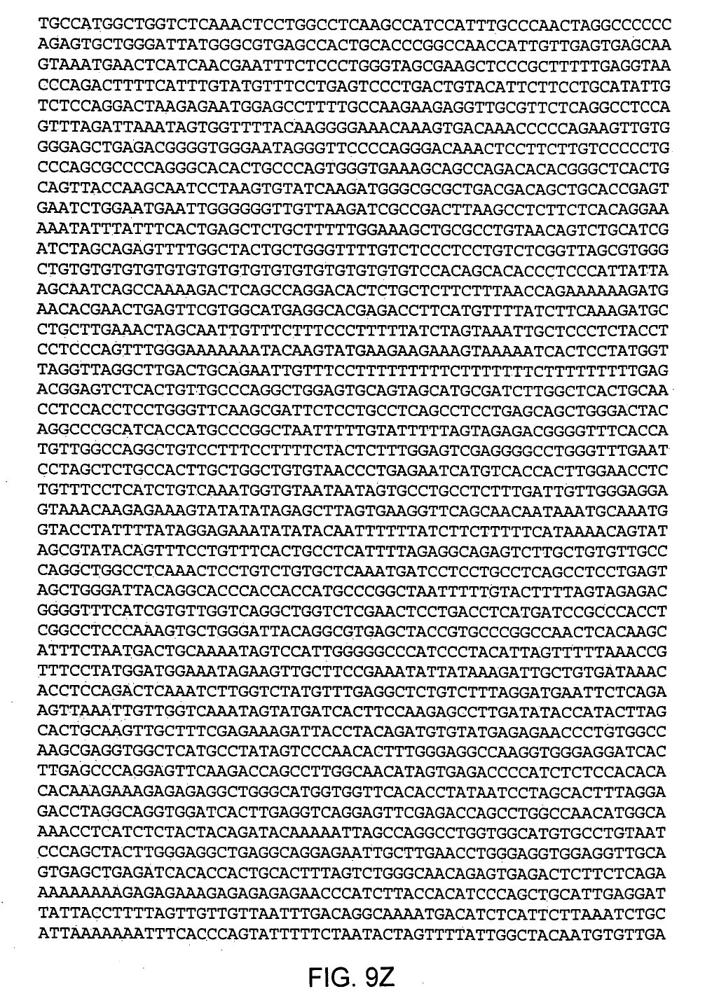

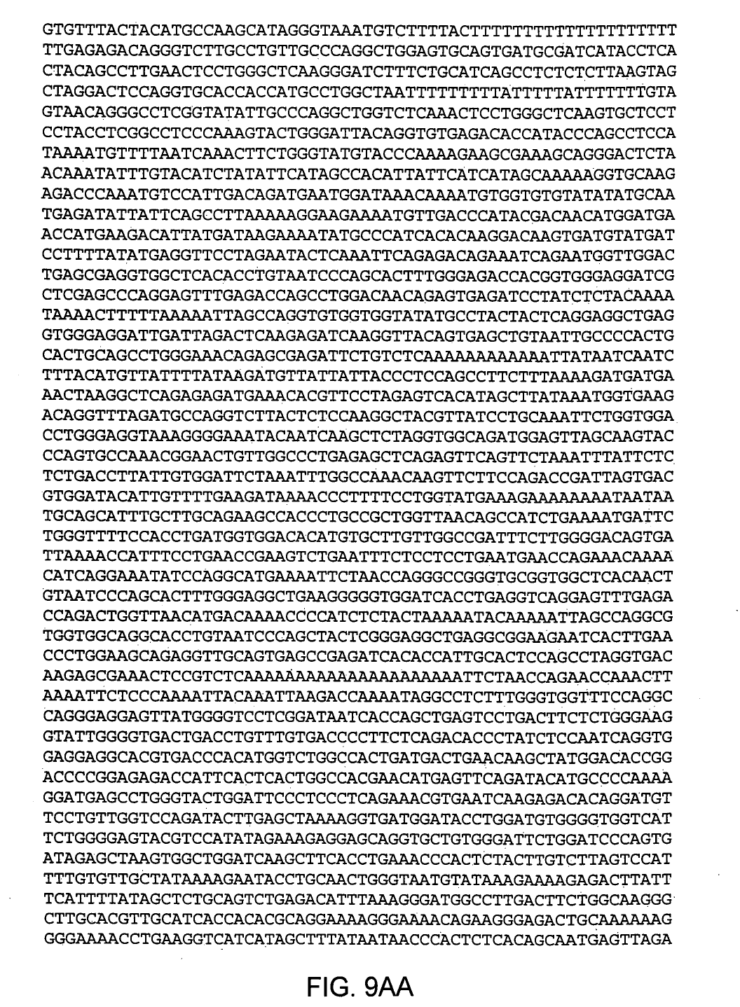

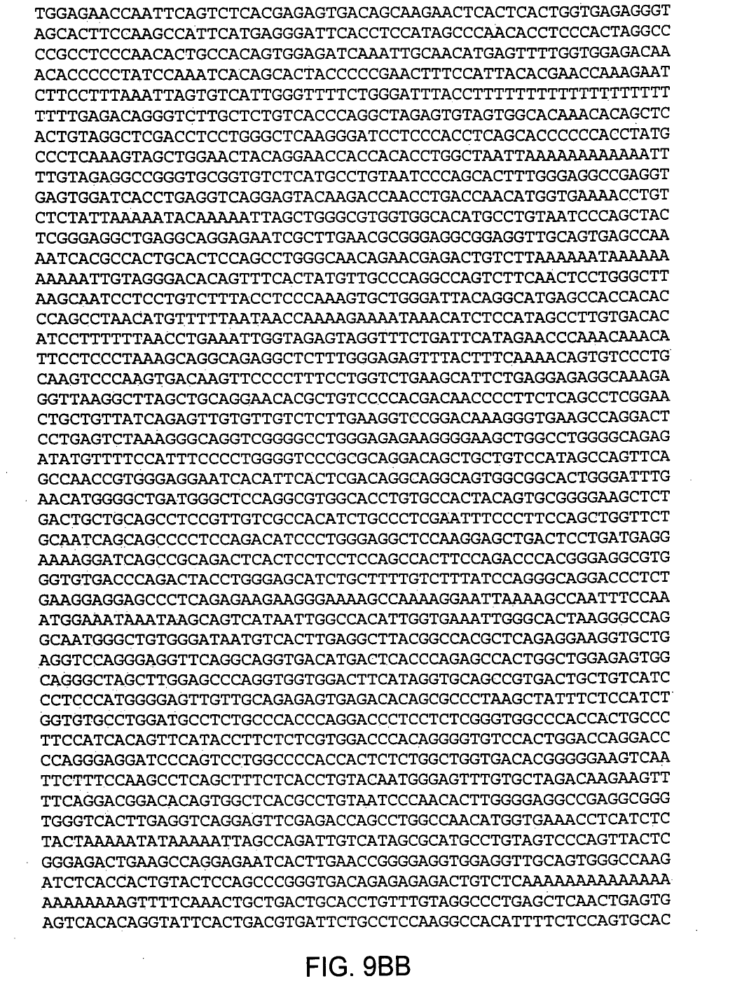

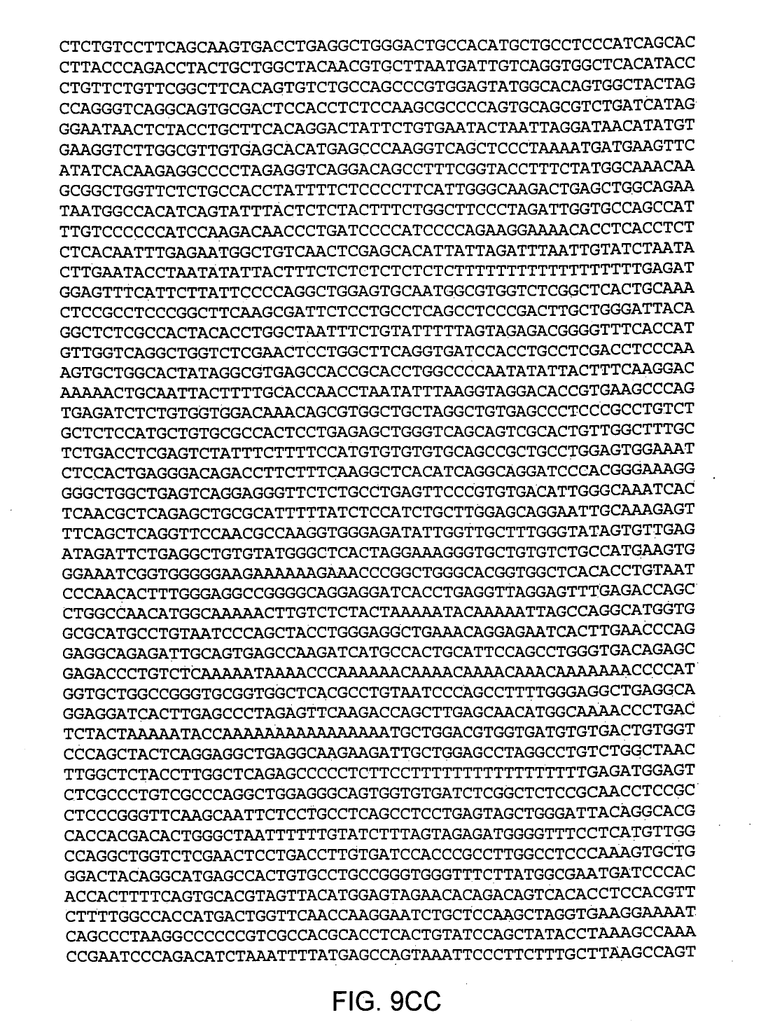

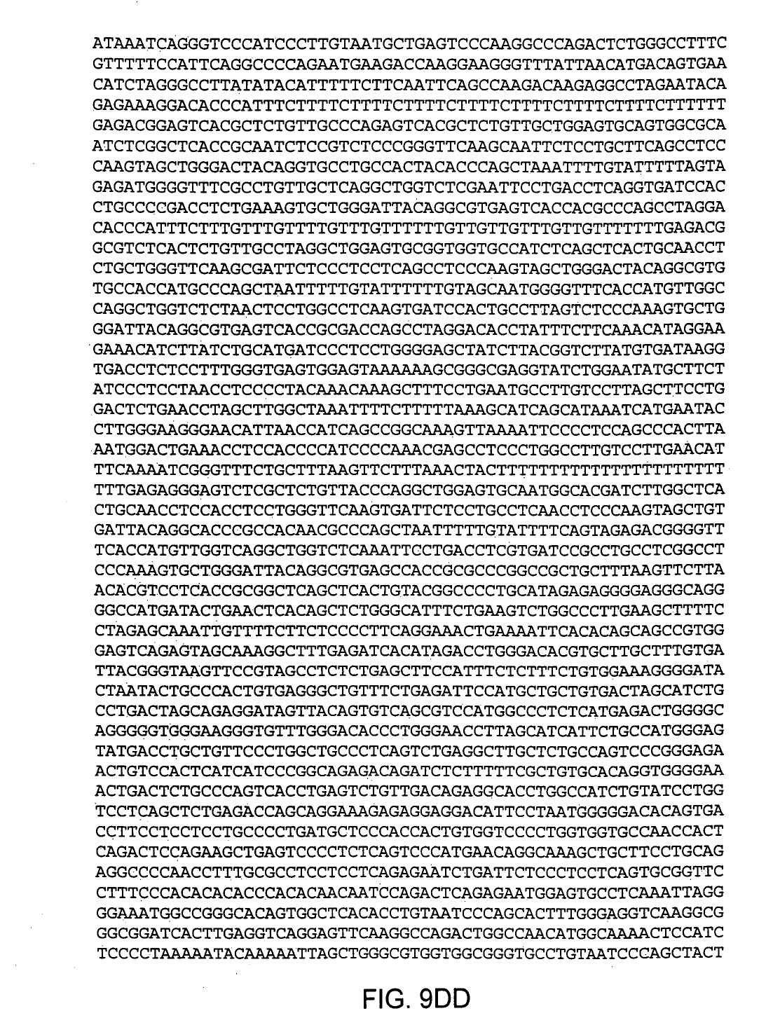

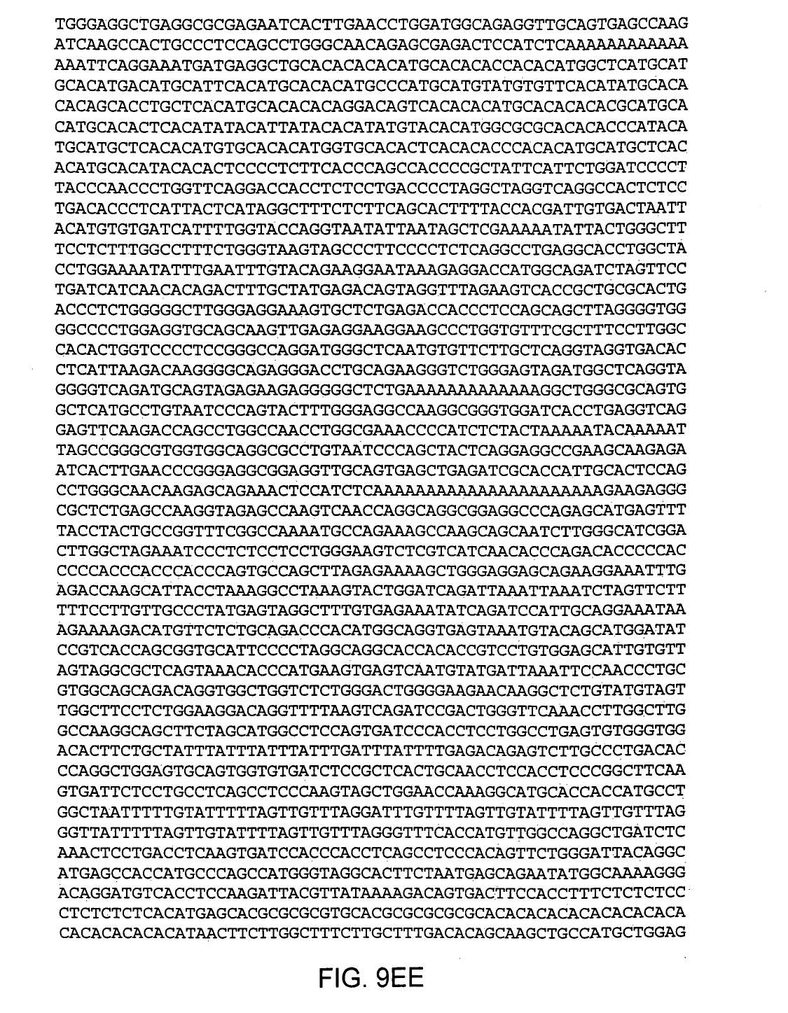

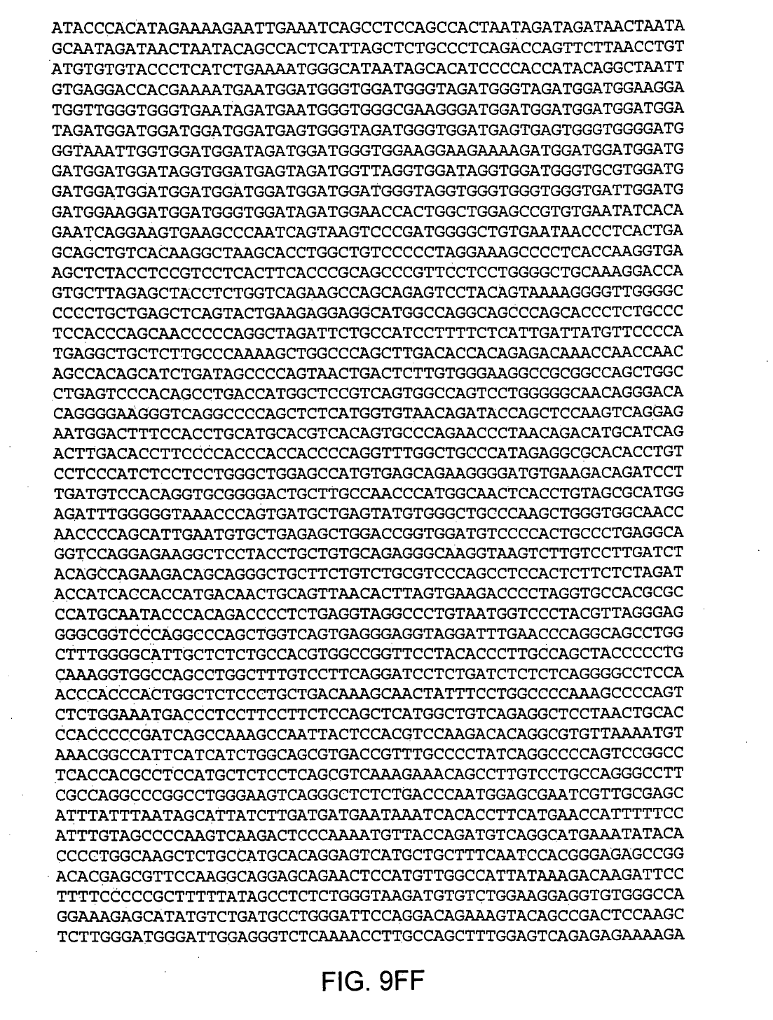

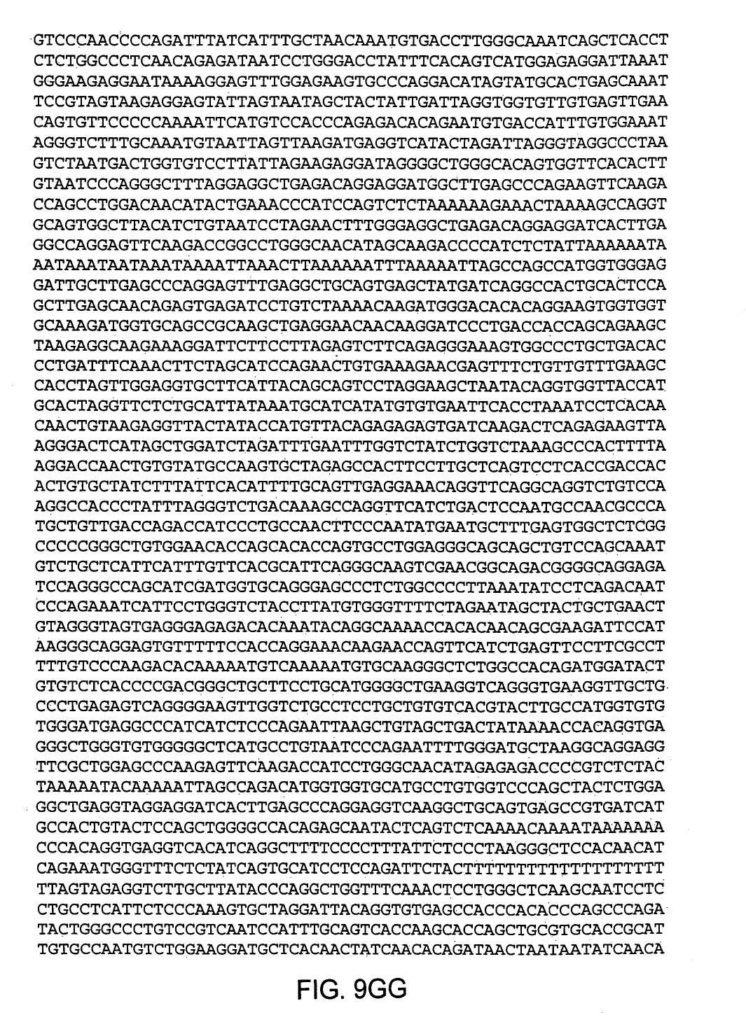

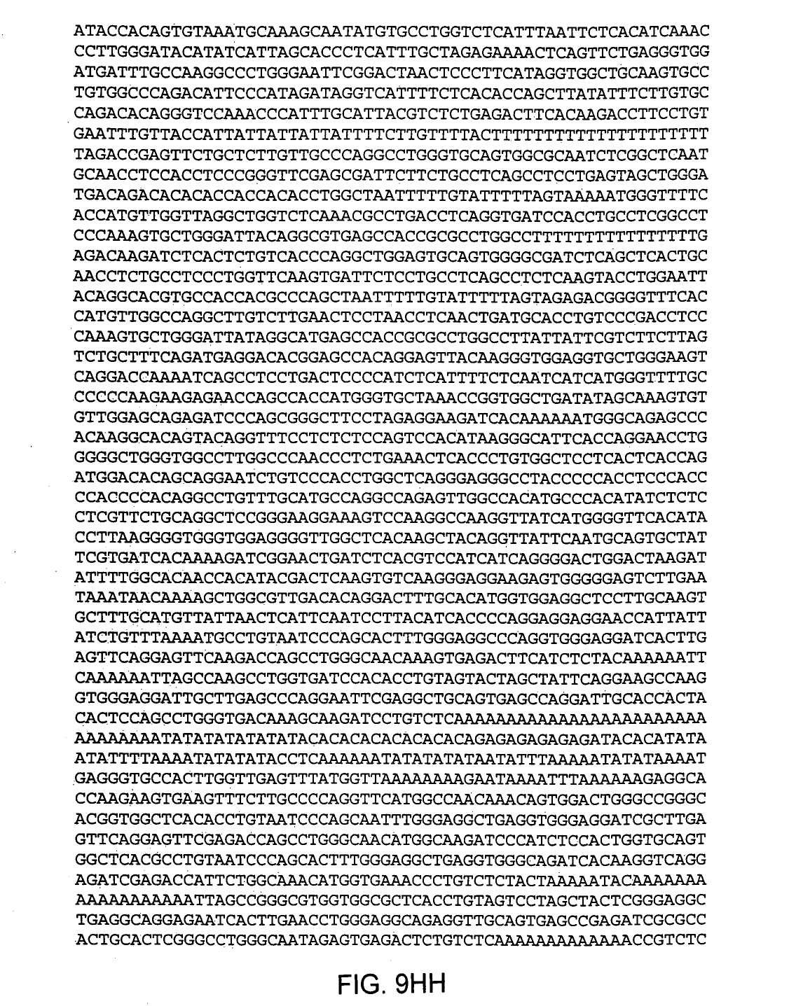

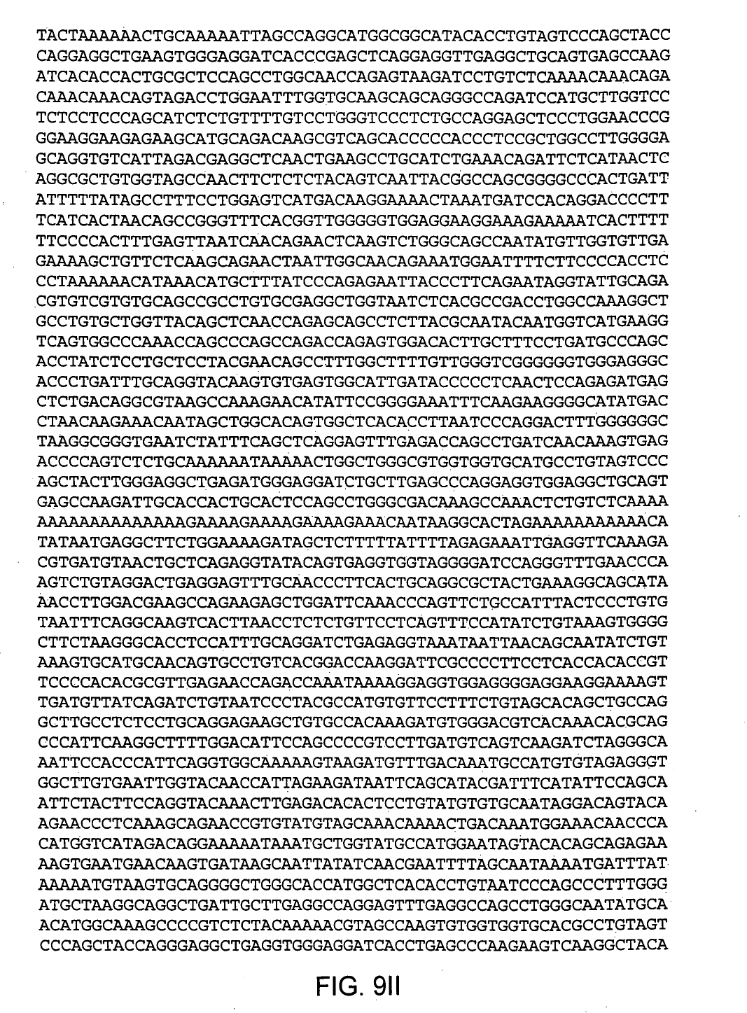

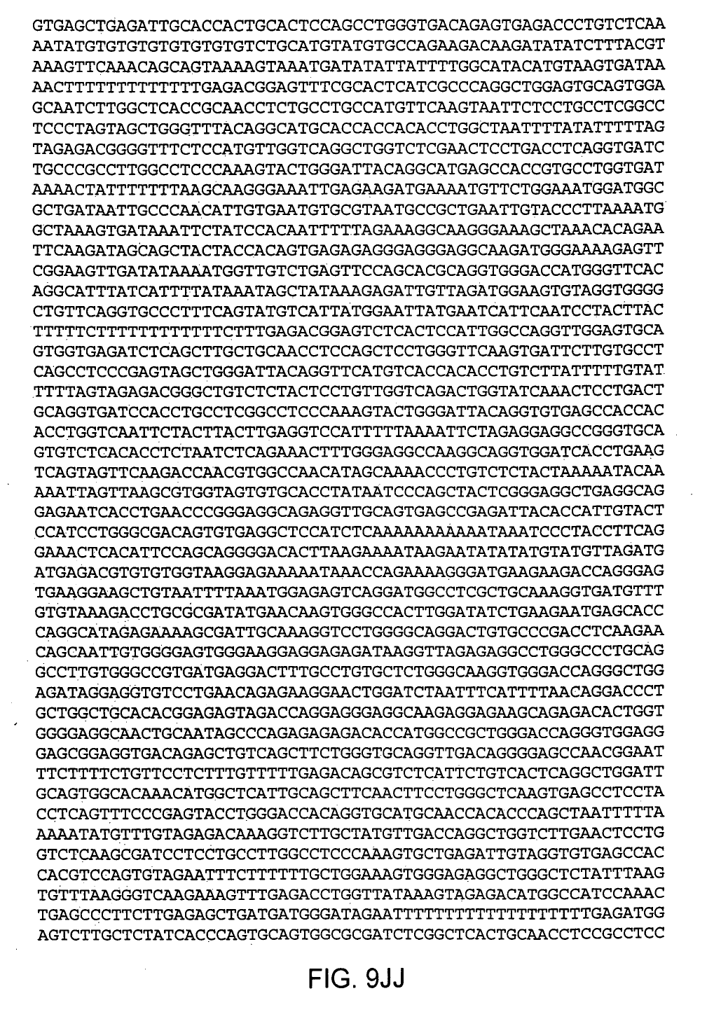

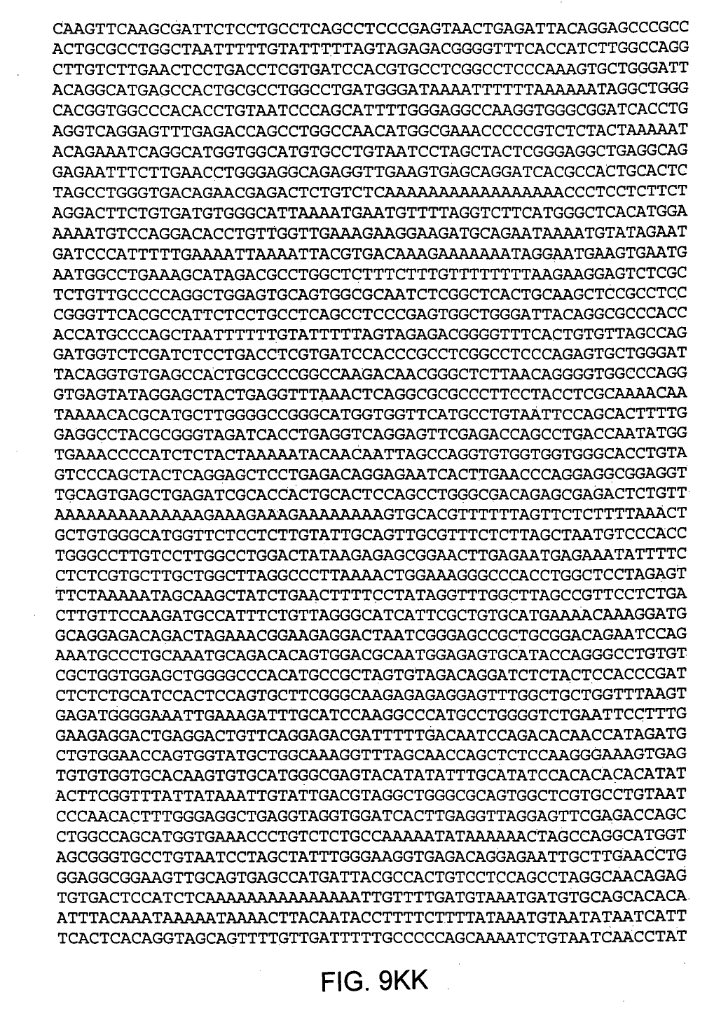

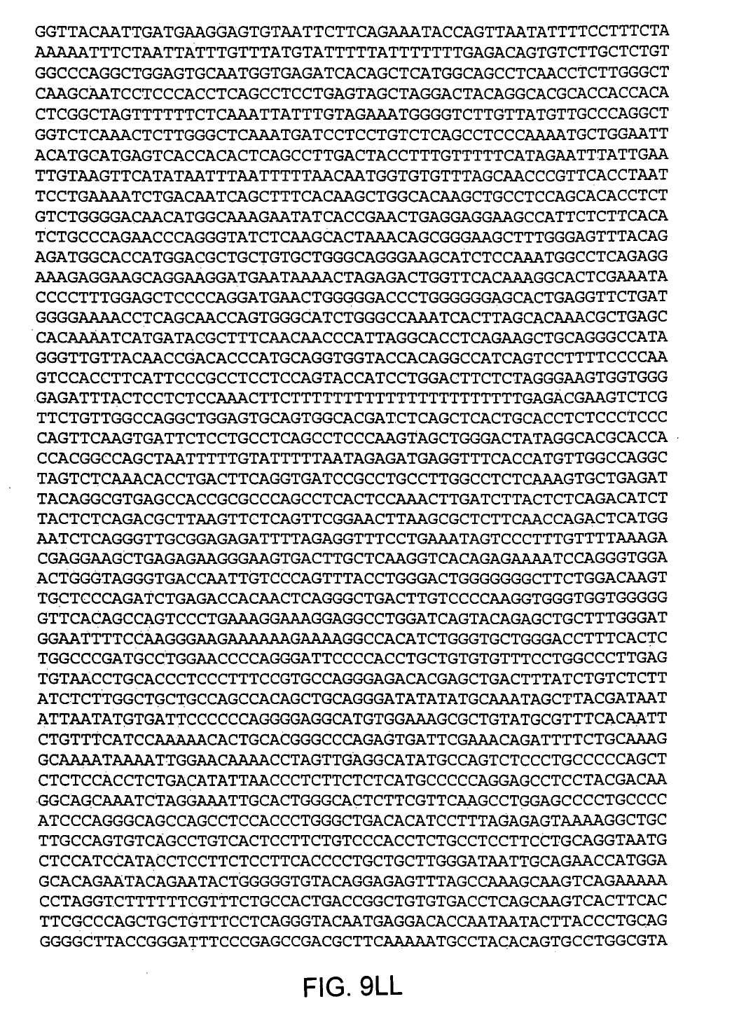

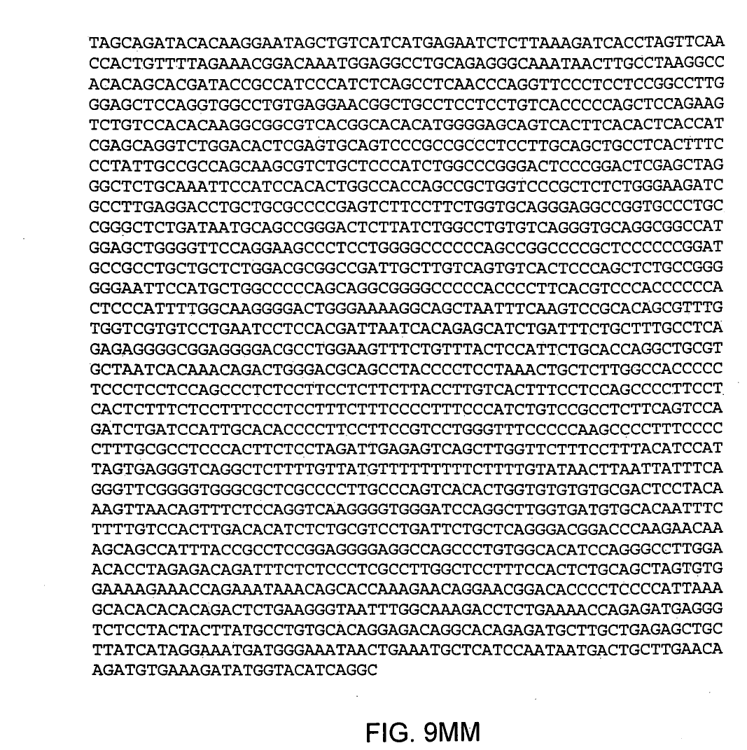

FIG. 6 shows an alignment of Diff40-Long form (SEQ ID NO:37) with T2DM-1a (SEQ ID NO:2). FIG. 7 shows the alignment of Diff40-short form (SEQ ID NO:39) with T2DM-1b. The amino and carboxyl termini of the Diff40 Long form show very significant similarity to T2DM-1a and probably present conserved domains. The central region of the proteins, i.e., Diff40: amino acid residues 355 to 726 of SEQ ID NO:2, T2DM-1: 356-602 is least well conserved, and contains pronounced [S,P,E] compositional biases. The short isoforms of these homologs terminate in the middle of this central region.

Transmembrane Domains: A Kyte-Doolittle hydropathy analysis using GREASE/TGREASE indicates no significant region of hydrophobicity in T2DM-1, long or short form. T2DM-1 is not predicted to cross the transmembrane domain or be a receptor.

Coiled-coils: The leucine-rich nature of the first coiled-coil region in Diff40 is preserved in T2DM-1. The ‘near-leucine zipper’ motif LX₇LX₆LX₆LX₈L (SEQ ID NO:7) is preserved with intervening leucines in hydrophobic heptad positions.

PROSITE Motif Search: The following common motifs are found in T2DM-1:

N-glycosylation Sites

SEQ ID NO:2 residues 640-643 NLSR Long only

SEQ ID NO:2 residues 849-852 NRSF Long only

cAMP- and cGMP-dependent protein kinase phosphorylation sites

SEQ ID NO:2 residues 61-64 RKGS

SEQ ID NO:2 residues 107-110 RRNS

SEQ ID NO:2 residues 337-340 RKGS

Protein Kinase C Phosphorylation Sites

SEQ ID NO:2 residues 2-4 SVR

SEQ ID NO:2 residues 33-35 SRR

SEQ ID NO:2 residues 44-46 SVR

SEQ ID NO:2 residues 53-55 SSK

SEQ ID NO:2 residues 59-61 TLR

SEQ ID NO:2 residues 100-102 SGR

SEQ ID NO:2 residues 106-108 TRR

SEQ ID NO:2 residues 290-292 TTR

SEQ ID NO:2 residues 305-307 TIK

SEQ ID NO:2 residues 329-331 TGK

SEQ ID NO:2 residues 336-338 SRK

SEQ ID NO:2 residues 351-353 SFR

SEQ ID NO:2 residues 392-394 SLR

SEQ ID NO:2 residues 603-605 SLK Long only

SEQ ID NO:2 residues 607-609 SSR Long only

SEQ ID NO:2 residues 820-822 TLR Long only

SEQ ID NO:2 residues 832-834 TPR Long only

SEQ ID NO:2 residues 840-842 SAR Long only

SEQ ID NO:2 residues 851-853 SFR Long only

Casein Kinase II Phosphorylation Sites

11 Short, 23 Long, Short Form 11 present in both

N-Myristoylation Sites

8 Short, 10 Long, 7 present in both

Amidation Sites

SEQ ID NO:2 residues 807-810 QGKR Long only

T2DM-1 Blast Searches: A Blastp search was performed using T2DM-1a and 1b against the NCBI protein databases. Apart from matches to human and murine Diff40, there is a match to C elegans C27H2.3 (accession T19532, NP_{—}502680.11) in the same regions as Diff40. This suggests these regions contain important (possibly orthologous) domains that are conserved across invertebrate and non-invertebrate species. The exact function of this protein in C. elegans is unknown.

T2DM-1 is 27.4% identical to AAB53946, the human homolog of the mouse FOSB gene, E=0.0089, 27.4% identity, T2DM-1 range=385-616. There were also matches to mouse and canine homologs. Hsa is also known as GOS3 (putative G0/G1 switch regulatory gene 3) and is a member of the FOS family. These genes encode leucine zipper proteins that can dimerize with proteins of the JUN family, thereby forming the transcription factor complex AP-1. As such, the FOS proteins have been implicated as regulators of cell proliferation, differentiation, and transformation. The second part of the FOSB matching region to T2DM-1a encompasses the known basic leucine zipper domain of FOSB, although T2DM-1a does not contain a repeating leucine motif in the aligned sequence (amino acids 558-616 of SEQ ID NO:2).

T2DM-1 is 26.3% identical to AAL99670 the semaphorin 6C short isoform 2 (SEM6C) [Mus musculus] E=0.064, 26.3% identity, over the range 304-617. The corresponding matching region in SEM6C is within the semaphorin domain of the protein. Semaphorins are a family of signaling genes that act to provide guidance cues for growing axons to guide their development trajectory.

### Comparison of Diff40 and T2DM-1 Sequences

Table 1 shows a summary of some of the predicted properties of Diff40 and T2DM-1. Unless otherwise specified, the residue interval is for Diff40-Long isoform (SEQ ID NO:23).

TABLE 1

| Region | | |
|---|---|---|
| (residues) | Protein | Structure Element |
| 1-46 | Diff40 + T2DM-1 | Serine-rich region |
| 1-321 | Diff40 + T2DM-1 | High similarity between Diff40 & T2DM-1. |
| | | Homologous domain(s). |
| 76-112, 117-145 | Diff40 | Coiled-coil region |
| 86-118 | Diff40 + T2DM-1 | Leucine-rich region. Coincides with predicted |
| | | Coiled-coil region in Diff40. |
| 147-300 | Diff40 | Remote C2 domain similarity |
| 253-282 | Diff40 | Possible transmembrane domain |
| 350-590 | Diff40 | Serine-rich region (less so in T2DM-1) |
| 350-530 | Diff40 | Proline-rich region ~8% P (less so in T2DM-1) |
| 380-464 | Diff40 | Remote similarity to IL4R precursor |
| 407-586 | Diff40 | Glutamate-rich region ~14% E (less so in |
| | | T2DM-1) |
| 645-670 | Diff40 | Serine-rich region (less so in T2DM-1) |
| 725-1048 | Diff40 + T2DM-1 | High similarity between Diff40 & T2DM-1. |
| | | Homologous domain(s). |
| 870-930 | Diff40 | Serine-rich region (less so in T2DM-1) |
| 900-1037 | Diff40 + T2DM-1 | Leucine-rich region |

### T2DM-1 or T2DM-2 Polymorphisms

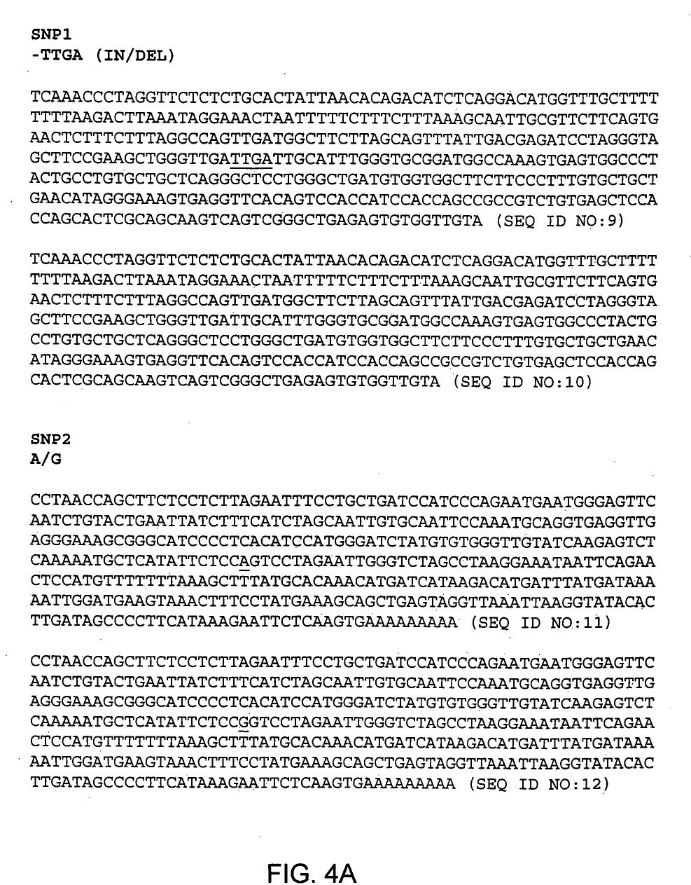

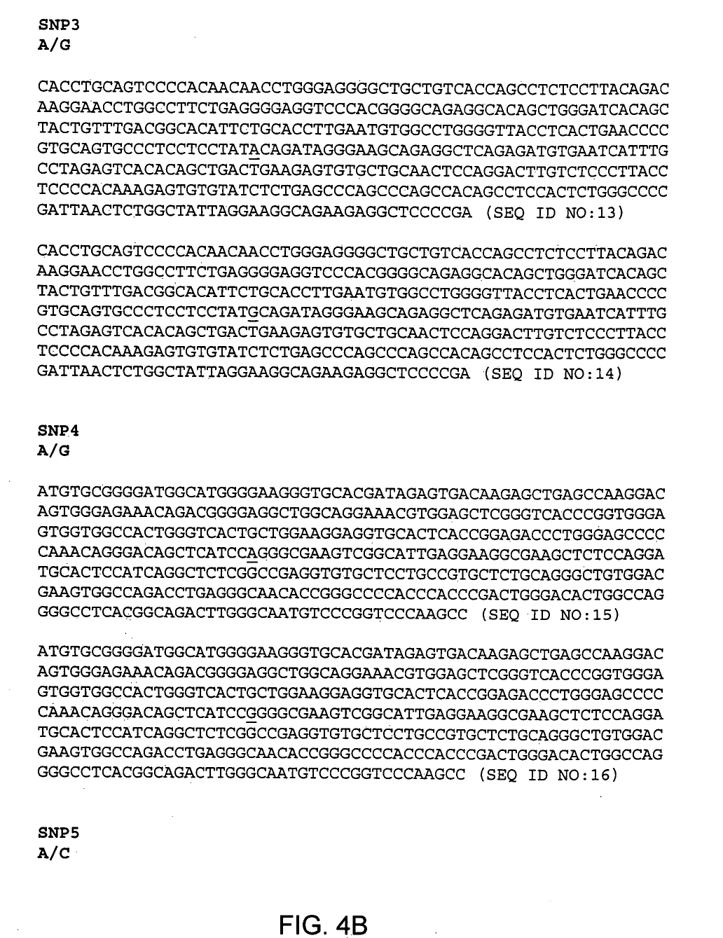

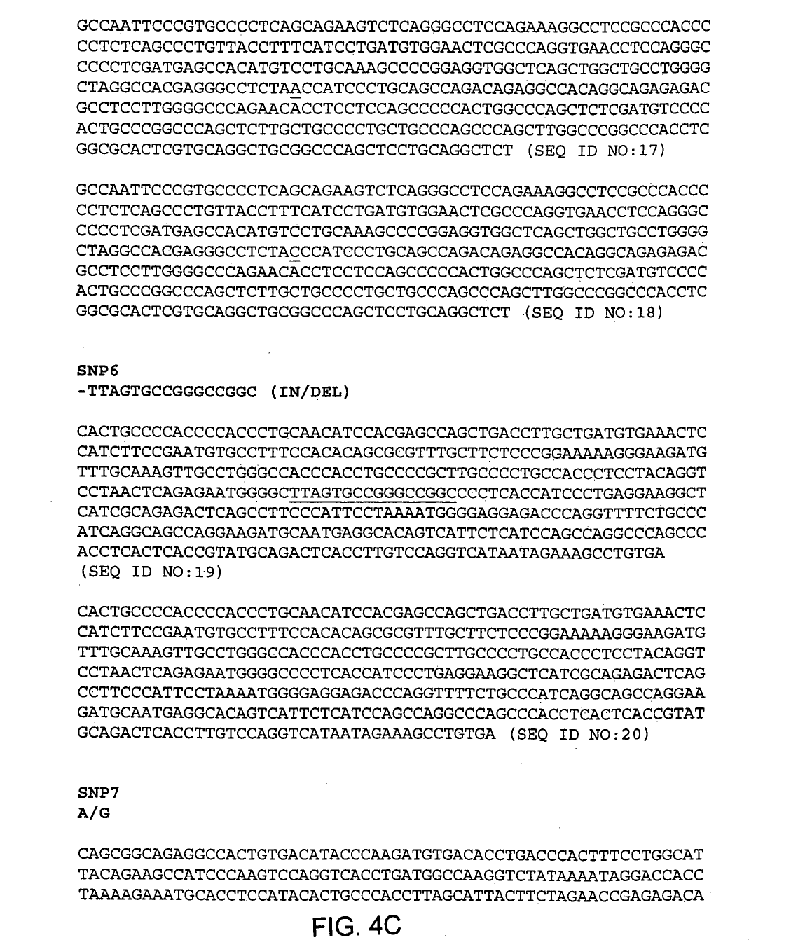

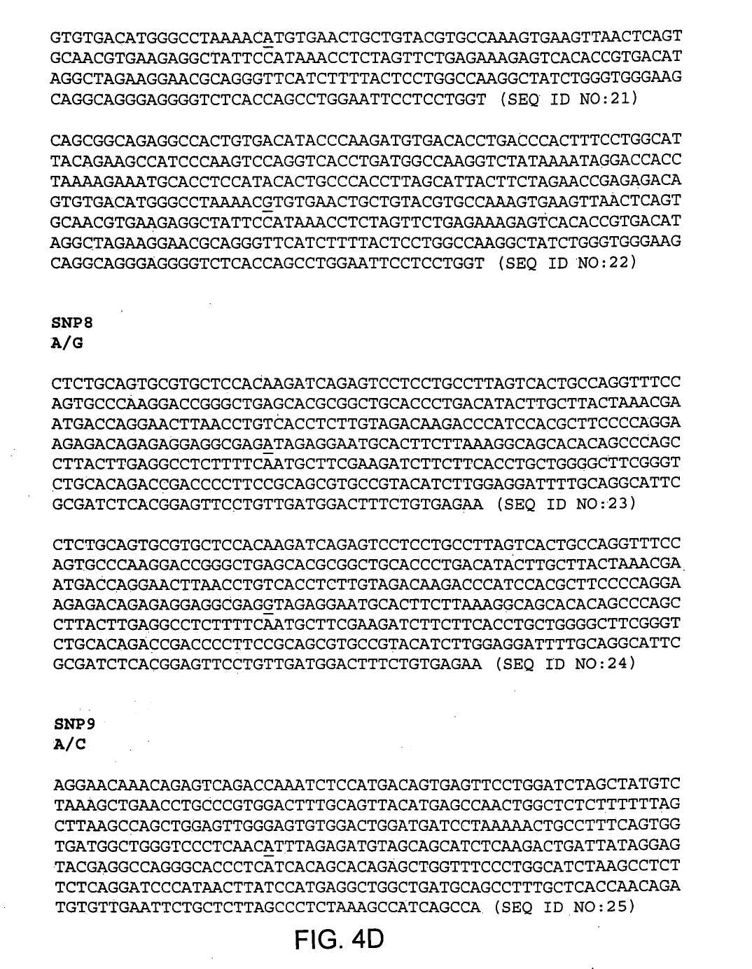

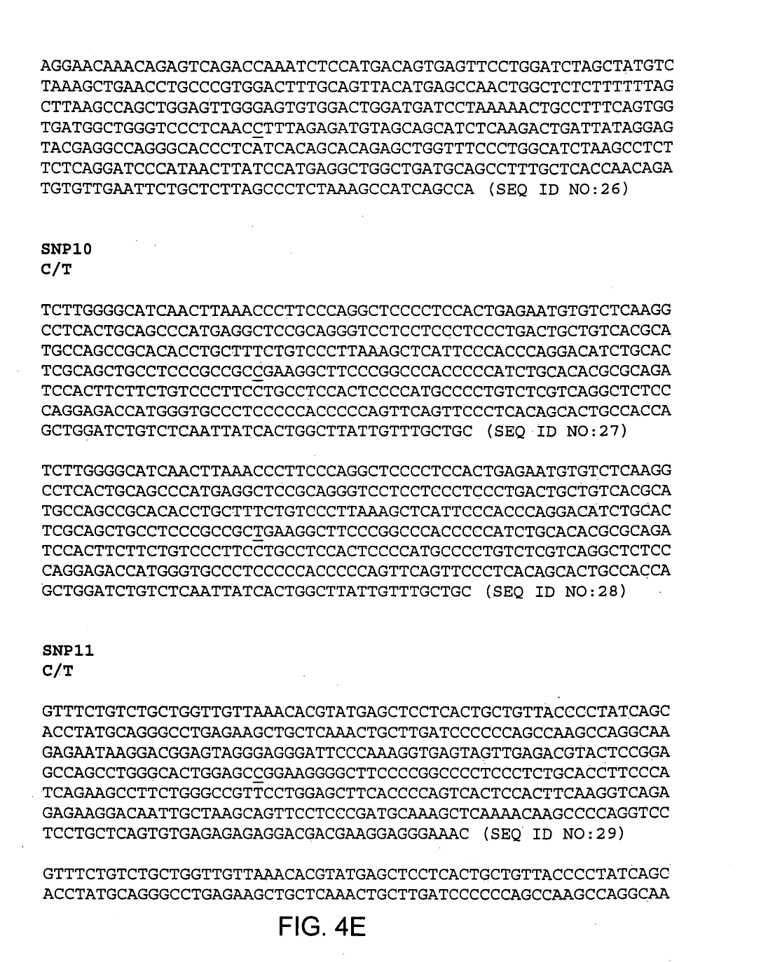

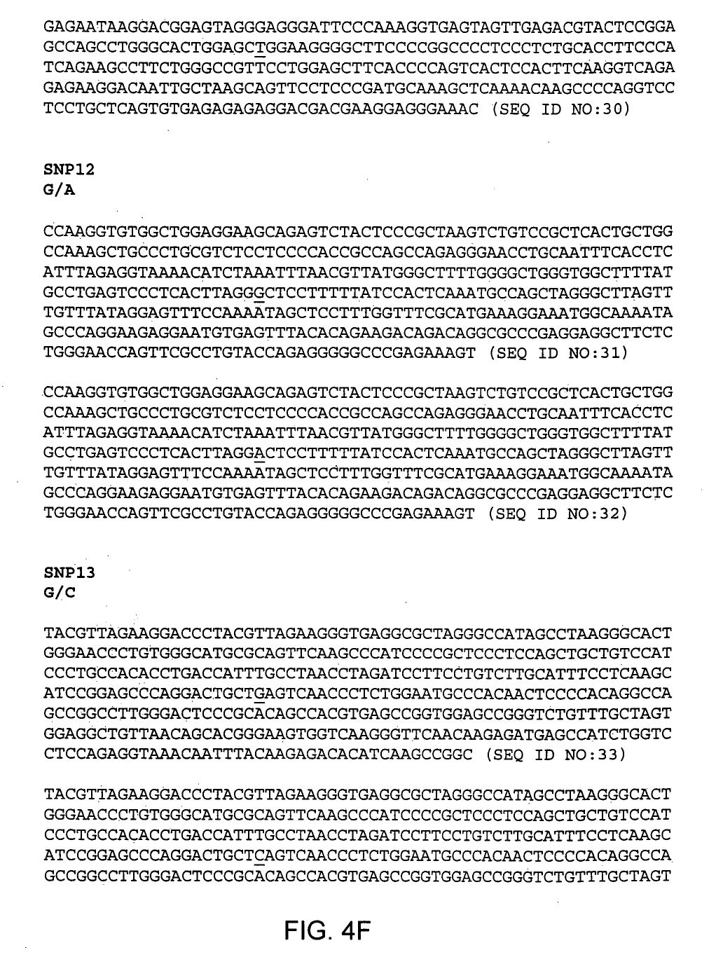

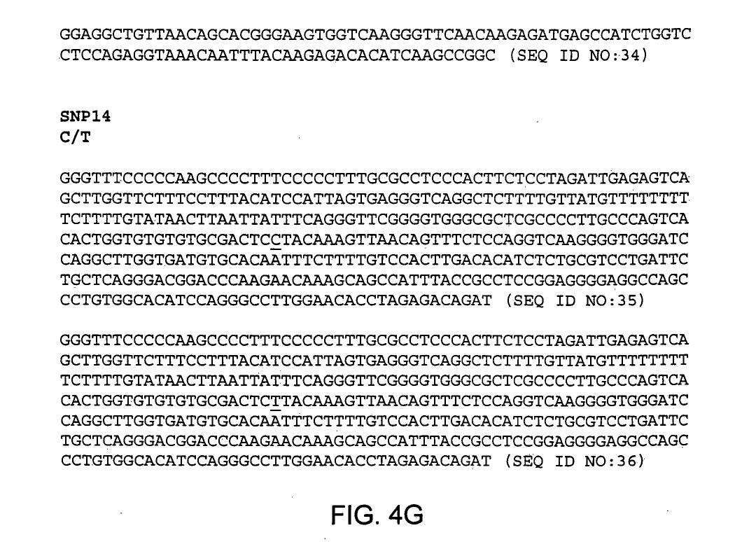

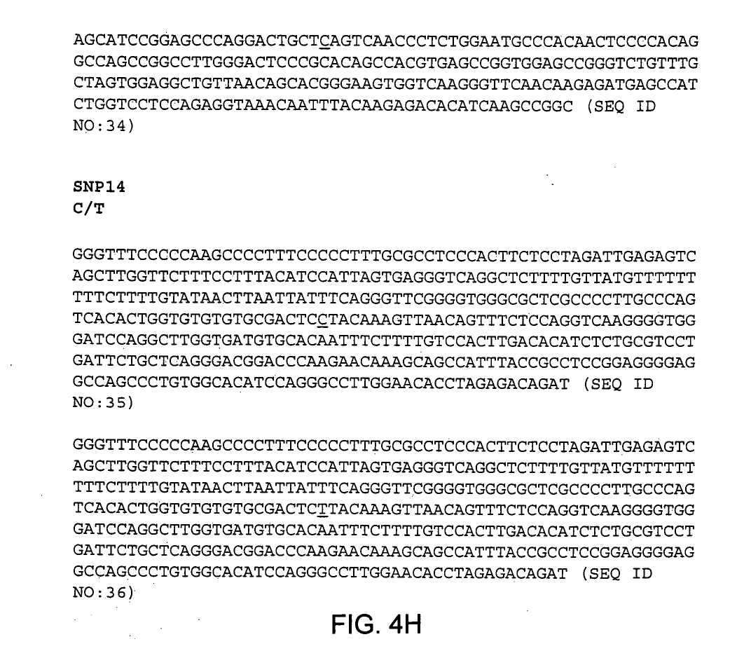

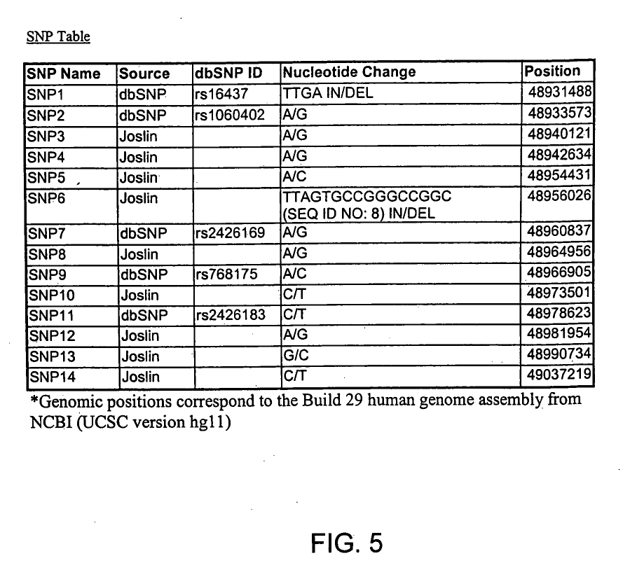

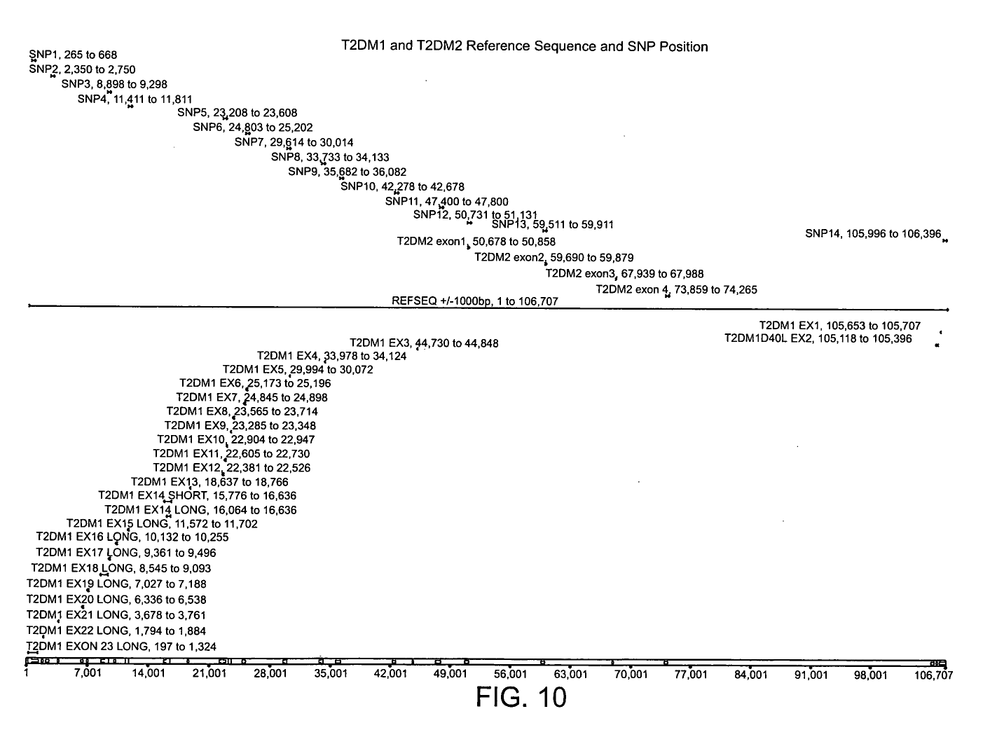

At least 16 chromosomes from 8 type 2 diabetes patients were evaluated and 14 polymorphisms, including 12 single nucleotide polymorphisms (SNPs) were identified that are associated with susceptibility to type 2 diabetes. See FIGS. 4 and 10. The number of chromosomes analyzed was sufficient to pick up most common polymorphisms. Diagnostic and prognostic methods, e.g., diagnostic and prognostic methods described herein can include evaluating one or more T2DM-1 or -2 polymorphisms.

Methods described herein provide for determining whether a subject carries a polymorphism of the T2DM-1 or T2DM-2 gene. For example, methods are provided for determining which allele or alleles of the human T2DM-1 or T2DM-2 gene a subject carries. Polymorphisms can be detected in a target nucleic acid from an individual. Samples that include T2DM-1 or T2DM-2 or the T2DM-1 or T2DM-2 gene can be utilized, e.g., blood samples. Genomic DNA, cDNA, mRNA, and/or proteins can be used to determine which of a plurality of polymorphisms are present in a subject.

Amplification of DNA from target samples can be accomplished by methods known to those of skill in the art, e.g., polymerase chain reaction (PCR). See, e.g., U.S. Pat. No. 4,683,202 (which is incorporated herein by reference in its entirety), ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA).

The methods with which a polymorphism is detected can depend on whether it is known that the polymorphism exists. If it is unknown whether a polymorphism exists, de novo characterization can be employed. This analysis compares target sequences in different individuals to identify points of variation, i.e., polymorphic sites. Analyzing groups of individuals that exhibit high degrees of diversity, e.g., ethnic diversity (in humans), or breed and species variety (in other organisms, e.g., non-human animals and plants), allows the identification of patterns characteristic of the most common alleles of the locus. Further, the frequencies of such populations within the population can be determined. Allelic frequencies can be determined for subpopulations characterized by other criteria, e.g., gender.

When it is known that a polymorphism exists, there are a variety of suitable procedures that can be employed to detect the polymorphism, described in further detail below.

Allele-Specific Probes

The design and use of allele-specific probes for analyzing polymorphisms is known in the art (see, e.g., Dattagupta, EP 235,726, Saiki, WO 89/11548). Allele-specific probes can be designed to hybridize differentially, e.g., to hybridize to a segment of DNA from one individual but not to a corresponding segment from another individual, based on the presence of polymorphic forms of the segment. Relatively stringent hybridization conditions can be utilized to cause a significant difference in hybridization intensity between alleles, and possibly to obtain a condition wherein a probe hybridizes to only one of the alleles. Probes can be designed to hybridize to a segment of DNA such that the polymorphic site aligns with a central position of the probe.

Allele-specific probes can be used in pairs, wherein one member of the pair matches perfectly to a reference form of a target sequence, and the other member of the pair matches perfectly to a variant of the target sequence. The use of several pairs of probes immobilized on the same support may allow simultaneous analysis of multiple polymorphisms within the same target sequence.

Tiling Arrays

Polymorphisms can also be identified by hybridization to nucleic acid arrays (see, e.g., WO 95/11995). WO 95/11995 also describes subarrays that are optimized for detection of variant forms of a precharacterized polymorphism. Such a subarray contains probes designed to be complementary to a second reference sequence, which is an allelic variant of the first reference sequence. The second group of probes is designed to exhibit complementarily to the second reference sequence. The inclusion of a second group (or further groups) can be particularly useful for analyzing short subsequences of the primary reference sequence in which multiple mutations are expected to occur within a short distance commensurate with the length of the probes (i.e., two or more mutations within 9 to 21 bases).

Allele-Specific Primers

An allele-specific primer hybridizes to a site on target DNA overlapping a polymorphism and only primes amplification of an allelic form to which the primer exhibits perfect complementarily. See, e.g., Gibbs, Nucleic Acid Res. 17, 2427-2448 (1989). Such a primer can be used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method can be optimized by including the mismatch in the 3′-most position of the oligonucleotide aligned with the polymorphism because this position is most destabilizing to elongation from the primer. See, e.g., WO 93/22456.

Direct-Sequencing

The direct analysis of the sequence of polymorphisms of the present invention can be accomplished using either the dideoxy chain termination method or the Maxam Gilbert method (see Sambrook et al. Molecular Cloning: A Laboratory Manual, 3d ed., 2001, Cold Spring Harbor, which is hereby incorporated in its entirety; Zyskind et al., Recombinant DNA Laboratory Manual, (Acad. Press, 1988)).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

### Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Other methods of detecting polymorphisms, e.g., SNPs, are known, e.g., as described in U.S. Pat. No. 6,410,231; U.S. Pat. No. 6,361,947; U.S. Pat. No. 6,322,980; U.S. Pat. No. 6,316,196; U.S. Pat. No. 6,258,539.

Detection of Variations or Mutations

Alterations or mutations in a T2DM-1 or T2DM-2 gene can be identified by a number of methods known in the art, to thereby identify other polymorphisms that may be associated with susceptibility for type 2 diabetes mellitus. In preferred embodiments, the methods include detecting, in a sample from the subject, the presence or absence of a genetic alteration characterized by an alteration affecting the integrity of a gene encoding a T2DM-1 or T2DM-2 protein, or the mis-expression of the T2DM-1 or T2DM-2 gene. For example, such genetic alterations can be detected by ascertaining the existence of at least one of 1) a deletion of one or more nucleotides from a T2DM-1 or T2DM-2 gene; 2) an addition of one or more nucleotides to a T2DM-1 or T2DM-2 gene; 3) a substitution of one or more nucleotides of a T2DM-1 or T2DM-2 gene, 4) a chromosomal rearrangement of a T2DM-1 or T2DM-2 gene; 5) an alteration in the level of a messenger RNA transcript of a T2DM-1 or T2DM-2 gene, 6) aberrant modification of a T2DM-1 or T2DM-2 gene, such as of the methylation pattern of the genomic DNA, 7) the presence of a non-wild type splicing pattern of a messenger RNA transcript of a T2DM-1 or T2DM-2 gene, 8) a non-wild type level of a T2DM-1 or T2DM-2-protein, 9) allelic loss of a T2DM-1 or T2DM-2 gene, and 10) inappropriate post-translational modification of a T2DM-1 or T2DM-2-protein.

An alteration can be detected with or without a probe/primer in a polymerase chain reaction, e.g., by anchor PCR or RACE PCR, or, alternatively, in a ligation chain reaction (LCR), the latter of which can be particularly useful for detecting point mutations in the T2DM-1 or T2DM-2-gene. This method can include the steps of collecting a sample of cells from a subject, isolating nucleic acid (e.g., genomic, mRNA or both) from the sample, contacting the nucleic acid sample with one or more primers which specifically hybridize to a T2DM gene under conditions such that hybridization and amplification of the T2DM-1 or T2DM-2-gene (if present) occurs, and detecting the presence or absence of an amplification product, or detecting the size of the amplification product and comparing the length to a control sample. PCR and/or LCR can be used as a preliminary amplification step in conjunction with any of the techniques used for detecting mutations described herein. Alternatively, other amplification methods described herein or known in the art can be used.

In another embodiment, mutations in a T2DM-1 or T2DM-2 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in T2DM-1 or T2DM-2 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a T2DM-1 or T2DM-2 nucleic acid or a putative variant (e.g., allelic variant) thereof. A probe can have one or more mismatches to a region of a T2DM-1 or T2DM-2 nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in T2DM-1 or T2DM-2 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the T2DM-1 or T2DM-2 gene and detect mutations by comparing the sequence of the sample T2DM-1 or T2DM-2 with the corresponding wild-type (control) sequence. Automated sequencing procedures can be utilized when performing the diagnostic assays ((1995) Biotechniques 19:448), including sequencing by mass spectrometry.

Other methods for detecting mutations in the T2DM-1 or T2DM-2 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in T2DM-1 or T2DM-2 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in T2DM-1 or T2DM-2 genes. For example, single strand conformation polymorphism (SSCP) may be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control T2DM-1 or T2DM-2 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci. USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

### Isolated Nucleic Acid Molecules

In one aspect, the invention provides, an isolated or purified, nucleic acid molecule that encodes a T2DM polypeptide described herein, e.g., a full-length T2DM-1 or T2DM-2 protein or a fragment thereof, e.g., a biologically active portion of T2DM-1 or T2DM-2 protein. Also included is a nucleic acid fragment suitable for use, e.g., as a primer (e.g., for the amplification or mutation of nucleic acid molecules) or hybridization probe; or as an antisense reagent, e.g., a ssRNA, dsRNA, siRNA, dsDNA, or mRNA-cDNA hybrid fragments.

In one embodiment, an isolated nucleic acid molecule of the invention includes the nucleotide sequence shown in SEQ ID NO:1, 3, 5, 6 or a portion of any of these nucleotide sequences. In one embodiment, the nucleic acid molecule includes sequences encoding the human T2DM protein (i.e., “the coding region” of SEQ ID NO:1, 3, 5 or 6, and alternatively spliced variants thereof), as well as 5′ untranslated sequences. Alternatively, the nucleic acid molecule can include only the coding region of SEQ ID NO:1, 3, 5 or 6 and, e.g., no flanking sequences which normally accompany the subject sequence.

In another embodiment, an isolated nucleic acid molecule of the invention includes a nucleic acid molecule which is a complement of the nucleotide sequence shown in SEQ ID NO:1, 3, 5, or 6 or a portion of any of these nucleotide sequences. In other embodiments, the nucleic acid molecule of the invention is sufficiently complementary to the nucleotide sequence shown in SEQ ID NO:1, 3, 5, or 6, such that it can hybridize (e.g., under a stringency condition described herein) to the nucleotide sequence shown in SEQ ID NO:1, 3, 5, or 6, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more identical to the entire length of the nucleotide sequence shown in SEQ ID NO:1, 3, 5, or 6 or a portion, preferably of the same length, of any of these nucleotide sequences.

In another embodiment, an isolated nucleic acid molecule of the present invention includes a nucleotide sequence which encodes a polypeptide comprising the sequence of SEQ ID NO: 2 or 4, but having up to 50, preferably up to 40, 30, 25, 20, 15 or up to 10 amino acid additions, deletion and/or substitutions.

Nucleic Acid Fragments

A nucleic acid molecule of the invention can include only a portion of the nucleic acid sequences of SEQ ID NO:1, 3, 5, or 6. For example, such a nucleic acid molecule can include a fragment which can be used as a probe or primer or a fragment encoding a portion of a T2DM protein, e.g., an immunogenic or biologically active portion of a T2DM protein. For example, a fragment can comprise those nucleotides of SEQ ID NO:1, 3, 5, or 6 which encode a leucine rich or serine rich domain of human T2DM. The nucleotide sequence determined from the cloning of the T2DM gene allows for the generation of probes and primers designed for use in identifying and/or cloning other T2DM family members, or fragments thereof, as well as T2DM homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 20, e.g., 50 amino acids in length. Fragments also include nucleic acid sequences corresponding to specific amino acid sequences described above or fragments thereof. Nucleic acid fragments should not be construed as encompassing those fragments that may have been disclosed prior to the invention.

A nucleic acid fragment can include a sequence corresponding to a domain, region, or functional site described herein. A nucleic acid fragment can also include one or more domains, regions, or functional sites described herein. Thus, for example, a T2DM nucleic acid fragment can include a sequence corresponding to a sequence encoding a leucine rich domain or a serine rich domain.

T2DM probes and primers are provided. Typically a probe/primer is an isolated or purified oligonucleotide. The oligonucleotide typically includes a region of nucleotide sequence that hybridizes under a stringency condition described herein to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, SEQ ID NO:3, SEQ ID NO:5, or SEQ ID NO:6, or of a naturally occurring allelic variant or mutant of SEQ ID NO:1, SEQ ID NO:3, SEQ ID NO:5, or SEQ ID NO:6, e.g., a sequence comprising a T2DM polymorphic sequence described herein. Preferably, an oligonucleotide is less than about 200, 150, 120, or 100 nucleotides in length.

In one embodiment, the probe or primer is attached to a solid support, e.g., a solid support described herein.

One exemplary kit of primers includes a forward primer that anneals to the coding strand and a reverse primer that anneals to the non-coding strand. The forward primer can anneal to the start codon, e.g., the nucleic acid sequence encoding amino acid residue 1 of SEQ ID NO:2. The reverse primer can anneal to the ultimate codon, e.g., the codon immediately before the stop codon. In a preferred embodiment, the annealing temperatures of the forward and reverse primers differ by no more than 5, 4, 3, or 2° C.

In a preferred embodiment the nucleic acid is a probe which is at least 10, 12, 15, 18, 20 and less than 200, more preferably less than 100, or less than 50, nucleotides in length. It should be identical, or differ by 1, or 2, or less than 5 or 10 nucleotides, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes T2DM-1 or T2DM-2.

In another embodiment a set of primers is provided, e.g., primers suitable for use in a PCR, which can be used to amplify a selected region of a T2DM sequence, e.g., a domain, region, site or other sequence described herein, e.g., a SNP described herein. The primers should be at least 5, 10, or 50 base pairs in length and less than 100, or less than 200, base pairs in length. The primers should be identical, or differ by one base from a sequence disclosed herein or from a naturally occurring variant.

A nucleic acid fragment can encode an epitope bearing region of a polypeptide described herein.

A nucleic acid fragment encoding a “biologically active portion of a T2DM polypeptide” can be prepared by isolating a portion of the nucleotide sequence of SEQ ID NO:1, 3, 5, or 6 which encodes a polypeptide having a T2DM biological activity (e.g., the biological activities of the T2DM proteins are described herein, expressing the encoded portion of the T2DM protein (e.g., by recombinant expression in vitro) and assessing the activity of the encoded portion of the T2DM protein. For example, a nucleic acid fragment encoding a biologically active portion of T2DM includes a leucine rich domain, or a serine rich domain. A nucleic acid fragment encoding a biologically active portion of a T2DM polypeptide, may comprise a nucleotide sequence which is greater than 300 or more nucleotides in length.

In preferred embodiments, a nucleic acid includes a nucleotide sequence which is about 100, 200, 300, 400, 500, 600, 700, 800, 900, 1000, 1100, 1200, 1300 or more nucleotides in length and hybridizes under a stringency condition described herein to a nucleic acid molecule of SEQ ID NO:1, SEQ ID NO:3, SEQ ID NO:5, or SEQ ID NO:6.

T2DM Nucleic Acid Variants

The invention further encompasses nucleic acid molecules that differ from the nucleotide sequences shown in SEQ ID NO:1, 3, 5, or 6. Such differences can be due to degeneracy of the genetic code (and result in a nucleic acid which encodes the same T2DM proteins as those encoded by the nucleotide sequence disclosed herein). In another embodiment, an isolated nucleic acid molecule of the invention has a nucleotide sequence encoding a protein having an amino acid sequence which differs, by at least 1, but less than 5, 10, 20, 50, or 100 amino acid residues that are shown in SEQ ID NO:2 or 4. If alignment is needed for this comparison the sequences should be aligned for maximum homology. The encoded protein can differ by no more than 5, 4, 3, 2, or 1 amino acid. “Looped” out sequences from deletions or insertions, or mismatches, are considered differences.

Nucleic acids of the inventor can be chosen for having codons, which are preferred, or non-preferred, for a particular expression system. For example, the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 5 or 6 e.g., as follows: by at least one but less than 10, 20, 30, or 40 nucleotides; at least one but less than 1%, 5%, 10% or 20% of the nucleotides in the subject nucleic acid. The nucleic acid can differ by no more than 5, 4, 3, 2, or 1 nucleotide. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:1, 3, 5, or 6 or a fragment of this sequence. Such nucleic acid molecules can readily be identified as being able to hybridize under a stringency condition described herein, to the nucleotide sequence shown in SEQ ID NO:1, 3, 5, or 6 or a fragment of the sequence. Nucleic acid molecules corresponding to orthologs, homologs, and allelic variants of the T2DM cDNAs of the invention can further be isolated by mapping to the same chromosome or locus as the T2DM gene.

Specific hybridization conditions referred to herein are as follows: 1) low stringency: hybridization in 6× sodium chloride/sodium citrate (SSC) at about 45° C., followed by two washes in 0.2×SSC, 0.1% SDS at least at 50° C.; 2) medium stringency: hybridization in 6×SSC at about 45° C., followed by one or more washes in 0.2×SSC, 0.1% SDS at 60° C.; 3) high stringency: hybridization in 6×SSC at about 45° C., followed by one or more washes in 0.2×SSC, 0.1% SDS at 65° C.; and 4) very high stringency: hybridization in 0.5M sodium phosphate, 7% SDS at 65° C., followed by one or more washes at 0.2×SSC, 1% SDS at 65° C.

Preferred variants include those that are correlated with susceptibility to type 2 diabetes.

Allelic variants of T2DM, e.g., human T2DM, include both functional and non-functional proteins. Functional allelic variants are naturally occurring amino acid sequence variants of the T2DM protein within a population that maintain activity. Functional allelic variants will typically contain only conservative substitution of one or more amino acids of SEQ ID NO:2 or 4, or substitution, deletion or insertion of non-critical residues in non-critical regions of the protein. Non-functional allelic variants are naturally-occurring amino acid sequence variants of the T2DM, e.g., human T2DM-1 or T2DM-2, protein within a population that do not have a wildtype activity. Non-functional allelic variants will typically contain a non-conservative substitution, a deletion, or insertion, or premature truncation of the amino acid sequence of SEQ ID NO:2 or 4 or a substitution, insertion, or deletion in critical residues or critical regions of the protein.

Moreover, nucleic acid molecules encoding other T2DM family members and, thus, which have a nucleotide sequence which differs from the T2DM sequences of SEQ ID NO:1, 3, 5, or 6 are intended to be within the scope of the invention.

### Antisense Nucleic Acid Molecules, Ribozymes and Modified T2DM Nucleic Acid Molecules

In another aspect, the invention features, an isolated nucleic acid molecule which is antisense to T2DM. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire T2DM coding strand, or to only a portion thereof. In another embodiment, the antisense nucleic acid molecule is antisense to a “noncoding region” of the coding strand of a nucleotide sequence encoding T2DM (e.g., the 5′ and 3′ untranslated regions).

An antisense nucleic acid can be designed such that it is complementary to the entire coding region of T2DM mRNA, but more preferably is an oligonucleotide which is antisense to only a portion of the coding or noncoding region of T2DM mRNA. For example, the antisense oligonucleotide can be complementary to the region surrounding the translation start site of T2DM mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a T2DM protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual 13-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a T2DM-encoding nucleic acid can include one or more sequences complementary to the nucleotide sequence of a T2DM nucleic acid disclosed herein (i.e., SEQ ID NO:1, 3, 5, or 6), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a T2DM-encoding mRNA. See, e.g., Cech et al. U.S. Pat. No. 4,987,071; and Cech et al. U.S. Pat. No. 5,116,742. Alternatively, T2DM mRNA can be used to select a catalytic RNA having a specific ribonuclease activity from a pool of RNA molecules. See, e.g., Bartel, D. and Szostak, J. W. (1993) Science 261:1411-1418.

T2DM gene expression can be inhibited by targeting nucleotide sequences complementary to the regulatory region of the T2DM (e.g., the T2DM promoter and/or enhancers to form triple helical structures that prevent transcription of the T2DM gene in target cells. See generally, Helene, C. (1991)Anticancer Drug Des. 6:569-84; Helene, C. i (1992)Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A T2DM nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of T2DM nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of T2DM nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. W089/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a T2DM nucleic acid of the invention, two complementary regions one having a fluorophore and one a quencher such that the molecular beacon is useful for quantitating the presence of the T2DM nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

RNAi

Double stranded nucleic acid molecules that can silence a T2DM-1 or T2DM-2 gene can also be used as an agent which inhibits expression of T2DM-1 or T2DM-2. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature 2001 May 24; 411(6836):494-8). In one embodiment, gene silencing can be induced in mammalian cells by enforcing endogenous expression of RNA hairpins (see Paddison et al., 2002, PNAS USA 99:1443-1448). In another embodiment, transfection of small (21-23 nt) dsRNA specifically inhibits gene expression (reviewed in Caplen (2002) Trends in Biotechnology 20:49-51).

Briefly, RNAi is thought to work as follows. dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA ˜12 nucleotides from the 3′ terminus of the siRNA (reviewed in Sharp et al (2001) Genes Dev 15: 485-490; and Hammond et al. (2001) Nature Rev Gen 2: 110-119).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun 2001 Mar. 2; 281(3):639-44), providing yet another strategy for gene silencing.

Isolated T2DM-1 And T2DM-2 Polypeptides In another aspect, the invention features, an isolated T2DM protein, e.g., T2DM-1 or T2DM-2 or fragment, e.g., a biologically active portion, for use as immunogens or antigens to raise or test (or more generally to bind) anti-T2DM antibodies. T2DM protein can be isolated from cells or tissue sources using standard protein purification techniques. T2DM-1 or T2DM-2 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In a preferred embodiment, a T2DM polypeptide has one or more of the following characteristics:

(i) it affects susceptibility to type 2 diabetes;

(ii) it modulates insulin function;

(iii) it modulates pancreatic β-cell function, development and/or differentiation;

(iv) it is recognized by an anti-T2DM antibody described herein;

(v) it has a molecular weight, e.g., a deduced molecular weight, preferably ignoring any contribution of post translational modifications, amino acid composition or other physical characteristic of SEQ ID NO:2 or 4;

(vi) it has an overall sequence similarity of at least 50%, preferably at least 60%, more preferably at least 70, 80, 90, or 95%, with a polypeptide a of SEQ ID NO: 2 or 4 or an alternatively spliced variant thereof;

(vii) it can be found in liver, islet cells, kidney, muscle, brain, testis, or adipose tissue.

In a preferred embodiment the T2DM, e.g., T2DM-1 or T2DM-2 protein, or fragment thereof, differs from the corresponding sequence in SEQ ID: 2 or 4. In one embodiment it differs by at least one but by less than 50, 40, 30, 25, 20, 15, 10 or 5 amino acid residues (e.g., has at least one but by less than 50, 40, 30, 25, 20, 15, 10 or 5 amino acid substitutions (e.g., conservative amino acid substitutions), deletions or additions. In another it differs from the corresponding sequence in SEQ ID NO:2 or 4 by at least one residue but less than 20%, 15%, 10% or 5% of the residues in it differ from the corresponding sequence in SEQ ID NO: 2 or 4. (If this comparison requires alignment, the sequences should be aligned for maximum homology. “Looped” out sequences from deletions or insertions, or mismatches, are considered differences.) The differences are, preferably, differences or changes at a non essential residue or a conservative substitution.

Other embodiments include a protein that contain one or more changes in amino acid sequence, e.g., a change in an amino acid residue which is not essential for activity. Such T2DM proteins differ in amino acid sequence from SEQ ID NO: 2 or 4, yet retain biological activity. In another embodiment, the protein contains one or more changes in amino acid sequence, e.g., a change in an amino acid residue which is essential for activity, e.g., it is encoded by a polymorphic T2DM-1 or T2DM-2 sequence that alters the sequence of one or more amino acids of the protein.

In one embodiment, the protein includes an amino acid sequence at least about 50%, 55%, 60%, 65%, 70%, 75%, 80%, 85%, 90%, 95%, 98% or more homologous to SEQ ID NO:2 or 4 or an alternatively spliced variant thereof described herein.

In one embodiment, a biologically active portion of a T2DM protein includes a serine rich or leucine rich domain or other T2DM domain or motif described herein. Moreover, other biologically active portions, in which other regions of the protein are deleted, can be prepared by recombinant techniques and evaluated for one or more of the functional activities of a native T2DM protein.

In a preferred embodiment, the T2DM-1 or -2 protein has an amino acid sequence shown in SEQ ID NO: 2 or 4. In other embodiments, the T2DM protein is substantially identical to SEQ ID NO: 2 or 4. In yet another embodiment, the T2DM protein is substantially identical to SEQ ID NO:2 or 4 and retains the functional activity of the protein of SEQ ID NO: 2 or 4, as described in detail in the subsections above.

### T2DM-1 And T2DM-2 Chimeric Or Fusion Proteins

In another aspect, the invention provides T2DM, e.g., T2DM-1 or T2DM-2 chimeric or fusion proteins. As used herein, a T2DM “chimeric protein” or “fusion protein” includes a T2DM polypeptide, or functional fragment thereof, linked to a non-T2DM polypeptide. A “non-T2DM polypeptide” refers to a polypeptide having an amino acid sequence corresponding to a protein which is not substantially homologous to the T2DM protein, e.g., a protein which is different from the T2DM protein and which is derived from the same or a different organism. The T2DM polypeptide of the fusion protein can correspond to all or a portion, e.g., a fragment described herein of a T2DM amino acid sequence. In a preferred embodiment, a T2DM fusion protein includes at least one (or two) biologically active portion of a T2DM protein. The non-T2DM polypeptide can be fused to the N-terminus or C-terminus of the T2DM polypeptide.

The fusion protein can include a moiety which has a high affinity for a ligand. For example, the fusion protein can be a GST-T2DM fusion protein in which the T2DM sequences are fused to the C-terminus of the GST sequences. Such fusion proteins can facilitate the purification of recombinant T2DM. Alternatively, the fusion protein can be a T2DM protein containing a heterologous signal sequence at its N-terminus. In certain host cells (e.g., mammalian host cells), expression and/or secretion of T2DM can be increased through use of a heterologous signal sequence.

Fusion proteins can include all or a part of a serum protein, e.g., an IgG constant region, or human serum albumin.

The T2DM fusion proteins of the invention can be incorporated into pharmaceutical compositions and administered to a subject in vivo. The T2DM fusion proteins can be used to affect the bioavailability of a T2DM substrate. T2DM fusion proteins may be useful therapeutically for the treatment of type 2 diabetes.

Moreover, the T2DM-1 or T2DM-2 fusion proteins of the invention can be used as immunogens to produce anti-T2DM antibodies in a subject, to purify T2DM ligands and in screening assays to identify molecules which inhibit the interaction of T2DM with a T2DM substrate.

Expression vectors are commercially available that already encode a fusion moiety (e.g., a GST polypeptide). A T2DM-encoding nucleic acid can be cloned into such an expression vector such that the fusion moiety is linked in-frame to the T2DM protein.

### Variants of T2DM-1 and T2DM-2 Proteins

In another aspect, the invention also features a variant of a T2DM-1 or T2DM-2 polypeptide, e.g., a T2DM-1 or T2DM-2 polypeptide which functions as an agonist (mimetic) or as an antagonist. Variants of the T2DM-1 or T2DM-2 proteins can be generated by mutagenesis, e.g., discrete point mutation, the insertion or deletion of sequences or the truncation of a T2DM-1 or T2DM-2 protein. An agonist of the T2DM-1 or T2DM-2 proteins can retain substantially the same, or a subset, of the biological activities of the naturally occurring form of a T2DM-1 or T2DM-2 protein. An antagonist of a T2DM-1 or T2DM-2 protein can inhibit one or more of the activities of the naturally occurring form of the T2DM-1 or T2DM-2 protein by, for example, competitively modulating a T2DM-1 or T2DM-2-mediated activity of a T2DM-1 or T2DM-2 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the T2DM-1 or T2DM-2 protein.

Variants of a T2DM-1 or T2DM-2 protein can be identified by screening combinatorial libraries of mutants, e.g., truncation mutants, of a T2DM-1 or T2DM-2 protein for agonist or antagonist activity.

Libraries of fragments, e.g., N terminal, C terminal, or internal fragments, of a T2DM-1 or T2DM-2 protein coding sequence can be used to generate a variegated population of fragments for screening and subsequent selection of variants of a T2DM-1 or T2DM-2 protein. Variants in which cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of T2DM-1 or T2DM-2 proteins. Recursive ensemble mutagenesis (REM), a technique which enhances the frequency of functional mutants in the libraries, can be used in combination with the screening assays to identify T2DM-1 or T2DM-2 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated T2DM-1 or T2DM-2 library. For example, a library of expression vectors can be transfected into a cell line, e.g., a cell line, which ordinarily responds to T2DM-1 or T2DM-2 in a substrate-dependent manner. The transfected cells are then contacted with T2DM-1 or T2DM-2 and the effect of the expression of the mutant on signaling by the T2DM-1 or T2DM-2 substrate can be detected, e.g., by assaying insulin function or signaling. Plasmid DNA can then be recovered from the cells which score for inhibition, or alternatively, potentiation of signaling by the T2DM-1 or T2DM-2 substrate, and the individual clones further characterized.

In another aspect, the invention features a method of making a T2DM-1 or T2DM-2 polypeptide, e.g., a peptide having a non-wild type activity, e.g., an antagonist, agonist, or super agonist of a naturally occurring T2DM-1 or T2DM-2 polypeptide, e.g., a naturally occurring T2DM-1 or T2DM-2 polypeptide. The method includes: altering the sequence of a T2DM-1 or T2DM-2 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a T2DM-1 or T2DM-2 polypeptide, e.g., having a biological activity of a naturally occurring T2DM-1 or T2DM-2 polypeptide. The method includes: altering the sequence, e.g., by substitution or deletion of one or more residues, of a T2DM polypeptide, e.g., altering the sequence of a non-conserved region, or a domain or residue described herein, and testing the altered polypeptide for the desired activity.

### Anti-T2DM Antibodies

In another aspect, the invention provides an anti-T2DM-1 or anti-T2DM-2, e.g., anti-T2DM-1a, -1b, -2a or -2b, antibody, or a fragment thereof (e.g., an antigen-binding fragment thereof). The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The anti-T2DM-1 or T2DM-2 antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to the antigen, e.g., T2DM polypeptide or fragment thereof. Examples of antigen-binding fragments of the anti-T2DM antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

The anti-T2DM-1 or T2DM-2 antibody can be a polyclonal or a monoclonal antibody. In other embodiments, the antibody can be recombinantly produced, e.g., produced by phage display or by combinatorial methods.

Phage display and combinatorial methods for generating anti-T2DM antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-T2DM antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-T2DM antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a T2DM or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a T2DM-1 or -2 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

A full-length T2DM-1 or T2DM-2 protein or, antigenic peptide fragment of T2DM-1 or T2DM-2 can be used as an immunogen or can be used to identify anti-T2DM-1 or T2DM-2 antibodies made with other immunogens, e.g., cells, membrane preparations, and the like. The antigenic peptide of T2DM-1 or T2DM-2 should include at least 8 amino acid residues of the amino acid sequence shown in SEQ ID NO: 2 or 4 and encompasses an epitope of T2DM-1 or T2DM-2. Preferably, the antigenic peptide includes at least 10 amino acid residues, more preferably at least 15 amino acid residues, even more preferably at least 20 amino acid residues, and most preferably at least 30 amino acid residues.

Antibodies reactive with, or specific for, any of these regions, or other regions or domains described herein are provided.

Antibodies which bind only native T2DM-1 or T2DM-2 protein, only denatured or otherwise non-native T2DM-1 or T2DM-2 protein, or which bind both, are within the invention. Antibodies with linear or conformational epitopes are within the invention. Conformational epitopes can sometimes be identified by identifying antibodies which bind to native but not denatured T2DM-1 or T2DM-2 protein.

Preferred epitopes encompassed by the antigenic peptide are regions of T2DM-1 or T2DM-2 are located on the surface of the protein, e.g., hydrophilic regions, as well as regions with high antigenicity. For example, an Emini surface probability analysis of the human T2DM-1 or T2DM-2 protein sequence can be used to indicate the regions that have a particularly high probability of being localized to the surface of the T2DM-1 or T2DM-2 protein and are thus likely to constitute surface residues useful for targeting antibody production.

The anti-T2DM-1 or T2DM-2 antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target T2DM-1 or T2DM-2 protein.

In a preferred embodiment the antibody has effector function and/or can fix complement. In other embodiments the antibody does not recruit effector cells; or fix complement.

In a preferred embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

In a preferred embodiment, an anti-T2DM-1 or T2DM-2 antibody alters (e.g., increases or decreases) a T2DM-1 or T2DM-2 activity described herein.

The antibody can be coupled to a toxin, e.g., a polypeptide toxin, e.g. ricin or diphtheria toxin or active fragment hereof, or a radioactive nucleus, or imaging agent, e.g. a radioactive, enzymatic, or other, e.g., imaging agent, e.g., a NMR contrast agent. Labels which produce detectable radioactive emissions or fluorescence are preferred.

An anti-T2DM-1 or T2DM-2 antibody (e.g., monoclonal antibody) can be used to isolate T2DM-1 or T2DM-2 by standard techniques, such as affinity chromatography or immunoprecipitation. Moreover, an anti-T2DM-1 or T2DM-2 antibody can be used to detect T2DM-1 or T2DM-2 protein (e.g., in a cellular lysate or cell supernatant) in order to evaluate the abundance and pattern of expression of the protein. Anti-T2DM-1 or T2DM-2 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-T2DM-1 or T2DM-2 antibody, e.g., an anti-T2DM-1 or T2DM-2 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-T2DM-1 or T2DM-2 antibody, e.g., and antibody described herein, and method of using said cells to make a T2DM-1 or T2DM-2 antibody.

### Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a T2DM-1 or T2DM-2 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., T2DM-1 or T2DM-2 proteins, mutant forms of T2DM-1 or T2DM-2 proteins, fusion proteins, and the like).

The recombinant expression vectors of the invention can be designed for expression of T2DM-1 or T2DM-2 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in T2DM-1 or T2DM-2 activity assays, (e.g., direct assays or competitive assays described in detail below), or to generate antibodies specific for T2DM-1 or T2DM-2 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The T2DM-1 or T2DM-2 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a T2DM-1 or T2DM-2 nucleic acid molecule within a recombinant expression vector or a T2DM-1 or T2DM-2 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a T2DM-1 or T2DM-2 protein can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman (1981) Cell I 23:175-182)). Other suitable host cells are known to those skilled in the art.

Host cells for methods of producing insulin as described herein can include glucose responsive and non-glucose responsive cells. Embryonic stem cells, pancreatic precursor cells, primary beta-cells or cell lines derived from islet beta-cells or insulinomas are examples of cells that can be used for glucose responsive production of insulin. Expression of glucokinase and glucose transporter activity (e.g., GLUT-2) in these cells can aid in glucose sensing. In addition, cells that normally lack glucose-stimulated peptide release may be engineered for this function. The use of these genes as a general tool for engineering of glucose sensing has been described in, e.g., Newgard, U.S. Pat. No. 5,427,940. Neuroendocrine cells that can be engineered to be glucose sensitive include AtT-20 cells, which are derived from ACTH secreting cells of the anterior pituitary; PC12, a neuronal cell line (ATCC CRL 1721); and GH3, an anterior pituitary cell line that secretes growth hormone (ATCC CCL82.1).

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a T2DM-1 or T2DM-2 protein. Accordingly, the invention further provides methods for producing a T2DM-1 or T2DM-2 protein using the host cells of the invention. In one embodiment, the method includes culturing the host cell of the invention (into which a recombinant expression vector encoding a T2DM-1 or T2DM-2 protein has been introduced) in a suitable medium such that a T2DM-1 or T2DM-2 protein is produced. In another embodiment, the method further includes isolating a T2DM-1 or T2DM-2 protein from the medium or the host cell.

In another aspect, the invention features, a cell or purified preparation of cells which include a T2DM-1 or T2DM-2 transgene, or which otherwise misexpress T2DM-1 or T2DM-2. The cell preparation can consist of human or non-human cells, e.g., rodent cells, e.g., mouse or rat cells, rabbit cells, or pig cells. In preferred embodiments, the cell or cells include a T2DM-1 or T2DM-2 transgene, e.g., a heterologous form of a T2DM-1 or T2DM-2, e.g., a gene derived from humans (in the case of a non-human cell). The T2DM-1 or T2DM-2 transgene can be misexpressed, e.g., overexpressed or underexpressed. In other preferred embodiments, the cell or cells include a gene that mis-expresses an endogenous T2DM-1 or T2DM-2, e.g., a gene the expression of which is disrupted, e.g., a knockout. Such cells can serve as a model for studying disorders that are related to mutated or mis-expressed T2DM-1 or T2DM-2 alleles or for use in drug screening.

In another aspect, the invention features, a human cell (e.g., a pancreatic islet cell, β-cell, β-cell precursor cell, kidney cell, liver cell, brain cell, testis cell, muscle cell, adult or embryonic stem cell, a human neuroendocrine cell, pancreatic ductal cell or cell line, pancreatic acinar cell or cell line, pancreatic endocrine cell or cell line, enteroendocrine cell or cell line, hepatic cell, fibroblast, endothelial cell, or muscle cell) transformed with nucleic acid which encodes a subject T2DM-1 or T2DM-2 polypeptide.

Also provided are cells, preferably human cells, e.g., stem cells, pancreatic cells, e.g., pancreatic islet cell, β-cell, β-cell precursor cells, kidney cells, liver cells, brain cells, testis cells, muscle cells, in which an endogenous T2DM-1 or T2DM-2 is under the control of a regulatory sequence that does not normally control the expression of the endogenous T2DM-1 or T2DM-2 gene. The expression characteristics of an endogenous gene within a cell, e.g., a cell line or microorganism, can be modified by inserting a heterologous DNA regulatory element into the genome of the cell such that the inserted regulatory element is operably linked to the endogenous T2DM-1 or T2DM-2 gene. For example, an endogenous T2DM-1 or T2DM-2 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a T2DM-1 or T2DM-2 polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of a T2DM-1 or T2DM-2 polypeptide can be regulated in the subject by administering an agent (e.g., a steroid hormone) to the subject. In another preferred embodiment, the implanted recombinant cells express and secrete an antibody specific for a T2DM-1 or T2DM-2 polypeptide. The antibody can be any antibody or any antibody derivative described herein.

### Transgenic Animals

The invention provides non-human transgenic animals. Such animals are useful for studying the function and/or activity of a T2DM-1 or T2DM-2 protein and for identifying and/or evaluating modulators of T2DM-1 or T2DM-2 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous T2DM-1 or T2DM-2 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a T2DM-1 or T2DM-2 protein to particular cells. A transgenic founder animal can be identified based upon the presence of a T2DM-1 or T2DM-2 transgene in its genome and/or expression of T2DM-1 or T2DM-2 mRNA in tissues or cells of the animals. A transgenic founder animal can then be used to breed additional animals carrying the transgene. Moreover, transgenic animals carrying a transgene encoding a T2DM-1 or T2DM-2 protein can further be bred to other transgenic animals carrying other transgenes.

T2DM-1 or T2DM-2 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep. Animal models of diabetes include the NOD Mouse and its related strains, BB Rat, Leptin or Leptin Receptor mutant rodents, Zucker Diabetic Fatty (ZDF) Rat, Sprague-Dawley rats, Obese Spontaneously Hypertensive Rat (SHROB, Koletsky Rat), Wistar Fatty Rat, New Zealand Obese Mouse, NSY Mouse, Goto-Kakizaki Rat, OLETF Rat, JCR:LA-cp Rat, Neonatally Streptozotocin-Induced (n-STZ) Diabetic Rats, Rhesus Monkey, Psammomys obesus (fat sand rat), C57Bl/6J. Mouse.

The invention also includes a population of cells from a transgenic animal, as discussed, e.g., below.

### Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); c) methods of treatment (e.g., therapeutic and prophylactic); and d) biomaterials.

The isolated nucleic acid molecules of the invention can be used, for example, to express a T2DM-1 or T2DM-2 protein (e.g., via a recombinant expression vector in a host cell in gene therapy applications), to detect a T2DM-1 or T2DM-2 mRNA (e.g., in a biological sample) or a genetic alteration in a T2DM-1 or T2DM-2 gene, and to modulate T2DM-1 or T2DM-2 activity, as described further below. The T2DM-1 or T2DM-2 proteins can be used to treat type 2 diabetes mellitus. In addition, the T2DM-1 or T2DM-2 proteins can be used to screen for naturally occurring T2DM-1 or T2DM-2 substrates, to screen for drugs or compounds which modulate T2DM-1 or T2DM-2 activity, as well as to treat disorders characterized by insufficient or excessive production of T2DM-1 or T2DM-2 protein or production of T2DM-1 or T2DM-2 protein forms which have decreased, aberrant or unwanted activity compared to T2DM-1 or T2DM-2 wild type protein. Moreover, the anti-T2DM-1 or T2DM-2 antibodies of the invention can be used to detect and isolate T2DM-1 or T2DM-2 proteins, regulate the bioavailability of T2DM-1 or T2DM-2 proteins, and modulate T2DM-1 or T2DM-2 activity.

A method of evaluating a compound for the ability to interact with, e.g., bind, a subject T2DM-1 or T2DM-2 polypeptide is provided. The method includes: contacting the compound with the subject T2DM-1 or T2DM-2 polypeptide; and evaluating ability of the compound to interact with, e.g., to bind or form a complex with the subject T2DM-1 or T2DM-2 polypeptide. This method can be performed in vitro, e.g., in a cell free system, or in vivo, e.g., in a two-hybrid interaction trap assay. This method can be used to identify naturally occurring molecules that interact with subject T2DM-1 or T2DM-2 polypeptide. It can also be used to find natural or synthetic inhibitors of subject T2DM-1 or T2DM-2 polypeptide. Screening methods are discussed in more detail below.

### Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to T2DM-1 or T2DM-2 proteins, have a stimulatory or inhibitory effect on, for example, T2DM-1 or T2DM-2 expression or T2DM-1 or T2DM-2 activity, or have a stimulatory or inhibitory effect on, for example, the expression or activity of a T2DM-1 or T2DM-2 substrate. Compounds thus identified can be used to modulate the activity of target gene products (e.g., T2DM-1 or T2DM-2 genes) in a therapeutic protocol, to elaborate the biological function of the target gene product, or to identify compounds that disrupt normal target gene interactions.

Such screening assays can include: providing a T2DM-1 or T2DM-2 protein or nucleic acid, e.g., T2DM-1a, T2DM-1b, T2DM-2a, or T2DM-2b protein or nucleic acid or a functional fragment thereof; contacting the protein or nucleic acid with a test compound, and determining if the test compound modulates the T2DM protein or nucleic acid. A test compound may modulate a T2DM-1 or T2DM-2 activity by, e.g., binding to the T2DM protein and facilitating or inhibiting its biological activity. The compound can be, e.g., an antibody, e.g., an inhibitory T2DM-1 or T2DM-2 antibody or an antibody that stabilizes or assists a T2DM-1 or T2DM-2 activity. A test compound may also modulate a T2DM-1 or T2DM-2 activity by binding to a T2DM nucleic acid or fragment thereof. For example, the test compound may bind to the T2DM-1 or T2DM-2 promoter region and increase T2DM-1 or T2DM-2 transcription; the test compound may bind to a T2DM-1 or T2DM-2 nucleic acid and inhibit transcription of the gene; or the test compound may bind to a T2DM-1 or T2DM-2 nucleic acid and inhibit translation of the T2DM-1 or T2DM-2 mRNA. In a preferred embodiment, the compound is a small molecule that binds to the T2DM-1 or T2DM-2 promoter region to modulate transcription.

A test compound may also compete with the endogenous T2DM-1 or T2DM-2 protein for binding to a T2DM-1 or T2DM-2 binding partner. The test agent can be, e.g., a protein or peptide, an antibody, a small molecule, a nucleotide sequence. For example, the agent can be an agent identified through a library screen described herein.

The screening assays described herein can be performed in vitro or in vivo. If performed in vitro, the assay can further include administering the test compound to an experimental animal, e.g., an animal model of diabetes, e.g., a model described herein.

The test compounds of the screening assays described herein can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a T2DM-1 or T2DM-2 protein or biologically active portion thereof is contacted with a test compound, and the ability of the test compound to modulate T2DM-1 or T2DM-2 activity is determined. Determining the ability of the test compound to modulate T2DM-1 or T2DM-2 activity can be accomplished by monitoring, for example, binding to an endogenous binding partner, a nucleic acid, protein. The cell, for example, can be of mammalian origin, e.g., human.

The ability of the test compound to modulate T2DM-1 or T2DM-2 binding to a compound, e.g., a T2DM-1 or T2DM-2 substrate, or to bind to T2DM-1 or T2DM-2 can also be evaluated. This can be accomplished, for example, by coupling the compound, e.g., the substrate, with a radioisotope or enzymatic label such that binding of the compound, e.g., the substrate, to T2DM-1 or T2DM-2 can be determined by detecting the labeled compound, e.g., substrate, in a complex. Alternatively, T2DM-1 or T2DM-2 could be coupled with a radioisotope or enzymatic label to monitor the ability of a test compound to modulate T2DM-1 or T2DM-2 binding to a T2DM-1 or T2DM-2 substrate in a complex. For example, compounds (e.g., T2DM substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with T2DM-1 or T2DM-2 with or without the labeling of any of the interactants can be evaluated. For example, a microphysiometer can be used to detect the interaction of a compound with T2DM-1 or T2DM-2 without the labeling of either the compound or the T2DM. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and T2DM-1 or T2DM-2.

In yet another embodiment, a cell-free assay is provided in which a T2DM-1 or T2DM-2 protein or biologically active portion thereof is contacted with a test compound and the ability of the test compound to bind to the T2DM-1 or T2DM-2 protein or biologically active portion thereof is evaluated. Preferred biologically active portions of the T2DM proteins to be used in assays of the present invention include fragments which participate in interactions with non-T2DM molecules, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the T2DM-1 or T2DM-2 protein to bind to a target molecule can be accomplished using real-time Biomolecular Interaction Analysis (BIA) (see, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either T2DM-1 or T2DM-2, an anti-T2DM-1 or T2DM-2 antibody or its target molecule to facilitate separation of complexed from uncomplexed forms of one or both of the proteins, as well as to accommodate automation of the assay. Binding of a test compound to a T2DM-1 or T2DM-2 protein, or interaction of a T2DM-1 or T2DM-2 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/T2DM fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or T2DM-1 or T2DM-2 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of T2DM binding or activity determined using standard techniques.

Other techniques for immobilizing either a T2DM-1 or T2DM-2 protein or a target molecule on matrices include using conjugation of biotin and streptavidin. Biotinylated T2DM-1 or T2DM-2 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with T2DM-1 or T2DM-2 protein or target molecules but which do not interfere with binding of the T2DM-1 or T2DM-2 protein to its target molecule. Such antibodies can be derivatized to the wells of the plate, and unbound target or T2DM-1 or T2DM-2 protein trapped in the wells by antibody conjugation. Methods for detecting such complexes, in addition to those described above for the GST-immobilized complexes, include immunodetection of complexes using antibodies reactive with the T2DM-1 or T2DM-2 protein or target molecule, as well as enzyme-linked assays which rely on detecting an enzymatic activity associated with the T2DM-1 or T2DM-2 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the T2DM-1 or T2DM-2 protein or biologically active portion thereof with a known compound which binds T2DM to form an assay mixture, contacting the assay mixture with a test compound, and determining the ability of the test compound to interact with a T2DM-1 or T2DM-2 protein, wherein determining the ability of the test compound to interact with a T2DM-1 or T2DM-2 protein includes determining the ability of the test compound to preferentially bind to T2DM-1 or T2DM-2 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the T2DM genes herein identified. In an alternative embodiment, the invention provides methods for determining the ability of the test compound to modulate the activity of a T2DM protein through modulation of the activity of a downstream effector of a T2DM target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the T2DM-1 or T2DM-2 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with T2DM-1 or T2DM-2 (“T2DM-binding proteins” or “T2DM-bp”) and are involved in T2DM-1 or T2DM-2 activity. Such T2DM-bps can be activators or inhibitors of signals by the T2DM-1 or T2DM-2 proteins or T2DM-1 or T2DM-2 targets as, for example, downstream elements of a T2DM-1 or T2DM-2-mediated signaling pathway.

The two-hybrid system is based on the modular nature of most transcription factors, which consist of separable DNA-binding and activation domains. Briefly, the assay utilizes two different DNA constructs. In one construct, the gene that codes for a T2DM-1 or T2DM-2 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: T2DM protein can be the fused to the activator domain.) If the “bait” and the “prey” proteins are able to interact, in vivo, forming a T2DM-1 or T2DM-2-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the T2DM protein.

In another embodiment, modulators of T2DM-1 or T2DM-2 expression are identified. For example, a cell or cell free mixture is contacted with a candidate compound and the expression of T2DM-1 or T2DM-2 mRNA or protein evaluated relative to the level of expression of T2DM-1 or T2DM-2 mRNA or protein in the absence of the candidate compound. When expression of T2DM-1 or T2DM-2 mRNA or protein is greater in the presence of the candidate compound than in its absence, the candidate compound is identified as a stimulator of T2DM-1 or T2DM-2 mRNA or protein expression. Alternatively, when expression of T2DM-1 or T2DM-2 mRNA or protein is less (statistically significantly less) in the presence of the candidate compound than in its absence, the candidate compound is identified as an inhibitor of T2DM-1 or T2DM-2 mRNA or protein expression. The level of T2DM-1 or T2DM-2 mRNA or protein expression can be determined by methods described herein for detecting T2DM mRNA or protein.

In another aspect, the invention pertains to a combination of two or more of the assays described herein. For example, a modulating agent can be identified using a cell-based or a cell free assay, and the ability of the agent to modulate the activity of a T2DM protein can be confirmed in vivo, e.g., in an animal such as an animal model for a β-cell or insulin related disorder, e.g., β-cell dysfunction, diabetes (e.g., insulin-dependent diabetes mellitus or non insulin-dependent diabetes mellitus) and its associated disorders, e.g., hypertension, retinopathy, persistent hyperinsulinemic hypoglycemia of infancy (PHHI), insulin resistance, hyperglycemia, glucose intolerance, glucotoxicity.

This invention further pertains to novel agents identified by the above-described screening assays. Accordingly, it is within the scope of this invention to further use an agent identified as described herein (e.g., a T2DM-1 or T2DM-2 modulating agent, an antisense T2DM-1 or T2DM-2 nucleic acid molecule, a T2DM-1 or T2DM-2-specific antibody, or a T2DM-1 or T2DM-2-binding partner) in an appropriate animal model to determine the efficacy, toxicity, side effects, or mechanism of action, of treatment with such an agent. Furthermore, novel agents identified by the above-described screening assays can be used for treatments as described herein.

### Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate T2DM with a disease; (ii) identify an individual from a minute biological sample (tissue typing); and (iii) aid in forensic identification of a biological sample. These applications are described in the subsections below.

### Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes T2DM-1 or T2DM-2. Such disorders include, e.g., a disorder associated with the misexpression of T2DM-1 or T2DM-2 gene; a disorder of the insulin metabolism or pancreatic tissue system, e.g., diabetes (e.g., insulin-dependent diabetes mellitus or non insulin-dependent diabetes mellitus) and its associated disorders, e.g., hypertension and retinopathy, persistent hyperinsulinemic hypoglycemia of infancy (PHHI), insulin resistance, hyperglycemia, glucose intolerance, glucotoxicity, or β-cell dysfunction.

Diagnostic and prognostic assays of the invention include methods for assessing the expression level of T2DM-1 or T2DM-2 molecules and, preferably, methods for identifying variations and mutations in the sequence of T2DM-1 or T2DM-2 molecules.

The presence, level, or absence of T2DM protein or nucleic acid in a biological sample can be evaluated by obtaining a biological sample from a test subject and contacting the biological sample with a compound or an agent capable of detecting T2DM-1 or T2DM-2 protein or nucleic acid (e.g., mRNA, genomic DNA) that encodes T2DM-1 or T2DM-2 protein such that the presence of T2DM-1 or T2DM-2 protein or nucleic acid is detected in the biological sample. The term “biological sample” includes tissues, cells and biological fluids isolated from a subject, as well as tissues, cells and fluids present within a subject. A preferred biological sample is serum. The level of expression of the T2DM-1 or T2DM-2 gene can be measured in a number of ways, including, but not limited to: measuring the mRNA encoded by the T2DM-1 or T2DM-2 genes; measuring the amount of protein encoded by the T2DM-1 or T2DM-2 genes; or measuring the activity of the protein encoded by the T2DM-1 or T2DM-2 genes.

The level of mRNA corresponding to the T2DM gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length T2DM nucleic acid, such as the nucleic acid of SEQ ID NO:1, 3, 5, or 6 or a portion thereof, such as an oligonucleotide of at least 7, 15, 30, 50, 100, 250 or 500 nucleotides in length and sufficient to specifically hybridize under stringent conditions to T2DM-1 or T2DM-2 mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the T2DM genes.

The level of mRNA in a sample that is encoded by one of the T2DM-1 or T2DM-2 genes can be evaluated with nucleic acid amplification, e.g., by RT-PCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes one of the T2DM-1 or T2DM-2 genes being analyzed.

In another embodiment, the methods further contacting a control sample with a compound or agent capable of detecting T2DM-1 or T2DM-2 mRNA, or genomic DNA, and comparing the presence of T2DM-1 or T2DM-2 mRNA or genomic DNA in the control sample with the presence of T2DM-1 or T2DM-2 mRNA or genomic DNA in the test sample. In still another embodiment, serial analysis of gene expression, as described in U.S. Pat. No. 5,695,937, is used to detect T2DM-1 or T2DM-2 transcript levels.

A variety of methods can be used to determine the level of protein encoded by T2DM-1 or T2DM-2. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect T2DM-1 or T2DM-2 protein in a biological sample in vitro as well as in vivo. In vitro techniques for detection of T2DM-1 or T2DM-2 protein include enzyme linked immunosorbent assays (ELISAs), immunoprecipitations, immunofluorescence, enzyme immunoassay (EIA), radioimmunoassay (RIA), and Western blot analysis. In vivo techniques for detection of T2DM-1 or T2DM-2 protein include introducing into a subject a labeled anti-T2DM-1 or T2DM-2 antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-T2DM-1 or T2DM-2 antibody positioned on an antibody array (as described below). The sample can be detected, e.g., with avidin coupled to a fluorescent label.

In another embodiment, the methods further include contacting the control sample with a compound or agent capable of detecting T2DM-1 or T2DM-2 protein, and comparing the presence of T2DM-1 or T2DM-2 protein in the control sample with the presence of T2DM-1 or T2DM-2 protein in the test sample.

The invention also includes kits for detecting the presence of T2DM-1 or T2DM-2 in a biological sample. For example, the kit can include a compound or agent capable of detecting T2DM-1 or T2DM-2 protein or mRNA in a biological sample; and a standard. The compound or agent can be packaged in a suitable container. The kit can further comprise instructions for using the kit to detect T2DM-1 or T2DM-2 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted T2DM-1 or T2DM-2 expression or activity. As used herein, the term “unwanted” includes an unwanted phenomenon involved in a biological response such as pancreatic tissue formation and maintenance.

In one embodiment, a disease or disorder associated with aberrant or unwanted T2DM expression or activity, e.g., type 2 diabetes mellitus, is identified. A test sample is obtained from a subject and T2DM-1 or T2DM-2 protein or nucleic acid (e.g., mRNA or genomic DNA) is evaluated, wherein the level, e.g., the presence or absence, of T2DM-1 or T2DM-2 protein or nucleic acid, or the genotype of T2DM-1 or T2DM-2, is diagnostic for a subject having or at risk of developing type 2 diabetes. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted T2DM-1 or T2DM-2 expression or activity.

In another aspect, the invention features a computer medium having a plurality of digitally encoded data records. Each data record includes a value representing the level of expression of T2DM-1 or T2DM-2 in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than T2DM-1 or T2DM-2 (e.g., other genes associated with a T2DM-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of T2DM-1 or T2DM-2 expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a β-cell or insulin related disorder, e.g., diabetes (e.g., insulin-dependent diabetes mellitus or non insulin-dependent diabetes mellitus) and its associated disorders, e.g., hypertension, retinopathy, persistent hyperinsulinemic hypoglycemia of infancy (PHHI), insulin resistance, hyperglycemia, glucose intolerance, glucotoxicity in a subject wherein an increase or decrease in T2DM expression is an indication that the subject has or is disposed to having a β-cell or insulin related disorder described herein. The method can be used to monitor a treatment for type 2 diabetes in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of T2DM-1 or T2DM-2 expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression or genotype profile for the sample. Optionally, the method further includes either or both of steps: c) comparing the subject expression or genotype profile to one or more reference expression or genotype profiles; and d) selecting the reference expression or genotype profile most similar to the subject expression or genotype profile. The subject reference profiles can include a value representing the level of T2DM-1 or T2DM-2 expression or T2DM-1 or T2DM-2 genotype. A variety of routine statistical measures can be used to compare two profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression or genotype profile, a result of a comparison of the subject expression or genotype profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression or genotype profile; access a database of reference expression or genotype profiles; and either i) select a matching reference profile most similar to the subject expression or genotype profile or ii) determine at least one comparison score for the similarity of the subject expression or genotype profile to at least one reference profile. The subject expression or genotype profile, and the reference expression or genotype profiles each include a value representing the level of T2DM-1 or T2DM-2 expression or an identifier for a T2DM-1 or T2DM-2 genotype.

### Arrays And Uses Thereof

In another aspect, the invention features an array that includes a substrate having a plurality of addresses. At least one address of the plurality includes a capture probe that binds specifically to a T2DM-1 or T2DM-2 molecule (e.g., a T2DM-1 or T2DM-2 nucleic acid or a T2DM-1 or T2DM-2 polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a T2DM-1 or T2DM-2 nucleic acid, e.g., the sense or anti-sense strand. The nucleic acid capture probe can hybridize specifically to a nucleic acid that represents a particular polymorphism, haplotype or genotype of T2DM-1 or T2DM-2. In one preferred embodiment, a subset of addresses of the plurality of addresses has a nucleic acid capture probe for a nucleic acid capture probe that hybridizes specifically to a T2DM-1 or T2DM-2 nucleic acid. Each address of the subset can include a capture probe that hybridizes to a different region of a T2DM-1 or T2DM-2 nucleic acid. In another preferred embodiment, addresses of the subset include a capture probe for a T2DM-1 or T2DM-2 nucleic acid. Each address of the subset is unique, overlapping, and complementary to a different variant of T2DM-1 or T2DM-2 (e.g., a SNP, an allelic variant, or all possible hypothetical variants). The array can be used to sequence T2DM-1 or T2DM-2 by hybridization (see, e.g., U.S. Pat. No. 5,695,940), or to genotype a subject's DNA.

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a T2DM-1 or T2DM-2 polypeptide or fragment thereof. The polypeptide can be a naturally-occurring interaction partner of T2DM-1 or T2DM-2 polypeptide. Preferably, the polypeptide is an antibody, e.g., an antibody described herein (see “Anti-T2DM Antibodies,” above), such as a monoclonal antibody or a single-chain antibody.

In another aspect, the invention features a method of analyzing the expression of T2DM-1 or T2DM-2. The method includes providing an array as described above; contacting the array with a sample and detecting binding of a T2DM-1 or T2DM-2-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of T2DM-1 or T2DM-2. If a sufficient number of diverse samples is analyzed, clustering (e.g., hierarchical clustering, k-means clustering, Bayesian clustering and the like) can be used to identify other genes which are co-regulated with T2DM-1 or T2DM-2. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on T2DM-1 or T2DM-2 expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of type 2 diabetes. The method can also evaluate the treatment and/or progression of type 2 diabetes.

The array is also useful for ascertaining differential expression patterns of one or more genes in normal and abnormal cells. This provides a battery of genes (e.g., including T2DM-1 or T2DM-2) that could serve as a molecular target for diagnosis or therapeutic intervention.

In another aspect, the invention features an array having a plurality of addresses. Each address of the plurality includes a unique polypeptide. At least one address of the plurality has disposed thereon a T2DM-1 or T2DM-2 polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80, 85, 90, 95 or 99% identical to a T2DM-1 or T2DM-2 polypeptide or fragment thereof. For example, multiple variants of a T2DM-1 or T2DM-2 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a T2DM-1 or T2DM-2 binding compound, e.g., an antibody in a sample from a subject with specificity for a T2DM-1 or T2DM-2 polypeptide or the presence of a T2DM-1 or T2DM-2-binding protein or ligand.

The array is also useful for ascertaining the effect of the expression of a gene on the expression of other genes in the same cell or in different cells (e.g., ascertaining the effect of T2DM-1 or T2DM-2 expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express T2DM-1 or T2DM-2 or from a cell or subject in which a T2DM-1 or T2DM-2 mediated response has been elicited, e.g., by contact of the cell with T2DM nucleic acid or protein, or administration to the cell or subject T2DM nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express T2DM-1 or T2DM-2 (or does not express as highly as in the case of the T2DM-1 or T2DM-2 positive plurality of capture probes) or from a cell or subject which in which a T2DM mediated response has not been elicited (or has been elicited to a lesser extent than in the first sample); contacting the array with one or more inquiry probes (which is preferably other than a T2DM nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express T2DM-1 or T2DM-2 or from a cell or subject in which a T2DM-1 or T2DM-2-mediated response has been elicited, e.g., by contact of the cell with T2DM-1 or T2DM-2 nucleic acid or protein, or administration to the cell or subject T2DM-1 or T2DM-2 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express T2DM-1 or T2DM-2 (or does not express as highly as in the case of the T2DM-1 or T2DM-2 positive plurality of capture probes) or from a cell or subject which in which a T2DM-1 or T2DM-2 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing T2DM-1 or T2DM-2, e.g., analyzing structure, function, or relatedness to other nucleic acid or amino acid sequences. The method includes: providing a T2DM-1 or T2DM-2 nucleic acid or amino acid sequence; comparing the T2DM-1 or T2DM-2 sequence with one or more preferably a plurality of sequences from a collection of sequences, e.g., a nucleic acid or protein sequence database; to thereby analyze T2DM-1 or T2DM-2.

### Use of T2DM-10R T2DM-2 Molecules as Surrogate Markers

The T2DM-1 or T2DM-2 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the T2DM-1 or T2DM-2 molecules of the invention may be detected, and may be correlated with one or more biological states in vivo. For example, the T2DM-1 or T2DM-2 molecules of the invention may serve as surrogate markers for type 2 diabetes. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The T2DM-1 or T2DM-2 molecules of the invention, e.g., the polymorphic T2DM-1 or T2DM-2 molecules are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a T2DM-1 or T2DM-2 marker) transcription or expression, the amplified marker may be in a quantity which is more readily detectable than the drug itself. Also, the marker may be more easily detected due to the nature of the marker itself; for example, using the methods described herein, anti-T2DM-1 or T2DM-2 antibodies may be employed in an immune-based detection system for a T2DM-1 or T2DM-2 protein marker, or T2DM-1 or T2DM-2-specific radiolabeled probes may be used to detect a T2DM-1 or T2DM-2 mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The T2DM-1 or T2DM-2 molecules of the invention are also useful as pharmacogenomic markers. As used herein, a “pharmacogenomic marker” is an objective biochemical marker that correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., T2DM-1 or T2DM-2 protein or RNA) for specific tumor markers in a subject, a drug or course of treatment may be selected that is optimized for the treatment of the specific tumor likely to be present in the subject. Similarly, the presence or absence of a specific sequence mutation in T2DM-1 or T2DM-2 DNA may correlate with a specific drug response. The use of pharmacogenomic markers therefore permits the application of the most appropriate treatment for each subject without having to administer the therapy.

### Pharmaceutical Compositions

The nucleic acid and polypeptides, fragments thereof, as well as anti-T2DM-1 or T2DM-2 antibodies (also referred to herein as “active compounds”) of the invention can be incorporated into pharmaceutical compositions for administration to a subject, e.g., a human, a non-human animal, e.g., an animal model for a pancreatic or insulin related disorder, e.g., a nod mouse, a Zucker rat, a fructose fed rodent, an Israeli sand rat. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

A number of embodiments of the invention have been described. Nevertheless, it will be understood that various modifications may be made without departing from the spirit and scope of the invention. Accordingly, other embodiments are within the scope of the following claims and the Summary (above).

All patents and references cited herein are hereby incorporated by reference in their entirety. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. An isolated nucleic acid molecule selected from the group consisting of:
a) a nucleic acid molecule comprising a nucleotide sequence which is at least 80% identical to the nucleotide sequence of SEQ ID NO:1, 3, 5 or 6 or a fragment thereof;
b) a nucleic acid molecule comprising a sequence that hybridizes under high stringency conditions to a nucleic acid sequence consisting of SEQ ID NO:1, 3, 5 or 6, or its complement thereof;
c) a nucleic acid molecule that encodes a polypeptide at least 80% identical to the polypeptide of SEQ ID NO: 2 or 4; and
d) a fragment of (a), (b) or (c) at least 20 nucleotides in length.

2. A vector comprising the nucleotide molecule of claim 1.

3. A host cell harboring the nucleic acid molecule of claim 1.

4. The host cell of claim 3, wherein the host cell is a mammalian cell.

5. The host cell of claim 3, wherein the host cell is a human cell.

6. An isolated nucleic acid comprising one of:
(i) at least 20 contiguous nucleotides of SEQ ID NO:10, or its complement, wherein the nucleic acid includes nucleotides 203 and 204 (CA) of SEQ ID NO:10, or the complement thereof;
(ii) at least 20 contiguous nucleotides of SEQ ID NO:12, or its complement, wherein the nucleic acid includes nucleotide 201 (G) of SEQ ID NO:12, or the complement thereof;
(iii) at least 20 contiguous nucleotides of SEQ ID NO:14, or its complement, wherein the nucleic acid includes nucleotide 201 (G) of SEQ ID NO:14, or the complement thereof;
(iv) at least 20 contiguous nucleotides of SEQ ID NO:16, or its complement, wherein the nucleic acid includes 201 (G) of SEQ ID NO:16, or the complement thereof;
(v) at least 20 contiguous nucleotides of SEQ ID NO:18, or its complement, wherein the nucleic acid includes nucleotide 201 (C) of SEQ ID NO:18, or the complement thereof.
(vi) at least 20 contiguous nucleotides of SEQ ID NO:20, or its complement, wherein the nucleic acid includes nucleotides 199 to 202 (GCCC) of SEQ ID NO:20, or the complement thereof;
(vii) at least 20 contiguous nucleotides of SEQ ID NO:22, or its complement, wherein the nucleic acid includes nucleotide 201 (G) of SEQ ID NO:22, or the complement thereof;
(viii) at least 20 contiguous nucleotides of SEQ ID NO:24, or its complement, wherein the nucleic acid includes nucleotide 201 (G) of SEQ ID NO:24, or the complement thereof;
(ix) at least 20 contiguous nucleotides of SEQ ID NO:26, or its complement, wherein the nucleic acid includes nucleotide 201 (C) of SEQ ID NO:26, or the complement thereof;
(x) at least 20 contiguous nucleotides of SEQ ID NO:28, or its complement, wherein the nucleic acid includes nucleotide 201 (T) of SEQ ID NO:28, or the complement thereof;
(xi) at least 20 contiguous nucleotides of SEQ ID NO:30, or its complement, wherein the nucleic acid includes nucleotide 201 (T) of SEQ ID NO:30, or the complement thereof;
(xii) at least 20 contiguous nucleotides of SEQ ID NO:32, or its complement, wherein the nucleic acid includes nucleotide 201 (A) of SEQ ID NO:32, or the complement thereof;
(xiii) at least 20 contiguous nucleotides of SEQ ID NO:34, or its complement, wherein the nucleic acid includes nucleotide 201 (C) of SEQ ID NO:34, or the complement thereof;
(xiv) at least 15 contiguous nucleotides of SEQ ID NO:36, or its complement, wherein the nucleic acid includes nucleotide 201 (T) of SEQ ID NO:36, or the complement thereof;

7. The nucleic acid of claim 6, wherein the nucleic comprises at least 50 contiguous nucleotides of SEQ ID NO:18, or its complement, wherein the nucleic acid includes nucleotide 201 (c) of SEQ ID NO:18, or the complement thereof.

8. The nucleic acid of claim 6, wherein the nucleic comprises at least 50 contiguous nucleotides of SEQ ID NO:20, or its complement, wherein the nucleic acid includes nucleotides 199 to 202 (GCCC) of SEQ ID NO:20, or the complement thereof.

9. A nucleic acid probe or primer comprising at least 15 contiguous nucleotides of SEQ ID NO:1, 3, 5 or 7.

10. An isolated polypeptide comprising a sequence at least 80% identical to the amino acid sequence of SEQ ID NO: 2 or 4, or a fragment thereof comprising at least 15 contiguous amino acids.

11. A fusion protein comprising the polypeptide of claim 10.

12. An antibody which selectively binds to the polypeptide of claim 10.

13. A method of producing a polypeptide, the method comprising culturing the host cell of claim 3 under conditions in which the nucleic acid molecule is expressed.

14. A method of determining if a subject is at risk for type 2 diabetes the method comprising evaluating the level, activity, expression and/or genotype of a T2DM-1 or T2DM-2 molecule in a subject, thereby determining if a subject is at risk for type 2 diabetes.

15. The method of claim 14, further comprising diagnosing a subject as being at risk for or having type 2 diabetes.

16. The method of claim 14, wherein the method comprises detecting, in a biological sample of the subject, the presence or absence of a mutation in a T2DM-1 or T2DM-2 gene.

17. The method of claim 14, wherein the method comprises detecting the presence or absence of a T2DM-1 or T2DM-2 polymorphism in the subject's T2DM-1 or T2DM-2 gene.

18. The method of claim 17, wherein the polymorphism is selected from a polymorphism shown in FIG. 4 and FIG. 10.

19. The method of claim 14, wherein the determining step comprises one or more of:
(i) amplifying at least a portion of a T2DM-1 or T2DM-2 nucleic acid molecule of the subject;
(ii) sequencing at least a portion of a T2DM-1 or T2DM-2 nucleic acid molecule of the subject; or
(iii) hybridizing a T2DM-1 or T2DM-2 nucleic acid molecule of the subject with a probe or primer described herein.

20. An array of nucleic acid molecules capable of detecting a T2DM-1 or T2DM-2 polymorphism described herein.

21. A set of oligonucleotides comprising a plurality of oligonucleotides, each of which is at least 70% complementary to a T2DM-1 or T2DM-2 nucleic acid.

22. A method of evaluating a subject, the method comprising:
providing a nucleic acid sample from the subject;
evaluating a genotype of the T2DM-1 or T2DM-2 gene of the subject; and
providing a determination of the subject's susceptibility to type 2 diabetes.

23. A method of identifying a T2DM-1 or T2DM-2 allele in a subject, the method comprising: identifying the presence or absence of two or more polymorphisms in the T2DM-1 or T2DM-2 gene of the subject

24. A method of treating a subject, the method comprising modulating the expression, level, or activity of a T2DM-1 or T2DM-2 molecule in the subject.

25. The method of claim 24, wherein the subject is identified as having or being at risk for type 2 diabetes an associated condition.

26. The method of claim 24, wherein T2DM-1 or T2DM-3 expression, level or activity is increased in the subject.

27. A method of screening for a compound that affects type 2 diabetes susceptibility, the method comprising:
providing a T2DM-1 or T2DM-2 protein or nucleic acid;
contacting the T2DM-1 or T2DM-2 protein or nucleic acid with a test compound, and
determining if the test compound modulates the T2DM-1 or T2DM-2 protein or nucleic acid.

28. The method of claim 27, wherein the method includes
(1) providing a genetically engineered cell, tissue, or subject, comprising a nucleic acid that encodes a reporter molecule functionally linked to a control region of a T2DM-1 or T2DM-2 gene;
(2) contacting the cell, tissue or subject with a test agent; and
(3) evaluating a signal produced by the reporter molecule, the presence or strength of which is correlated with the effect of the test agent on the T2DM-1 or T2DM-2 control region.

29. The method of claim 27, further comprising administering the test compound to an experimental animal.

30. A transgenic non-human mammal comprising a T2DM-1 or T2DM-2 transgene.
